(19) United States
(12) Patent Application Publication
(10) Pub. No.: US 2012/0135091 A1
(43) Pub. Date: May 31, 2012

(54) METHODS AND COMPOSITIONS FOR ENHANCING LIFESPAN INVOLVING SIRTUIN-MODULATING COMPOUNDS AND CHALCOGENIDES

(76) Inventors: Mark B. Roth, Seattle, WA (US); Dana L. Miller, Seattle, WA (US)

(21) Appl. No.: 12/016,886

(22) Filed: Jan. 18, 2008

Related U.S. Application Data
(60) Provisional application No. 60/885,619, filed on Jan. 18, 2007.
(60) Provisional application No. 60/991,717, filed on Dec. 1, 2007.

(51) Int. Cl. A61K 33/04; A61K 31/09; A61K 31/4402; A61P 7/04; A61K 31/58; A61K 31/65; A61K 31/519; A61K 33/06; A61K 31/055; A61K 31/505

(52) U.S. Cl. 424/696; 514/733; 514/720; 514/277; 514/256; 514/171; 514/154; 514/265.1; 424/706; 424/708; 424/702; 424/600

(57) ABSTRACT

The present invention concerns the use of active compounds, including chalcogenides and sirtuin-modulating compounds, either alone or in combination for increasing or enhancing survivability and/or longevity in biological matter. In general aspects, the chalcogenides and other active compounds may modulate one or more sirtuin proteins. It includes compositions, methods, articles of manufacture and apparatuses for enhancing survivability in any of these biological materials, so as to preserve and/or protect them. In specific embodiments, there are also therapeutic methods and apparatuses for aging or stress, diabetes, obesity, neurodegenerative diseases, cardiovascular disease, blood clotting disorders, inflammation, cancer, organ transplantation, hyperthermia, wound healing, hemorrhagic shock, cardioplegia for bypass surgery, neurodegeneration, hypothermia, and cancer using the active compounds described.

This application claims priority to U.S. Provisional Patent Application 60/885,619, filed Jan. 18, 2007, and U.S. Provisional Patent Application 60/991,717, filed on Dec. 1, 2007, both of which are hereby incorporated by reference in their entirety.

This invention was made with government support under Contract No. W81XWH-05-02-0035 awarded by the Department of Defense (DARPA) and grant no. R01 GM48435 awarded by the National Institutes of Health. The government has certain rights in the invention.

### BACKGROUND OF THE INVENTION

1. Field of the Invention

The present invention relates generally to compounds and methods that increase lifespan, enhance survivability and treat and protect cells and animals from injury, disease, and premature death. Such compounds include chalcogenides and sirtuin-modulating compounds. Methods include combinations of sirtuin modulating compounds and chalcogenides, including the co-administration of pharmaceutical compositions comprising sirtuin modulating compounds with pharmaceutical compositions comprising chalcogenides, as well as stable pharmaceutical compositions comprising both sirtuin modulating compounds and chalcogenides. Accordingly, in certain embodiments, the present invention is drawn to methods and compositions related to enhancing survivability and/or increasing longevity of biological matter via the modulation of one or more sirtuin proteins using one or more compounds alone or in combination.

2. Description of Related Art

Molecular genetic and pharmacological studies in a wide variety of animals have demonstrated that organismal lifespan is subject to control by genetic and environmental factors. For example, lifespan is significantly prolonged by reduced caloric intake (or caloric restriction—CR) in yeast, roundworms, fruit flies, rodents and primates. Also, single gene mutations or deletions can cause lifespan extension in yeast, roundworms fruit flies and rodents. Conversely, single gene mutations in mammals including humans causes accelerated aging (progeria) and decreased lifespan (see: Longo et al., Cell (2006), 126:257; Navarro et al, Human Molecular Genetics (2006) 2:R151-R161). One skilled in the art recognizes that the molecular genetic and pharmacological mechanisms that control aging and lifespan are highly conserved across millions of years of evolution, and that responses of lower organisms (e.g., C. elegans, D. melanogaster, S. cerevisae) to genetic and pharmacologic lifespan enhancing interventions are predictive of their effects in higher mammals including humans. A need exists in the art to identify pharmaceutical compositions that mimic CR or lifespan-extending genetic mutations to effect lifespan extension in mammals, preferably humans and companion or agricultural animals.

The Silent Information Regulator (SIR) family of genes is a highly conserved group of genes present in the genomes of organisms from archaebacteria to eukaryotes (Frye, 2000). SIR proteins are involved in diverse processes from regulation of gene silencing to DNA repair. The proteins encoded by members of the SIR gene family are highly conserved in a 250 amino acid core domain. Sirtuins have been the focus of intense interest since the discovery that Sir2, acts as a yeast longevity factor (Kaeberlein et al., 1999) and a similar gene, sir2.1, functions similarly to extend lifespan in C. elegans (Tissenbaum and Guarente, 2001; Guarente, 2005). Functioning as either deacetylases or ADP ribosylases, sirtuins are regulated by the cofactor NAD and may serve as sensors of the metabolic state of the cell and organism. In the budding yeast, increasing the activity of Sir2, a member of the conserved sirtuin family of NAD+-dependent deacetylases, increases replicative longevity. In yeast and C. elegans, lifespan is extended by extra copies of the SIR2 gene or by small molecule sirtuin agonists. In mammals, SIRT1 (the analog of Sir2) is an important regulator of cell defenses and cell survival in response to stress. Sirtuins also play a key role in an organism's response to stressors such as heat or starvation. For example, yeast cell starvation lead to extended lifespans, resulting in increases in Sir2 activity; removal of the sir2 gene eliminated the life-extending effect of calorie restriction (Guarente, 2005). Examples of sirtuin proteins in mammals include SIRT1, SIRT2, SIRT3, SIRT4, SIRT5, SIRT6 and SIRT7. Sirtuins and the modulation thereof have also been associated with protection from ischemia/reperfusion injury and chemoprotection.

It has been recently shown that chalcogenides and other active compounds enhance survivability in cells, tissues, and/or organs in vivo or in an organism overall, as well as induce stasis or pre-stasis. In these studies, chalcogenides and other active compounds were used in biological materials to preserve and/or protect them from hypoxic and ischemic injury (see, e.g., PCT Publication No. WO 2006/113914).

There is a need in the art for methods, compositions, articles of manufacture and apparatuses that enhance longevity and/or modulate sirtuin activity in biological matter. The present invention provides these and more to extend the lifespan of a cell or tissue, the lifespan of cells, tissues and organs located within or derived from an organism, as well as the organism itself.

### SUMMARY OF THE INVENTION

The present invention provides methods and compositions involving active compounds that may be used to increase lifespan, increase longevity and enhance survivability. In certain embodiments, active compounds modulate sirtuin activity. Active compounds comprise chalcogenides and sirtuin-modulating compounds, as described herein. The compounds and methods of the present invention may also be used in the treatment and prevention of disease, disorders, and conditions that benefit from treatment with active compounds. These methods and compositions may be utilized for a variety of purposes and may be administered to various biological matter, including cells, tissues, organs, organisms, and animals, including humans and other mammals in vivo or in vitro.

Accordingly, in one embodiment, the present invention provides a method of enhancing lifespan in biological matter, comprising administering chalcogenides to the biological matter. In certain embodiments, the biological matter consists of or comprises a cell.

In one embodiment, the present invention provides a method of enhancing lifespan in biological matter, comprising administering to the biological matter a sirtuin-modulating compound in combination with a chalcogenide. In certain embodiments, the biological matter consists of or comprises a cell.

In certain embodiments, the present invention provides methods and compositions for enhancing the lifespan in a mammal due to, for example, a disease or adverse medical condition. Such methods may comprise providing to the mammal an effective amount of a chalcogenide or a combination of a chalcogenide and a sirtuin-modulating compound.

In another embodiment, the present invention provides a method of reducing a cytotoxic effect of sulfide in biological matter, comprising administering to the biological matter a chalcogenide or a combination of a chalcogenide and a sirtuin-modulating compound.

In certain embodiments, a chalcogenide may be of any compound described herein, such as a compound of formula (I) or (IV) (described below). The chalcogenide may comprise sulfur. The chalcogenide may be sulfide. The chalcogenide may be a sulfide salt, such as sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), or barium sulfide (BaS). In particular embodiments, the chalcogenide may be H2S, H2Se, H₂Te or H₂Po.

A sirtuin-modulating compound may be that of any known in the art. In certain embodiments, the sirtuin-modulating compound is selected from the group consisting of any one of formula 1-188. In another embodiment, the sirtuin-modulating compound is selected from the group consisting of nicotinic acid, resveratrol, butein, fisetin, piceatannol, isoliquiritigenin and quercetin.

In certain embodiments, a sirtuin-modulating compound and a chalcogenide are administered as gases. In other embodiments, a sirtuin-modulating compound and a chalcogenide are administered as liquids. In one embodiment, the sirtuin-modulating compound is administered as a gas and the chalcogenide is administered as a liquid. In another embodiment, the sirtuin-modulating compound is administered as a liquid and the chalcogenide is administered as a gas. In another embodiment, the chalcogenide is administered as a solid oral dosage form and the sirtuin modulating compound is administered as a solid oral dosage form. In particular embodiments, a sirtuin-modulating compound and a chalcogenide are administered concurrently. In one embodiment, a chalcogenide is administered prior to administration of a sirtuin-modulating compound. In one embodiment, the sirtuin-modulating compound is administered prior to administration of a chalcogenide.

In one related embodiment, the present invention includes a method of treating or preventing a disease, disorder, or condition that benefits from treatment with sirtuin-modulating compounds comprising administering to a patient an effective amount of a chalcogenide or a combination of a chalcogenide and a sirtuin-modulating compound. In particular embodiments, the disease, disorder or condition is a respiratory, cardiovascular, neurological, pulmonary, or blood disease or disorder, a tumor, an infection, inflammation, shock, sepsis, or stroke, in a patient.

In particular embodiments, the disease, disorder, condition, or adverse medical condition is selected from the group consisting of: aging, progeria, stress, diabetes, obesity, neurodegenerative diseases, cardiovascular disease, blood clotting disorders, inflammation, cancer, hemorrhagic shock, myocardial infarction, acute coronary syndrome, cardiac arrest, neonatal hypoxia/ischemia, ischemic reperfusion injury, unstable angina, post-angioplasty, aneurysm, trauma, stroke, coronary artery bypass graft (CABG) surgery and blood loss.

In particular embodiments, the disease, disorder or medical condition is selected from the group consisting of: increasing radiosensitivity or chemosensitivity, increasing the amount of apoptosis, treatment of cancer, stimulation of appetite and stimulation of weight gain.

In particular embodiments, the disease, disorder or condition is selected from the group comprising: hemochromatosis, acquired iron overload, sickle-cell anemia, juvenile hemochromatosis, sickle cell disease, HIV, African siderosis, thalassemia, porphyria cutanea tarda, sideroblastic anemia, iron-deficiency anemia and anemia of chronic disease.

In one embodiment, a therapeutically effective amount of a sirtuin-modulating compound is administered in combination with an amount of a chalcogenide sufficient to reduce cytotoxicity or another undesirable side-effect associated with sirtuin-modulating compounds.

In some embodiments, methods involve identifying a patient in need of a sirtuin-modulating compound. In certain instances, this may be accomplished by recognizing that the patient needs the effect(s) of a sirtuin-modulating compound and/or a chalcogenide or by recognizing that the patient has symptoms or a disease/condition that can be addressed particularly by a sirtuin-modulating compound and/or a chalcogenide. Other embodiments involve testing the patient for an effect attributable to the chalcogenide, the sirtuin-modulating compound, or the combination of both, after administration to the patient.

Methods of reducing cellular damage in a mammal from surgery comprising providing to the mammal an effective amount of a chalcogenide or a combination of a chalcogenide and a sirtuin-modulating compound are also contemplated.

In certain embodiments, the present invention provides for a method of enhancing survivability of biological matter under hypoxic or ischemic conditions, the conditions caused by disease, injury, or a medical procedure, comprising providing to the biological matter a composition comprising a chalcogenide or a combination of a chalcogenide and a sirtuin-modulating compound.

In a further embodiment, the present invention provides a method of preventing or reducing injury to, or enhancing survivability of a biological material exposed to ischemic or hypoxic conditions, comprising contacting the biological material with an effective amount of a chalcogenide in combination with a sirtuin-modulating compound. For example, methods for preventing or reducing damage to biological matter under adverse conditions comprising administering to the biological matter an effective amount of formula (I) and/or formula (IV), or a salt or prodrug thereof, in combination with a sirtuin-modulating compound, wherein damage is prevented or reduced are contemplated. In one embodiment, the biological material is contacted with a therapeutically effective amount of a chalcogenide in combination with an amount of a sirtuin-modulating compound sufficient to reduce cytotoxicity or an undesirable side-effect associated with a chalcogenide. In one embodiment, the biological material is contacted with a chalcogenide and a sirtuin-modulating compound before being exposed to the ischemic or hypoxic conditions. In another embodiment, the biological material is contacted with a chalcogenide and a sirtuin-modulating compound during exposure to the ischemic or hypoxic conditions. In yet another embodiment, the biological material is contacted with a chalcogenide and a sirtuin-modulating compound after being exposed to the ischemic or hypoxic conditions.

In particular embodiments of methods of the present invention, the ischemic or hypoxic conditions result from an injury to the biological material, the onset or progression of a disease that adversely affects the biological material, or hemorrhaging of the biological material. In certain embodiments, the biological material is contacted with a chalcogenide and a sirtuin-modulating compound before the injury, before the onset or progression of the disease, or before hemorrhaging of the biological material. In one embodiment, the injury is from an external physical source.

In certain embodiment of methods of the present invention, the biological material is to be transplanted. In others, the biological material is at risk for reperfusion injury or hemorrhagic shock.

In particular embodiments of the present invention, a combination of sirtuin-modulating compounds and chalcogenides is administered at a therapeutically effective amount. In certain instances, the amount of either or both sirtuin-modulating compounds and chalcogenides present in a therapeutically effective amount of a combination is less than the amount of sirtuin-modulating compounds of chalcogenides that is therapeutically effective when administered alone. In other embodiments, the amount of either or both sirtuin-modulating compounds and chalcogenides is administered in an amount that is greater than the amount of sirtuin-modulating compounds or chalcogenides that may be safely administered alone.

In various embodiments of methods of the present invention, the sirtuin-modulating compound and chalcogenide are administrated to a patient or other biological matter, or biological matter is contacted by inhalation, e.g., through the use of a nebulizer, injection, catheterization, immersion, lavage, perfusion, topical application, absorption, adsorption, or oral administration. In particular embodiments of methods of the present invention, administering or contacting is performed intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularly, mucosally, intrapericardially, intraumbilically, intraocularally, orally, topically, locally, by inhalation, by injection, by infusion, by continuous infusion, by localized perfusion, via a catheter, or via a lavage. In certain methods, biological matter, such as a patient, may be provided with a chalcogenide, such as a compound of formula (I) or (IV), or a combination of a chalcogenide and a sirtuin-modulating compound for a period of about five minutes or less.

In one particular embodiment, the present invention provides a method for treating or preventing a cardiovascular disease or disorder in a patient in need thereof comprising administering a therapeutically effective amount of a gas or liquid composition comprising a sirtuin-modulating compound and a chalcogenide to a patient. In certain embodiments, the cardiovascular disease is myocardial or heart failure.

In another embodiment, the present invention includes a method for treating or preventing inflammatory disease or disorder in a patient in need thereof administration of a gas or liquid composition comprising a sirtuin-modulating compound and a chalcogenide composition to a patient.

In a further related embodiment, the present invention provides a method for treating or preventing a blood disorder in a patient in need thereof comprising administering a therapeutically effective amount of a gas or liquid composition comprising a sirtuin-modulating compound and a chalcogenide to a patient. In one embodiment, the blood disorder is sickle cell disease. In one embodiment, the blood disorder is thalassemia.

In another embodiment, the present invention provides for a method of modulating sirtuin activity in biological matter comprising providing the biological matter with a chalcogenide. The chalcogenide may be a compound of formula (I) or (IV) or salt thereof, described herein. Such methods may further comprise providing the biological matter with a sirtuin-modulating compound.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

It is contemplated that any embodiment discussed in this specification can be implemented with respect to any method, compound or composition of the invention, and vice versa. Furthermore, compounds and compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. C. elegans exposed to a low concentration of H2S can survive subsequent exposure to high concentrations of H2S. Each bar represents one experimental condition, with time spent in room air indicated by open areas and filled-in sections representing time spent in a low concentration of H2S (50 ppm). The percentage of animals that survive in the high concentration of H2S (150 ppm) is noted next to each bar. The gray bar at the bottom indicates approximate time of development through larval stages (L1-L4).

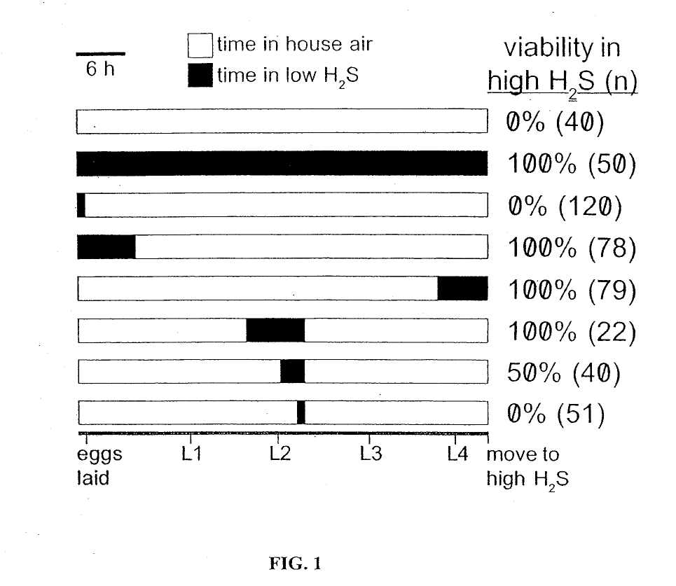

FIGS. 2A-C. FIG. 2A: Animals exposed to H2S survive longer than untreated controls at high temperature. In this panel, nematodes were moved to 35 C in the same gaseous atmosphere in which they had been cultured (diagrammed in FIG. 9). The mean survival time of animals grown in H2S was 65.5 h (solid line; n=136), compared to 9.1 h (n=96) for untreated controls (dashed line). FIG. 2B: Prior exposure to H2S is required to survive high temperature in H2S. In this panel, all animals were grown in room air without H2S and then moved to 35 C in the presence or absence of 50 ppm H2S. Animals first exposed to H2S at high temperature had a mean survival time of 2.1 h (n=20; solid line), whereas the control group exposed in room air survived for 7.3 h (dashed line; n=20). FIG. 2C: The continuous presence of H2S in the atmosphere is required for increased survival at high temperature. In this panel, all animals were exposed to 35 C in room air. Animals grown in H2S before heat-shock survived 7.3 h (solid line; n=20), which is not significantly longer than untreated controls (dashed line; 7.0 h, n=20). Indicated p-values were determined by log-rank analysis.

FIGS. 3A-C. FIG. 3A: Animals grown in H2S live longer than untreated controls. In this panel, the lifespan of animals was monitored in the same conditions in which they had developed. The mean lifespan of animals in H2S was 22.6±1.0 days (solid line; n=80), compared to 13.0±1.0 days for untreated controls in room air (dashed line; n=40). Maximum lifespan was also increased. FIG. 3B: Exposure to H2S beginning as L4 does not increase lifespan. In this panel, all animals were from populations grown in room air. The lifespan of animals moved into H2S-containing environments at the beginning of the lifespan experiment (solid line) is 14.8±0.3 days (n=73), which is slightly shorter than controls that remained in house air (dashed line; mean lifespan 18.2±0.4 days, n=48). FIG. 3C: Increased lifespan requires continuous exposure to H2S. In this panel, the lifespan of all animals was monitored in room air. The lifespan of animals raised in H2S until L4 (solid line; 12.8±0.7 days, n=52) was indistinguishable from untreated controls (dashed line; 13.2±0.7 days, n=59). All lifespan experiments were performed at room temperature.

FIGS. 4A-B. sir-2.1 is required for increased thermotolerance and lifespan in H2S. FIG. 4A: H2S does not increase thermotolerance of animals that have a deletion in sir-2.1. The mean survival time of sir-2.1(ok434) animals grown in H2S and exposed to high temperature in H2S (solid line) is 9.8±0.3 h (n=20), which is not significantly longer than untreated controls in room air (dashed line; mean survival 9.6±0.3 h, n=20). B. H2S does not increase the lifespan of sir-2.1(ok434) animals. The lifespan of sir-2.1(ok434) animals raised in H2S is 20.0±1.6 days (solid line; n=47), statistically indistinguishable from control animals in room air (dashed line; 22.2±1.2 days, n=26). Indicated p-values were determined by log-rank analysis.

FIG. 5. The rate of body core temperature drop is dependent upon the concentration of hydrogen sulfide given to the mice. All lines represent core body temperature of a single mouse as determined by radiotelemetry. Mice subjected to 20 ppm and 40 ppm H2S exhibit minor drops in core temperature. Exposure to 60 ppm induced a substantial drop in temperature beginning at approximately hour 4:00. The mouse exposed to 80 ppm exhibited a substantial drop in temperature beginning at approximately hour 2:00.

FIG. 6. A Kaplan Meier graph comparing the survival rate measured over time of C57BL/6 mice exposed to hypoxia (4% O₂) that were either infused with vehicle or treated with test article.

FIG. 7. Survival of mice in 5% oxygen. Mice were exposed to either 30 minutes of room air before exposure to 5% O₂ (control; black line; n=9) or 10 minutes of room air followed by 20 minutes of 150 ppm H2S before exposure to 5% O₂ (experimental; red line; n=20) and their length of survival measured. Experiments were stopped at 60 minutes and if the animals were still alive (all of the experimental, none of the controls) they were returned to their cage.

FIGS. 8A-C. Thermotolerance of canonical long-lived mutants is increased by H2S. Just as H2S increases thermotolerance of wild-type worms (FIGS. 2 and 10), long-lived mutants in canonical pathways that influence lifespan (Rea et al., 2005) are also more thermotolerant when grown in H2S. In all panels, animals grown in H2S were challenged with high temperature in H2S (solid line), whereas the thermotolerance of untreated controls was assayed in room air (dashed line). FIG. 8A: H2S effects are genetically independent of insulin/IGF signaling (IIS). The thermotolerance of daf-2(e1370) animals can be enhanced by exposure to H2S, and daf-16(m26) mutants, which are defective in IIS, become thermotolerant when grown in H2S. Note that to facilitate the experiments shown in this figure, strains which show intrinsic thermotolerance, such as daf-2(e1370) (Gems et al., 1998), were tested at a slightly higher temperature both in room air and H2S. However, the thermotolerance at 35 C is also increased when the animals are grown in H2S (not shown). FIG. 8B: H2S-induced thermotolerance is observed in isp-1(gk267) and clk-1(qm30) animals that are long-lived as a result of mitochondrial dysfunction. FIG. 8C: H2S-induced thermotolerance is observed in eat-2(ad1116) mutant animals, which have defects in pharangeal pumping that result in dietary restriction.

FIGS. 9A-B. Experimental system to produce H2S-containing atmospheres. FIG. 9A: Schematic of experimental system. As described in Example 7 below, H2S was continuously mixed into room air from a 5000 ppm source tank (red) using mass flow controllers (MFC). Atmospheric chambers (boxed) were continuously perfused with freshly-mixed 50 ppm H2S in room air distributed by flow tubes (yellow) and then hydrated by bubbling through a gas wash bottle (blue). The entire apparatus was in a fume hood, so that H2S exhaust could flow freely from the atmospheric chambers. FIG. 9B: 50 ppm H2S in room air is stable over the course of the experiment. To monitor the rate of H2S oxidation in room air, samples of the H2S-containing room air atmosphere were collected in gas sampling bags from the exhaust of the large atmospheric chamber. The bags were incubated at room temperature for various times before the concentration of H2S remaining was determined. At each measurement, a bag was filled with the H2S containing room air and the concentration of H2S was immediately determined (the 0 h measurements). No decrease in the concentration of H2S was observed over several hours, and in fact a decrease of less than 15 ppm after 72 h could be detected. The gaseous environment of the atmospheric chambers was replaced by freshly-mixed H2S in room air every 20-30 minutes at the flow rates used.

FIGS. 10A-B. Stress response genes are not induced by H2S FIG. 10A: The hsp-16.2::GFP transgene is induced by many environmental stresses (Krauth-Siegel, et al., 1989; Behnke et al., 2006; Lowicka and Beltowski, 2007; Stipanuk, 2004), but is not induced in animals grown in H2S (top row), as the level of GFP expression is indistinguishable from untreated controls (middle row). This transgene is strongly induced by heat, especially in the vulva and pharynx (bottom row). Each image of fluorescence is matched with a corresponding Nomarsky image to the right. The left set of images shows vulval expression, and the right set shows expression in the pharynx. FIG. 10B: The hsp-4::GFP transgene, a marker for ER stress (Kapulkin et al., 2005; Drano et al., 2002), is expressed at similar levels in larvae grown in H2S and untreated controls. The left set of images shows expression of hypodermal seam cells, and the right set shows expression in the anterior intestine. For all experiments, fluorescence was visualized for treated and untreated animals on the same day with the same camera settings. Also, H2S did not alter the level of GFP expression for animals carrying hsp-3::GFP, hsp-70::GFP, hsp-6::GFP, stc-1::GFP or sod-3::GFP transgenes (not shown).

FIGS. 11A-B. Robust effect of H2S on thermotolerance. FIG. 11A: The survival of animals grown in H2S at high temperature is variable, but repeatedly longer than untreated controls. Each point represents the fraction of H2S-treated animals that remained alive when the last untreated control animal had expired in one independent experiment (data from 15 experiments is shown). The conditions of each experiment varied slightly (for example, the age of the nematodes ranged from L4 to 3^{rd} day adults and hot temperatures from 33 to 37 C) but were always the same for H2S treated animals and controls. The ends of the box define the 25th and 75th percentile, the whiskers indicate the 10th and 90th percentiles and the line is the median (the mean was 0.8). In six experiments, all of the animals grown in H2S were still alive when all of the untreated animals had died (fraction alive=1). FIG. 11B: Gompertz analysis suggests that H2S delays the initiation of aging rather than decreasing the rate of aging. According to the Gompertz model, the rate of mortality increases exponentially with chronological age, such that μ(x)=Ae^{Gx} (Gompertz, 1825; for examples see Johnson, 1990 and Khazaeli et al., 1998) where μ(x) is the probability of death at a given time, A is the initial mortality hazard and G is the rate of the exponential increase in mortality. When plotted on a semi-log scale, this function is a line with slope G and intercept A. After fitting lines to the lifespan data plotted in this manner (hazard=probability of death/# animals remaining alive), it was observed that the slope of the lines fit to the data from H2S-treated animals (0.17±0.03, n=75) was quite similar to untreated controls (0.16±0.01, n=99), although the intercept was different (−5.83±0.8 for H2S, −3.52±0.22 in room air). Data from two independent lifespan experiments were combined for this analysis.

FIGS. 12A-B. H2S alters activity, rather than expression, of SIR-2.1. FIG. 12A: Quantitative RT-PCR indicates that the level of sir-2.1 transcripts is H2S-treated animals is not distinguishable from untreated controls. Primers to two different sites of the sir-2.1 transcript (primer set 1 and 2) were used to amplify cDNA from animals grown in 50 ppm H2S (4 independent samples) or room air (5 independent samples). Each reaction was performed in replicate. The average threshold cycle (Cₜ) was not different in the two conditions. Error bars show the standard deviation of the average value from all samples and replicates. FIG. 12B: H2S increases the thermotolerance of geIn3 animals (strain LG100) that overexpress sir-2.1. Animals that overexpress sir-2.1 were cultured in 50 ppm H2S. These animals survived at high temperature longer than untreated controls, similar to wild-type nematodes (FIG. 2) but distinct from sir-2.1(ok434) mutant animals (FIG. 4).

### DETAILED DESCRIPTION OF THE INVENTION

The present inventors have surprisingly discovered an interrelationship between increased survivability and enhanced lifespan (longevity), using chalcogenide compounds. For example, chalcogenide compounds were shown to enhance survivability in vivo and to enhance or extend lifespan in biological matter in a sirtuin-dependent manner. In one embodiment, the chalcogenide is sulfide and increases lifespan. Accordingly, in certain embodiments, the present invention contemplates compounds and methods that enhance survivability and increase lifespan (longevity). Without wanting to be limited to a particular mechanism or mode of action, in one embodiment the present invention contemplates compounds and methods that enhance survivability and extend or increase lifespan (longevity) in either a sirtuin-dependent manner or by sirtuin modulation.

In some embodiments, compositions of the present invention increase or enhance longevity of biological matter by modulation of sirtuin activity. In some embodiments, compositions of the present invention increase or enhance thermotolerance of biological matter via modulation of sirtuin activity. Such sirtuin modulation may further optionally increase survivability and/or enhance longevity.

Accordingly, any one or more active compounds and methods of the present invention may serve to enhance survivability and increase longevity and/or increase thermotolerance. All permutations and combinations are envisioned: administration of one or more active compounds of the present invention for any one or more of these or other effects as described herein is specifically envisioned.

As used herein, the terms “enhance lifespan,” “enhance longevity,” “increase lifespan,” “increase longevity,” “extend longevity”, “enhance survivability” “increase survivability,” “extend lifespan,” “lifespan extension” and variants thereof are equivalent unless otherwise noted.

### I. COMPOUNDS OF THE PRESENT INVENTION

### Active Compounds

The invention is based, in part, on studies with compounds that were determined to have a protective function, and thus, serve as protective agents via modulation of one or more sirtuin proteins. Compounds, proteins and other agents (e.g., genes) that modulate sirtuin activity (“sirtuin modulators,” as used herein) are known in the art. Sirtuin modulators refer to agents that may either up regulate (e.g., activate or stimulate), down regulate (e.g., inhibit or suppress) or otherwise change a functional property or biological activity of a sirtuin protein. In certain embodiments, a sirtuin-modulator may be a sirtuin-activating compound or a sirtuin-inhibiting compound. Sirtuin-modulators may act to modulate a sirtuin protein either directly (by interacting with or contacting a sirtuin protein directly) or indirectly.

“Active compounds,” as used herein, may refer to “chalcogenide compounds” and “sirtuin-modulating compounds” as exemplified by formulas (I) and (IV) and formulas I-188, respectively. In one embodiment, an “active compound” refers to modulation of one or more sirtuin proteins using a chalcogenide compound of the present invention. In another embodiment, an “active compound” refers to modulation of one or more sirtuin proteins using a combination of a chalcogenide compound of the present invention and a sirtuin-modulating compound, described herein. Carbon monoxide (CO) may be an active compound of the present invention. Active compounds also include, in some embodiments, methanol (CH3OH) and/or ethanol (CH3CH2OH).

“Chalcogenide compounds,” as used herein, refer to compounds satisfying any one of formulas (I) or (IV). Chalcogenide compounds may modulate sirtuin activity, though not necessarily.

“Sirtuin-modulating compounds” refer to compounds that preferably modulate sirtuin activity. Exemplary sirtuin-modulating compounds satisfy any one of formulas I-188, and comprise “sirtuin-activating compounds” and sirtuin-inhibiting compounds.”

It is specifically contemplated that any subset of chalcogenide compounds or sirtuin modulating compounds identified by name or structure may be used in methods, compositions and articles of manufacture of the present invention. It is also specifically contemplated that any subset of these compounds may be disclaimed as not constituting embodiments of the invention.

As used herein, “sirtuin-activating compound” refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. A sirtuin-activating compound may be an active compound. In other embodiments, the invention provides methods for using sirtuin-modulating compounds wherein the sirtuin-modulating compounds increase sirtuin activity, e.g., increase the level and/or activity of a sirtuin protein.

As used herein, “sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. A sirtuin-inhibiting compound may be an active compound. In other embodiments, the invention provides methods for using sirtuin-modulating compounds wherein the sirtuin-modulating compounds decrease sirtuin activity, e.g., decrease the level and/or activity of a sirtuin protein.

Active compounds as described herein may contain one or more asymmetric centers and thus can occur as racemates and racemic mixtures, single enantiomers, diastereomeric mixtures and individual diasteromers. All possible stereoisomers of the all the active compounds described herein, unless otherwise noted, are contemplated as being within the scope of the present invention. The chiral centers of the compounds of the present invention can have the S- or the R-configuration, as defined by the IUPAC 1974 Recommendations. The present invention is meant to comprehend all such isomeric forms of active compounds.

A variety of chemical structures and compounds are described herein. The following definitions apply to terms used to described these structures and compounds discussed herein, unless otherwise noted:

“Alkyl,” where used, either alone or within other terms such as “arylalkyl”, “aminoalkyl”, “thioalkyl”, “cyanoalkyl” and “hydroxyalkyl”, refers to linear or branched radicals having one to about twenty carbon atoms. The term “lower alkyl” refers to C₁-C₆ alkyl radicals. As used herein the term alkyl includes those radicals that are substituted with groups such as hydroxy, halo (such as F, Cl, Br, I), haloalkyl, alkoxy, haloalkoxy, alkylthio, cyano, isocyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—R—SO₃Na). Examples of such radicals include, but are not limited to, methyl, ethyl, n-propyl, isopropyl, n-butyl, isobutyl, sec-butyl, tert-butyl, pentyl, iso-amyl, hexyl and the like.

“Hydroxyalkyl” refers to an alkyl radical, as defined herein, substituted with one or more hydroxyl radicals. Examples of hydroxyalkyl radicals include, but are not limited to, hydroxymethyl, 2-hydroxyethyl, 2-hydroxypropyl, 3-hydroxypropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl, 2-(hydroxymethyl)-3-hydroxypropyl, and the like.

“Arylalkyl” refers to the radical R′R— wherein an alkyl radical, “R” is substituted with an aryl radical “R′.” Examples of arylalkyl radicals include, but are not limited to, benzyl, phenylethyl, 3-phenylpropyl, and the like.

“Aminoalkyl” refers to the radical H₂NR′—, wherein an alkyl radical is substituted with am amino radical. Examples of such radicals include aminomethyl, amino ethyl, and the like. “Alkylaminoalkyl” refers to an alkyl radical substituted with an alkylamino radical.

“Alkylsulfonamido” refers to a sulfonamido group (—S(O)₂—NRR′) appended to an alkyl group, as defined herein.

“Thioalkyl” refers to wherein an alkyl radical is substituted with one or more thiol radicals. “Alkylthioalkyl” refers to wherein an alkyl radical is substituted with one or more alkylthio radicals. Examples include, but are not limited to, methylthiomethyl, ethylthioisopropyl, and the like. “Arylthioalkyl” refers to wherein an alkyl radical, as herein defined, is substituted with one or more arylthio radicals.

“Carboxyalkyl” refers to the radicals —RCO₂H, wherein an alkyl radical is substituted with a carboxyl radical. Example include, but are not limited to, carboxymethyl, carboxyethyl, carboxypropyl, and the like.

“Alkylene” refers to bridging alkyl radicals.

The term “alkenyl” refers to an unsaturated, acyclic hydrocarbon radical in so much as it contains at least one double bond. Such alkenyl radicals contain from about 2 to about 20 carbon atoms. The term “lower alkenyl” refers to C₁-C₆ alkenyl radicals. As used herein, the term alkenyl radicals includes those radicals substituted as for alkyl radicals. Examples of suitable alkenyl radicals include propenyl, 2-chloropropenyl, buten-1-yl, isobutenyl, pent-1-en-1-yl, 2-2-methyl-1-buten-1-yl, 3-methyl-1-buten-1-yl, hex-2-en-1-yl, 3-hydroxyhex-1-en-1-yl, hept-1-en-1-yl, and oct-1-en-1-yl, and the like.

The term “alkynyl” refers to an unsaturated, acyclic hydrocarbon radical in so much as it contains one or more triple bonds, such radicals containing about 2 to about 20 carbon atoms. The term “lower alkynyl” refers to C₁-C₆ alkynyl radicals. As used herein, the term alkynyl radicals includes those radicals substituted as for alkyl radicals. Examples of suitable alkynyl radicals include ethynyl, propynyl, hydroxypropynyl, but-1-yn-1-yl, but-1-yn-2-yl, pent-1-yn-1-yl, pent-1-yn-2-yl, 4-methoxypent-1-yn-2-yl, 3-methylbut-1-yn-1-yl, hex-1-yn-1-yl, hex-1-yn-2-yl, hex-1-yn-3-yl, 3,3-dimethyl-1-butyn-1-yl radicals and the like

“Alkoxy,” refers to the radical R′O—, wherein R′ is an alkyl radical as defined herein. Examples include, but are not limited to, methoxy, ethoxy, propoxy, butoxy, isopropoxy, tert-butoxy alkyls, and the like. Alkoxyalkyl” refers to alkyl radicals substituted by one or more alkoxy radicals. Examples include, but are not limited to, methoxymethyl, ethoxyethyl, methoxyethyl, isopropoxyethyl, and the like.

“Alkoxycarbonyl” refers to the radical R—O—C(O)—, wherein R is an alkyl radical as defined herein. Examples of alkoxycarbonyl radicals include, but are not limted to, methoxycarbonyl, ethoxycarbonyl, sec-butoxycarbonyl, isoprpoxycarbonyl, and the like. Alkoxythiocarbonyl refers to R—O—C(S)—.

“Aryl” refers to the monovalent aromatic carbocyclic radical consisting of one individual ring, or one or more fused rings in which at least one ring is aromatic in nature, which can optionally be substituted with one or more, preferably one or two, substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), unless otherwise indicated. Alternatively two adjacent atoms of the aryl ring may be substituted with a methylenedioxy or ethylenedioxy group. Examples of aryl radicals include, but are not limited to, phenyl, naphthyl, biphenyl, indanyl, anthraquinolyl, tert-butyl-phenyl, 1,3-benzodioxolyl, and the like.

“Arylsulfonamido” refers to a sulfonamido group, as defined herein, appended to an aryl group, as defined herein.

“Thioaryl” refers to an aryl group substituted with one or more thiol radicals.

“Alkylamino” refers to amino groups that are substituted with one or two alkyl radicals. Examples include monosubstituted N-alkylamino radicals and N,N-dialkylamino radicals. Examples include N-methylamino, N-ethylamino, N,N-dimeythylamino N,N-diethylamino, N-methyl, N-ethyl-amino, and the like.

“Aminocarbonyl” refers to the radical H₂NCO—. “Aminocarbonylalkyl” refers to the substitution of an alkyl radical, as herein defined, by one or more aminocarbonyl radicals.

“Amidyl” refers to RCO—NH—, wherein R is a H or alkyl, aryl, or heteroaryl, as defined herein.

“Imino carbonyl” refers to a carbon radical having two of the four covalent bond sites shared with an imino group. Examples of such imino carbonyl radicals include, for example, —C═NH, —C═NCH₃, —C═NOH, and —C═NOCH₃. The term “alkylimino carbonyl” refers to an imino radical substituted with an alkyl group, The term “amidino” refers to a substituted or unsubstituted amino group bonded to one of two available bonds of an iminocarbonyl radical. Examples of such amidino radicals include, for example, —NH₂—C═NH, NH₂—C═NCH₃, —NH—C═NOCH₃ and —NH(CH₃) —C═NOH. The term “guanidino” refers to an amidino group bonded to an amino group as defined above where the amino group can be bonded to a third group. Examples of such guanidino radicals include, for example, NH₂—C(NH) —NH—, NH₂—C(NCH₃)—NH—, NH₂—C(NOCH₃)—NH—, and CH₃NH—C(NOH)—NH—. The term “hydrazino” refers to —NH—NRR′, where R and R′ are independently hydrogen, alkyl and the like. “Hydrazide” refers to —C(═O)—NH—NRR′.

The term “heterocyclyl” refers to saturated and partially saturated heteroatom-containing ring-shaped radicals having from 4 through 15 ring members, herein referred to as “C₄-C₁₅ heterocyclyl” selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals include saturated 3 to 6-membered heteromonocyclic group containing 1 to 4 nitrogen atoms [e.g., pyrrolidinyl, imidazolidinyl, piperidino, piperazinyl, etc.]; saturated 3 to 6-membered heteromonocyclic group containing 1 to 2 oxygen atoms and 1 to 3 nitrogen atoms [e.g., morpholinyl, etc.]; saturated 3 to 6-membered heteromonocyclic group containing 1 to 2 sulfur atoms and 1 to 3 nitrogen atoms [e.g., thiazolidinyl, etc.]. Examples of partially saturated heterocyclyl radicals include dihydrothiophene, dihydropyran, dihydrofuran and dihydrothiazole. Non-limiting examples of heterocyclic radicals include 2-pyrrolinyl, 3-pyrrolinyl, pyrrolidinyl, 1,3-dioxolanyl, 2H-pyranyl, 4H-pyranyl, piperidinyl, 1,4-dioxanyl, morpholinyl, 1,4-dithianyl, thiomorpholinyl, and the like. Such heterocyclyl groups may be optionally substituted with groups such as substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—RSO₃Na).

“Hetroaryl” refers to monovalent aromatic cyclic radicals having one or more rings, preferably one to three rings, of four to eight atoms per ring, incorporating one or more heteroatoms, preferably one or two, within the ring (chosen from nitrogen, oxygen, or sulfur), which can optionally be substituted with one or more, preferably one or two substituents selected from substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidono, hydrazino, hydrazide, sodium sulfonyl (—SO₃Na), sodium sulfonylalkyl (—RSO₃Na), unless otherwise indicated. Examples of heteroaryl radicals include, but are not limited to, imidazolyl, oxazolyl, thiazolyl, pyrazinyl, thienyl, furanyl, pyridinyl, quinolinyl, isoquinolinyl, benzofuryl, benzothiophenyl, benzothiopyranyl, benzimidazolyl, benzoxazolyl, benzothiazolyl, benzopyranyl, indazolyl, indolyl, isoindolyl, quinolinyl, isoquinolinyl, naphthyridinyl, benezenesulfonyl-thiophenyl, and the like.

“Heteroaryloxy” refers to heteroaryl radicals attached to an oxy radical. Examples of such radicals include, but are not limited to, 2-thiophenyloxy, 2-pyrimidyloxy, 2-pyridyloxy, 3-pyridyloxy, 4-pyridyloxy, and the like

“Heteroaryloxyalkyl” refers to alkyl radicals substituted with one or more heteroaryloxy radicals. Examples of such radicals include 2-pyridyloxymethyl, 3-pyridyloxyethyl, 4-pyridyloxymethyl, and the like.

“Cycloalkyl” refers to monovalent saturated carbocyclic radicals consisting of one or more rings, typically one or two rings, of three to eight carbons per ring, which can typically be substituted with one or more, substitutents hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alykamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), unless otherwise indicated. Examples of cycloalkyl radicals include, but are not limited to, cyclopropyl, cyclobutyl, 3-ethylcyclobutyl, cyclopentyl, cycloheptyl, and the like. “Cycloalkenyl” refers to radicals having three to ten carbon atoms and one or more carbon-carbon double bonds. Typical cycloalkenyl radicals have three to seven carbon atoms. Examples include cyclobutenyl, cyclopentenyl, cyclohexenyl, cycloheptenyl, and the like. “Cycloalkenylalkyl” refers to radicals wherein an alkyl radical, as defined herein, is substituted by one or more cycloalkenyl radicals.

“Cylcoalkoxy” refers to cycloalkyl radicals attached to an oxy radical. Examples include, but are not limited to, cyclohexoxy, cyclopentoxy, and the like.

“Cylcoalkoxyalkyl” refers to alkyl radicals substituted one or more cycloalkoxy radicals. Examples include cyclohexoxyethyl, cyclopentoxymethyl, and the like.

“Sulfinyl” refers to —S(O)—.

“Sulfonyl” refers to —S(O)₂—, wherein “alkylsulfonyl” refers to a sulfonyl radical substituted with an alkyl radical, RSO₂—, arylsulfonyl refers to aryl radicals attached to a sulfonyl radical. “Sulfonamido” refers to —S(O)₂—NRR′.

“Sulfonic acid” refers to —S(O)₂OH. “Sulfonic ester” refers to —S(O)₂OR, wherein R is a group such as an alkyl as in sulfonic alkyl ester.

“Thio” refers to —S—. “Alkylthio” refers to RS— wherein a thiol radical is substituted with an alkyl radical R. Examples include methylthio, ethylthio, butylthio, and the like. “Arylthio” refers to R′S—, wherein a thio radical is substituted with an aryl radical, as herein defined. “Examples include, but are not limited to, phenylthio, and the like. Examples include, but are not limited to, phenylthiomethyl and the like. “Alkylthiosulfonic acid” refers to the radical HO₃SR′S—, wherein an alkylthioradical is substituted with a sulfonic acid radical.

“Thiosulfenyl” refers to —S—SH.

“Acyl”, alone or in combination, refers to a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The term “acylthiol” and “acyldisulfide” refers to the radicals RCOS— and RCOSS— respectively.

The term “thiocarbonyl” refers to the compounds and moieties which contain a carbon connected with a double bond to a sulfur atom —C(═S)—. “Alkylthiocarbonyl” refers to wherein a thiocarbonyl group is substituted with an alkyl radical, R. as defined herein, to form the monovalent radical RC(═S)—. “Aminothiocarbonyl” refers to a thiocarbonyl group substituted with an amino group, NH₂C(═S)—.

“Carbonyloxy” refers to —OCOR.

“Alkoxycarbonyl” refers to —COOR.

“Carboxyl” refers to —COOH.

The claimed invention is also intended to encompass salts of any of active compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps.

Hydrates of each of the active compounds are also contemplated, such as monohydrates, dihydrates and hemihydrates.

Also included in the methods presented herein are prodrugs of the active compounds. “Pro-drugs” generally refers to any compound that releases an active parent drug in vivo when such prodrug is administered.

The methods comprise, for example, administering to a subject in need thereof an effective amount of a sirtuin-modulating compound, e.g., a sirtuin-activating compound.

In certain embodiments, the invention provides methods for using active compounds of the present invention wherein the compounds activate a sirtuin protein, e.g., increase the level and/or activity of a sirtuin protein. Active compounds of the present invention that increase the level and/or activity of a sirtuin protein may be useful for a variety of therapeutic applications including, for example, increasing the lifespan of a cell, tissue, organ or organism, and treating and/or preventing a wide variety of diseases and disorders including, for example, diseases or disorders related to aging or stress, diabetes, obesity, neurodegenerative diseases, cardiovascular disease, blood clotting disorders, inflammation, cancer, and flushing. The methods comprise, for example, administering to a biological matter in need thereof an effective amount of a sirtuin-modulating compound, e.g., a sirtuin-activating compound.

In other embodiments, the invention provides methods for using active compounds of the present invention wherein the compounds decrease sirtuin activity, e.g., decrease the level and/or activity of a sirtuin protein. Active compounds of the present invention that decrease the level and/or activity of a sirtuin protein may be useful for a variety of therapeutic applications including, for example, increasing cellular sensitivity to stress (including increasing radiosensitivity and/or chemosensitivity), increasing the amount and/or rate of apoptosis, treatment of cancer (optionally in combination another chemotherapeutic agent), stimulation of appetite, and/or stimulation of weight gain.

In certain embodiments, at least one active compound provided to biological matter modulates sirtuin activity to enhance lifespan in the biological matter. For example, a sirtuin-modulating compound that is a sirtuin-activating compound may be provided to biological matter to increase the activity of at least one sirtuin protein, thereby enhancing the lifespan of the biological matter. In certain embodiments, at least one chalcogenide compound and one sirtuin-modulating compound are both provided to biological matter in effective amounts to enhance lifespan in the biological matter.

In certain embodiments, at least one active compound is provided to biological matter wherein the active compound is a chalcogenide. In particular embodiments, the chalcogenide comprises sulfide as in particular, hydrogen sulfide has been shown to increase survivability and enhance lifespan.

Without wishing to be bound to any particular theory, Applicants suggest that sulfide may act as a sirtuin-modulating compound—for example, sulfide may serve to maintain or enhance sirtuin activity.

A. Chalcogenide Compounds

Active compounds of the present invention comprise chalcogenides. Chalcogenides of the present invention may, in certain embodiments, modulate sirtuin activity, whereas in other embodiments, chalcogenides of the present invention may not modulate sirtuin activity.

Compounds containing a chalcogen element—those in Group 6 of the periodic table, but excluding oxides—are commonly termed “chalcogenides” or “chalcogenide compounds (used interchangeably herein). These elements are sulfur (S), selenium (Se), tellurium (Te) and polonium (Po). Common chalcogenides contain one or more of S, Se and Te, in addition to other elements. Chalcogenides include elemental forms such as colloidal, micronized and/or nanomilled particles of S and Se (see, e.g., Example 5). Chalcogenide compounds can be employed as reducing agents. Chalcogenides may be provided in, for example, liquid, solid, semi-solid, or gaseous forms.

Other applications discuss chalcogenides and their use in enhancing survivability, such as U.S. Provisional Patent Application 60/869,054, filed on Dec. 7, 2006, U.S. patent application Ser. No. 11/408,734, filed on Apr. 20, 2006, which claims priority to U.S. Provisional Patent Applications 60/673,037 and 60/673,295, both filed on Apr. 20, 2005, and is further related to U.S. Provisional Patent Application 60/713,073, filed Aug. 31, 2005, U.S. Provisional Patent Application 60/731,549, filed Oct. 28, 2005, and U.S. Provisional Patent Application 60/762,462, filed on Jan. 26, 2006, all of which are hereby incorporated by reference in their entirety.

In one embodiment, the ability of chalcogenides to enhance survivability in cells, enhance lifespan and to permit modulation of core body temperature in animals, stems from the binding of these molecules to cytochrome oxidase (at least in part). In so doing, chalcogenides inhibit or reduce the activity of oxidative phosphorylation. The ability of chalcogenides to block autonomous thermoregulation, i.e., to permit core body temperatures of “warm-blooded” animals to be manipulated through control of environmental temperatures, is believed to stem from the same mechanism (at least in part) as set forth above—binding to cytochrome oxidase, and blocking or reducing the activity of oxidative phosphorylation. The present inventors, though not bound by any particular theory, suggest that in one embodiment, the ability of chalcogenides to enhance survivability in cells, enhance lifespan and to permit modulation of core body temperature in animals may be due to adaptation or an adaptive response. In one embodiment, an adaptive response may modify or activate a signaling pathway. In one embodiment, the signaling pathway may activate or intersect the effectors that activate or inhibit sirtuin proteins. In one embodiment, the adaptive response may modify a second messenger (see: Donaldson and Anderson, 2005).

“Adaptation” refers to physiological process or behavioral trait of an organism that has evolved over a period of time. In one embodiment, adaptation increases the expected long-term reproductive success of the organism. In one embodiment, adaptation may result from exposure to environmental stressors (e.g., oxygen reduction or oxygen deprivation). An “adaptive response” refers to the response of the organism to adaptation. In one embodiment, the adaptive response is a physical, physiological or behavioral change that enhances survival of the organism in anoxic conditions, reduced oxygen conditions, or any other atmospheric change (see: Cohen et al. 1986).

Chalcogenides can be toxic, and at some levels lethal, to mammals. In accordance with the present invention, it is anticipated that the levels of chalcogenide should not exceed lethal levels in the appropriate environment. Lethal levels of chalcogenides may be found, for example in Material Safety Data Sheets for each chalcogenide or from information sheets available from the Occupational Safety and Health Administration (OSHA) of the US Government.

In certain embodiments, a chalcogenide compound of the present invention comprises sulfur, while in others embodiments, it comprises selenium, tellurium, or polonium. In certain embodiments, a chalcogenide compound contains one or more exposed sulfide groups. It is contemplated that a chalcogenide compound may contain 1, 2, 3, 4, 5, 6 or more exposed sulfide groups. In particular embodiments, a sulfide-containing compound is CS₂ (carbon disulfide).

Moreover, in some methods of the invention, longevity is induced in biological matter by exposing the biological matter to a compound that has a chemical structure of (referred to as formula (I)):

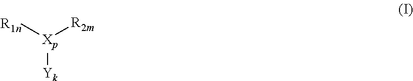

wherein X is N, O, Po, S, Se, or Te;
wherein Y is N or O;
wherein R₁ is H, C, lower alkyl, a lower alcohol, or CN;
wherein R₂ is H, C, lower alkyl, or a lower alcohol, or CN;
wherein n is 0 or 1;
wherein m is 0 or 1;
wherein k is 0, 1, 2, 3, or 4; and,
wherein p is 1 or 2.

The terms “lower alkyl” and “lower alcohol” are used according to their ordinary meanings and the symbols are the ones used to refer to chemical elements. This chemical structure may be referred to as the “reducing agent structure” and any compound having this structure may be referred to as a reducing agent structure compound.

In additional embodiments, k is 0 in formula (I). Moreover, in other embodiments, the R₁ and/or R₂ groups can be an amine or lower alkyl amine. In others, R₁ and/or R₂ could be a short chain alcohol or a short chain ketone. Additionally, R₁ and R₂ may be a linear or branched chain bridge and/or the compound may be a cyclic compound. In still further embodiments, X may also be a halogen. The term “lower” is meant to refer to 1, 2, 3, 4, 5, or 6 carbon atoms. Moreover, R₁ and/or R₂ may be other small organic groups, including, C₂-C₅ esters, amides, aldehydes, ketones, carboxylic acids, ethers, nitriles, anhydrides, halides, acyl halides, sulfides, sulfones, sulfonic acids, sulfoxides, and/or thiols. Such substitutions are clearly contemplated with respect to R₁ and/or R₂. In certain other embodiments, R₁ and/or R₂ may be short chain versions of the small organic groups discussed above. “Short chain” means 1, 2, 3, 4, 5, 6, 7, 8, 9, 10, 11, or 12 carbon molecules.

It is contemplated that a compound of formula (I) can be a chalcogenide compound in some cases. In certain embodiments, the chalcogenide compound has an alkyl chain with an exposed chalcogenide. In others, the chalcogenide compound has a chalcogenide that becomes exposed once it is taken up by the biological matter. In this respect, the chalcogenide compound is similar to a prodrug. Therefore, one or more sulfur, selenium, oxygen, tellurium, polonium, or ununhexium molecules on the compound may become available subsequent to exposure of the biological matter to the chalcogenide compound. In this context, “available” means that the sulfur, selenide, oxygen, tellurium, polonium, or ununhexium will retain a negative charge. Compounds satisfying formula (I) may also behave as reducing agents.

Exemplary compounds that satisfy formula (I) include H2S, ethanol and methanol.

While the embodiments of the present invention described herein are primarily directed to sulfur-containing compounds, it is understood that in other embodiments, the present invention may be practiced using chalcogenides other than sulfur. In certain embodiments, the chalcogenide compound comprises sulfur, while in others it comprises selenium, tellurium, or polonium. In certain embodiments, a chalcogenide compound contains one or more exposed sulfide groups. In particular embodiments, it is contemplated that this chalcogenide compound contains 1, 2, 3, 4, 5, 6 or more exposed sulfide groups. In particular embodiments, such a sulfide-containing compound is CS₂ (carbon disulfide).

“Sulfide” refers to sulfur in its −2 valence state, either as H2S or as a salt thereof (e.g., NaHS, Na₂S, etc.). “H2S” is generated by the spontaneous dissociation of the chalcogenide salt and H2S donor, sodium hydrosulfide (NaHS), in aqueous solution according to the equations:

NaHS→Na++HS—

2HS—⇄H2S+S2—

HS—+H+⇄H2S

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs2S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like.

“Chalcogenide precursor” refers to compounds and agents that can yield a chalcogenide, e.g., hydrogen sulfide (H2S), under certain conditions, such as upon exposure, or soon thereafter, to biological matter. Such precursors yield H2S or another chalcogenide upon one or more enzymatic or chemical reactions. In certain embodiments, the chalcogenide precursor is dimethylsulfoxide (DMSO), dimethylsulfide (DMS), methylmercaptan (CH₃SH), mercaptoethanol, thiocyanate, hydrogen cyanide, methanethiol (MeSH), sodium thiosulfate (Na₂S2O₃), or carbon disulfide (CS₂). In certain embodiments, the chalcogenide precursor is CS₂, MeSH, or DMS. Compounds on the order of the size of these molecules are particularly contemplated (that is, within about 50% of their molecular weights). A working example describing the use of sodium thiosulfate as a precursor to a chalcogenide is provided as Example 5 herein.

“Chalcogenide” or “chalcogenide compound” refers to compounds containing a chalcogen element, i.e., those in Group 6 of the periodic table, but excluding oxides. These elements are sulfur (S), selenium (Se), tellurium (Te) and polonium (Po). Specific chalcogenides and salts thereof include, but are not limited to: H2S, Na₂S, NaHS, K₂S, KHS, Rb₂S, CS₂S, (NH₄)₂S, (NH₄)HS, BeS, MgS, CaS, SrS, BaS, H2Se, Na₂Se, NaHSe, K₂Se, KHSe, Rb₂Se, CS₂Se, (NH₄)₂Se, (NH₄)HSe, BeSe, MgSe, CaSe, SrSe, PoSe and BaSe.

In like fashion, embodiments of the present invention encompass, but are not limited to, corresponding selenide and telluride salts. It is specifically contemplated that the invention includes compositions containing a chalcogenide salt (chalcogenide compound that is a salt) with a pharmaceutically acceptable carrier or prepared as a pharmaceutically acceptable formulation. In still further embodiments, a compound of formula (I) is selected from the group consisting of H2S, H2Se, H₂Te, and H₂Po. In some cases, the compound of formula (I) has an X that is an S. In others, X is Se, or X is Te, or X is Po, or X is O. Furthermore, k in formula (I) is 0 or 1 in some embodiments. In certain embodiments, the compound of formula (I) is dimethylsulfoxide (DMSO), dimethylsulfide (DMS), carbon monoxide, methylmercaptan (CH₃SH), mercaptoethanol, thiocyanate, hydrogen cyanide, methanethiol (MeSH), or CS₂. In particular embodiments, the chalcogenide is H2S, H2Se, CS₂, MeSH, or DMS. Compounds on the order of the size of these molecules are particularly contemplated (that is, within 50% of the average of their molecular weights).

In certain embodiments, a selenium-containing compound such as H2Se is employed. The amount of H2Se may be in the range of 1 to 1000 parts per billion in some embodiments of the invention. It is further contemplated that any embodiment discussed in the context of a sulfur-containing compound may be implemented with a selenium-containing compound. This includes substituting one of more sulfur atoms in a sulfur-containing molecule with a corresponding selenium atom.

A further aspect of the invention encompasses compounds represented by formula (IV):

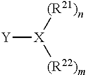

wherein:
X is N, O, P, Po, S, Se, Te, O—O, Po—Po, S—S, Se—Se, or Te—Te;
n and m are independently 0 or 1; and
R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, halo alkylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is 5, SS, Po, Po—Po, Se, Se—Se, Te, or Te—Te, and R²⁴ is defined as for R²¹ herein, or Y is

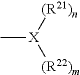

wherein X, R²¹ and R²², are as defined herein.

Moreover, it is contemplated that in some embodiments of the invention, biological matter is provided with a precursor compound that becomes the active version of a formula (I) or (IV) compound by exposure to biological matter, such as by chemical or enzymatic means. In addition, a compound may be provided to the biological matter as a salt of the compound in the form of a free radical, or a negatively charged, positively charged or multiply charged species. Some compounds qualify as both a formula (I) and a formula (IV) compound and in such cases, the use of the phrase “formula (I) or formula (IV)” is not intended to connote the exclusion of such compounds. This reasoning holds true also for “sirtuin-modulating compounds,” as described below, as certain chalcogenides may also be sirtuin-modulating compounds, and as such, use of phrases such as “a chalcogenide or a sirtuin-modulating compound” is not intended to connote the exclusion of such compounds.

A compound identified by the structure of formula (I) or formula (IV) may also, in certain embodiments, be characterized as a chalcogenide, protective metabolic agent, or a precursor, prodrug, or salt thereof. It is further contemplated that the compound need not be characterized as such or qualify as such to be a compound used in the invention, so long as it achieves a particular method of the invention. It is specifically contemplated that any compound identified by the structure of formula (I) or formula (IV) or set forth in this disclosure may be used instead of or in addition to a chalcogenide in methods, compositions, and apparatuses of the invention; similarly, any embodiments discussed with respect to any of structure having formula (I) or formula (IV) or otherwise set forth in this disclosure may be may be used instead of or in addition to a chalcogenide. Moreover, any compound identified by the structure of formulas (I) or (IV) or set forth in this disclosure may be combined with any chalcogenide or any other active compound described herein. It is also contemplated that any combination of such compounds may be provided or formulated together, sequentially (either concurrently or overlapping or non-overlapping), and/or in an overlapping sequential manner (the administration of one compound is initiated and before that is complete, administration of another compound is initiated) in methods, compositions, and other articles of manufacture of the invention to achieve the desired effects set forth herein.

In certain embodiments, more than one compound with the structure of formula (I) or formula (IV) is provided. In certain embodiments, multiple different compounds with a structure from the same formula (i.e., formula (I) or formula (IV)) are employed, while in other embodiments, when multiple different compounds are employed, they are from different formulas.

B. Sirtuin-Modulating Compounds

Active compounds of the present invention comprise sirtuin-modulating compounds. Preferably, such compounds modulate the activity of one or more sirtuin proteins.

The methods of the present invention comprise administering to a subject in need thereof an effective amount of a sirtuin-modulating compound. In certain embodiments, the sirtuin-modulating compounds described herein may be administered alone or in combination with other compounds, such as a chalcogenide compound as described herein. In one embodiment, a mixture of two or more active compounds may be selected from chalcogenide compounds or sirtuin-modulating compounds and may be administered to biological matter in need thereof, wherein sirtuin activity is modulated.

In another embodiment, a sirtuin-modulating compound that increases the level and/or activity of a sirtuin protein may be administered with one or more of the following compounds: resveratrol, butein, fisetin, piceatannol, or quercetin. In an exemplary embodiment, a sirtuin-modulating compound that increases the level and/or activity of a sirtuin protein may be administered in combination with nicotinic acid.

In another embodiment, a sirtuin-activating compound that decreases the level and/or activity of a sirtuin protein may be administered with one or more of the following compounds: nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone), sirtinol; and splitomicin (see e.g., Howitz et al. (2003) Nature 425:191; Grozinger et al. (2001) J. Biol. Chem. 276:38837; Bedalov et al. (2001) PNAS 98:15113; and Hirao et al. (2003) J. Biol. Chem. 278:52773).

In one embodiment, the sirtuin-modulating compound is an aryl-substituted cyclic compound (see: WO 2006/094248, incorporated herein by reference). In one embodiment, the sirtuin-modulating compound is acridine or quinoline or analogs thereof (see: WO 2006/094248, incorporated herein by reference). In one embodiment, the sirtuin-modulating compound is a histone deaceetylase inhibitor (see: WO 2004/009536, incorporated herein by reference). In one embodiment, the sirtuin-modulating compound is an N-phenyl benzamide derivative (see: WO 2006/0094236, incorporated herein by reference).

Exemplary active compounds, such as sirtuin-activating compounds or sirtuin-inhibiting compounds, are described in the following patent applications and patents in the next two paragraphs, each of which is incorporated by reference in its entirety.

A variety of sirtuin-activating compounds and agents are well-known in the art. Non-limiting examples of such sirtuin-activating compounds can be found in the following published patent applications, each of which is specifically incorporated herein by reference: WO 2006/001982 and related US 2006/0002914; WO 2005/002527 and related US 2005/0136429; WO 2005/016342; WO 2006/066244; WO 2005/004814; WO 2006/078941; WO 2006/068656; WO 2006/105440 and related US 2006/0229265 (describing nicotinamide riboside and analogs); WO 2006/094248 (describing aryl-substituted cyclic compounds); WO 2006/094235 (describing fused heterocyclic compounds); WO 2006/094237 (describing acridine and quinoline analogs); WO 2006/094209 (describing N-benzimidazolylalkyl-substituted amide compounds); WO 2006/094236 (describing N-phenyl benzamides); WO 2005/065667 and related applications US 2006/0111435 and US 2005/0171027; WO 2006/007411 and related US 2006/0084085; WO 2005/002555 and related WO 2005/002672, US 2005/0096256 and US 2005/0136537; WO 2006/096780 and related US 2006/0025337.

A variety of sirtuin-inhibitory compounds and agents are well-known in the art. Non-limiting examples of such sirtuin-inhibiting compounds can be found in the following published patent applications, each of which is specifically incorporated herein by reference: WO 2003/046207 and related US 2005/0079995; WO 2005/062952 and related US 2005/0287597; WO 2005/002527 and related US 2005/0136429; WO 2005/078091; WO 2006/006171; WO 2006/031894; WO 2004/009536 and related US 2005/0176686; WO 2003/007722 and related WO 2003/024442, US 2004/0087652, US 2005/0038113, EP 1 293 205; EP 1 427 403 and EP 1 602 371; WO 2006/094248 (describing aryl-substituted cyclic compounds); WO 2006/094235 (describing fused heterocyclic compounds); WO 2006/094237 (describing acridine and quinoline analogs); WO 2006/094209 (describing N-benzimidazolylalkyl-substituted amide compounds); WO 2006/094236 (describing N-phenyl benzamides); WO 2005/065667 and related US 2005/0171027 and US 2006/0111435; WO 2005/065667 and related applications US 2006/0111435 and US 2005/0171027; WO 2006/007411 and related US 2006/0084085; WO 2005/002555 and related WO 2005/002672, US 2005/0096256 and US 2005/0136537; and WO 2006/086454.

Several studies show that nicotinamide adenine dinucleotide (NAD) metabolism regulates sirtuin functioning (see: Porcu, M. and Chiarugi A., TIPS (2005) 26:94). Accordingly, in one embodiment, a chalcogenide compound modulates NAD metabolism. In another embodiment, a sirtuin-modulating compound modulates NAD metabolism. In another embodiment, at least one chalcogenide compound and at least one sirtuin-modulating compound modulate NAD metabolism. In one embodiment, the active compound is nicotinamide riboside or an analog thereof (see: PCT WO 2006/105440, incorporated herein by reference in its entirety).

Nicotinamide riboside and its analogs may directly or indirectly activate sirtuins, such as the human protein SIRT1. In one embodiment, the active compound is a nicotinamide riboside or an analog thereof, which directly or indirectly activates a sirtuin.

In certain embodiments of the invention, the invention is directed to analogs of nicotinamide riboside, particularly compounds that are metabolized, hydrolyzed or otherwise converted to nicotinamide riboside in vivo. Known sirtuin modulators include sirtuin inhibitors (i.e., NAD derivatives (NADH, nicotinamide, carbamido-NAD, dihydrocoumarin derivatives (i.e., dihydrocoumarin, A3), naphthyopyranone drivatives (i.e., splitomicin), 2-hydroxy-naphthaldehyde derivatives (i.e., 2-OH-naphthaldehyde and sirtinol and M15). Sirtuin activators include trans-stilbene derivatives (i.e., piceatannol and resveratrol), chalcone derivatives (i.e., butein and isoliquiritigenin) and flavones (i.e., Fistein 5,7,3,4,5 pentahydroxyflavone, luteolin, quercetin) (see: Porcu, M. and Chiarugi A., TIPS (2005) 26:94).

In certain embodiments, a sirtuin-activating compound is nicotinamide riboside or an analog thereof. See, e.g., US 2006/0229265 and related WO 2006/105440, each of which is incorporated herein by reference in its entirety. Such compounds include formulas 1-13, shown below.

Accordingly, in certain embodiments, compounds for use in the methods described herein are represented by formulas (1) or (2):

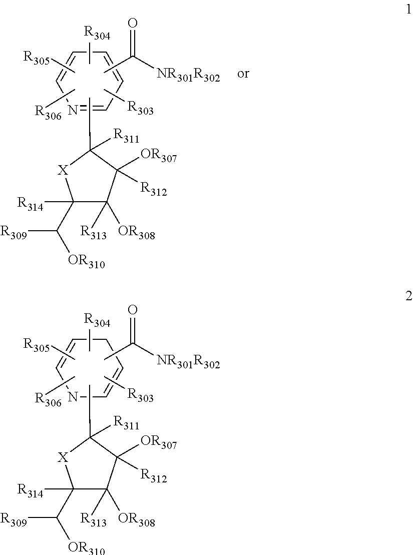

wherein:
R301 and R302 are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R301 and R302 taken together with the atom to which they are attached form a substituted or unsubstituted non-aromatic heterocyclic group;
R303, R304, R305 and R306 are independently selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)nOR, —S(O)ₙNRR′, —NRR′, —NRC(O)OR′, —NO2 and —NRC(O)R′;
R307, R308 and R310 are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;
R309 is selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′ and —NRC(O)R;
R311, R312, R313 and R314 are independently selected from the group consisting of —H, a substituted or unsubstituted alkyl group, a substituted or unsubstituted aryl group, a substituted or unsubstituted non-aromatic heterocyclic group, halogen, —CN, —CO2R, —OCOR, —OCO2R, —C(O)NRR′, —OC(O)NRR′, —C(O)R, —COR, —OSO3H, —S(O)ₙR, —S(O)ₙOR, —S(O)ₙNRR′, —NRR′, —NRC(O)OR′, —NO2 and —NRC(O)R′;
R and R′ are independently —H, a substituted or unsubstituted alkyl group, a substituted or unsubstituted aryl group or a substituted or unsubstituted non-aromatic heterocyclic group;
X is O or S; and
n is 1 or 2.

A group of suitable compounds encompassed by formulas (1) and (2) is represented by formulas (3) and (4):

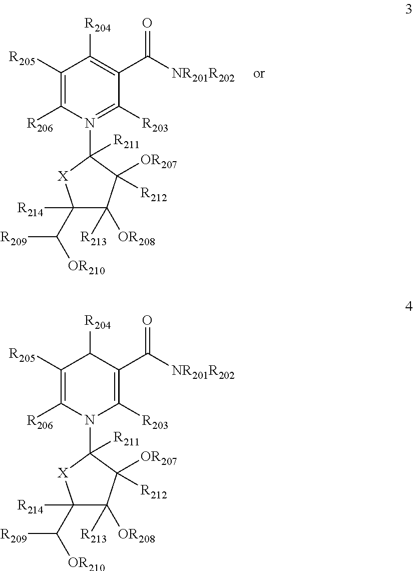

or a pharmaceutically acceptable salt thereof, where:
R201 and R202 are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R201 and R202 taken together with the atom to which they are attached form a substituted or unsubstituted non-aromatic heterocyclic group;
R203, R204, R205 and R206 are independently selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′, —NO2 and —NRC(O)R;
R207, R208 and R210 are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;
R209 is selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′ and —NRC(O)R′;
R211, R212, R213 and R214 are independently selected from the group consisting of —H, a substituted or unsubstituted alkyl group, a substituted or unsubstituted aryl group, a substituted or unsubstituted non-aromatic heterocyclic group, halogen, —CN, —CO2R, —OCOR, —OCO2R, —C(O)NRR′, —OC(O)NRR′, —C(O)R, —OSO3H, —S(O)ₙR, —S(O)ₙOR, —S(O)ₙNRR′, —NRR′, —NRC(O)OR′, —NO2 and —NRC(O)R;
R and R′ are independently —H, a substituted or unsubstituted alkyl group, a substituted or unsubstituted aryl group or a substituted or unsubstituted non-aromatic heterocyclic group;
X is O or S, preferably O; and
n is 1 or 2.

In a particular group of compounds represented by formulas (3) or (4), at least one of R207, R208 and R210 is a substituted or unsubstituted alkyl group, a substituted or unsubstituted aryl group, —C(O)R, —C(O)OR, —C(O)NHR, —C(S)R, —C(S)OR or —C(O)SR. Typically, at least one of R207, R208 and R210 is —C(O)R or —C(O)OR. More typically, at least one of R207, R208 and R210 is —C(O)R. In such compounds, R is preferably a substituted or unsubstituted alkyl, particularly an unsubstituted alkyl group such as methyl or ethyl.

In another particular group of compounds represented by formulas (3) or (4), R204 is a halogen (e.g., fluorine, bromine, chlorine) or hydrogen (including a deuterium and/or tritium isotope). Suitable compounds include those where at least one of R207, R208 and R210 is a substituted or unsubstituted alkyl group, a substituted or unsubstituted aryl group, —C(O)R, —C(O)OR, —C(O)NHR, —C(S)R, —C(S)OR or —C(O)SR and R204 is a halogen or hydrogen.

Typically, for compounds represented by formulas (3) and (4), R203—R206 are —H. In addition, R209 and R211-R214 are typically —H. Particular compounds represented by formulas (3) and (4) are selected such that R203-R206, R209 and R211-R214 are all —H. For these compounds, R204, R207, R208 and R210 have the values described above.

R201 and R202 are typically —H or a substituted or unsubstituted alkyl group, more typically —H. In compounds having these values of R201 and R202, R203-R206, R209 and R211-R214 typically have the values described above.

In certain embodiments, compounds for use in the methods described herein are represented by formula (5) or (6):

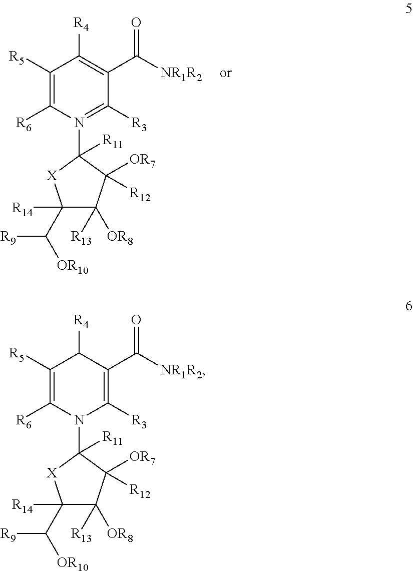

wherein:
R1 and R2 are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R1 and R2 taken together with the atom to which they are attached form a substituted or unsubstituted non-aromatic heterocyclic group, provided that when one of R1 and R2 is —H, the other is not an alkyl group substituted by—C(O)OCH2CH3;
R3, R4 and R5 are independently selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′, —NO2 and —NRC(O)R′;
R6 is selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRC(O)OR′, —NO2 and —NRC(O)R;
R7, R8 and R10 are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;
R9 selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′ and —NRC(O)R′;
R11, R12, R13 and R14 are independently selected from the group consisting of —H, a substituted or unsubstituted alkyl group, a substituted or unsubstituted aryl group, a substituted or unsubstituted non-aromatic heterocyclic group, halogen, —CN, —CO2R, —OCOR, —OCO2R, —C(O)NRR′, —OC(O)NRR′, —C(O)R, —COR, —OSO3H, —S(O)—R, —S(O)ₙOR, —S(O)—NRR′, —NRR′, —NRC(O)OR′, —NO2 and —NRC(O)R;
R and R′ are independently —H, a substituted or unsubstituted alkyl group, a substituted or unsubstituted aryl group or a substituted or unsubstituted non-aromatic heterocyclic group;
X is O or S, preferably O; and
n is 1 or 2.

In certain embodiments, R1 is —H. In certain embodiments, R7, R8 and R10 are independently —H, —C(O)R or —C(O)OR, typically —H or —C(O)R such as —H or —C(O)CH3. In particular embodiments, R1 is —H and R7, R8 and R10 are independently —H, —C(O)R or —C(O)OR.

In certain embodiments, R9 is —H. In particular embodiments, R9 is —H when R1 is —H and/or R7, R8 and R10 are independently —H, —C(O)R or —C(O)OR. In certain embodiments, R2 is —H. In particular embodiments, R2 is —H when R9 is —H, R1 is —H and/or R7, R8 and R10 are independently —H, —C(O)R or —C(O)OR. Typically, R2 is —H when R9 is —H, R1 is —H and R7, R8 and R10 are independently —H, —C(O)R or —C(O)OR. In certain embodiments, R4 is —H or a halogen, such as deuterium or fluorine.

In certain embodiments, compounds for use in the methods described herein are represented by formula (7) or (8):

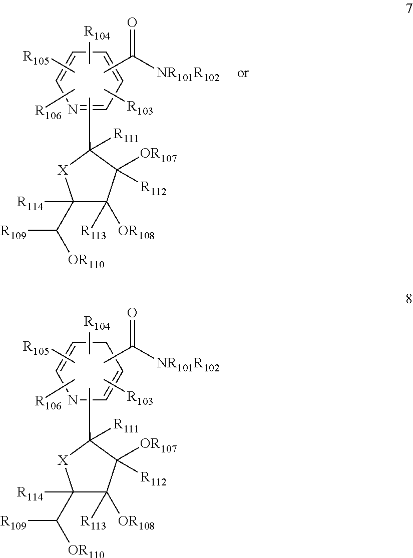

wherein:
R101 and R102 are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R101 and R102 taken together with the atom to which they are attached form a substituted or unsubstituted non-aromatic heterocyclic group;
R103, R104, R105 and R106 are independently selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)nNRR′, —NRR′, —NRC(O)OR′, —NO2 and —NRC(O)R;
R107 and R108 are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R107 and R108 is a substituted or unsubstituted alkyl group, a substituted or unsubstituted aryl group, —C(O)R, —C(O)OR, —C(O)NHR, —C(S)R, —C(S)OR or —C(O)SR;
R109 is selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′ and —NRC(O)R′;
R110 is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R110 is not —C(O)C6H5;
R111, R112, R113 and R114 are independently selected from the group consisting of —H, a substituted or unsubstituted alkyl group, a substituted or unsubstituted aryl group, a substituted or unsubstituted non-aromatic heterocyclic group, halogen, —CN, —CO2R, —OCOR, —OCO2R, —C(O)NRR′, —OC(O)NRR′, —C(O)R, —COR, —OSO3H, —S(O)ₙR, —S(O)ₙOR, —S(O)ₙNRR′, —NRR′, —NRC(O)OR′, —NO2 and —NRC(O)R;
R and R′ are independently —H, a substituted or unsubstituted alkyl group, a substituted or unsubstituted aryl group or a substituted or unsubstituted non-aromatic heterocyclic group;
X is O or S; and
n is 1 or 2.

In another embodiment, compounds for use in the methods described herein are represented by formula (9) or (10):

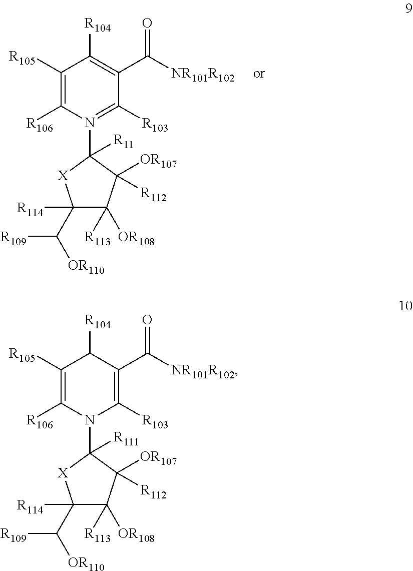

wherein:
R101 and R102 are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R101 and R102 taken together with the atom to which they are attached form a substituted or unsubstituted non-aromatic heterocyclic group;
R103, R104, R105 and R106 are independently selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)nOR, —S(O)ₙNRR′, —NRR′, —NRC(O)OR′, —NO2 and —NRC(O)R′;
R107 and R108 are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R107 and R108 is a substituted or unsubstituted alkyl group, a substituted or unsubstituted aryl group, —C(O)R, —C(O)OR, —C(O)NHR, —C(S)R, —C(S)OR or —C(O)SR;
R109 is selected from the group consisting of —H, a substituted or unsubstituted alkyl group, a substituted or unsubstituted aryl group, a substituted or unsubstituted non-aromatic heterocyclic group, halogen, —OR, —CN, —CO2R, —OCOR, —OCO2R, —C(O)NRR′, —OC(O)NRR′, —C(O)R, —COR, —SR, —OSO3H, —S(O)ₙR, —S(O)ₙOR, —S(O)ₙNRR′, —NRR′, —NRC(O)OR′ and —NRC(O)R′;
R110 is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R110 is not —C(O)C6H5;
R111, R112, R113 and R114 are independently selected from the group consisting of —H, a substituted or unsubstituted alkyl group, a substituted or unsubstituted aryl group, a substituted or unsubstituted non-aromatic heterocyclic group, halogen, —CN, —CO2R, —OCOR, —OCO2R, —C(O)NRR′, —OC(O)NRR′, —C(O)R, —COR, —OSO3H, —S(O)ₙR, —S(O)ₙOR, —S(O)ₙNRR′, —NRR′, —NRC(O)OR′, —NO2 and —NRC(O)R′;
R and R′ are independently —H, a substituted or unsubstituted alkyl group, a substituted or unsubstituted aryl group or a substituted or unsubstituted non-aromatic heterocyclic group;
X is O or S; and
n is 1 or 2.

For compounds represented by formulas (7)-(10), typically at least one of R107 and R108 is —C(O)R, such as —C(O)CH3. In particular embodiments, R107, R108 and R110 are independently —H or —C(O)R (e.g., —C(O)CH3).

In certain embodiments, such as when R107, R108 and R110 have the values described above, R101 and R102 are each —H. In certain embodiments, R109 is —H. In certain embodiments, R103—R106 are each —H. In certain embodiments, R111—R114 are each —H. In particular embodiments, R107, R108 and R110 have the values described above and R101-R106, R109 and R111-R114 are each —H. In certain embodiments, R104 is —H or a halogen, typically deuterium or fluorine. The remaining values are as described above.

For compounds represented by formula (II) or (12), below, R4 in certain embodiments is —H (e.g., deuterium, tritium) or a halogen (e.g., fluorine, bromine, chlorine):

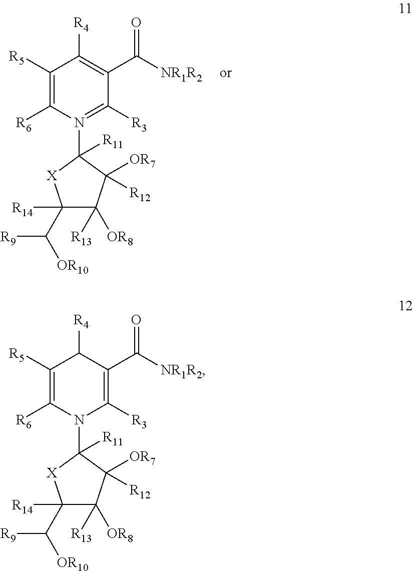

In embodiments of the invention where R1-R6 can each be —H, they typically are each —H. In embodiments of the invention where one of R1-R6 is not —H, typically the remaining values are each —H and the non-H value is a substituted or unsubstituted alkyl group or a halogen (R1 and R2 are typically a substituted or unsubstituted alkyl group)

In certain embodiments, R11-R14 are each —H. When R11-R14 are each —H, R1-R6 typically have the values described above. In certain embodiments, R9 is —H. When R9 is —H, typically R11-R14 are each —H and R1-R6 have the values described above.

For compounds having structures 1-12, the following definitions apply:

An “alkyl group” is a straight chained, branched or cyclic non-aromatic hydrocarbon which is completely saturated. Typically, a straight chained or branched alkyl group has from 1 to about 20 carbon atoms, preferably from 1 to about 10, and a “cyclic alkyl group” has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An “alkenyl group” is a straight chained, branched or cyclic non-aromatic hydrocarbon which contains one or more double bonds. Typically, the double bonds are not located at the terminus of the alkenyl group, such that the double bond is not adjacent to another functional group.

An “alkynyl group” is a straight chained, branched or cyclic non-aromatic hydrocarbon which contains one or more triple bonds. Typically, the triple bonds are not located at the terminus of the alkynyl group, such that the triple bond is not adjacent to another functional group.

A “cyclic ring” (e.g., a 5- to 7-membered ring) includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

“Aromatic (aryl) groups” include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl;

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl;

“Non-aromatic heterocyclic rings” are non-aromatic carbocyclic rings which include one or more heteroatoms such as nitrogen, oxygen or sulfur in the ring. The ring can be five, six, seven or eight-membered. Examples include tetrahydrofuranyl, tetrahydrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein.

A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R³, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —C0N(R^{a}R^{b}), —NHCOR^{a}, —NRC0R^{a}, —NHCONH₂, —NHC0NR^{a}H, —NHCON(R³R¹³), —NR⁰CONH₂, —NR^{C}CONRH, —NR^{c}C0N(R^{a}R^{b}), —CC═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{C})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR₃, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂′. R^{a}-R^{d} are each independently an aliphatic, substituted aliphatic, benzyl, substituted benzyl, aromatic or substituted aromatic group, preferably an alkyl, benzylic or aryl group.

In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

Sirtuin-activating compounds of formulas 1-12 and any other compounds of the present invention having hydroxyl substituents, unless otherwise indicated, also include the related secondary metabolites, particularly sulfate, acyl (e.g., acetyl, fatty acid acyl) and sugar (e.g., glucurondate, glucose) derivatives. In other words, substituent groups —OH also include —OSO₃⁻M⁺, where M⁺ is a suitable cation (preferably H⁺, NH₄⁺ or an alkali metal ion such as Na⁺ or K⁺) and sugars such as:

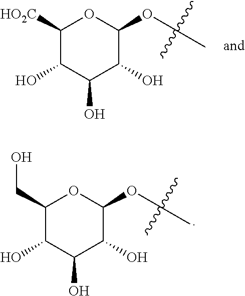

These groups are generally cleavable to —OH by hydrolysis or by metabolic (e.g., enzymatic) cleavage.

Double bonds indicated in any structure described herein as:

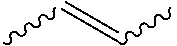

are intended to include both the (E)- and (Z)-configuration. Preferably, double bonds are in the (E)-configuration.

In certain embodiments, a sirtuin-activating compound is compound as described in WO 2006/078941, incorporated herein by reference in its entirety. Such compounds include formulas 13-31, shown below.

Accordingly, in certain embodiments, compounds for use in the methods described herein are represented by formula (13):

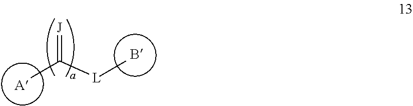

wherein:
Ring A′ is a 5- to 7-membered ring optionally fused to a second 5- to 7-membered ring, which is optionally substituted with one to three functional groups selected from the group consisting of halogen, —OR, —CN, —CO₂R, —OCOR, —OCO₂R₅—C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; Ring B′ is a 5- to 7-membered ring optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; J is O or S;
L is —C═C— or —NH—(CH₂)ₖ—;
R and R′ are independently —H, a substituted or unsubstituted alkyl group, a substituted or unsubstituted alkenyl group, a substituted or unsubstituted non-aromatic heterocyclic group or a substituted or unsubstituted aryl group; a is 0 or 1; k is an integer from 1 to 4; and n is 1 or 2.

Preferably, one or both of Ring A′ and Ring B′ are aromatic, more preferably, both are aromatic. Suitable aromatic groups include, but are not limited to, pyridyl, phenyl, thienyl, furanyl, indolyl, pyrrolyl, imidazolyl, oxazolyl and thiazolyl. Particularly suitable aromatic groups are phenyl and pyridyl.

For one class of compounds encompassed by formula (Ia), a is 0. When a is 0, L is typically —CH═CH—. For another class of compounds encompassed by formula (Ia), a is 1.

When a is 1, J is typically 0. When a is 1 and J is 0, k is typically 1.

Typically, the hydrogen bond donating group is —OR, —OCOR, —OSO₃H, —COOH, —SH or —NHR. Preferably, the hydrogen bond donating group is —OR, —OCOR, or —OSO₃H. When the hydrogen bond donating group is —OR, the group is preferably hydrolyzable or metabolically cleavable to —OH (e.g., R is a sugar).

In another embodiment, sirtuin-activating compounds of the invention are represented by formula (14):

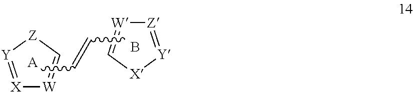

wherein:
W is CH or N;
X is CH or N; Y is CH or N; Z is S, O or NH; W is CH or N; X′ is CH or N;
Y′ is CH or N; Z′ is S, O or NH;
R and R′ are independently —H, a substituted or unsubstituted alkyl group, a substituted or unsubstituted alkenyl group, a substituted or unsubstituted non-aromatic heterocyclic group or a substituted or unsubstituted aryl group; n is 1 or 2;
Ring A may be substituted with at least one hydrogen bond donating group and is optionally substituted with one to three functional groups selected from the group consisting of halogen, —OR, —CN, —CO₂R, —OCOR, —OCO₂R₅—C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; and
Ring B may be optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

One group of sirtuin-activating compounds encompassed by formula (14) is represented by formula (15):

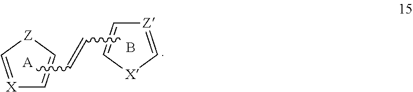

Particular compounds represented by formula (15) include those where X is CH and Z is NH, O or S, or X is N and Z is S; and X′ is CH and Z′ is NH, O or S, or X is N and Z is S.

One group of sirtuin-activating compounds encompassed by formula (15) is represented by formula (16):

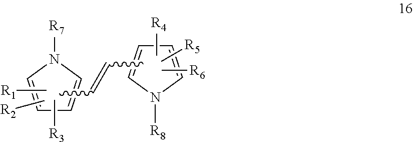

wherein:
R₁ is —OR, —OSO₃H, —SH, —NHR or —COOR;
R₂, R₃, R₄, R₅ and R₆ are independently —H, halogen, —OR, —CN, —CO₂R, —OCOR, —OCO₂R, —C(O)NRR′, —OC(O)NRR′, —C(O)R, —CORₙ—SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl; and
R₇ and R₈ are independently —H, a substituted or unsubstituted alkyl group, a substituted or unsubstituted alkenyl group or a substituted or unsubstituted aryl group.

A particular group of sirtuin-activating compounds encompassed by formula (16) is represented by formula (17):

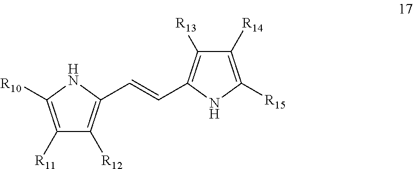

wherein:
R₁₀, R₁₁ and R₁₂ are independently —H, halogen, —OR, —CN, —CO₂R, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl, provided that at least one of R₁₀, R₁₁ and R₁₂ is —OH, —NHR, —SH or —COOR;
R₁₃, R₁₄ and R₁₅ are independently —H, halogen, —OR, —CN, —CO₂R, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl or substituted or unsubstituted aryl.

In a preferred embodiment, at least one of R₁₀, R₁₁ and R₁₂ is —OR, —OCOR, or —OSO₃H, such as where two of these variables are —OR, —OCOR, or —OSO₃H. When the hydrogen bond donating group is —OR, the group is preferably hydrolyzable or metabolically cleavable to —OH (e.g., R is a sugar).

In another embodiment, at least one of R₁₀, R₁₁ and R₁₂ is a dihalomethyl group, such as a dihalomethyl (e.g., difluoromethyl, dichloromethyl) group. When R₁₀, R₁₁ and R₁₂ have the values described above, in certain embodiments at least one of R₁₃, R₁₄ and R₁₅ is —OR, —OCOR, —OSO₃H, —NHR, —SH or —COOR, preferably —OR, —OCOR, or —OSO₃H. When the hydrogen bond donating group is —OR, the group is preferably hydrolyzable or metabolically cleavable to —OH (e.g., R is a sugar).

In another embodiment, sirtuin-activating compounds of the invention are represented by formula (18):

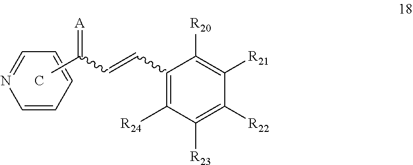

wherein:
A is O, NH or S;
R₂₀, R₂₁, R₂₂, R₂₃ and R₂₄ are independently —H, halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic or substituted or unsubstituted aryl;
R and R′ are independently —H, a substituted or unsubstituted alkyl group, a substituted or unsubstituted alkenyl group, a substituted or unsubstituted non-aromatic heterocyclic group or a substituted or unsubstituted aryl group;
n is 1 or 2; and
Ring C is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.

One group of sirtuin-activating compounds encompassed by formula (18) is represented by formula (19):

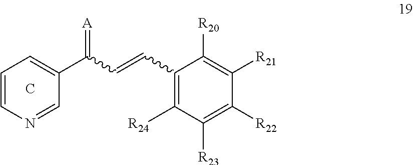

One group of sirtuin-activating compounds encompassed by formula (19) is represented by formula (20):

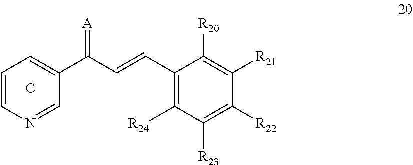

Typically, Ring C is unsubstituted. When Ring C is unsubstituted, A is typically O.

When Ring C and A have the values described above, one or two of R₂₀, R₂₁, R₂₂, R₂₃ and R₂₄ can be —OR, —OCOR, —OSO₃H, —NHR, —SH or —COOR, preferably —OR, —OCOR, or —OSO₃H, and the remainder Of R₂₀, R₂₁, R₂₂, R₂₃ and R₂₄ can be —H. In another embodiment, one or two of R₂₀, R₂₁, R₂₂, R₂₃ and R₂₄ is a dihalomethyl group, preferably a difluoromethyl group. When the hydrogen bond donating group is —OR, the group is preferably hydrolyzable or metabolically cleavable to —OH (e.g., R is a sugar).

In one embodiment where compounds having the values of A, R₂₀, R₂₁, R₂₂, R₂₃ and R₂₄ described above and where Ring C is substituted or unsubstituted, one or two of R₂₁, R₂₂ and R₂₃ are —OR, —OCOR, or —OSO₃H. When the hydrogen bond donating group is —OR, the group is preferably hydrolyzable or metabolically cleavable to —OH (e.g., R is a sugar).

In a further embodiment, sirtuin-activating compounds of the invention are represented by formula (21):

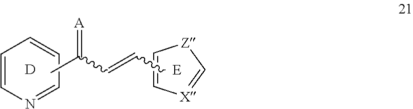

wherein:
A is O, NH or S; X″ is CH or N; Z″ is NH, O or S; R and R′ are independently —H, a substituted or unsubstituted alkyl group, a substituted or unsubstituted alkenyl group, a substituted or unsubstituted non-aromatic heterocyclic group or a substituted or unsubstituted aryl group;
n is 1 or 2;
Ring D is optionally substituted with one to four functional groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl; and
Ring E is optionally substituted with one to three functional groups selected from the group consisting of halogen, —OR, —CN, —CO₂R, —OCO₂R₅—OCOR, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl.
Typically, A is O. When A is O, X″ can be CH.

One group of sirtuin-activating compounds encompassed by formula (21) is represented by formula (22):

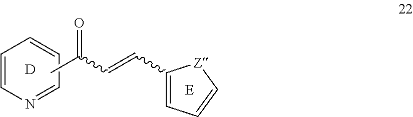

One group of sirtuin-activating compounds encompassed by formula (22) is represented by formula (23):

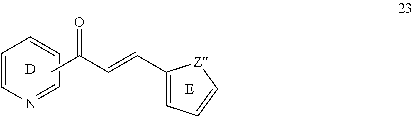

A particular group of sirtuin-activating compounds encompassed by formula (23) is represented by formula (24):

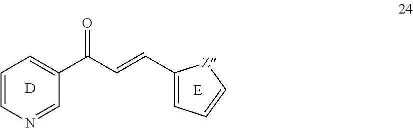

Typically, Z″ for compounds of formulas (21)-(24) is NH. When Z″ is NH, Ring D is preferably unsubstituted and Ring E is optionally substituted with one or two —OR, —OCOR, —OSO₃H, —NHR, —SH or —COOR groups, preferably one or two —OR, —OCOR, or —OSO₃H groups. When the hydrogen bond donating group is —OR, the group is preferably hydrolyzable or metabolically cleavable to —OH (e.g., R is a sugar).

In another group of compounds of the invention, Z″ and Ring D are as described above, and Ring E is substituted with one or two dihalomethyl groups. Preferably, the dihalomethyl group is a difluoromethyl group.

Further sirtuin-activating compounds of the invention are represented by formula (25):

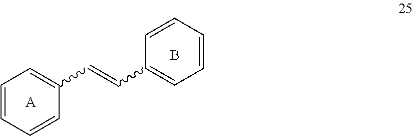

wherein:
Ring A is substituted with at least one dihalomethyl group and at least one group capable of donating hydrogen bonds; and
Ring B is optionally substituted.

When Ring B is substituted, one or more of the substituents are preferably a group capable of donating hydrogen bonds.

For both Ring A and Ring B, typical hydrogen bond donating groups are —OR, —OCOR₅—OSO₃H, —NHR, —SH and —COOR (where R is as defined above), preferably —OR, OCOR, or —OSO₃H. When the hydrogen bond donating group is —OR, the group is preferably hydrolyzable or metabolically cleavable to —OH (e.g., R is a sugar). Suitable dihalomethyl groups include dichloromethyl, dibromomethyl and difluoromethyl, preferably difluoromethyl.

A particular sirtuin-activating compound encompassed by formula (25) is represented by formula (26):

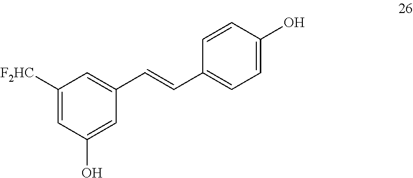

In one embodiment, sirtuin-activating compounds of the invention are represented by formula (27):

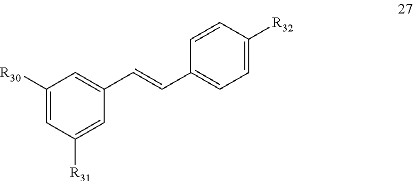

wherein:
R₃₀ is —OR_{ZJ}—OCH₃, —Cl, —OC₆H₅ or —CH₃;
R₃₁ is —H, —OR₂, —OCH₃, —F or —CH₃; R₃₂ is −OR₂, —OCHF₂, —OCHCl₂, —OCHBr₂ or —OCH₃; and
R₂ is —SO₃H, an acyl group (e.g., acetyl or the acyl group of a fatty acid) or a sugar, provided that R₃₂ is —OCHF₂, —OCHCl₂, —OCHBr₂ or —OCH₃ when R₃₀ and R₃₁ are both —OH.

Particular sirtuin-activating compounds encompassed by formula (27) are represented by the following structural formulas:

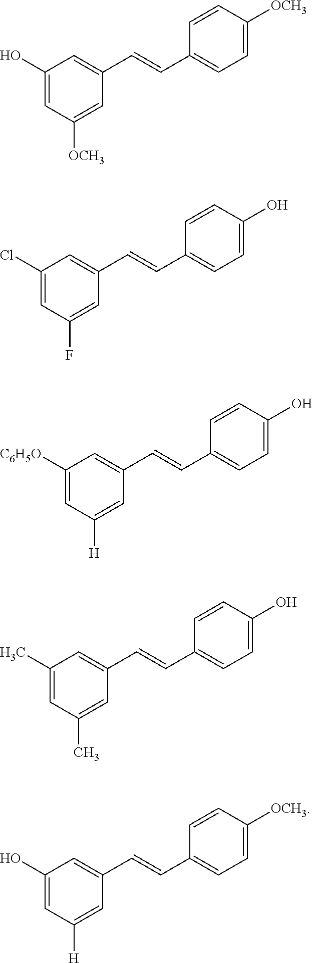

The hydroxyl groups of these compounds can be replaced with —OSO₃H or —OR₂, where R₂ is an acyl group (e.g., acetyl or the acyl group of a fatty acid) or a naturally or non-naturally occurring sugar.

Additional sirtuin-activating compounds encompassed by formula (27) are represented by the following formulas:

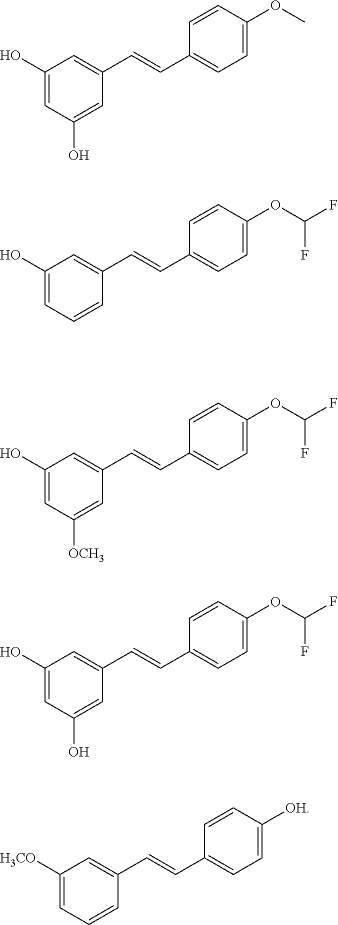

The hydroxyl groups of these compounds can be replaced with —OSO₃H or —OR₂, where R_{z} is an acyl group (e.g., acetyl or the acyl group of a fatty acid) or a naturally or non-naturally occurring sugar.

In another embodiment, sirtuin-activating compounds of the invention are represented by formula (28):

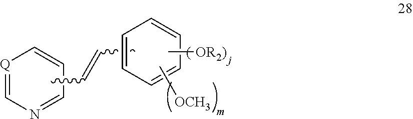

wherein:
j is 1 or 2; m is 0 or 1;
Q is CH or N; and R_{z}′ is —H, —SO₃H, acyl or a sugar, provided that the compound is not 4-((E)-2-(pyridin-4-yl)vinyl)phenol.

One group of sirtuin-activating compounds encompassed by formula (28) is represented by formula (29):

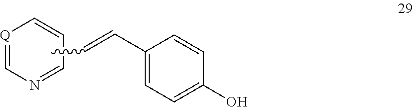

wherein Q is CH or N. The hydroxyl groups of these compounds can be replaced with —OSO₃H or —OR₂, where R₂ is an acyl group (e.g., acetyl or the acyl group of a fatty acid) or a naturally or non-naturally occurring sugar.

Particular sirtuin-activating compounds encompassed by formula (29) are represented by the following structural formulas:

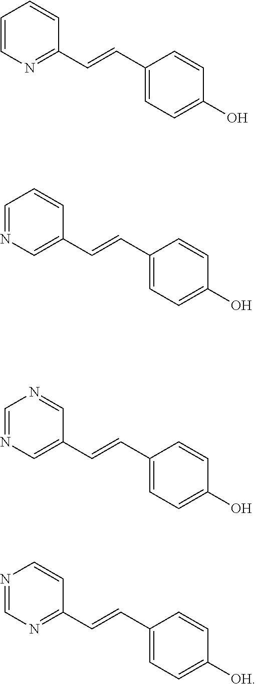

The hydroxyl groups of these compounds can be replaced with —OSO₃H or —OR₂, where R_{z} is an acyl group (e.g., acetyl or the acyl group of a fatty acid) or a naturally or non-naturally occurring sugar.

Another group of sirtuin-activating compounds encompassed by formula (28) is represented by formula (30):

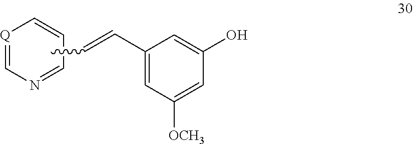

wherein Q is CH or N. The hydroxyl groups of these compounds can be replaced with —OSO₃H or —OR₂, where R₂ is an acyl group (e.g., acetyl or the acyl group of a fatty acid) or a naturally or non-naturally occurring sugar.

Particular sirtuin-activating compounds encompassed by formula (30) are represented by the following structural formulas:

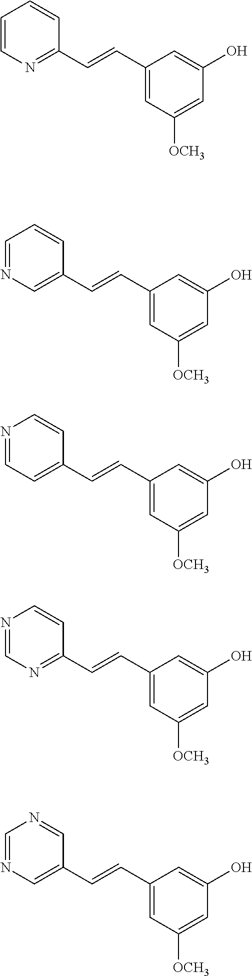

The hydroxyl groups of these compounds can be replaced with —OSO₃H or —OR₂, where R₂ is an acyl group (e.g., acetyl or the acyl group of a fatty acid) or a naturally or non-naturally occurring sugar.

Other particular sirtuin-activating compounds encompassed by formula (28) are represented by the following:

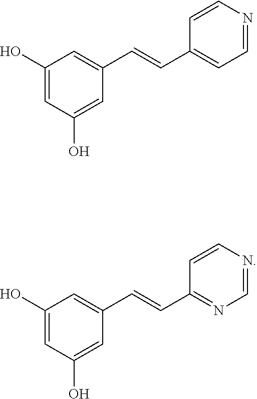

The hydroxyl groups of these compounds can be replaced with —OSO₃H or —OR₂, where R₂ is an acyl group (e.g., acetyl or the acyl group of a fatty acid) or a naturally or non-naturally occurring sugar.

In yet another embodiment, sirtuin-activating compounds of the invention are represented by formula (31):

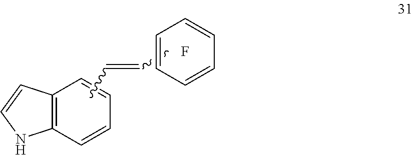

wherein:
Ring F is substituted with at least one hydrogen bond donating group and the compound is optionally substituted with one or more groups selected from the group consisting of halogen, —OR, —CN, —CO₂R, —OCOR, —OCO₂R, —C(O)NRR′, —OC(O)NRR′, —C(O)R, —COR, —SR, —S(O)ₙR, —S(O)ₙOR, —S(O)ₙNRR′, —NRR′, —NRC(O)OR, —NRC(O)R, —NO₂, —OSO₃H, substituted or unsubstituted alkyl, substituted or unsubstituted alkenyl, substituted or unsubstituted non-aromatic heterocyclic and substituted or unsubstituted aryl;
R and R′ are independently —H, a substituted or unsubstituted alkyl group, a substituted or unsubstituted alkenyl group, a substituted or unsubstituted non-aromatic heterocyclic group or a substituted or unsubstituted aryl group; and n is 1 or 2.

The hydrogen bonding donating group on Ring F is typically —OR, —OSO₃H, —SH, —NHR or —COOR, preferably —OR or —OSO₃H. When the hydrogen bond donating group is —OR, the group is preferably hydrolyzable or metabolically cleavable to —OH (e.g., R is a sugar or an acyl group).

Groups of sirtuin-activating compounds encompassed by formula (31) are represented by the following structural formulas:

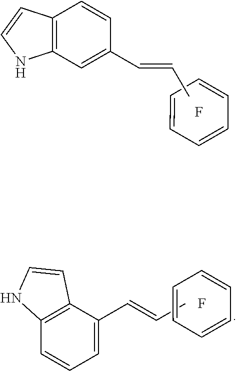

In certain embodiments, a sirtuin-activating compound is a polyphenol, e.g., a flavone, stilbene, flavanone, isoflavones, catechins, chalcone, tannin, or anthocyanidin. For example, the agent may be a trans-stilbene, e.g., resveratrol. The agent may also be a nucleic acid that encodes a SIRT1 polypeptide or a functional domain thereof, e.g., the core domain.

In certain embodiments, a sirtuin-activating compound is a compound as described in WO 2005/004814, incorporated herein by reference in its entirety. Such compounds include formulas 32-35, shown below.

Accordingly, in certain embodiments, compounds for use in the methods described herein are represented by formulas (32):

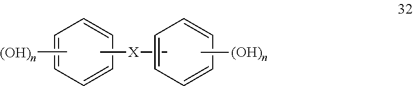

wherein;
X is alkenyl, C(O)CH═CH, or a hydroxy pyranone fused to one of the phenyl moieties to form a flavone; and each n is independently 1-3.

For example, the compound of formula 32 can be a polyhydroxy stilbene (e.g., polyhydroxy-trans-stilbene) as shown in formula (33), a polyhydroxy chalcone as shown in formula (34), or a polyhydroxyflavone as shown in formula (35). In general, the compound is substituted with at least 2, preferably 3, 4, of 5 hydroxy moieties. Exemplary compounds include resveratrol (3,5,4′-trihydroxy-trans-stilbene), butein (3,4,2′,4′-tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). See, e.g., Howitz (2003) Nature 425: 191-196 (also discussed below).

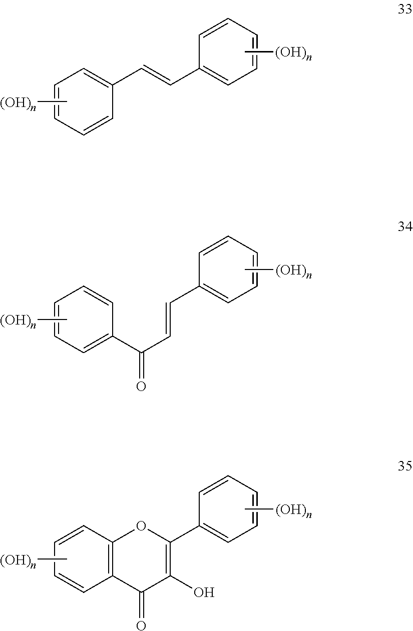

In certain embodiments, the sirtuin-activating compound is selected from the group consisting of oxaloacetate, oxaloacetic acid, an oxaloacetate salt, alpha-ketoglutarate and aspartate. See, e.g., WO 2006/066244, incorporated herein by reference in its entirety.

In certain embodiments, the sirtuin-activating compound is a compound that inhibits SIR2 (or other sirtuin protein) base exchange more than deacetylation. See, e.g., WO 2005/016342. Such compounds promote a net increase in deacetylation, thus effectively increasing the deacetylation activity of SIR2. Thus, in some embodiments, the invention is directed to compounds that inhibit base exchange more than deacetylation by a SIR2 enzyme. Without being limited to any particular mechanism, the compounds are believed to inhibit base exchange by displacing nicotinamide from the SIR2 active site.

Accordingly, certain compounds of the present invention have structural characteristics similar to nicotinamide, for example the following structures of formulas 36-40, where formula 36 has one of structures a-h:

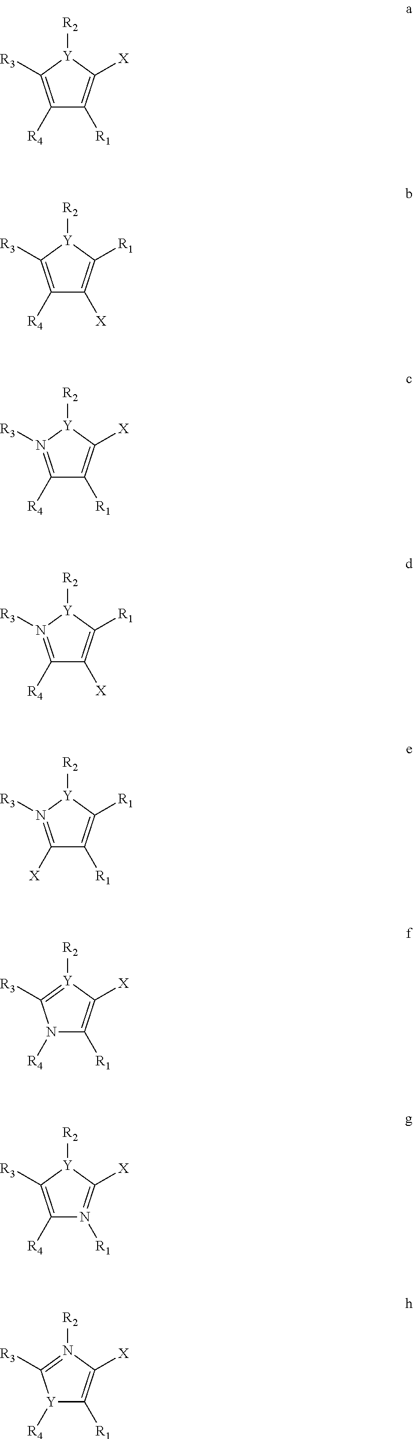

where R1, R2, R3 and R4 are independently H, F, Cl, Me, OH, NH2, CF3 or Me; X is CONHMe, COCH3, COCH2CH3, COCF3, CH2OH or CH, NH; and Y is N, O, or S; when Y═S or O, the corresponding R is not defined.

Formula 37 has one of structures i-r:

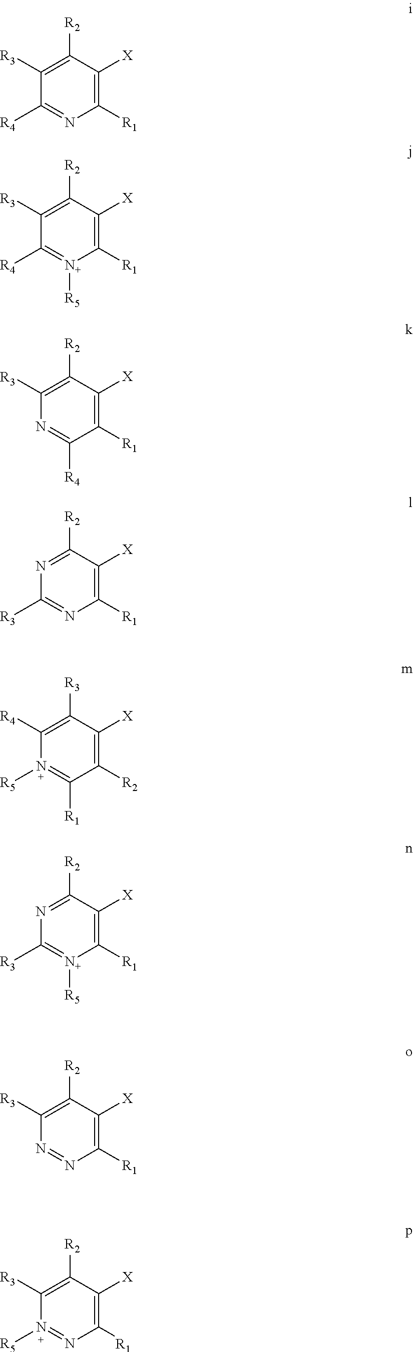

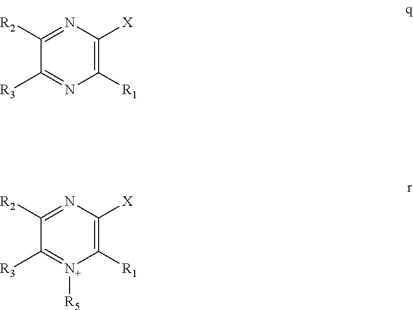

where R1, R2, R3 and R4 are independently H, F, Cl, OH, NH2, Me or CF3; X is CONH2, CONHMe, COCH3, COCH2CH3, COCF3, CH2OH or CH2NH2; and R5 is Me, CF3, O or NH2, and wherein formula 37 is not nicotinamide.

Formula 38 has one of structures v or w:

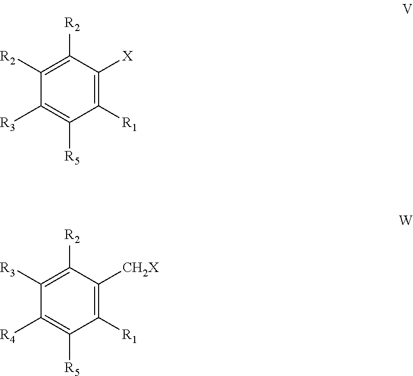

where R1, R2, R3, R4, and R5 are independently H, F, Cl, OH, NH2, Me or CF3; and X is CON, CONHMe, COCH3, COCH2CH3, COCF3, CH2OH or CH2NH2.

Formula 39 has one of structures x or y:

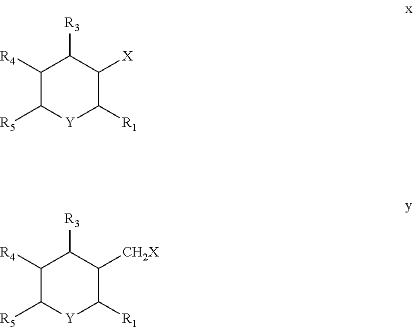

where the ring may comprise zero, one or two double bonds; R1, R2, R3, R4 and R5 are independently H, F, Cl, OH, NH2, Me or CF3; and X is CONH, CONHMe, COCH3, COCH2CH3, COCF3, CH2OH or CH2NH2; and Y is N, O or S.

Formula 40 has one of structures z or aa:

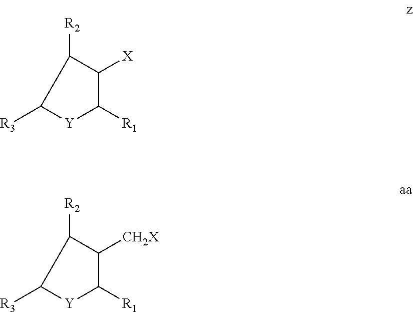

where the ring may comprise zero or one double bond; R1, R2, and R3 are independently H, F, Cl, OH, NH2, Me or CF3; and X is CONH2, CONHMe, COCH3, COCH2CH3, COCF3, CH2OH or CH2NH2; and Y is N, O or S.

In certain embodiments, the compound has one of structures a, b, f, x, y, z, or aa, where X is CONH2 and Y is N; structure i, where at least one of R1-R4 is F and X is CONH2; structure k, where R1, R2, R3 and R4 are independently H or F and X is CONH2; or structures v and w, where at least one of R1-R5 is F and X is CONH2.

In certain embodiments, the compound has one of structure a or b, where R2 is CH3, and R1, R3 and R4 is H; structure f, where R1, R3 and R4 is H and R2 is CH3 or H; structure i, where R1 is F, R2-R4 is H, and X is CONH2 (2-fluoronicotinamide); other fluoronicotinamides, or structure k, wherein R1-R4 is H and X is CONH2 (isonicotinamide). In certain embodiments, the compound is isonicotinamide or a fluoronicotinamide such as 2-fluoronicotinamide.

Other sirtuin-activating compounds are described in WO 2006/001982 and related US 2006/0002914 (each of which is incorporated herein by reference in its entirety), such as polyphenols, some of which have been described above (see, e.g., Howitz et al., Nature 425:191-196, 2003 and supplementary information that accompanies the paper, all of which is incorporated herein by reference). Such compounds can include stilbenes such as resveratrol, piceatannol, deoxyrhapontin, trans-stilbene and rhapontin; chalcone such as butein, isoliquiritigen and 3,4,2′,4′,6′-pentahydroxychalcone and chalcone; flavones such as fisetin, 5,7,3′,4′,5′-pentahydroxyflavone, luteolin, 3,6,3′,4′-tetrahydroxyflavone, quercetin, 7,3′,4′,5′-tetrahydroxyflavone, kaempferol, 6-hydroxyapigenin, apigenin, 3,6,2′,4′-tetrahydroxyflavone, 7,4′-dihydroxyflavone, 7,8,31,4′-tetrahydroxyflavone, 3,6,2′,3′-tetrahydroxyflavone, 4′-hydroxyflavone, 5,4′-dihydroxyflavone, 5,7-dihydroxyflavone, morin, flavone and 5-hydroxyflavone; isoflavones such as daidzein and genistein; fiavanones such as naringenin, 3,5,7,3′,4′-pentahydroxyflavanone, and flavanone or catechins such as (−)-epicatechin, (−)-catechin, (−)-gallocatechin, (+)-catechin and (+)-epicatechin. Additional polyphenols or other substance that increase sirtuin deacetylase activity can be identified using assay systems described in the art and commercially available assays such as fluorescent enzyme assays (Biomol International LP, Plymouth Meeting, Pa.). Sinclair et al. also disclose substances that can increase sirtuin activity (Sinclair et al., WO2005/02672 which is incorporated in its entirety by reference).

In certain embodiments, a sirtuin-activating compound is compound as described in US 2006/0084135, incorporated herein by reference in its entirety. Such compounds include formulas 41-66, shown below.

Accordingly, in certain embodiments, compounds for use in the methods described herein are represented by formula (41):

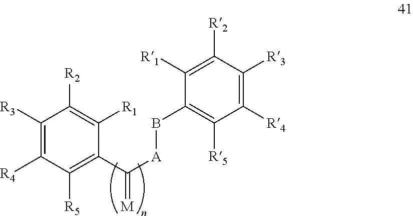

wherein:
R1, R2, R3, R4, R5, R′1, R′2, R′3, R′4, and R′S represent H, alkyl, aryl, heteroaryl, alkaryl, heteroaralkyl, halide, NO2, SR, OR, N(R)2, or carboxyl;
R represents H, alkyl, or aryl;
M represents O, NR, or S;
A-B represents a bivalent alkyl, alkenyl, alkynyl, amido, sulfonamido, diazo, ether, alkylamino, alkylsulfide, or hydrazine group; and
n is 0 or 1.

In a further embodiment, the methods comprise a compound of formula 41 and the attendant definitions, wherein n is 0. In a further embodiment, the methods comprise a compound of formula 41 and the attendant definitions, wherein n is 1. In a further embodiment, the methods comprise a compound of formula 41 and the attendant definitions, wherein A-B is ethenyl. In a further embodiment, the methods comprise a compound of formula 41 and the attendant definitions, wherein A-B is —CH2CH(Me)CH(Me)CH2-. In a further embodiment, the methods comprise a compound of formula 41 and the attendant definitions, wherein M is O. In a further embodiment, the methods comprises a compound of formula 41 and the attendant definitions, wherein R1, R2, R3, R4, R5, R′1, R′2, R′3, R′4, and R′5 are H. In a further embodiment, the method comprise a compound of formula 41 and the attendant definitions, wherein R2, R4, and R′3 are OH. In a further embodiment, the methods comprise a compound of formula 41 and the attendant definitions, wherein R2, R4, R′2 and R′3 are OH. In a further embodiment, the methods comprise a compound of formula 41 and the attendant definitions, wherein R3, R5, R′2 and R′3 are OH. In a further embodiment, the methods comprise a compound of formula 41 and the attendant definitions, wherein R1, R3, R5, R′2 and R′3 are OH. In a further embodiment, the methods comprise a compound of formula 41 and the attendant definitions, wherein R2 and R′2 are OH; R4 is O-β-D-glucoside; and R′3 is OCH3. In a further embodiment, the methods comprise a compound of formula 41 and the attendant definitions, wherein R2 is OH; R4 is O-β-D-glucoside, and R′3 is OCH3.

In a further embodiment, the methods comprise a compound of formula 41 and the attendant definitions, wherein n is 0; A-B is ethenyl; and R1, R2, R3, R4, R5, R′1, R′2, R′3, R′4, and R′5 are H (trans-stilbene). In a further embodiment, the methods comprise a compound of formula 41 and the attendant definitions, wherein n is 1; A-B is ethenyl; M is O; and R1, R2, R3, R4, R5, R′1, R′2, R′3, R′4, and R′5 are H (chalcone). In a further embodiment, the methods comprise a compound of formula 41 and the attendant definitions, wherein n is 0; A-B is ethenyl; R2, R4, and R′3 are OH; and R1, R3, R5, R′1, R′2, R′4, and R′5 are H (resveratrol). In a further embodiment, the methods comprise a compound of formula 41 and the attendant definitions, wherein n is 0; A-B is ethenyl; R2, R4, R′2 and R′3 are OH; and R1, R3, R5, R′1, R′4 and R′5 are H (piceatannol). In a further embodiment, the methods comprise a compound of formula 41 and the attendant definitions, wherein n is 41; A-B is ethenyl; M is O; R3, R5, R′2 and R′3 are OH; and R1, R2, R4, R′1, R′4, and R′S are H (butein). In a further embodiment, the methods comprise a compound of formula 41 and the attendant definitions, wherein n is 1; A-B is ethenyl; M is O; R1, R3, R5, R′2 and R′3 are OH; and R2, R4, R′1, R′4, and R′5 are H (3,4,2′,4′,6′-pentahydroxychalcone). In a further embodiment, the methods comprise a compound of formula 41 and the attendant definitions, wherein n is 0; A-B is ethenyl; R2 and R′2 are OH, R4 is O-β-D-glucoside, R′3 is OCH3; and R1, R3, R5, R′1, R′4, and R′5 are H (rhapontin). In a further embodiment, the methods comprise a compound of formula 41 and the attendant definitions, wherein n is 0; A-B is ethenyl; R2 is OH, R4 is O-β-D-glucoside, R′3 is OCH3; and R1, R3, R5, R′1, R2, R′4, and R′5 are H (deoxyrhapontin). In a further embodiment, the methods comprise a compound of formula 41 and the attendant definitions, wherein n is 0; A-B is —CH2CH(Me)CH(Me)CH2—; R2, R3, R′2, and R′3 are OH; and R1, R4, R5, R′1, R′4, and R′5 are H(NDGA).

In another embodiment, methods for activating a sirtuin protein comprise an activating compound that is a flavanone compound of formula 42:

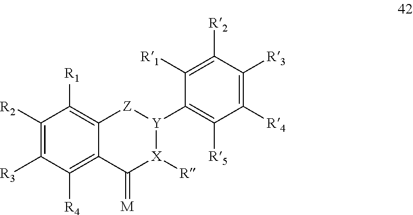

wherein:
R1, R2, R3, R4, R5, R′1, R′2, R′3, R′4, R′5, and R″ represent H, alkyl, aryl, heteroaryl, alkaryl, heteroaralkyl, halide, NO2, SR, OR, N(R)2, or carboxyl;
R represents H, alkyl, or aryl;
M represents H2, O, NR, or S;
Z represents CR, O, NR, or S; and
X represents CR or N; and
Y represents CR or N.

In a further embodiment, the methods comprise a compound of formula 42 and the attendant definitions, wherein X and Y are both CH. In a further embodiment, the methods comprise a compound of formula 42 and the attendant definitions, wherein M is O. In a further embodiment, the methods comprise a compound of formula 42 and the attendant definitions, wherein. M is H2. In a further embodiment, the methods comprise a compound of formula 42 and the attendant definitions, wherein Z is O. In a further embodiment, the methods comprise a compound of formula 42 and the attendant definitions, wherein R″ is H. In a further embodiment, the methods comprise a compound of formula 42 and the attendant definitions, wherein R″ is OH. In a further embodiment, the methods comprise a compound of formula 42 and the attendant definitions, wherein R″ is an ester. In a further embodiment, the methods comprise a compound of formula 42 and the attendant definitions, wherein R1 is

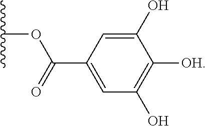

In a further embodiment, the methods comprise a compound of formula 42 and the attendant definitions, wherein R1, R2, R3, R4, R′1, R′2, R′3, R′4, R′S and R″ are H. In a further embodiment, the methods comprise a compound of formula 42 and the attendant definitions, wherein R2, R4, and R′3 are OH. In a further embodiment, the methods comprise a compound of formula 42 and the attendant definitions, wherein R4, R′2, R′3, and R″ are OH. In a further embodiment, the methods comprise a compound of formula 42 and the attendant definitions, wherein R2, R4, R′2, R′3, and R″ are OH. In a further embodiment, the methods comprise a compound of formula 42 and the attendant definitions, wherein R2, R4, R′2, R′3, R′4, and R″ are OH.

In a further embodiment, the methods comprise a compound of formula 42 and the attendant definitions, wherein X and Y are CH; M is O; Z and O; R″ is H; and R1, R2, R3, R4, R′1, R′2, R′3, R′4, R′5 and R″ are H (flavanone). In a further embodiment, the methods comprise a compound of formula 42 and the attendant definitions, wherein X and Y are CH; M is O; Z and O; R″ is H; R2, R4, and R′3 are OH; and R1, R3, R′1, R′2, R′4, and R′5 are H (naringenin). In a further embodiment, the methods comprise a compound of formula 42 and the attendant definitions, wherein X and Y are CH; M is O; Z and O; R″ is OH; R2, R4, R′2, and R′3 are OH; and R1, R3, R′1, R′4, and R′5 are H (3,5,7,3′,4′-pentahydroxyflavanone). In a further embodiment, the methods comprise a compound of formula 42 and the attendant definitions, wherein X and Y are CH; M is H2; Z and O; R″ is OH; R2, R4, R′2, and R′3, are OH; and R1, R3, R′1, R′4 and R′5 are H (epicatechin). In a further embodiment, the methods comprise a compound of formula 42 and the attendant definitions, wherein X and Y are CH; M is H2; Z and O; R″ is OH; R2, R4, R′2, R′3, and R′4 are OH; and R1, R3, R′1, and R′S are H (gallocatechin). In a further embodiment, the methods comprise a compound of formula 42 and the attendant definitions, wherein X and Y are CH; M is H2; Z and O; R″ is

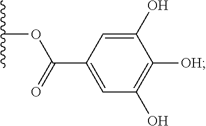

R2, R4, R′2, R′3, R′4, and R″ are OH; and R1, R3, R′1, and R′5 are H (epigallocatechin gallate).

In another embodiment, methods for activating a sirtuin protein comprise an activating compound that is an isoflavanone compound of formula 43:

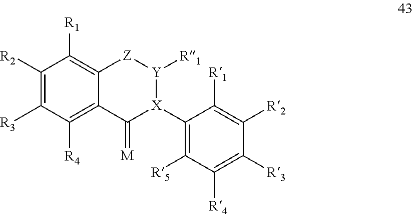

wherein:
R1, R2, R3, R4, R5, R′1, R′2, R′3, R′4, R′5, and R″1, represent H, alkyl, aryl, heteroaryl, alkaryl, heteroaralkyl, halide, NO2, SR, OR, N(R)2, or carboxyl;
R represents H, alkyl, or aryl;
M represents H2, O, NR, or S;
Z represents CR, O, NR, or S; and
X represents CR or N; and
Y represents CR or N.

In another embodiment, methods for activating a sirtuin protein comprise an activating compound that is a flavone compound of formula 44:

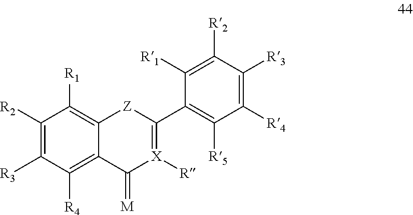

wherein:
R1, R2, R3, R4, R5, R′1, R′2, R′3, R′4, and R′S, represent H, alkyl, aryl, heteroaryl, alkaryl, heteroaralkyl, halide, NO2, SR, OR, N(R)2, or carboxyl;
R″ is absent or represents H, alkyl, aryl, heteroaryl, alkaryl, heteroaralkyl, halide, NO2, SR, OR, N(R)2, or carboxyl;
R represents H, alkyl, or aryl;
M represents H2, O, NR, or S;
Z represents CR, O, NR, or S; and
X represents CR or N when R″ is absent or C when R″ is present.

In a further embodiment, the methods comprise a compound of formula 44 and the attendant definitions, wherein X is C. In a further embodiment, the methods comprise a compound of formula 44 and the attendant definitions, wherein X is CR. In a further embodiment, the methods comprise a compound of formula 44 and the attendant definitions, wherein Z is O. In a further embodiment, the methods comprise a compound of formula 44 and the attendant definitions, wherein M is O. In a further embodiment, the methods comprise a compound of formula 44 and the attendant definitions, wherein R″ is H. In a further embodiment, the methods comprise a compound of formula 44 and the attendant definitions, wherein R″ is OH. In a further embodiment, the methods comprise a compound of formula 44 and the attendant definitions, wherein R1, R2, R3, R4, R5, R′1, R′2, R′3, R′4, and R′5 are H. In a further embodiment, the methods comprise a compound of formula 44 and the attendant definitions, wherein R2, R′2, and R′3 are OH. In a further embodiment, the methods comprise a compound of formula 44 and the attendant definitions, wherein R2, R4, R′2, R′3, and R′4 are OH. In a further embodiment, the methods comprise a compound of formula 44 and the attendant definitions, wherein R2, R4, R′2, and R′3 are OH. In a further embodiment, the methods comprise a compound of formula 44 and the attendant definitions, wherein R3, R′2, and R′3 are OH. In a further embodiment, the methods comprise a compound of formula 44 and the attendant definitions, wherein R2, R4, R′2, and R′3 are OH. In a further embodiment, the methods comprise a compound of formula 44 and the attendant definitions, wherein R2, R′2, R′3, and R′4 are OH. In a further embodiment, the methods comprise a compound of formula 44 and the attendant definitions, wherein R2, R4, and R′3 are OH. In a further embodiment, the methods comprise a compound of formula 44 and the attendant definitions, wherein R2, R3, R4, and R′3 are OH. In a further embodiment, the methods comprise a compound of formula 44 and the attendant definitions, wherein R2, R4, and R′3 are OH. In a further embodiment, the methods comprise a compound of formula 44 and the attendant definitions, wherein R3, R′1, and R′3 are OH. In a further embodiment, the methods comprise a compound of formula 44 and the attendant definitions, wherein R2 and R′3 are OH. In a further embodiment, the methods comprise a compound of formula 44 and the attendant definitions, wherein R1, R2, R′2, and R′3 are OH. In a further embodiment, the methods comprise a compound of formula 44 and the attendant definitions, wherein R3, R′1, and R′2 are OH. In a further embodiment, the methods comprise a compound of formula 44 and the attendant definitions, wherein R′3 is OH. In a further embodiment, the methods comprise a compound of formula 44 and the attendant definitions, wherein R4 and R′3 are OH. In a further embodiment, the methods comprise a compound of formula 44 and the attendant definitions, wherein R2 and R4 are OH. In a further embodiment, the methods comprise a compound of formula 44 and the attendant definitions, wherein R2, R4, R′1, and R′3 are OH. In a further embodiment, the methods comprise a compound of formula 44 and the attendant definitions, wherein R4 is OH. In a further embodiment, the methods comprise a compound of formula 44 and the attendant definitions, wherein R2, R4, R′2, R′3, and R′4 are OH. In a further embodiment, the methods comprise a compound of formula 44 and the attendant definitions, wherein R2, R′2, R′3, and R′4 are OH. In a further embodiment, the methods comprise a compound of formula 44 and the attendant definitions, wherein R1, R2, R4, R′2, and R′3 are OH.

In a further embodiment, the methods comprise a compound of formula 44 and the attendant definitions, wherein X is CH; R″ is absent; Z is O; M is O; and R1, R2, R3, R4, R5, R′1, R′2, R′3, R′4, and R′S are H (flavone). In a further embodiment, the methods comprise a compound of formula 44 and the attendant definitions, wherein X is C; W′ is OH; Z is O; M is O; R2, R′2, and R′3 are OH; and R1, R3, R4, R′1, R′4, and R′5 are H (fisetin). In a further embodiment, the methods comprise a compound of formula 44 and the attendant definitions, wherein X is CH; R″ is absent; Z is O; M is O; R2, R4, R′2, R′3, and R′4 are OH; and R1, R3, R′1, and R′S are H (5,7,3′,4′,5′-pentahydroxyflavone). In a further embodiment, the methods comprise a compound of formula 44 and the attendant definitions, wherein X is CH; R″ is absent; Z is O; M is O; R2, R4, R′2, and R′3 are OH; and R1, R3, R′1, R′4, and R′5 are H (luteolin). In a further embodiment, the methods comprise a compound of formula 44 and the attendant definitions, wherein X is C, R″ is OH; Z is O; M is O; R3, R′2, and R′3 are OH; and R1, R2, R4, R′1, R′4, and R′5 are H (3,6,3′,4′-tetrahydroxyflavone). In a further embodiment, the methods comprise a compound of formula 44 and the attendant definitions, wherein X is C, R″ is OH; Z is O; M is O; R2, R4, R′2, and R′3 are OH; and R1, R3, R′1, R′4, and R′5 are H (quercetin). In a further embodiment, the methods comprise a compound of formula 44 and the attendant definitions, wherein X is CH; R″ is absent; Z is O; M is O; R2, R′2, R′3, and R′4 are OH; and R1, R3, R4, R′1, and R′S are H. In a further embodiment, the methods comprise a compound of formula 44 and the attendant definitions, wherein X is C; R″ is OH; Z is O; M is O; R2, R4, and R′3 are OH; and R1, R3, R′1, R′2, R′4, and R′S are H. In a further embodiment, the methods comprise a compound of formula 44 and the attendant definitions, wherein X is CH; R″ is absent; Z is O; M is O; R2, R3, R4, and R′3 are OH; and R1, R′1, R′2, R′4, and R′S are H. In a further embodiment, the methods comprise a compound of formula 44 and the attendant definitions, wherein X is CH; R″ is absent; Z is O; M is O; R2, R4, and R′3 are OH; and R1, R3, R′1, R′2, R′4, and R′S are H. In a embodiment, the methods comprise a compound of formula 44 and the attendant definitions, wherein X is C, R″ is OH; Z is O; M is O; R3, R′1, and R′3 are OH; and R1, R2, R4, R′2, R′4, and R′5 are H. In a further embodiment, the methods comprise a compound of formula 44 and the attendant definitions, wherein X is CH; R″ is absent; Z is O; M is O; R2 and R′3 are OH; and R1, R3, R4, R′1, R′2, R′4, and R′S are H. In a further embodiment, the methods comprise a compound of formula 44 and the attendant definitions, wherein X is C, R″ is OH; Z is O; M is O; R1, R2, R′2, and R′3 are OH; and R1, R2, R4, R′3, R′4, and R′5 are H. In a further embodiment, the methods comprise a compound of formula 44 and the attendant definitions, wherein X is C; R″ is OH; Z is O; M is O; R3, R′1, and R′2 are OH; and R1, R2, R4; R′3, R′4, and R′5 are H. In a further embodiment, the methods comprise a compound of formula 44 and the attendant definitions, wherein X is CH; R″ is absent; Z is O; M is O; R′3 is OH; and R1, R2, R3, R4, R′1, R′2, R′4, and R′5 are H. In a further embodiment, the methods comprise a compound of formula 44 and the attendant definitions, wherein X is CH; R″ is absent; Z is O; M is O; R4 and R′3 are OH; and R1, R2, R3, R′1, R′2, R′4, and R′5 are H. In a further embodiment, the methods comprise a compound of formula 44 and the attendant definitions, wherein X is CH; R″ is absent; Z is O; M is O; R2 and R4 are OH; and R1, R3, R′1, R′2, R′3, R′4, and R′5 are H. In a further embodiment, the methods comprise a compound of formula 44 and the attendant definitions, wherein X is C; R″ is OH; Z is O; M is O; R2, R4, R′1, and R′3 are OH; and R1, R3, R′2, R′4, and R′5 are H. In a further embodiment, the methods comprise a compound of formula 44 and the attendant definitions, wherein X is CH; R″ is absent; Z is O; M is O; R4 is OH; and R1, R2, R3, R′1, R2, R′3, R′4, and R′5 are H. In a further embodiment, the methods comprise a compound of formula 44 and the attendant definitions, wherein X is C; R″ is OH; Z is O; M is O; R2, R4, R′2, R′3, and R′4 are OH; and R1, R3, R′1, and R′5 are H. In a further embodiment, the methods comprise a compound of formula 44 and the attendant definitions, wherein X is C; R″ is OH; Z is O; M is O; R2, R′2, R′3, and R′4 are OH; and R1, R3, R4, R′1, and R′5 are H. In a further embodiment, the methods comprise a compound of formula 44 and the attendant definitions, wherein X is C; R″ is OH; Z is O; M is O; R1, R2, R4, R′2, and R′3 are OH; and R3, R′1, R′4, and R′5 are H.

In another embodiment, methods for activating a sirtuin protein comprise an activating compound that is an isoflavone compound of formula 45:

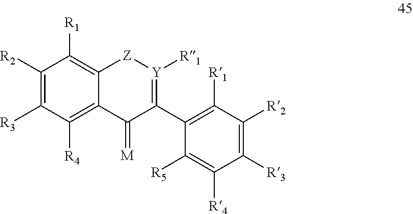

wherein:
R1, R2, R3, R4, R5, R′1, R′2, R′3, R′4, and R′5, represent H, alkyl, aryl, heteroaryl, alkaryl, heteroaralkyl, halide, NO2, SR, OR, N(R)2, or carboxyl;
R″ is absent or represents H, alkyl, aryl, heteroaryl, alkaryl, heteroaralkyl, halide, NO2, SR, OR, N(R)2, or carboxyl;
R represents H, alkyl, or aryl;
M represents H2, O, NR, or S;
Z represents CR, O, NR, or S; and
Y represents CR or N when R″ is absent or C when R″ is present.

In a further embodiment, the methods comprise a compound of formula 45 and the attendant definitions, wherein Y is CR. In a further embodiment, the methods comprise a compound of formula 45 and the attendant definitions, wherein Y is CH. In a further embodiment, the methods comprise a compound of formula 45 and the attendant definitions, wherein Z is O. In a further embodiment, the methods comprise a compound of formula 45 and the attendant definitions, wherein M is O. In a further embodiment, the methods comprise a compound of formula 45 and the attendant definitions, wherein R2 and R′3 are OH. In a further embodiment, the methods comprise a compound of formula 45 and the attendant definitions, wherein R2, R4, and R′3 are OH.

In a further embodiment, the methods comprise a compound of formula 45 and the attendant definitions, wherein Y is CH; R″ is absent; Z is O; M is O; R2 and R′3 are OH; and R1, R3, R4, R′1, R′2, R′4, and R′5 are H. In a further embodiment, the methods comprise a compound of formula 45 and the attendant definitions, wherein Y is CH; R″ is absent; Z is O; M is O; R2, R4, and R′3 are OH; and R1, R3, R′1, R′2, R′4, and R′S and H.

In another embodiment, methods for activating a sirtuin protein comprise an activating compound that is an anthocyanidin compound of formula 46:

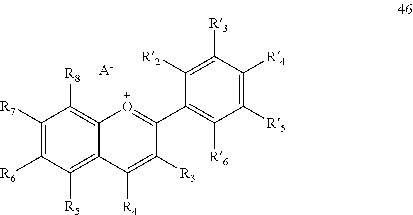

wherein:
R3, R4, R5, R6, R7, R8, R′2, R′3, R′4, R′5, and R′6 represent H, alkyl, aryl, heteroaryl, alkaryl, heteroaralkyl, halide, NO2, SR, OR, N(R)2, or carboxyl;
R represents H, alkyl, or aryl; and
A⁻ represents an anion selected from the following: Cl⁻, Br⁻, or I⁻.

In a further embodiment, the methods comprise a compound of formula 46 and the attendant definitions, wherein A⁻ is Cr. In a further embodiment, the methods comprise a compound of formula 46 and the attendant definitions, wherein R3, R5, R7, and R′4 are OH. In a further embodiment, the methods comprise a compound of formula 46 and the attendant definitions, wherein R3, R5, R7, R′3, and R′4 are OH. In a further embodiment, the methods comprise a compound of formula 46 and the attendant definitions, wherein R3, R5, R7, R′3, R′4, and R′5 are OH.

In a further embodiment, the methods comprise a compound of formula 46 and the attendant definitions, wherein A⁻ is Cl⁻; R3, R5, R7, and R′4 are OH; and R4, R6, R8, R′2, R′3, R′5, and R′6 are H. In a further embodiment, the methods comprise a compound of formula 46 and the attendant definitions, wherein A⁻ is Cl⁻; R3, R5, R7, R′3, and R′4 are OH; and R4, R6, R8, R′2, R′5, and R′6 are H. In a further embodiment, the methods comprise a compound of formula 46 and the attendant definitions, wherein A⁻ is Cl⁻; R3, R5, R7, R′3, R′4, and R′5 are OH; and R4, R6, R8, R′2, and R′6 are H.

Methods for activating a sirtuin protein may also comprise a stilbene, chalcone, or flavone compound represented by formula 47:

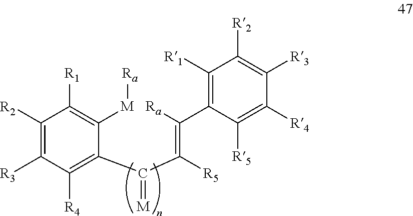

wherein:
M is absent or O;
R1, R2, R3, R4, R5, R′1, R′2, R′3, R′4, and R′5 represent H, alkyl, aryl, heteroaryl alkaryl, heteroaralkyl, halide, NO2, SR, OR, N(R)2, or carboxyl;
Ra represents H or the two Ra form a bond;
R represents H, alkyl, or aryl; and
n is 0 or 1.

In a further embodiment, the methods comprise an activating compound represented by formula 47 and the attendant definitions, wherein n is 0. In a further embodiment, the methods comprise an activating compound represented by formula 47 and the attendant definitions, wherein n is 1. In a further embodiment, the methods comprise an activating compound represented by formula 47 and the attendant definitions, wherein M is absent. In a further embodiment, the methods comprise an activating compound represented by formula 47 and the attendant definitions, wherein M is O. In a further embodiment, the methods comprise an activating compound represented by formula 47 and the attendant definitions, wherein Ra is H. In a further embodiment, the methods comprise an activating compound represented by formula 47 and the attendant definitions, wherein M is O and the two Ra form a bond.

In a further embodiment, the methods comprise an activating compound represented by formula 47 and the attendant definitions, wherein R5 is H. In a further embodiment, the methods comprise an activating compound represented by formula 47 and the attendant definitions, wherein R5 is OH. In a further embodiment, the methods comprise an activating compound represented by formula 47 and the attendant definitions, wherein R1, R3, and R′3 are OH. In a further embodiment, the methods comprise an activating compound represented by formula 47 and the attendant definitions, wherein R2, R4, R′2, and R′3 are OH. In a further embodiment, the methods comprise an activating compound represented by formula 47 and the attendant definitions, wherein R2, R′2, and R′3 are OH. In a further embodiment, the methods comprise an activating compound represented by formula 47 and the attendant definitions, wherein R2 and R4 are OH.

In a further embodiment, the methods comprise an activating compound represented by formula 47 and the attendant definitions, wherein n is 0; M is absent; Ra is H; R5 is H; R1, R3, and R′3 are OH; and R2, R4, R′1, R′2, R′4, and R′S are H. In a further embodiment, the methods comprise an activating compound represented by formula 47 and the attendant definitions, wherein n is 1; M is absent; Ra is H; R5 is H; R2, R4, R′2, and R′3 are OH; and R1, R3, R′1, R′4, and R′5 are H. In a further embodiment, the methods comprise an activating compound represented by formula 47 and the attendant definitions, wherein n is 1; M is O; the two Ra form a bond; R5 is OH; R2, R′2, and R′3 are OH; and R1, R3, R4, R′1, R′4, and R′5 are H.

Other compounds for activating sirtuin deacetylase protein family members include compounds having a formula of any one of formulas 48-66, set forth below.

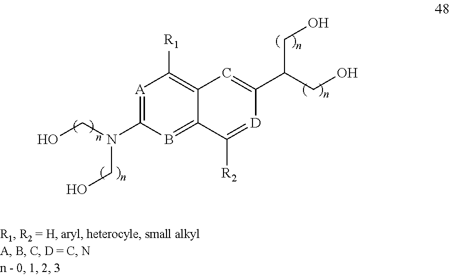

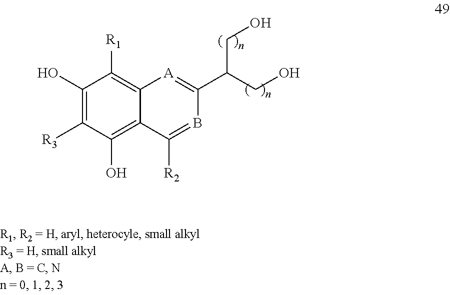

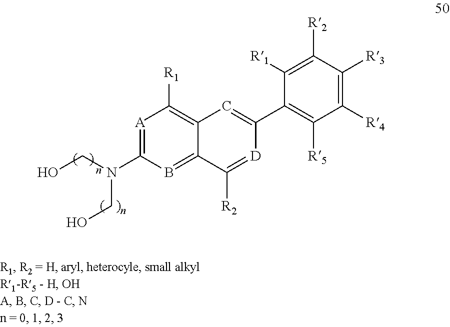

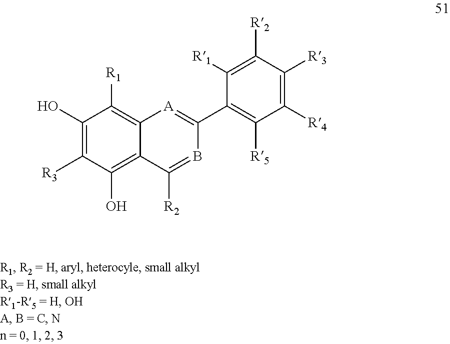

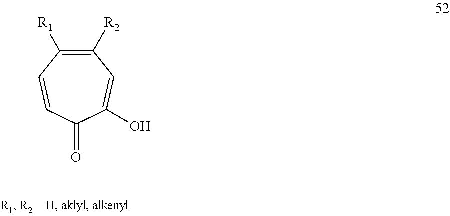

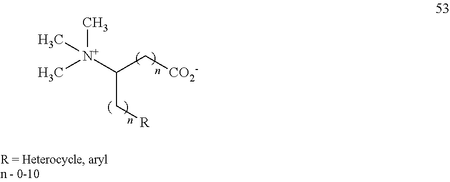

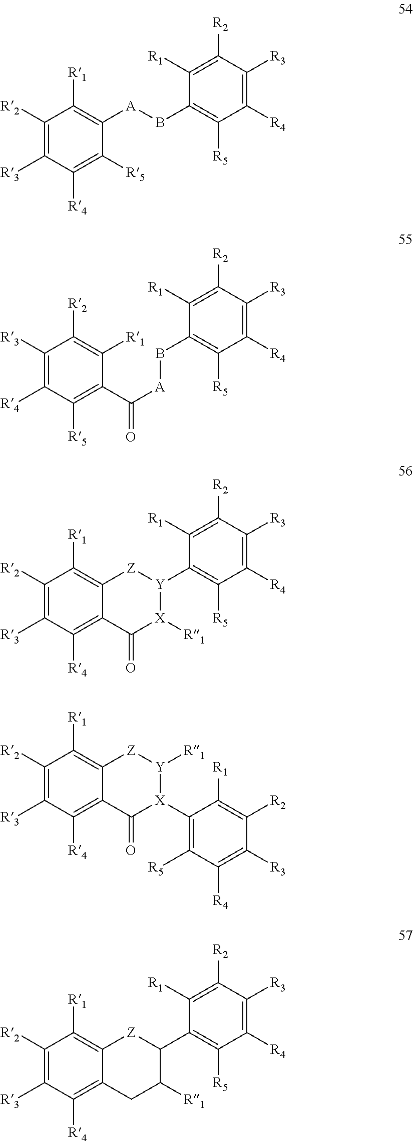

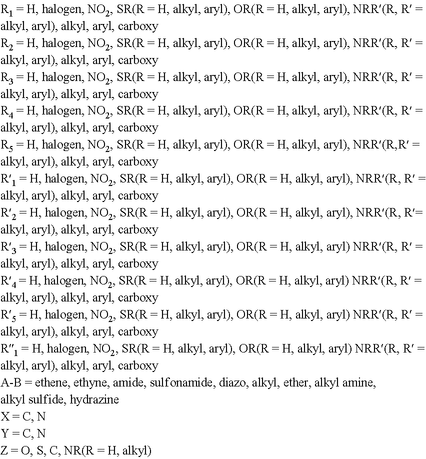

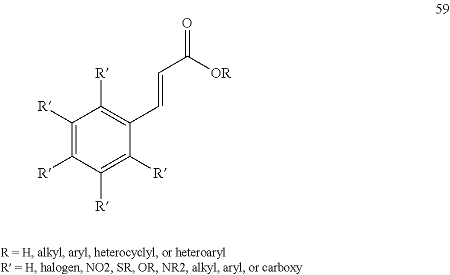

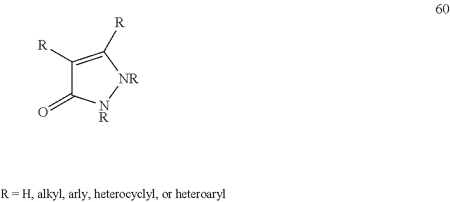

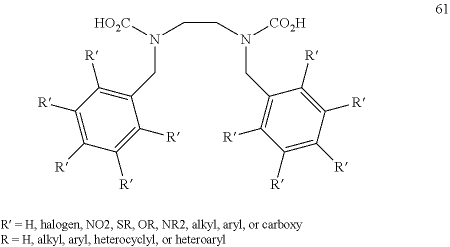

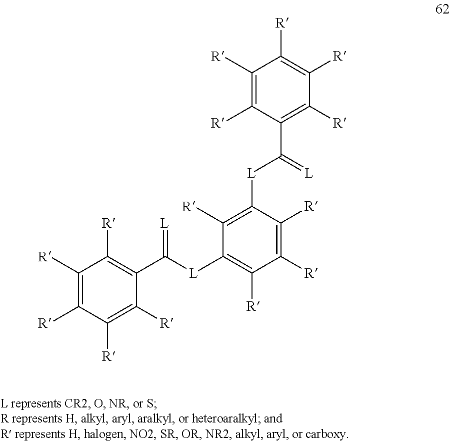

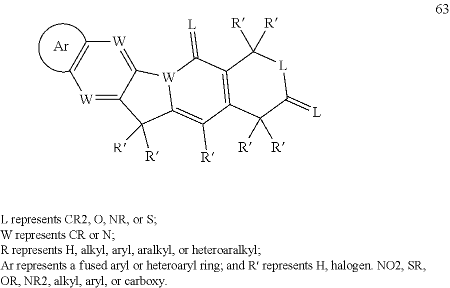

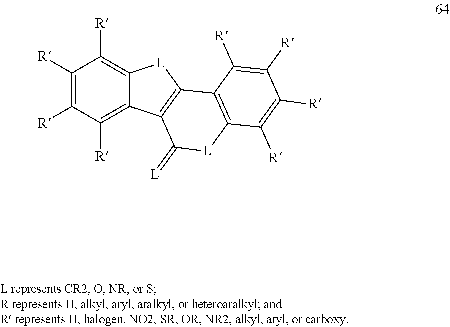

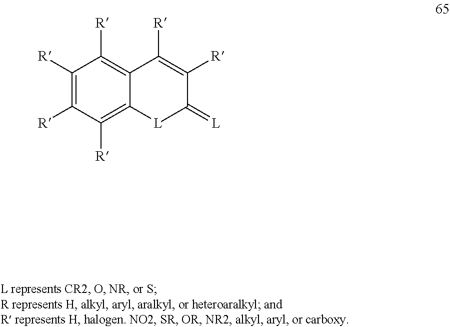

Methods for activating a sirtuin protein may also comprise a stilbene, chalcone, or flavone compound represented by formula 66:

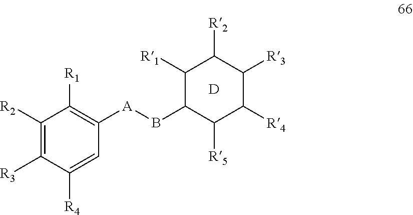

wherein:
D is a phenyl or cyclohexyl group;

R1, R2, R3, R4, R5, R′1, R′2, R′3, R′4, and R′S represent H, alkyl, aryl, heteroaryl, alkaryl, heteroaralkyl, halide, NO2, SR, OR, N(R)₂, carboxyl, azide, ether; or any two adjacent R or R′ groups taken together form a fused benzene or cyclohexyl group;
R represents H, alkyl, or aryl; and
A-B represents an ethylene, ethenylene, or imine group.

In particular embodiments, a sirtuin-activating compound may be selected from the group consisting of dipyridamole, hinokitiol; L-(+)-ergothioneine; and caffeic acid phenol ester.

Sirtuin-activating compounds similar to, as well as identical to, those of 41-66 are disclosed in WO 2006/096780 and related WO 2005/002555, WO 2005/002672, US 2005/0136537 and US 2006/0025337, each of which is incorporated herein by reference in its entirety. Definitions for compounds 67-118 are applicable to the compounds of formulas 41-66 as well. Such compounds include formulas 67-118, shown below.

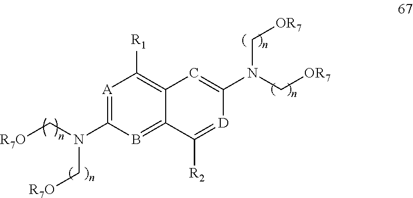

wherein:
R₁ and R₂ represent H, aryl, heterocycle, or small alkyl;
R₇ represents H, alkyl, aryl, heteroaryl, aralkyl, —SO₃H, monosaccharide, oligosaccharide, glycopyranosyl, glycopyranosyl, glucuronosyl, or glucuronide;
A, B, C, and D represent CR₁ or N;
and n is 0, 1, 2, or 3;

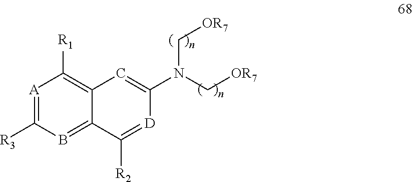

wherein:
R₁ and R₂ represent H, aryl, heterocycle, or small alkyl;
R₃ represents small alkyl;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C, and D represent CR₁ or N; and n is 0, 1, 2, or 3;

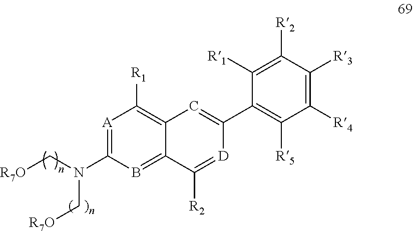

wherein:
R₁ and R₂ represent H, aryl, heterocycle, or small alkyl;
R′₁, R′₂, R′₃, R′₄, and R′₅ represent H or OR₇;
R₇ represents H, alkyl, aryl, heteroaryl, aralkyl, —SO₃H, monosaccharide, oligosaccharide, glucofuranosyl, glycopyranosyl, glucuronosyl, or glucuronide;
A, B, C, and D represent CR₁ or N; and n is 0, 1, 2, or 3;

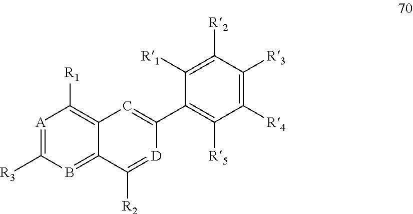

wherein:
R₁ and R₂ represent H, aryl, heterocycle, or small alkyl;
R₃ represents small alkyl;
R′₁, R′₂, R′₃, R′₄, and R′₅ represent H or OR₇;
R₇ represents H, alkyl, aryl, heteroaryl, aralkyl, —SO₃H, monosaccharide, oligosaccharide, glycofuranosyl, glycopyranosyl, glucuronosyl, or glucuronide;
A, B, C and D represent CR₁ or N; and n is 0, 1, 2, or 3;

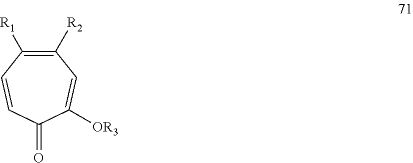

wherein:
R₁ and R₂ represent H, aryl, or alkenyl; and
R₇ represents H, —SO₃H, monosaccharide, oligosaccharide, glycofuranosyl, glycopyranosyl, glucuronosyl, or glucuronide;

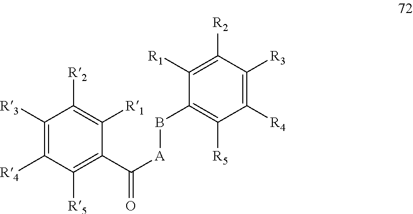

wherein:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide; and
A-B represents ethene, ethyne, amide, sulfonamide, diazo, alkyl, ether, alkyl amine, alkyl sulfide, hydroxyamine, or hydrazine;

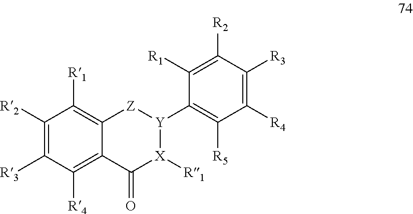

wherein:
R₁, R₂, R₃, R₄, R₅, R′₁, R′2, R′3, R′4, and R′₅ represents H, halogen, NO₂, SH, SR, OH, OR, NRR′, alkyl, aryl or carboxy;
R represents H₅ alkyl, aryl, heteroaryl, aralkyl, —SO3H, monosaccharide, oligosaccharide, glycofuranosyl, glycopyranosyl, glucuronosyl, or glucuronide; R′ represents H, alkyl, aryl, heteroaryl, aralkyl, —SO3H, monosaccharide, oligosaccharide, glycofuranosyl, glycopyranosyl, glucuronosyl, or glucuronide;
X represents CR₈ or N; Y represents CRs or N; Z represents O, S, C(R8)₂, or NR₈;
and R₈ represents alkyl, aryl or aralkyl;

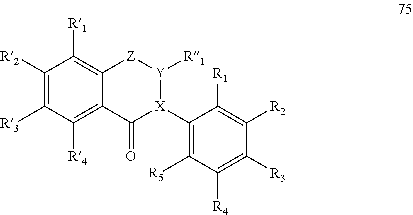

wherein:
R₁, R₂, R₃, R₄, R₅, R′1, R′2, R′3, R′4, and R′5 represents H, halogen, NO₂, SH, SR, OH, OR, NRR′, alkyl, aryl or carboxy;
R represents H, alkyl, aryl, heteroaryl, aralkyl, —SO₃H₃ monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide; X represents CR₈ or N;
Y represents CR₈ or N; Z represents O, S, C(R₈)₂, or NR₈; and R₈ represents alkyl, aryl or aralkyl;

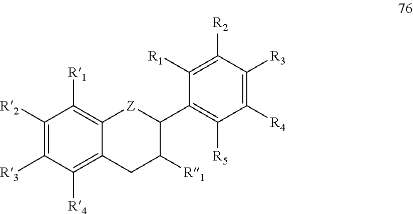

wherein:
R₁, R₂, R₃, R₄, R₅, R′₁, R′₂, R′₃, R′₄, and R′₅ represents H, halogen, NO₂, SH, SR, OH, OR, NRR′, alkyl, aryl or carboxy;
R represents H, alkyl, aryl, heteroaryl, aralkyl, —SO₃H, monosaccharide, oligosaccharide, glycofuranosyl, glycopyranosyl, glucuronosyl, or glucuronide;
R′ represents H, alkyl, aryl, heteroaryl, aralkyl, —SO₃H, monosaccharide, oligosaccharide, glycofuranosyl, glycopyranosyl, glucuronosyl, or glucuronide; Z represents O, S, C(R₈)₂, or NR₈; and
R₈ represents alkyl, aryl or aralkyl;

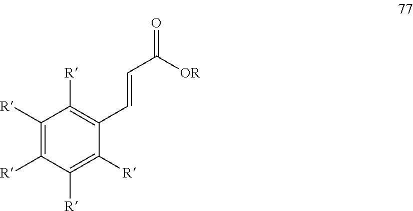

wherein:
R is H, alkyl, aryl, heterocyclyl, heteroaryl, aralkyl, —SO₃H, monosaccharide, oligosaccharide, glycofuranosyl, glycopyranosyl, glucuronosyl, or glucuronide; and
R′ is H, halogen, NO₂, SR, OR₅ NR₂, alkyl, aryl, or carboxy;

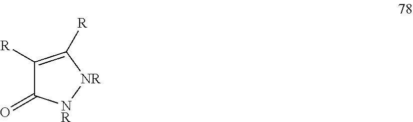

wherein:
R is H, alkyl, aryl, heterocyclyl, heteroaryl, aralkyl, —SO₃H, monosaccharide, oligosaccharide, glycofuranosyl, glycopyranosyl, glucuronosyl, or glucuronide;

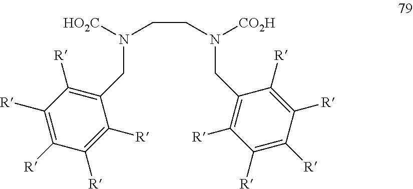

wherein:
R′ is H, halogen, NO₂, SR, OR, NR₂, alkyl, aryl, aralkyl, or carboxy; and R is H, alkyl, aryl, heterocyclyl, heteroaryl, aralkyl, —SO₃H, monosaccharide, oligosaccharide, glycofuranosyl, glycopyranosyl, glucuronosyl, or glucuronide;

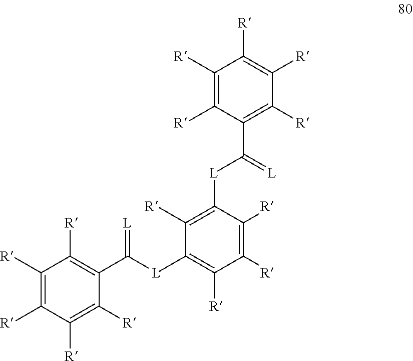

wherein:
L represents CR₂, O, NR, or S; R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy;

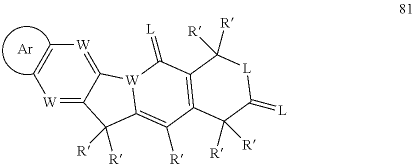

wherein:
L represents CR₂, O, NR, or S; W represents CR or N;
R represents H, alkyl, aryl, aralkyl, heteroaralkyl, —SO₃H, monosaccharide, oligosaccharide, glycofuranosyl, glycopyranosyl, glucuronosyl, or glucuronide;
Ar represents a fused aryl or heteroaryl ring; and
R′ represents H, halogen, NO₂, SR, OR, NR₂, alkyl, aryl, aralkyl, or carboxy;

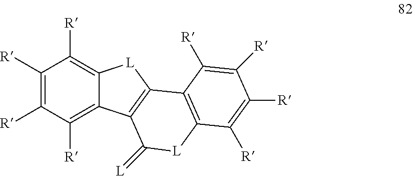

wherein:
L represents CR₂, O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy.

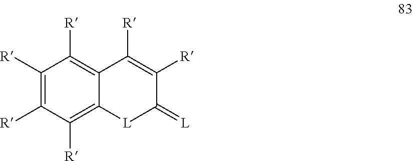

wherein:
L represents CR₂, O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, OR, NR₂, alkyl, aryl, aralkyl, or carboxy.

Methods for activating a sirtuin protein may also comprise using a stilbene, chalcone, or flavone compound represented by formula 84:

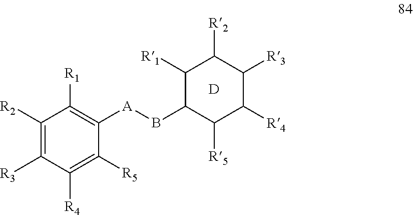

wherein:
D is a phenyl or cyclohexyl group;
R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R₁, R₂, R₃, R₄, R₅, R′i, R′₂, R′₃, R′₄, or R′₅ groups taken together form a fused benzene or cyclohexyl group;
R represents H, alkyl, aryl, aralkyl, —SO₃H, monosaccharide, oligosaccharide, glycofuranosyl, glycopyranosyl, glucuronosyl, or glucuronide; and A-B represents an ethylene, ethenylene, or imine group.

In another embodiment, methods for activating a sirtuin protein comprise using a compound of formula 85:

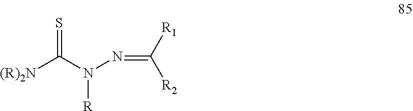

wherein:
R is H, or a substituted or unsubstituted alkyl, aryl, aralkyl, heterocyclyl, heterocyclylalkyl, heteroaryl, or heteroaralkyl; and
R₁ and R₂ are a substituted or unsubstituted alkyl, aryl, aralkyl, heterocyclyl, heterocyclylalkyl, heteroaryl, or heteroaralkyl.

In another embodiment, methods for activating a sirtuin protein comprise using a compound of formula 86:

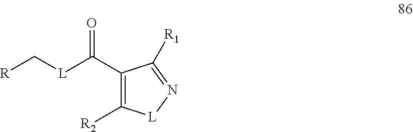

wherein:
R is H, or a substituted or unsubstituted alkyl, alkenyl, or alkynyl;
R₁ and R₂ are a substituted or unsubstituted alkyl, aryl, aralkyl, heterocyclyl, heterocyclylalkyl, heteroaryl, or heteroaralkyl; and L is O, S, or NR.

In another embodiment, methods for activating a sirtuin protein comprise using a compound of formula 87:

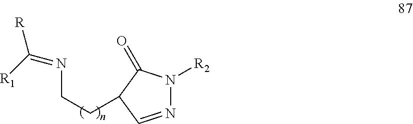

wherein:
R, R₁, and R₂ are H, or a substituted or unsubstituted alkyl, aryl, aralkyl, heterocyclyl, heterocyclylalkyl, heteroaryl, or heteroaralkyl; and
n is an integer from 0 to 5 inclusive. In a further embodiment, the methods comprise a compound of formula 34 and the attendant definitions wherein R is 3,5-dichloro-2-hydroxyphenyl.

In another embodiment, methods for activating a sirtuin protein comprise using a compound of formula 88:

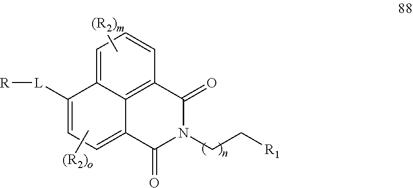

wherein:
R is H or a substituted or unsubstituted alkyl, aryl, aralkyl, heterocyclyl, heterocyclylalkyl, heteroaryl, or heteroaralkyl;
R1 is a substituted or unsubstituted alkyl, aryl, aralkyl, heterocyclyl, heterocyclylalkyl, heteroaryl, or heteroaralkyl;
R₂ is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₃ is alkyl, —SO₃H, monosaccharide, oligosaccharide, glycofuranosyl, glycopyranosyl, glucuronosyl, or glucuronide;
L is O, NR, or S; m is an integer from 0 to 3 inclusive; n is an integer from 0 to 5 inclusive; and o is an integer from 0 to 2 inclusive.

In another embodiment, methods for activating a sirtuin protein comprise using a compound of formula 89:

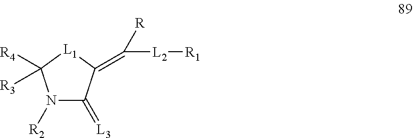

wherein:
R, R₃, and R₄ are H, hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₅ is alkyl, —SO₃H, monosaccharide, oligosaccharide, glycofuranosyl, glycopyranosyl, glucuronosyl, or glucuronide;
R1 and R₂ are H or a substituted or unsubstituted alkyl, aryl, aralkyl, heterocyclyl, heterocyclylalkyl, heteroaryl, heteroaralkyl;
L1 is O, NR1, S, C(R)₂, or SO₂;
and L₂ and L₃ are O, NR₁, S, or C(R)₂.

In another embodiment, methods for activating a sirtuin protein comprise using a compound of formula 90:

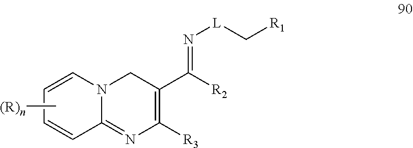

wherein:
R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, heteroaralkyl;
R₁ is H or a substituted or unsubstituted alkyl, aryl, aralkyl, heterocyclyl, heterocyclylalkyl, heteroaryl, heteroaralkyl;
R₂ and R₃ are H or a substituted or unsubstituted alkyl, aryl, aralkyl, heterocyclyl, heterocyclylalkyl, heteroaryl, heteroaralkyl;
R₄ is alkyl, —SO₃H, monosaccharide, oligosaccharide, glycofuranosyl, glycopyranosyl, glucuronosyl, or glucuronide; L is O, NR1, or S; and n is an integer from 0 to 4 inclusive.

In another embodiment, methods for activating a sirtuin protein comprise using a compound of formula 91:

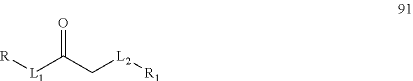

wherein:
R and R1 are H or a substituted or unsubstituted alkyl, aryl, aralkyl, heterocyclyl, heterocyclylalkyl, heteroaryl, or heteroaralkyl; and
L1 and L₂ are O, NR, or S.

In another embodiment, methods for activating a sirtuin protein comprise using a compound of formula 92:

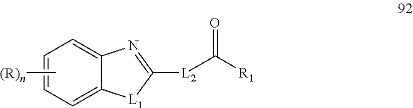

wherein:
R is H, hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R1 is H or a substituted or unsubstituted alkyl, aryl, alkaryl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide;
L₁ and L₂ are O, NR, or S; and n is an integer from 0 to 4 inclusive.

In another embodiment, methods for activating a sirtuin protein comprise using a compound of formula 93:

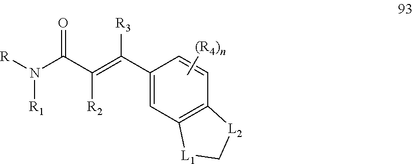

wherein:
R, R1, R₂, R₃ are H or a substituted or unsubstituted alkyl, aryl, alkaryl, heterocyclyl, heterocyclylalkyl, heteroaryl, or heteroaralkyl;
R₄ is hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide;
L₁ and L₂ are O, NR, or S; and n is an integer from 0 to 3 inclusive.

In another embodiment, methods for activating a sirtuin protein comprise using a compound of formula 94:

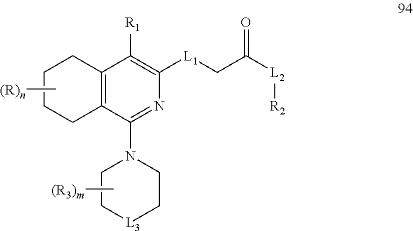

wherein:
R, R1, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is H or a substituted or unsubstituted alkyl, aryl, aralkyl, heterocyclyl, heterocyclylalkyl, heteroaryl, or heteroaralkyl;
R₄ is alkyl, —SO3H, monosaccharide, oligosaccharide, glycofuranosyl, glycopyranosyl, glucuronosyl, or glucuronide;
L1, L₂, and L₃ are O, NR₂, or S; and
m and n are integers from 0 to 8 inclusive.

In another embodiment, methods for activating a sirtuin protein comprise using a compound of formula 95:

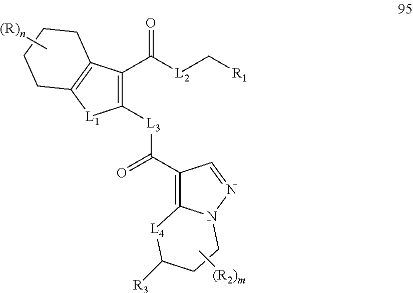

wherein:
R and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R1 and R₃ are H or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L₁, L₂, L₃, and L₄ are O, NR1, or S; m is an integer from 0 to 6 inclusive; and n is an integer from 0 to 8 inclusive.

In another embodiment, methods for activating a sirtuin protein comprise using a compound of formula 96:

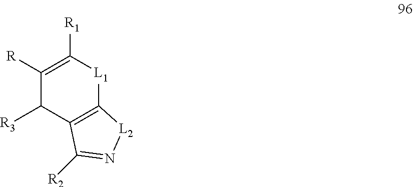

wherein:
R and R₁ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ and R₃ are H or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide; and
L1 and L₂ are O, NR₂, or S.

In another embodiment, methods for activating a sirtuin protein comprise using a compound of formula 97:

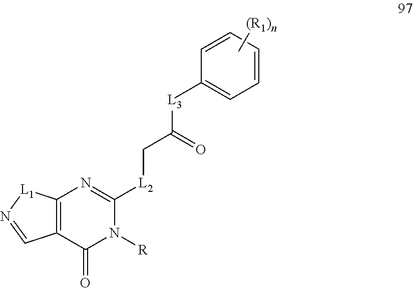

wherein:
R is H or a substituted or unsubstituted alkyl, aryl, aralkyl, heterocyclyl, heterocyclylalkyl, heteroaryl, or heteroaralkyl;
R1 is hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide;
L₁, L₂, and L₃ are O, NR, or S; and n is an integer from 0 to 5 inclusive.

In another embodiment, methods for activating a sirtuin protein comprise using a compound of formula 98:

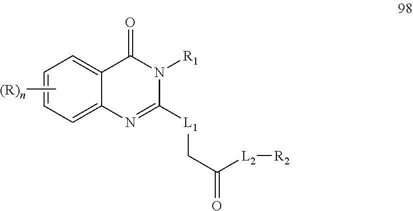

wherein:
R is hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R1 and R₂ are H or a substituted or unsubstituted alkyl, aryl, aralkyl, heterocyclyl, heterocyclylalkyl, heteroaryl, or heteroaralkyl;
R₃ is alkyl, —SO₃H, monosaccharide, oligosaccharide, glycofuranosyl, glycopyranosyl, glucuronosyl, or glucuronide;
L1 and L₂ are O, NR1, or S; and n is an integer from 0 to 4 inclusive.

In another embodiment, methods for activating a sirtuin protein comprise using a compound of formula 99:

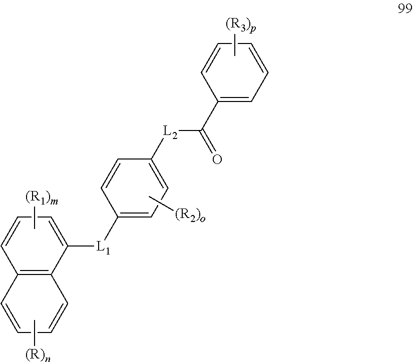

wherein:
R, R1, R₂, and R₃ are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide;
L1 and L₂ are O, NR₄, or S;
R₄ is H or a substituted or unsubstituted alkyl, aryl, aralkyl, heterocyclyl, heterocyclylalkyl, heteroaryl, or heteroaralkyl; n is an integer from 0 to 4 inclusive; m is an integer from 0 to 3 inclusive; o is an integer from 0 to 4 inclusive; and p is an integer from 0 to 5 inclusive.

In another embodiment, methods for activating a sirtuin protein comprise using a compound of formula 100:

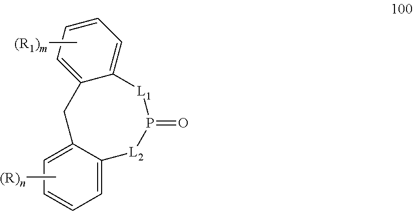

wherein:
R and R1 are hydroxy, amino, cyano, halide, OR₅, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L1 and L₂ are O, NR₄, or S; R₄ is H or a substituted or unsubstituted alkyl, aryl, aralkyl, heterocyclyl, heterocyclylalkyl, heteroaryl, or heteroaralkyl;
R₅ is alkyl, —SO₃H, monosaccharide, oligosaccharide, glycofuranosyl, glycopyranosyl, glucuronosyl, or glucuronide; and m and n are integers from 0 to 4 inclusive.

In another embodiment, methods for activating a sirtuin protein comprise using a compound of formula 101:

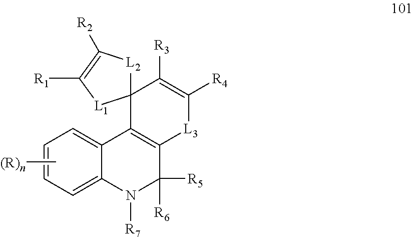

wherein:
R, R1, R₂, R₃, R₄, R₅, and R₆ are hydroxy, amino, cyano, halide, OR_{B}, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₇ is H or a substituted or unsubstituted alkyl, acyl, aryl, aralkyl, heterocyclyl, heterocyclylalkyl, heteroaryl, or heteroaralkyl;
R8 is alkyl, —SO₃H, monosaccharide, oligosaccharide, glycofuranosyl, glycopyranosyl, glucuronosyl, or glucuronide;
L₁, L₂, and L₃ are O, NR₇, or S and n is an integer from 0 to 4 inclusive.

In another embodiment, methods for activating a sirtuin protein comprise using a compound of formula 102:

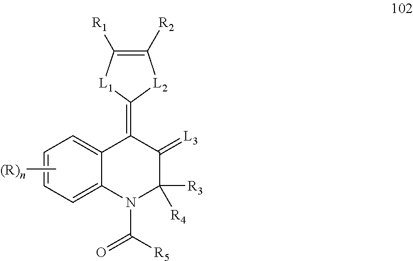

wherein:
R, R1, R₂, R₃, R₄, and R₅ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L1, L₂, and L₃ are O, NR₆, or S;
R₆ is H or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide; and n is an integer from 0 to 4 inclusive.

In another embodiment, methods for activating a sirtuin protein comprise using a compound of formula 103:

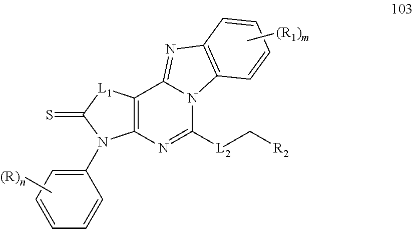

wherein:
R and R1 are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L1 and L₂ are O, NR₃, or S; R₃ is H or a substituted or unsubstituted alkyl, aryl, aralkyl, heterocyclyl, heterocyclylalkyl, heteroaryl, or heteroaralkyl;
n is an integer from 0 to 5 inclusive; and m is an integer from 0 to 4 inclusive.

In another embodiment, methods for activating a sirtuin protein comprise using a compound of formula 104:

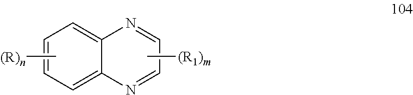

wherein:
R and R1 are hydroxy, amino, cyano, halide, OR₂, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkyl, —SO₃H, monosaccharide, oligosaccharide, glycofuranosyl, glycopyranosyl, glucuronosyl, or glucuronide; n is an integer from 0 to 4 inclusive; and m is an integer from 0 to 2 inclusive.

In another embodiment, methods for activating a sirtuin protein comprise a compound of formula 105:

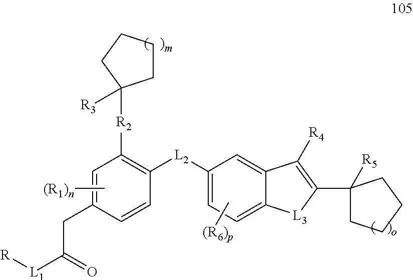

wherein:
R is H or a substituted or unsubstituted alkyl, aryl, aralkyl, heterocyclyl, heterocyclylalkyl, heteroaryl, or heteroaralkyl;
R1 and R₆ are hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₂ is alkylene, alkenylene, or alkynylene;
R₃, R₄, and R₅ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide;
L1, L₂, and L₃ are O, NR, or S; n and p are integers from 0 to 3 inclusive; and m and o are integers from 0 to 2 inclusive.

In another embodiment, methods for activating a sirtuin protein comprise using a compound of formula 106:

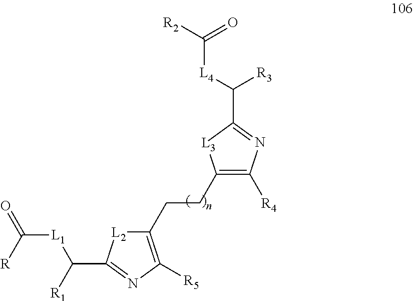

wherein:
R, R₁, R₂, R₃, R₄, and R₅ are H, hydroxy, amino, cyano, halide, OR₇, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
L1, L₂, L₃, and L₄ are O, NR₆, or S;
R₆ is and H, or a substituted or unsubstituted alkyl, aryl, aralkyl, heterocyclyl, heterocyclylalkyl, heteroaryl, or heteroaralkyl;
R₇ is alkyl, —SO₃H, monosaccharide, oligosaccharide, glycofuranosyl, glycopyranosyl, glucuronosyl, or glucuronide; and
n is an integer from 0 to 5 inclusive.

In another embodiment, methods for activating a sirtuin protein comprise using a compound of formula 107:

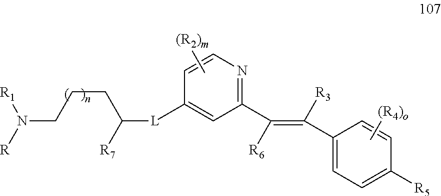

wherein:
R and R1 are H or a substituted or unsubstituted alkyl, aryl, aralkyl, heterocyclyl, heterocyclylalkyl, heteroaryl, or heteroaralkyl;
R₂, R₄, and R₅ are hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₃, R₆, and R₇ are H, hydroxy, amino, cyano, halide, OR₈, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₈ is alkyl, —SO₃H, monosaccharide, oligosaccharide, glycofuranosyl, glycopyranosyl, glucuronosyl, or glucuronide;
L is O, NR, or S;
n and o are integers from 0 to 4 inclusive; and m is an integer from 0 to 3 inclusive.

In another embodiment, methods for activating a sirtuin protein comprise using a compound of formula 108:

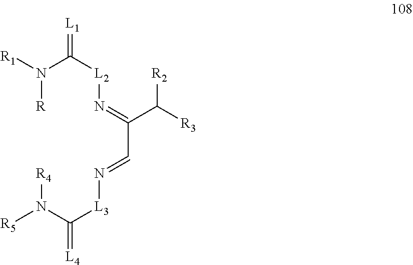

wherein:
R, R1, R₄, and R₅ are H or a substituted or unsubstituted alkyl, aryl, aralkyl, heterocyclyl, heterocyclylalkyl, heteroaryl, or heteroaralkyl;
R₂ and R₃ are H, hydroxy, amino, cyano, halide, OR6, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R6 is alkyl, —SO₃H, monosaccharide, oligosaccharide, glycofuranosyl, glycopyranosyl, glucuronosyl, or glucuronide; and
L1, L₂, L₃, and L₄ are O, NR, or S.

In another embodiment, methods for activating a sirtuin protein comprise using a compound of formula 109:

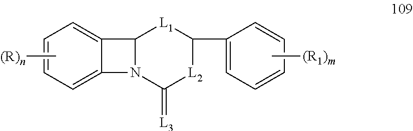

wherein:
R and R₁ are hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl; R₃ is alkyl, —SO₃H, monosaccharide, oligosaccharide, glycofuranosyl, glycopyranosyl, glucuronosyl, or glucuronide;
L1, L₂, and L₃ are O, NR₂, or S;
R₂ is H or a substituted or unsubstituted alkyl, aryl, aralkyl, heterocyclyl, heterocyclylalkyl, heteroaryl, or heteroaralkyl;
n is an integer from 0 to 4 inclusive; and m is an integer from 0 to 5 inclusive.

In another embodiment, methods for activating a sirtuin protein comprise using a compound of formula 110:

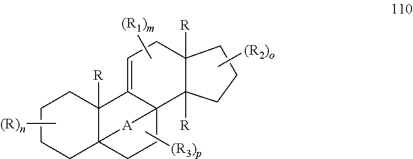

wherein:
R, R1, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₃ is alkyl, —SO₃H, monosaccharide, oligosaccharide, glycofuranosyl, glycopyranosyl, glucuronosyl, or glucuronide;

A is alkylene, alkenylene, or alkynylene; n is an integer from 0 to 8 inclusive; m is an integer from 0 to 3 inclusive; o is an integer from 0 to 6 inclusive; and p is an integer from 0 to 4 inclusive.

In another embodiment, methods for activating a sirtuin protein comprise using a compound of formula 111:

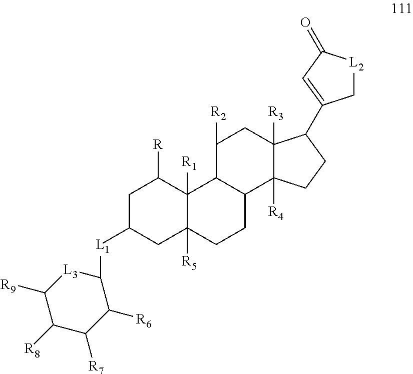

wherein:
R, R1, R₂, R₃, R₄, R₅, R₆, R₇, R₈, and R₉ are hydroxy, amino, cyano, halide, OR11, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₁ is alkyl, —SO₃H, monosaccharide, oligosaccharide, glycofuranosyl, glycopyranosyl, glucuronosyl, or glucuronide;
L1, L₂, and L₃ are O, NR₁₀, or S; and
R10 is H or a substituted or unsubstituted alkyl, aryl, aralkyl, heterocyclyl, heterocyclylalkyl, heteroaryl, or heteroaralkyl.

In another embodiment, methods for activating a sirtuin protein comprise using a compound of formula 112:

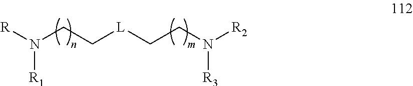

wherein:
R, R1, R₂, and R₃ are H or a substituted or unsubstituted alkyl, aryl, aralkyl, heterocyclyl, heterocyclylalkyl, heteroaryl, or heteroaralkyl;
L is O, NR, S, or Se; and n and m are integers from 0 to 5 inclusive.

In another embodiment, methods for activating a sirtuin protein comprise using a compound of formula 113:

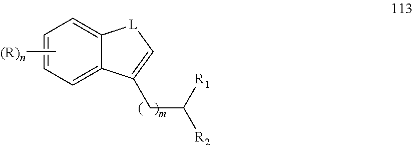

wherein:
R is hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R1 and R₂ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide;
L is O, NR₃, S, or SO₂; R₃ is H or a substituted or unsubstituted alkyl, aryl, aralkyl, heterocyclyl, heterocyclylalkyl, heteroaryl, or heteroaralkyl;
n is an integer from 0 to 4 inclusive; and m is an integer from 1 to 5 inclusive.

In another embodiment, methods for activating a sirtuin protein comprise using a compound of formula 114:

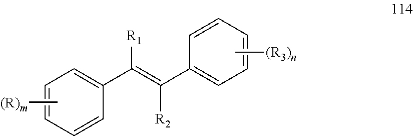

wherein:
R, R1, R₂, and R₃ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide; and n and m are integers from 0 to 5 inclusive.

In another embodiment, methods for activating a sirtuin protein comprise using a compound of formula 115:

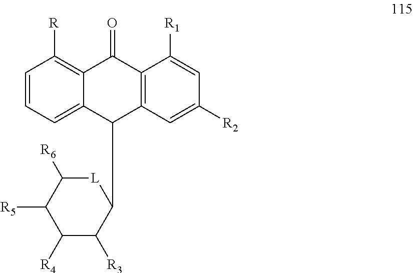

wherein:
R, R1, R₂, R₃, R₄, R₅, and R₆ are H, hydroxy, amino, cyano, OR₈, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R8 is alkyl, —SO₃H, monosaccharide, oligosaccharide, glycofuranosyl, glycopyranosyl, glucuronosyl, or glucuronide;
L is O, NR₇, or S; and R₇ is H or a substituted or unsubstituted alkyl, aryl, aralkyl, heterocyclyl, heterocyclylalkyl, heteroaryl, or heteroaralkyl.

In another embodiment, methods for activating a sirtuin protein comprise using a compound of formula 116:

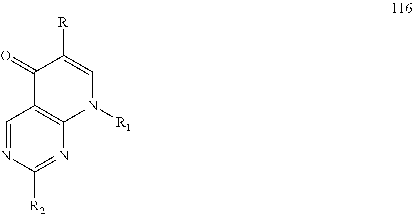

wherein:
R, R1, and R₂ are H, hydroxy, amino, cyano, halide, OR₃, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₃ is alkyl, —SO₃H, monosaccharide, oligosaccharide, glycofuranosyl, glycopyranosyl, glucuronosyl, or glucuronide.

In another embodiment, methods for activating a sirtuin protein comprise using a compound of formula 117:

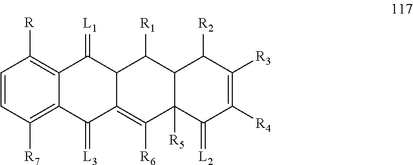

wherein:
R, R₁, R₂, R₃, R₄, R₅, R₆, and R₇ are H, hydroxy, amino, cyano, halide, OR₉, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₉ is alkyl, —SO₃H, monosaccharide, oligosaccharide, glycofuranosyl, glycopyranosyl, glucuronosyl, or glucuronide;
L₁, L₂, and L3 are CH₂, O, NR₈, or S; and
R₈ is H or a substituted or unsubstituted alkyl, aryl, aralkyl, heterocyclyl, heterocyclylalkyl, heteroaryl, or heteroaralkyl.

In another embodiment, methods for activating a sirtuin protein comprise using a compound of formula 118:

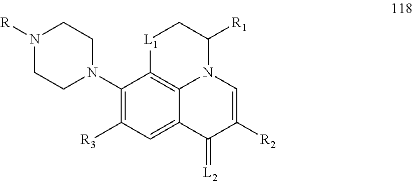

wherein:
R is H or a substituted or unsubstituted alkyl, aryl, aralkyl, heterocyclyl, heterocyclylalkyl, heteroaryl, or heteroaralkyl;
R1, R₂, and R₃ are hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₄ is alkyl, —SO₃H, monosaccharide, oligosaccharide, glycofuranosyl, glycopyranosyl, glucuronosyl, or glucuronide; and
L1 and L₂ are O, NR, or S.

In certain embodiments, any compound of the present invention may also be oxidized forms of the compounds recited herein. For example, an oxidized form of chlortetracyclin may be a sirtuin-activating compound.

Regarding compounds of formulae 67-118, the following definitions apply:

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls. The term “aralkyl” is art-recognized and refers to an alkyl group substituted with an aryl group (e.g., an aromatic or heteroaromatic group). The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, napthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls. The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles.

Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂″. “Halide” designates the corresponding anion of the halogens, and “pseudohalide” has the definition set forth on 560 of “Advanced Inorganic Chemistry” by Cotton and Wilkinson, incorporated herein by reference.

The terms “amine” and “amino” are art-recognized and refer to both unsubstituted and substituted amines, e.g., a moiety that may be represented by the general formulas:

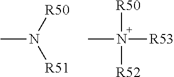

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

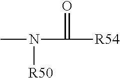

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

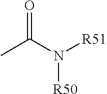

wherein R50 and R51 are as defined above. Certain embodiments of amides may not include imides which may be unstable. The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

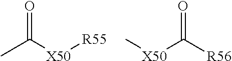

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O˜(CH₂)ₘ—R61, where m and R61 are described above.

The term “sulfonate” is art recognized and refers to a moiety that may be represented by the general formula:

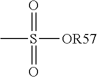

in which R57 is an electron pair, hydrogen, alkyl, cycloalkyl, or aryl.

The term “sulfate” is art recognized and includes a moiety that may be represented by the general formula:

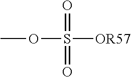

in which R57 is as defined above.

The term “sulfonamido” is art recognized and includes a moiety that may be represented by the general formula:

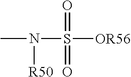

in which R50 and R56 are as defined above.

The term “sulfamoyl” is art-recognized and refers to a moiety that may be represented by the general formula:

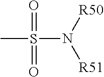

in which R50 and R51 are as defined above.

The term “sulfonyl” is art-recognized and refers to a moiety that may be represented by the general formula:

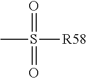

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

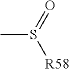

in which R58 is defined above.

The term “phosphoryl” is art-recognized and may in general be represented by the formula:

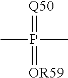

wherein Q 50 represents S or O, and R59 represents hydrogen, a lower alkyl or an aryl. When used to substitute, e.g., an alkyl, the phosphoryl group of the phosphorylalkyl may be represented by the general formulas:

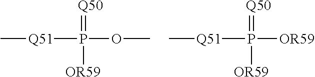

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

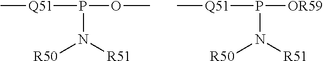

wherein Q51, R50, R51 and R59 are as defined above.

The term “phosphonamidite” is art-recognized and may be represented in the general formulas:

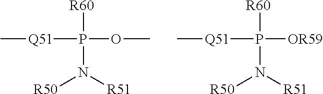

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g., alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

Also contemplated as sirtuin-activating compounds of the present invention are those disclosed in WO 2006/007411 and related US 2006/0084085, each of which is incorporated herein by reference in its entirety. Such compounds include Exemplary compounds that activate sirtuins are described in Howitz et al. (2003) Nature 425:191. These include: resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahydroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydroxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid-H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl). Analogs and derivatives thereof can also be used.

Also contemplated as sirtuin-activating compounds of the present invention are those disclosed in US 2005/0096256, incorporated herein by reference in its entirety. Exemplary compounds contemplated include 119-121:

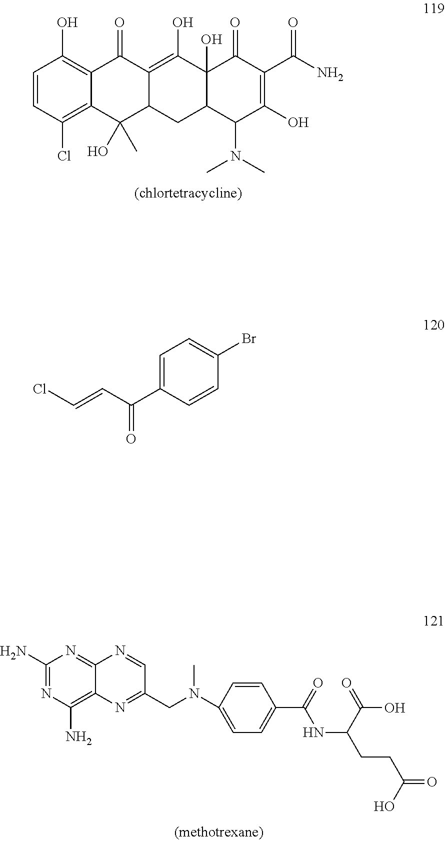

Also contemplated as sirtuin-activating compounds of the present invention are those disclosed in WO 2005/065667 and related US 2005/017027 and US 2006/0111435, each of which is incorporated herein by reference in its entirety. Exemplary compounds contemplated include 122-129, described below.

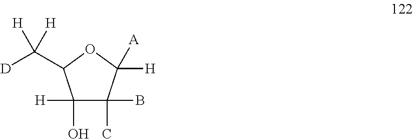

wherein A is a nitrogen-, oxygen-, or sulfur-linked aryl, alkyl, cyclic, or heterocyclic group. The A moieties thus described optionally have leaving group characteristics (a term well-known to those of skill in the art). In embodiments encompassed herein, A is further substituted with an electron contributing moiety (a term well-known to those of skill in the art). B and C are both hydrogen, or one of B or C is a halogen, amino, or thiol group and the other of B or C is hydrogen; and D is a primary alcohol, a hydrogen, or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

In one example, A is a substituted N-linked aryl or heterocyclic group, an O-linked aryl or heterocyclic group having the formula-O—Y, or an S-linked aryl or heterocyclic group having the formula-O—Y; both B and C are hydrogen, or one of B or C is a halogen, amino, or thiol group and the other of B or C is hydrogen; and D is a primary alcohol or hydrogen. Nonlimiting preferred examples of A are set forth below, where each R is H or an electron-contributing moiety and Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl.

Examples of A include i-xiv below:

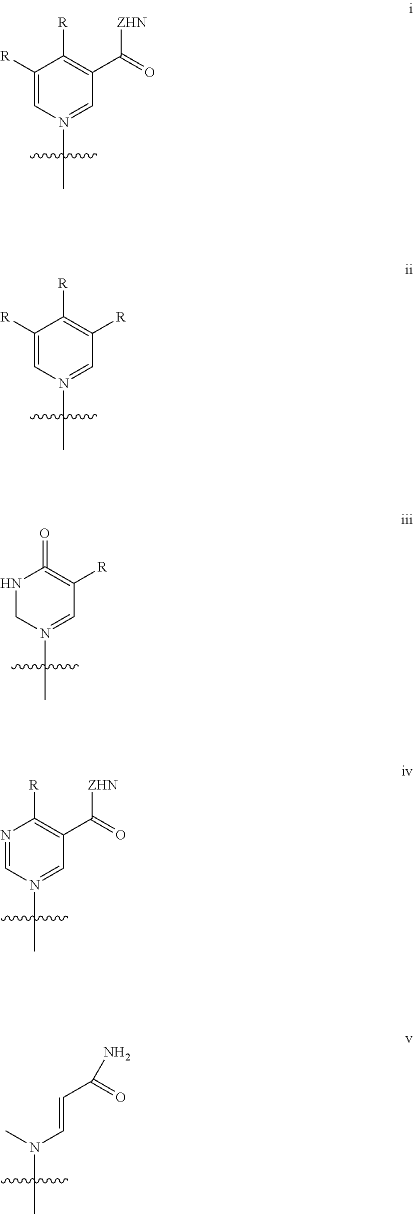

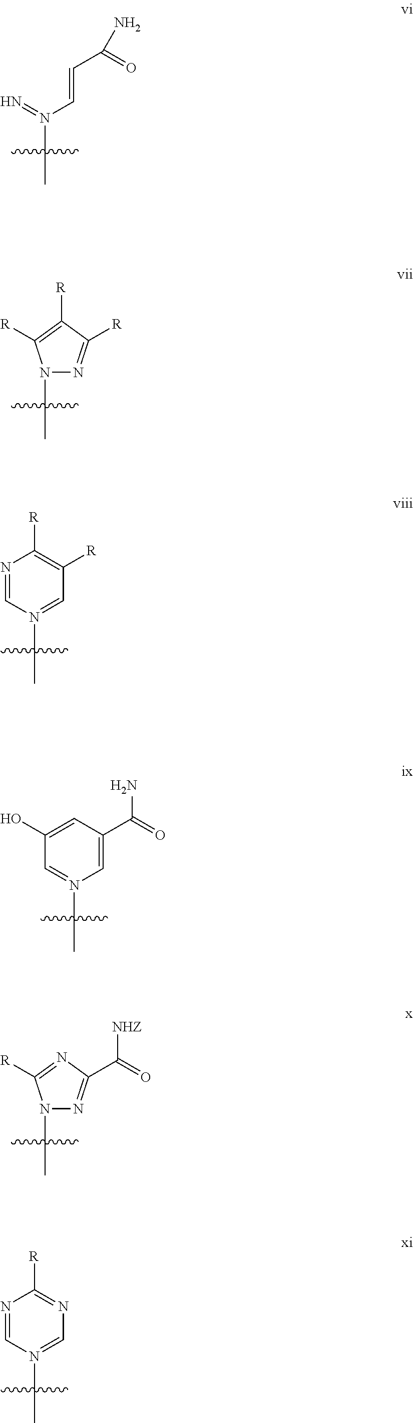

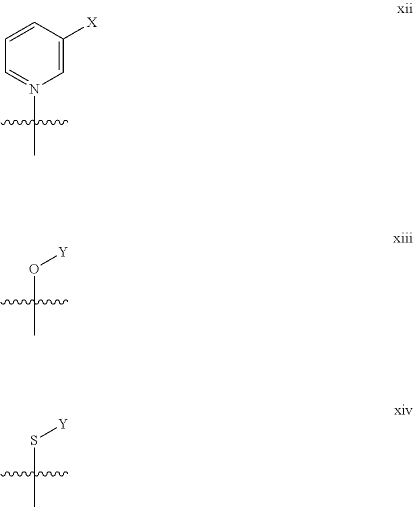

where Y=a group consistent with leaving group function.

Examples of Y include, but are not limited to, xv-xxvii below:

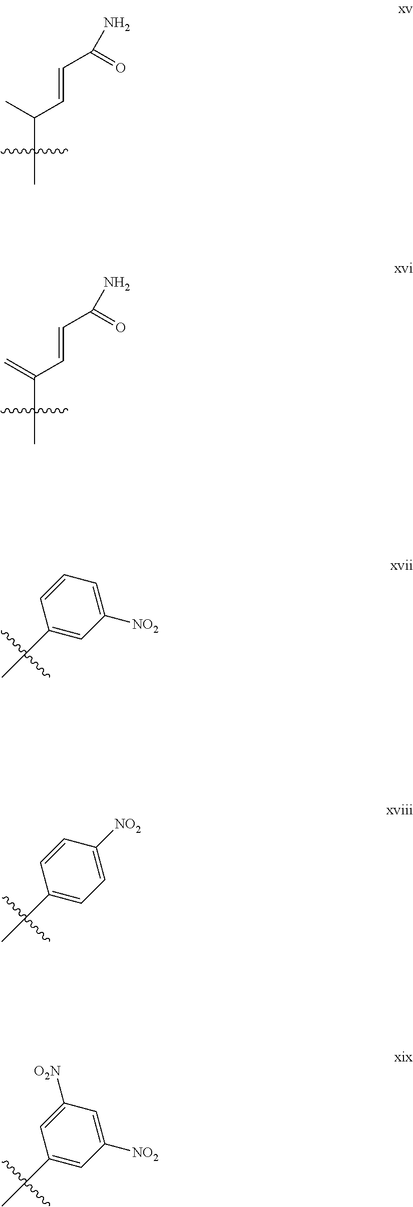

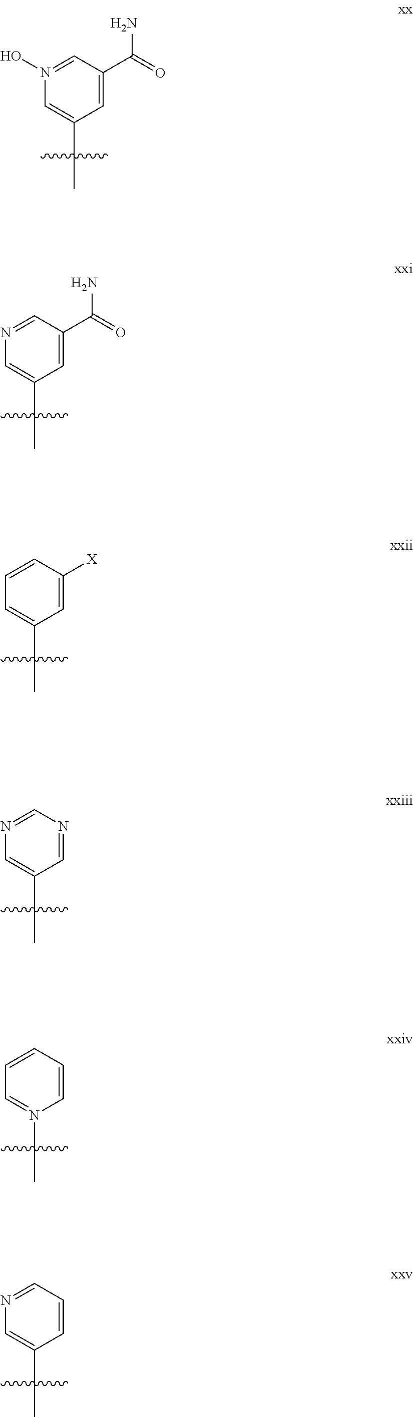

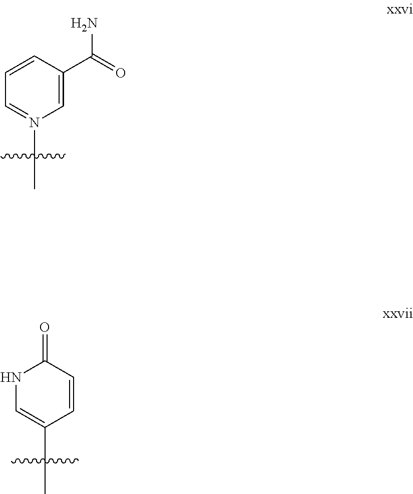

wherein, for i-xxvii, X is halogen, thiol, or substituted thiol, amino or substituted amino, oxygen or substituted oxygen, or aryl or alkyl groups or heterocycles.

In certain embodiments, A is a substituted nicotinamide group (i above, where Z is H), a substituted pyrazolo group (vii above), or a substituted 3-carboxamid-imidazolo group (x above, where Z is H). Additionally, both B and C may be hydrogen, or one of B or C is a halogen, amino, or thiol group and the other of B or C is hydrogen; and D is a primary alcohol or hydrogen.

In other embodiments one of B or G may be halogen, amino, or thiol group when the other of B or C is a hydrogen. Furthermore, D may be a hydrogen or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

Analogues of adenosine monophosphate or adenosine diphosphate also can replace the adenosine monophosphate or adenosine diphosphate groups.

In some embodiments, A has two or more electron contributing moieties.

In other embodiments, a sirtuin-activating compound is an isonicotinamide analog compound of formulas 123, 124, or 125 below.

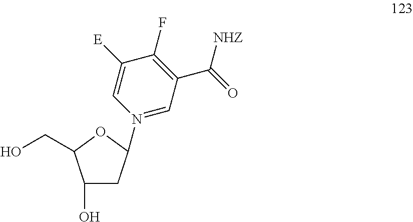

wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; E and F are independently H, CH3, OCHCH2CH3, NH2, OH, NHCOH, NHCOCH3, N(CH3)2, C(CH3)2, an aryl or a C3-C10 alkyl, preferably provided that when one of E or F is H, the other of E or F is not H;

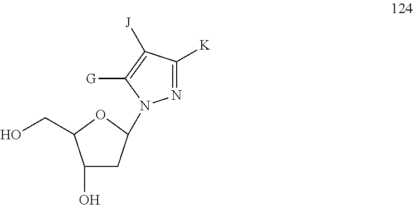

wherein G, J or K is CONHZ, Z is an alkyl, aryl, hydroxyl, OZ′ where Z′ is an alkyl or aryl, amino, NHZ ‘where Z’ is an alkyl or aryl, or NHZ′Z″ where Z′ and Z″ are independently an alkyl or aryl, and the other two of G, J and K is independently CH3, OCH3, CH2CH3, NH2, OH, NHCOH, NHCOCH3;

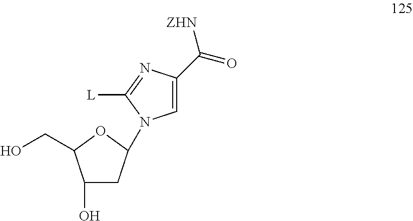

wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; and L is CH3, OCH3, CH2CH3, NH2, OH, NHCOH, or NHCOCH3.

In certain embodiments, the compound is formula 123 above, wherein E and F are independently H, CH3, OCH3, or OH, preferably provided that, when one of E or F is H, the other of E or F is not H. In certain embodiments, the compound is β-1′-5-methyl-nicotinamide-2′-deoxyribose, β-D-1′-5-methyl-nicotinamide-2′-deoxyribofuranoside, β-1′-4,5-dimethyl-nicotinamide-2′-de-oxyribose or β-D-1′-4,5-dimethyl-nicotinamide-2′-deoxyribofuranoside. In yet another embodiment, the compound is (3-1′-5-methyl-nicotinamide-2′-deoxyribose.

Without being bound to any particular mechanism, it is believed that the electron-contributing moiety on A stabilizes certain compounds of the invention such that they are less susceptible to hydrolysis from the rest of the compound. This improved chemical stability improves the value of the compound, since it is available for action for longer periods of time in biological systems due to resistance to hydrolytic breakdown. The skilled artisan could envision many electron-contributing moieties that would be expected to serve this stabilizing function as shown in formulas 122-129. Nonlimiting examples of suitable electron contributing moieties are methyl, ethyl, O-methyl, amino, NMe2, hydroxyl, CMe3, aryl and alkyl groups. Preferably, the electron-contributing moiety is a methyl, ethyl, O-methyl, amino group. In the most preferred embodiments, the electron-contributing moiety is a methyl group.

In other embodiments, exemplary sirtuin-activating compounds are O-acetyl-ADP-ribose analogs, including 2′-O-acetyl-ADP-ribose and 3′-O-acetyl-ADP-ribose, and analogs thereof. Exemplary O-acetyl-ADP-ribose analogs are described, for example, in U.S. Patent Publication Nos. 2004/0053944; 2002/0061898; and 2003/0149261, the disclosures of which are hereby incorporated by reference in their entirety.

In exemplary embodiments, sirtuin-activating compounds may be an O-acetyl-ADP-ribose analog having any of formulas 126-129 below.

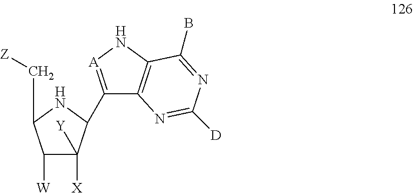

wherein:

A is selected from N, CH and C R, where R is selected from halogen, optionally substituted alkyl, aralkyl and aryl, OH, NH2, NHR1, NR1R2 and SR3, where R1, R2 and R3 are each optionally substituted alkyl, aralkyl or aryl groups;

B is selected from OH, NH2, NHR4, H and halogen, where R4 is an optionally substituted alkyl, aralkyl or aryl group;

D is selected from OH, NH2, NHR5, H, halogen and SCH3, where R5 is an optionally substituted alkyl, aralkyl or aryl group;

X and Y are independently selected from H, OH and halogen, with the proviso that when one of X and Y is hydroxy or halogen, the other is hydrogen;

Z is OH, or, when X is hydroxy, Z is selected from hydrogen, halogen, hydroxy, SQ and OQ, where Q is an optionally substituted alkyl, aralkyl or aryl group; and

W is OH or H, with the proviso that when W is OH, then A is CR where R is as defined above.

In certain embodiments, when B is NHR4 and/or D is NHR5, then R4 and/or R5 are C1-C4 alkyl. In other embodiments, when one or more halogens are present they are chosen from chlorine and fluorine. In another embodiment, when Z is SQ or OQ, Q is C1-C5 alkyl or phenyl. In an exemplary embodiment, D is H, or when D is other than H, B is OH. In another embodiment, B is OH, D is H, OH or NH2, X is OH or H, Y is H, most preferably with Z as OH, H, or methylthio, especially OH. In certain embodiments W is OH, Y is H, X is OH, and A is CR where R is methyl or halogen, preferably fluorine. In other embodiments, W is H, Y is H, X is OH and A is CH.

In other embodiments, a sirtuin-activating compound is an O-acetyl-ADP-ribose analog compound of formula 127:

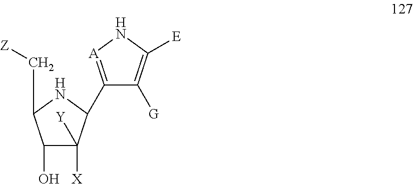

wherein:
A, X, Y, Z and R are defined for compounds of formula (126) where first shown above;
E is chosen from CO2H or a corresponding salt form, CO2R, CN, CONH2, CONHR or CONR2; and
G is chosen from NH2, NHCOR, NHCONHR or NHCSNHR.

In certain embodiments, E is CONH2 and G is NH2. In other embodiments, E is CONH2, G is NH2, X is OH or H, most preferable with Z as OH, H or methylthio, especially OH.

In certain embodiments, exemplary sirtuin-activating compounds may be selected from the group consisting of: (1S)-1,4-dideoxy-1-C-(4-hydroxypyrrolo[3,2-d]pyrimidin-7-yl)-1,4-imino-D-ribitol; (1S)-1-C-(2-amino-4-hydroxypyrrolo[3,2-d]pyrimidin-7-yl)-1,4-dideoxy-1,4-imino-D-ribitol; (1R)-1-C-(4-hydroxypyrrolo[3,2-d]pyrimidin-7-yl)-1,4-imino-1,2,4-trideoxy-D-erythro-pentitol; (1S)-1-C-(4-hydroxypyrrolo[3,2-d]pyrimidin-7-yl)-1,4-imino-1,4,5-trideoxy-D-ribitol; (1S)-1,4-dideoxy-1-C-(4-hydroxypyrrolo[3,2-d]pyrimidin-7-yl)-1,4-imino-5-methylthio-D-ribitol; (1S)-1,4-dideoxy-1-C-(2,4-dihydroxypyrrolo[3,2-d]pyrimidin-7-yl)-1,4-imino-D-ribitol; (1R)-1-C-(2,4-dihydroxypyrrolo[3,2-d]pyrimidin-7-yl)-1,4-imino-1,2,4-trideoxy-D-erthro-pentitol; (1S)-1-C-(2,4-dihydroxypyrrolo[3,2-d]pyrimidin-7-yl)-1,4-imino-1,4,5-trideoxy-D-ribitol; (1S)-1,4-dideoxy-1-C-(2,4-dihydroxypyrrolo[3,2-d]pyrimidin-7-yl)-1,4-imino-5-ethylthio-D-ribitol; (1R)-1-C-(2-amino-4-hydroxypyrrolo[3,2-d]pyrimidin-7-yl)-1,4-imino-1,2,4-trideoxy-D-erythro-pentitol; (1S)-1-C-(2-amino-4-hydroxypyrrolo[3,2-d]pyrimidin-7-yl)-1,4-imino-1,4,5-trideoxy-D-ribitol; (1S)-1-C-(2-amino-4-hydroxypyrrolo[3,2-d]pyrimidin-7-yl)-1,4-dideoxy-1,4-imino-5-methylthio-D-ribitol; (1S)-1,4-dideoxy-1-C-(7-hydroxypyrazolo[4,3-d]pyrimidin-3-yl)-1,4-imino-D-ribitol; (1R)-1-C-(7-hydroxypyrazolo[4,3-d]pyrimidin-3-yl)-1,4-imino-1,2,4-trideoxy-D-erythro-pentitol; (1S)-1-C-(7-hydroxypyrazolo[4,3-d]pyrimidin-3-yl)-1,4-imino-1,4,5-trideoxy-D-ribitol; (1S)-1,4-dideoxy-1-C-(7-hydroxypyrazolo[4,3-d]pyrimidin-3-yl)-1,4-imino-5-ethylthio-D-ribitol; (1S)-1,4-dideoxy-1-C-(5,7-dihydroxypyrazolo[4,3-d]pyrimidin-3-yl)-1,4-imino-D-ribitol; (1R)-1-C-(5,7-dihydroxypyrazolo[4,3-d]pyrimidin-3-yl)-1,4-imino-1,2,4-trideoxy-D-erythro-pentitol; (1S)-1-C-(5,7-dihydroxypyrazolo[4,3-d]pyrimidin-3-yl)-1,4-imino-1,4,5-trideoxy-D-ribitol; (1S)-1,4-dideoxy-1-C-(5,7-dihydroxypyrazolo[4,3-d]pyrimidin-3-yl)-1,4-imino-5-methylthio-D-ribitol; (1S)-1-C-(5-amino-7-hydroxypyrazolo[4,3-d]pyrimidin-3-yl)-1,4-dideoxy-1,4-imino-D-ribitol; (1R)-1-C—(S-amino-7-hydroxypyrazolo[4,3-d]pyrimidin-3-yl)-1,4-imino-1,2,4-trideoxy-D-erythro-pentitol; (1S)-1-C-(5-amino-7-hydroxypyrazolo[4,3-d]pyrimidin-3-yl)-1,4-imino-1,4,5-trideoxy-D-ribitol; (1S)-1-C-(5-amino-7-hydroxypyrazolo[4,3-d]pyrimidin-3-yl)-1,4-dideoxy-1,4-imino-5-methylthio-D-ribitol; (1S)-1-C-(3-amino-2-carboxamido-4-pyrrolyl)-1,4-dideoxy-1,4-imino-D-ribitol; (1S)-1,4-dideoxy-1-C-(4-hydroxypyrrolo[3,2-d]pyrimidin-7-yl)-1,4-imino-D-ribitol 5-phosphate; (1S)-1-C-(2-amino-4-hydroxypyrrolo[3,2-d]pyrimidin-7-yl)-1,4-imino-D-ribitol 5-phosphate; (1S)-1-C-(3-amino-2-carboxamido-4-pyrrolyl)-1,4-dideoxy-1,4-imino-D-ribitol.

In yet other embodiments, sirtuin-activating compounds are O-acetyl-ADP-ribose analog compounds of formula 128 and 129:

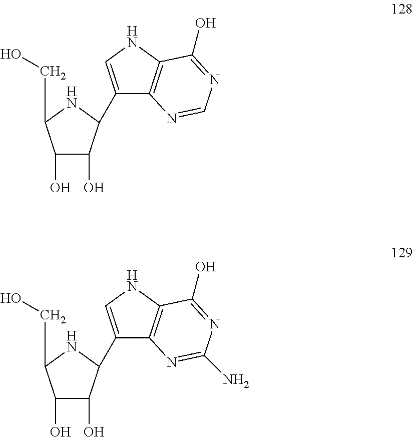

For compounds of formulae 122-129, the definitions of 67-118 apply.

Certain compounds of the present invention may act as a sirtuin-activating compound or a sirtuin-inhibiting compound. While any compound of the present invention may act as a sirtuin-activating compound or a sirtuin-inhibiting compound, compounds of formulae 130-143 are particularly contemplated as compounds that may behave as one or the other. It is to be noted that while Applicants do not wish to be bound by theory, it is believed that sirtuin activators and inhibitors may interact with a sirtuin at the same location within the sirtuin protein (e.g., active site or site affecting the Kₘ or Vₘₐₓ of the active site). It is believed that this is the reason why certain classes of sirtuin activators and inhibitors can have substantial structural similarity.

In certain embodiments, exemplary sirtuin-activating or sirtuin-inhibiting compounds are fused heterocyclic compounds as disclosed in WO 2006/094235, hereby incorporated by reference in its entirety. Such exemplary compounds include compounds of formulae 130-143, described below:

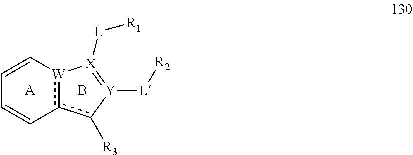

wherein:
Ring A is optionally substituted;
L is absent, substituted or unsubstituted phenylene, substituted or unsubstituted —O-phenylene, substituted or unsubstituted thienylene, substituted or unsubstituted pyrazolylene, substituted or unsubstituted benzothiazolylene, —NR₄—, —C(O)O—, —C(O)NR₄—, —NR₄C(O)—, —NR₄—C(O)—NR₅—, —S—, —CHR₆═CHR₇— or —CHR₆—C(O)—;
L′ is absent, substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —C(O)O—, —C(O)NR₄—, —NR₄C(O)—, —NR₄—C(O)—NR₅—, —S—, —CHR₆═CHR₇— or —CHR₈—C(O)—, provided that at least one of L and L′ is substituted or unsubstituted phenylene, substituted or unsubstituted —O-phenylene, substituted or unsubstituted thienylene, substituted or unsubstituted pyrazolylene, substituted or unsubstituted benzothiazolylene, substituted or unsubstituted indenedionylene, —NR₄—, —C(O)O—, —C(O)NR₄—, —NR₄C(O)—, —NR₄—C(O)—NR₅—, —S—, —CHR₆═CHR₇— or —CHR₈—C(O)—;
R₁ is absent, —H, —NR₄R₅, —N₄C(O)R₅, —OR₅, naphthyl or a heterocyclic group, provided that L and R₁ are not both absent unless X is N;
R₂ is —H, unsubstituted alkyl, —NR₄R₅, —NR₄C(O)R₅, —OR₅, substituted or unsubstituted phenyl, naphthyl or a heterocyclic group; R₃ is —H, —NR₄R₅, —N₄C(O)R₅, —OR₅ or a substituted or unsubstituted heterocyclic group, or R₂ and R₃, taken together with the atoms to which they are attached, form an optionally substituted heterocyclic group, or R₃ is absent when Z is O or S;
R₄ and R₅ are independently —H, a substituted or unsubstituted alkyl group, a substituted or unsubstituted aryl group or a substituted or unsubstituted non-aromatic heterocyclic group;
R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;
W is C or N;
X is C or N;
Y is C or N;
Z is C, N, O or S, provided that at least two of W, X₃ Y and Z are C; and
n is 1 or 2.

In another embodiment, sirtuin-modulating compounds of the invention are represented by formula (131):

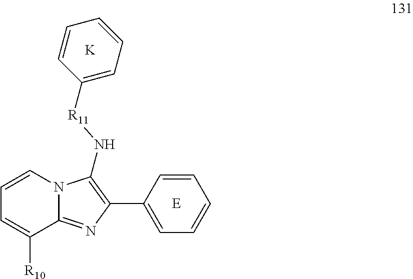

wherein:
R₁₀ is selected from —H, —C(O)—N(R₄₀)(R₅₀), —S(O)₂N(R₄₀)(R₅₀), or —CH₂—N(R₄₀)(R₅₀); wherein each of R₄₀ and R₅₀ is independently selected from —H, —C₁-C₃ straight or branched alkyl, —(C₁-C₃ straight or branched alkyl)-N(CH₃)₂, —(C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not H;
R₁₁ is selected from —C₁-C₃ straight or branched alkylene or —C(O)—;
and each of ring K and ring E is independently substituted with up to three substituents independently selected from halo, —CF₃, —O—(C₁-C₃ straight or branched alkyl), —S—(C₁-C₃ straight or branched alkyl), —N(R₄₀)(R₅₀), —S(O)₂—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In certain embodiments, one of R₄₀ or R₅₀ is H. In certain embodiments, ring K is substituted with up to 3 substituents independently selected from methyl, —O-methyl, —N(CH₃)₂, or —CF₃, but is unsubstituted in the positions ortho to the attachment to the rest of the molecule. In certain embodiments, such as where R₄₀ and/or R₅₀ have the values indicated above and/or ring K has the substitution pattern described above, ring E is substituted with up to 2 substituents independently selected from methyl, —O-methyl, —S(O)₂—N(CHs)₂, —O-methyl-morpholino, —O-ethyl-morpholino, fluoro, —CF₃, piperidyl, methylpiperidyl, pyrrolidyl, or methylpyrrolidyl. In certain embodiments, R₁₀ is selected from —H, —CH₂-piperazinyl, —CBb-methylpiperazinyl, —CH₂-pyrrolidyl, —CH₂-piperidyl, —CH₂-morpholino, —CH₂—N(CHs)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholmo, —C(O)—NH; (CH₂)ₙ-piperidyl, or —C(O)—NH—(CH₂)ₙ—N(CH₃)₂, wherein n is 1 or 2.

In particular embodiments, ring K is substituted with up to 3 substituents independently selected from methyl, O-methyl, N(CH₃)₂, CF₃, but is unsubstituted in the positions ortho to the attachment to the rest of the molecule; ring E is substituted with up to 2 substituents independently selected from methyl, O-methyl, —S(O)₂—N(CH₃)₂, —O-methyl-morpholino, —O-ethyl-morpholino, fluoro, —CF₃, methylpiperidyl, or pyrrolidyl; and R₁₀ is selected from —H, —CH₂-piperazinyl, —C(O)—NH—(CH₂)₂-piperazinyl, —C(O)—NH—(CH₂)₂-methylpiperazinyl, —C(O)—NH—(CH₂)₂-pyrrolidyl, or —C(O)—NH—(CH₂)₂—N(CH₃)₂.

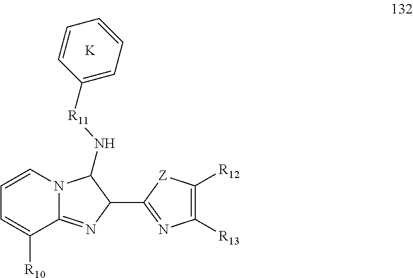

wherein:
Z is selected from O or S;
R₁₀ is selected from —H, —C(O)—N(R₄₀)(R₅₀), —S(O)₂N(R₄₀)(R₅₀), or —CH₂—N(R₄₀)(R₅₀); wherein each of R₄₀ and R₅₀ is independently selected from —H, —C₁-C₃ straight or branched alkyl, —(C₁-C₃ straight or branched alkyl)-N(CH₃)₂, (C₁-C₃ straight or branched alkyl)-heterocyclyl, —(C₁-C₃ straight or branched alkyl)-alkylheterocyclyl, or wherein R₄₀ and R₅₀ taken together with the N atom to which they are bound form a 5-6 membered heterocyclic ring that is optionally substituted with —(C₁-C₃ straight or branched alkyl), and wherein at least one of R₄₀ or R₅₀ is not H;
R₁₁ is selected from —C₁-C₃ straight or branched alkylene or —C(O)—;
each of R₁₂ and R₁₃ is independently selected from —H or —(C₁-C₃ straight or branched alkyl), or R₁₂ and R₁₃ are taken together to form a benzene ring that is substituted with up to two substituents independently selected from —(C₁-C₃ straight or branched alkyl), —CF₃ or halo; and
ring K is substituted with up to three substituents independently selected from halo, —CF₃, —O—(C₁-C₃ straight or branched alkyl), —S—(C₁-C₃ straight or branched alkyl), —N(R₄₀)(R₅₀), —S(O)₂—N(R₄₀)(R₅₀), heterocyclyl, (C₁-C₃ straight or branched alkyl)-heterocyclyl, —O—(C₁-C₃ straight or branched alkyl)-heterocyclyl, —S—(C₁-C₃ straight or branched alkyl)-heterocyclyl, or is optionally fused to a 5-6 membered heterocyclyl or heteroaryl, wherein any heterocyclcyl or heteroaryl is optionally substituted with —C₁-C₃ straight or branched alkyl.

In certain embodiments, R₁₀ is —H. In certain embodiments, ring K is substituted with up to 3 substituents independently selected from methyl, O-methyl, N(CH₃)₂, CF₃, and wherein ring K is unsubstituted in the positions ortho to the attachment to the rest of the molecule. In certain embodiments, such as when R₁₀ is —H and/or ring K has the substitution pattern described above, each of R₁₂ and R₁₃ is independently selected from —H, methyl, —O-methyl, —S(O)₂—N(CH₃)₂, —O-methyl-morpholino, —O-ethyl-morpholino, fluoro, —CF₃, piperidyl, methylpiperidyl, pyrrolidyl, or methylpyrrolidyl. In a preferred embodiment, each of R₁₂ and R₁₃ is methyl.

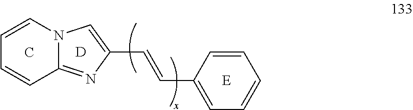

wherein:

Rings C, D and E are optionally substituted; and x is 0 or 1.

In certain embodiments x is 0. In certain embodiments (e.g., where x is 0), Ring C is substituted with a group that is capable of providing a trans configuration (e.g., an amide group, an optionally 2-substituted 1-alkenyl group).

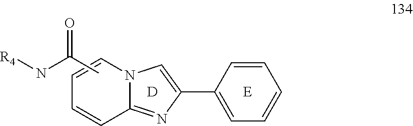

wherein:
Rings D and E are optionally substituted; and
R₄ is —H, a substituted or unsubstituted alkyl group, a substituted or unsubstituted aryl group or a substituted or unsubstituted non-aromatic heterocyclic group.

In certain embodiments, Ring E is substituted with an acylamino, heterocyclylcarbonylamino, lower alkyl or substituted or unsubstituted alkoxy group. In certain embodiments, Ring D is substituted with an amino group. In particular embodiments, Ring E is substituted with an acylamino, heterocyclylcarbonylamino, lower alkyl, or substituted or unsubstituted alkoxy group, and Ring D is substituted with an amino group. In certain embodiments, R₄ is a substituted alkyl group.

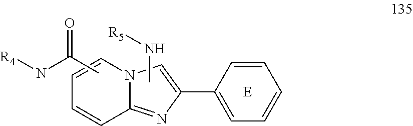

wherein:
Ring E is optionally substituted; and
R₄ and R₅ are independently —H, a substituted or unsubstituted alkyl group, a substituted or unsubstituted aryl group or a substituted or unsubstituted non-aromatic heterocyclic group.

In certain embodiments, Ring E is substituted with an acylamino, heterocyclylcarbonylamino, lower alkyl or substituted or unsubstituted alkoxy group. In certain embodiments, R₄ is a substituted alkyl group. In particular embodiments, Ring E is substituted with an acylamino, heterocyclylcarbonylamino, lower alkyl or substituted or unsubstituted alkoxy group and R₄ is a substituted alkyl group. In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group when Ring E is substituted with an acylamino, heterocyclylcarbonylamino, lower alkyl or substituted or unsubstituted alkoxy group and/or R₄ is a substituted alkyl group.

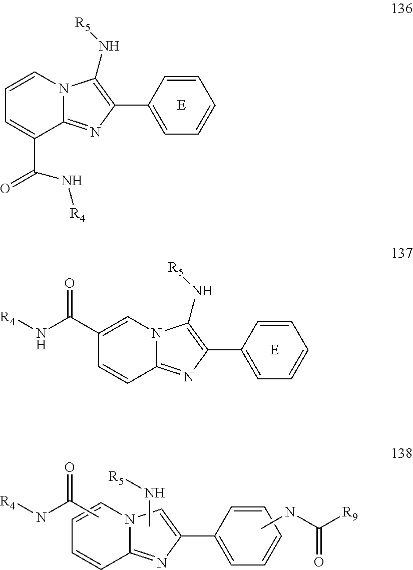

wherein, for compounds of formulae 136-138:
R₄, R₅ and R₉ are independently —H, a substituted or unsubstituted alkyl group, a substituted or unsubstituted aryl group or a substituted or unsubstituted non-aromatic heterocyclic group.

In certain embodiments, R₄ is a substituted alkyl group. In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group and R₄ is a substituted alkyl group. In certain embodiments, R₉ is a C₁₋₄ alkyl group (e.g., methyl, cyclopropyl), a substituted or unsubstituted aryl group (e.g., substituted or unsubstituted phenyl) or a substituted or unsubstituted non-aromatic heterocyclic group (e.g., furanyl, morpholino). In particular embodiments, R₉ is a C₁₋₄ alkyl group, a substituted or unsubstituted aryl group or a substituted or unsubstituted non-aromatic heterocyclic group when R₅ is a substituted or unsubstituted alkyl group and/or R₄ is a substituted alkyl group.

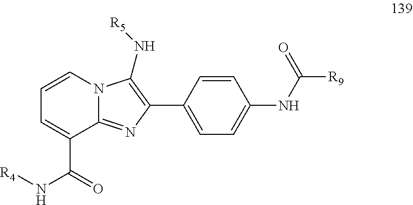

wherein:
R₄, R.₅ and R₉ are independently —H, a substituted or unsubstituted alkyl group, a substituted or unsubstituted aryl group or a substituted or unsubstituted non-aromatic heterocyclic group.

In certain embodiments, R₄ is a substituted alkyl group. In certain embodiments, R₅ is a substituted or unsubstituted alkyl group, such as an aralkyl or a cycloalkyl group (e.g., benzyl, cyclohexyl). In particular embodiments, R₅ is a substituted or unsubstituted alkyl group and R₄ is a substituted alkyl group. In certain embodiments, R₉ is a C₁₋₄ alkyl group (e.g., methyl, cyclopropyl), a substituted or unsubstituted aryl group (e.g., substituted or unsubstituted phenyl) or a substituted or unsubstituted non-aromatic heterocyclic group (e.g., furanyl, morpholino). In particular embodiments, R₉ is a C₁₋₄ alkyl group, a substituted or unsubstituted aryl group or a substituted or unsubstituted non-aromatic heterocyclic group when R₅ is a substituted or unsubstituted alkyl group and/or R₄ is a substituted alkyl group.

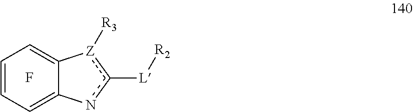

where:
Ring F is optionally substituted;
L′ is substituted or unsubstituted phenylene, substituted or unsubstituted thienylene, substituted or unsubstituted indonedionylene, —C(O)O—, —NR₄C(O)—, —S—, —CHR₆═CHR₇— or —CHR₈—C(O)—;
R₂ is —H, unsubstituted alkyl, —NR₄R₅, —NR₄C(O)R₅, —OR₅, substituted or unsubstituted phenyl or a heterocyclic group;
R₃ is —H, —NR₄R₅, —N₄C(O)R₅, —OR₅ or a heterocyclic group, or R₂ and R₃, taken together with the atoms to which they are attached, form an optionally substituted heterocyclic group, or R₃ is absent when Z is O or S;
R₄ and R₅ are independently —H, a substituted or unsubstituted alkyl group, a substituted or unsubstituted aryl group or a substituted or unsubstituted non-aromatic heterocyclic group;
R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;
Z is C, N, O or S; and n is 1 or 2.

In formula 140, the solid/dashed “double” bond represents a single or double bond. For the compounds represented by formula 141, both dashed bonds cannot be double bonds, but preferably one of the dashed bonds is a double bond and the other is a single bond.

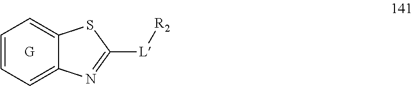

where:
Ring G is optionally substituted;
L′ is substituted or unsubstituted phenylene, substituted or unsubstituted —O-phenylene, substituted or unsubstituted thienylene, substituted or unsubstituted indonedionylene, —NR₄C(O)—, —C(O)O—, —S—, —CHR₆═CHR₇— or —CHR₈—C(O)—;
R₂ is —H, unsubstituted alkyl, —NR₄R₅, —NR₄C(O)R₅, —OR₅, substituted or unsubstituted phenyl or a heterocyclic group;
R₄ and R₅ are independently —H, a substituted or unsubstituted alkyl group, a substituted or unsubstituted aryl group or a substituted or unsubstituted non-aromatic heterocyclic group;
R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and n is 1 or 2.

In certain embodiments, L′ is substituted or unsubstituted —O-phenylene, substituted or unsubstituted thienylene or —CHR₈—C(O)—. In certain embodiments, R₂ is —NR₄C(O)R₅ or a heterocyclic group, such as —NR₄C(O)-substituted alkyl. In a particular embodiment, R₂ is —NR₄C(O)R₅ or a heterocyclic group and L′ is substituted or unsubstituted —O-phenylene, substituted or unsubstituted thienylene or —CHRs-C(O)—. In certain embodiments, Ring G is unsubstituted. In a particular embodiment, Ring G is unsubstituted when R₂ is —NR₄C(O)R₅ or a heterocyclic group and/or L′ is substituted or unsubstituted —O-phenylene, substituted or unsubstituted thienylene or —CHR₈—C(O)—. In particular embodiments, L′ is substituted or unsubstituted —O-phenylene or —CHR₈—C(O)—. In such embodiments, R₂ is preferably a heterocyclic group. In particular embodiments, L′ is a substituted or unsubstituted thienylene. In such embodiments, R₂ is —NR₄C(O)R₅.

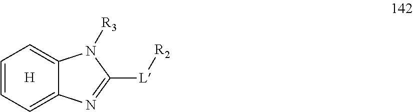

wherein:
Ring H is optionally substituted; L′ is substituted or unsubstituted phenylene, —S— or —CHR₆═CHR₇—;
R₂ is —NR₄R₅, —NR₄C(O)R₅ or a heterocyclic group;
R₃ is —H, or R₂ and R₃, taken together with the atoms to which they are attached, form an optionally substituted heterocyclic group;
R₄ and R₅ are independently —H, a substituted or unsubstituted alkyl group, a substituted or unsubstituted aryl group or a substituted or unsubstituted non-aromatic heterocyclic group;
R₆ and R₇ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and n is 1 or 2.

In certain embodiments, L′ is —S—. In particular embodiments, L′ is —S— and R₂ and R₃, taken together with the atoms to which they are attached, form an optionally substituted heterocyclic group. In certain embodiments, L′ is a substituted or unsubstituted phenylene. In particular embodiments, L′ is a substituted or unsubstituted phenylene and R₂ is —NR₄C(O)R₅. In certain embodiments, L′ is —CHR₆═CHR₇—, such as —CH₂═CH₂— or —C(CN)═CH₂—. In particular embodiments, L′ is —CHR₆═CHR₇— and R₂ is —NR₄R₅ or a substituted or unsubstituted aryl group.

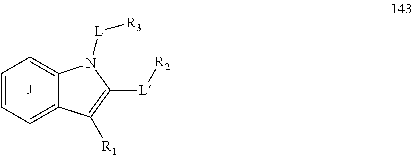

wherein:
Ring J is optionally substituted;
L is substituted or unsubstituted phenylene, substituted or unsubstituted —O-phenylene, substituted or unsubstituted thienylene, substituted or unsubstituted pyrazolylene, substituted or unsubstituted benzothiazolylene, —C(O)O—, —C(O)NR₄—, —NR₄C(O)—, —NR₄—C(O)—NR₅—, —S—, —CHR₆═CHR₇— or —CHR₆—C(O)—;
L′ is substituted or unsubstituted phenylene, substituted or unsubstituted —O-phenylene, substituted or unsubstituted thienylene, substituted or unsubstituted pyrazolylene, substituted or unsubstituted benzothiazolylene, —C(O)O—, —C(O)NR₄—, —NR₄C(O)—, —NR₄—C(O)—NR₅—, —S—, —CHR₆═CHR₇— or —CHR₈—C(O)—;
R₁ is —H, —NR₄R₅, —N₄C(O)R₅, —OR₅, naphthyl or a heterocyclic group;
R₂ is —H, unsubstituted alkyl, —NR₄R₅, —NR₄C(O)R₅, —OR₅, naphthyl or a heterocyclic group;
R₃ is —H, —NR₄R₅, —N₄C(O)R₅, —OR₅ or a substituted or unsubstituted heterocyclic group;
R₄ and R₅ are independently —H, a substituted or unsubstituted alkyl group, a substituted or unsubstituted aryl group or a substituted or unsubstituted non-aromatic heterocyclic group;
R₆, R₇ and R₈ are independently selected from the group consisting of halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; and n is 1 or 2.

In certain embodiments, L is —NR₄C(O)—. In certain embodiments, R₃ is a substituted or unsubstituted aryl group. In particular embodiments, R₃ is a substituted or unsubstituted aryl group and L is —NR₄C(O)—. In certain embodiments, L′ is —C(O)O—. In particular embodiments, L′ is —C(O)O— when R₃ is a substituted or unsubstituted aryl group and/or L is —NR₄C(O)—. In certain embodiments, R₂ is an unsubstituted alkyl group. In particular embodiments, R₂ is an unsubstituted alkyl group when L′ is —C(O)O—, R₃ is a substituted or unsubstituted aryl group and/or L is —NR₄C(O)—. In certain embodiments, Ring G is substituted, such as with one or more (e.g., two) alkoxy (e.g., methoxy, ethoxy) groups, for example, in the positions ortho to the bridgehead carbon atoms. In particular embodiments, Ring G is substituted when R₂ is an unsubstituted alkyl group, L′ is —C(O)O—, R₃ is a substituted or unsubstituted aryl group and/or L is —NR₄C(O)—.

For compounds of formulae 130-143, the following definitions apply:

An “alkyl group” is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An “alkenyl group” is a straight chained, branched or cyclic non-aromatic hydrocarbon which contains one or more double bonds. Typically, the double bonds are not located at the terminus of the alkenyl group, such that the double bond is not adjacent to another functional group. An alkynyl group is a straight chained, branched or cyclic non-aromatic hydrocarbon which contains one or more triple bonds. Typically, the triple bonds are not located at the terminus of the alkynyl group, such that the triple bond is not adjacent to another functional group.

A “cyclic group,” such as a 5- to 7-member ring, includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

“Aromatic (aryl) groups” include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

“Non-aromatic heterocyclic rings” are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R³, —CN₃—NO², —COOH, —COOR^{a}, —OCO₂R^{a}, —C(0)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR^{a}, —CON(R^{a}R^{b}), —NHCOR³, —NRC0R^{a}, —NHCONH₂, —NHC0NR^{a}H, —NHC0N(R^{a}R^{b}), —NR⁰CONH₂, —NR^{o}C0NR^{a}H, —NR^{o}C0N(R^{a}R^{b}), —C(═NH)—NH₂, —C(═NH)—NHR^{a}, —C(═NH)—N(R^{a}R^{b}), —C(═NR^{o}—NH₂, —C(═NR^{o})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{C})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{o})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR₃, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is O, 1 or 2), —S(O)ₖOR^{a} (k is O, 1 or 2) and —NH—C(═NH)—NH₂. R^{a}—R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

In certain embodiments, exemplary sirtuin-activating or sirtuin-inhibiting compounds are aryl-substituted cyclic compounds as disclosed in WO 2006/094248, hereby incorporated by reference in its entirety. Such exemplary compounds include compounds of formulae 144-147, described below:

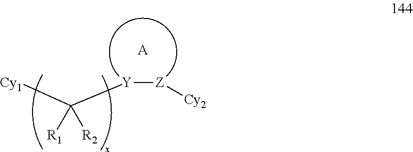

wherein:
Ring A is an optionally substituted, aliphatic, carbocyclic or heterocyclic ring;
R₁ and R₂ are independently halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁ and R₂ taken together are ═O, ═S or ═N;
R₄ and R₅ are independently —H, a substituted or unsubstituted alkyl group, a substituted or unsubstituted aryl group or a substituted or unsubstituted non-aromatic heterocyclic group;
Cy₁ and Cy₂ are independently cyclic groups, one or both of which are optionally fused to Ring A, typically none or one is/are fused;
Y and Z are independently CH, N, O or S, provided that O and S are not adjacent to another O or S; n is 1 or 2; and x is 0 or 1.

In certain embodiments, Ring A is a heterocyclic ring, preferably further substituted and/or aliphatic (e.g., non-aromatic). In a particular embodiment, Ring A is a further substituted, aliphatic, heterocyclic ring. Ring A can be 5- to 8-membered, but is preferably 5- or 6-membered, more preferably 5-membered. In certain embodiments, R₁ and R₂ taken together are ═O. In certain embodiments, Y and/or Z are each CH. In particular embodiments, Y and Z are each CH, such as when R₁ and R₂ taken together are ═O. In certain embodiments, Cy₁ and Cy₂ are each substituted or unsubstituted aryl groups, such as substituted or unsubstituted phenyl groups. In particular embodiments, Cy₁ and Cy₂ are each substituted or unsubstituted aryl groups when Y and Z are each CH and/or R₁ and R₂ taken together are ═O.

The stereochemistry of compounds represented by formula (144) is preferably as depicted in formula (145):

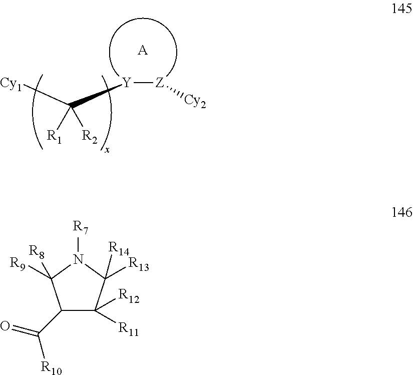

where:
R₄ and R₅ are independently —H, a substituted or unsubstituted alkyl group, a substituted or unsubstituted aryl group or a substituted or unsubstituted non-aromatic heterocyclic group;
R₇ is —H, a substituted or unsubstituted alkyl group, a substituted or unsubstituted aryl group, a substituted or unsubstituted non-aromatic heterocyclic group, a substituted or unsubstituted acyl group or a substituted or unsubstituted aminocarbonyl group, or R₇ and R₁3 and R_{H} taken together with the atoms to which they are attached are a substituted or unsubstituted heterocyclic ring;
R₈ is —H;
R₉ is a substituted or unsubstituted aryl group;
R₁₀ is a substituted or unsubstituted aryl group;
R₁₁ is —H;
R₁₂ is a substituted or unsubstituted aryl group;
R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO3H, —S(O)ₙR₄, —S(O)ₙOR₄, —S(O)ₙNR₄R₅, —NR₄R₅, —NR₄C(O)OR₅, —NR₄C(O)R₅ and —NO₂; and n is 1 or 2.

In certain embodiments, R₁₃ is —CO₂R₄, such as —CO₂H. In certain embodiments, R₁₂ is a substituted or unsubstituted alkyl group, such as an unsubstituted alkyl group. In particular embodiments, R₁₂′ is a substituted or unsubstituted alkyl group and R₁₃ is —CO₂R₄. In certain embodiments, R₁₀ and R₁₂ are independently substituted or unsubstituted phenyl or thienyl groups. Typically, R₁₀ is an unsubstituted phenyl group. Typically, R₁₂ is a nitrophenyl, methoxyphenyl, chlorophenyl or thienyl group. In particular embodiments, R₁₀ and R₁₁ are independently substituted or unsubstituted phenyl groups and R₁₃ is —CO₂R₄.

In certain embodiments, R₉ is an aryl group other than a pyrazolyl group, for example, a substituted or unsubstituted phenyl or thienyl group, such as an alkoxyphenyl (e.g., methoxyphenyl) group. In particular embodiments, R₉ is a substituted or unsubstituted phenyl or thienyl group when R₁₀ and R₁₂ are independently substituted or unsubstituted phenyl groups and/or R₁₃ is —CO₂R₄.

In certain embodiments, R₉ is a substituted or unsubstituted pyrazolyl group. In certain embodiments, —C(O)R₁₀ is located trans to —R₁₂.

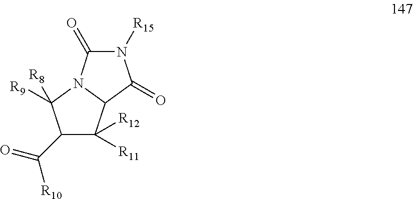

wherein:
R₄ and R₅ are independently —H, a substituted or unsubstituted alkyl group, a substituted or unsubstituted aryl group or a substituted or unsubstituted non-aromatic heterocyclic group; R₈ is —H;
R₉ is a substituted or unsubstituted aryl group (e.g., 4-methoxy-3-nitrophenyl, nitrophenyl);
R₁₀ is a substituted or unsubstituted aryl or substituted or unsubstituted alkyl group (e.g., thienyl, phenyl, methyl);
R₁₁ is —H; R₁₂ is a substituted or unsubstituted aryl group (e.g., cyanophenyl, dimethylaminophenyl);
R₁₅ is a substituted or unsubstituted alkyl, substituted or unsubstituted alkenyl or substituted or unsubstituted aryl group (e.g., methyl, ethyl, 2-propenyl).

For compounds of formulae 144-147, the following definitions apply:

An “acyl group” is an alkyl, alkenyl, alkynyl or aryl group that connects to another moiety through a carbonyl group attached thereto. An alkyl group is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An “alkenyl group” is a straight chained, branched or cyclic non-aromatic hydrocarbon which contains one or more double bonds. Typically, the double bonds are not located at the terminus of the alkenyl group, such that the double bond is not adjacent to another functional group.

An “alkynyl group” is a straight chained, branched or cyclic non-aromatic hydrocarbon which contains one or more triple bonds. Typically, the triple bonds are not located at the terminus of the alkynyl group, such that the triple bond is not adjacent to another functional group. A 5- to 7-membered ring includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other.

“Aromatic (aryl) groups” include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl. “Non-aromatic heterocyclic rings” are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars. A ring fused to a second ring shares at least one common bond.

Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR³, —OCO₂R³, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —C0NHR^{a}, —C0N(R^{a}R^{b}), —NHCOR³, —NRCOR³, —NHCONH₂, —NHCONR³H, —NHC0N(R^{a}R^{b}), —NR⁰CONH₂, —NR^{c}C0NR^{a}H, —NR^{c}CON(R^{a}R^{b}), —C(═NH)—NH₂, —C(═NH)—NHR^{a}, —C(═NH)—N(R^{a}R^{b}), —C(═NR^{C})—NH₂, —C(═NR^{c})—NHR^{a}, —C(═NR>N(R^{a}R^{b}), —NH—CC═NH)—NH₂, —NH—C(═NH)—NHR^{a}, —NH—C(═NH)—N(R³R^{b}), —NH—C(═NR^{C})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{o})—NH₂, —NR^{d}—C(═NR^{c})—NHR³, —NR^{d}—C(═NR^{o})—N(R³R^{b}), —NHNH₂, —NHNHR³, —NHR^{a}R^{b}, —SO₂NH₂, —SO₂NHR₃, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —S0ₖR³ (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

In certain embodiments, exemplary sirtuin-activating or sirtuin-inhibiting compounds are acridine and quinoline compounds and derivatives as disclosed in WO 2006/094237, hereby incorporated by reference in its entirety. Such exemplary compounds include compounds of formulae 148-152, described below:

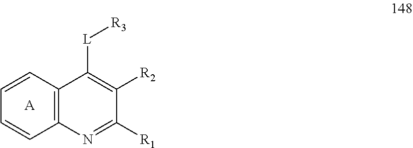

wherein:
Ring A is optionally substituted;
R₁ and R₂ are independently selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂, or R₁ and R₂ taken together with the atoms to which they are attached form an optionally substituted ring;
L is selected from —CH═CH—C(O)—, —CH₂—N(R₄)—C(O)—, —C(O)—CH₂—, —C(O)NR₄—, —C(O)—N(R₄)—C(O)—, —C(O)—N(R₄)—N(R₅)—, —C(O)—N(R₄)—N(R₅)—C(O)—, —CH₂—N(R₄)—N(R₅)—, —N(R₄)—S(O)₂—, —S(O)₂—N(R₄)—, —N(R₄)—N(R₅)—C(O)—,

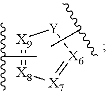

—N(R₄)—N(R₅)—CH₂, —N(R₄)—N(R₅)— or
R₃, R₄ and R₅ are, independently for each occurrence, —H, a substituted or unsubstituted alkyl group, a substituted or unsubstituted aryl group or a substituted or unsubstituted non-aromatic heterocyclic group; Y is selected from O, S, or NR₄; each of X₆, X₇, X₈ and X₉ is independently selected from CR₇, C, or N, wherein at least two of X₆, X₇, X₈ or X₉ are not N; each R₇ is independently selected from H or (C₁-C₃)-straight or branched alkyl; and n is 1 or 2.

In certain embodiments, L is —NR₄R₅—, —C(O)O—, —C(O)NR₄—, —NR₄C(O)—, —NR₄—NR₅—C(O)—, —C(O)—NR₄—NR₅— or —CHR₄═CHR₅—. In certain such embodiments, L is —C(O)NR₄—, —NR₄C(O)—, —NR₄—NR₅—C(O)—, —C(O)—NR₄—NR₅— or —CHR₄═CHR₅—. In certain embodiments, R₄ or R₅ when it appears in L is selected from H and (C₁-C₃)-straight or branched alkyl. In certain embodiments, R₄ and R₅ when they appear in L are H.

In certain embodiments, R₂ is selected from —H and —OH. In certain embodiments, R₂ is —H. In certain embodiments, R₃ is a substituted or unsubstituted non-aromatic heterocyclic group or a substituted or unsubstituted aryl group, such as a substituted or unsubstituted heteroaryl group. In certain embodiments, R₃ is an alkyl group substituted with a substituted or unsubstituted non-aromatic heterocyclic group or an alkyl group substituted with a substituted or unsubstituted aryl group.

In certain embodiments, R₁ and R₂ taken together with the atoms to which they are attached form an optionally substituted ring. In particular embodiments, the optionally substituted ring is aromatic, such as a 6-membered aromatic ring. In certain embodiments, R₁ is a substituted or unsubstituted aryl group, such as a substituted or unsubstituted heteroaryl group. In certain embodiments, R₁ is a substituted or unsubstituted alkyl group, such as a methyl or ethyl group.

In certain embodiments, Ring A is unsubstituted. An exemplary embodiment is where Ring A is unsubstituted and R₁ is a substituted or unsubstituted aryl group. In certain embodiments, Ring A is substituted, such as with a substituted or unsubstituted alkyl group. An exemplary embodiment is where Ring A is substituted and R₁ is a substituted or unsubstituted alkyl group.

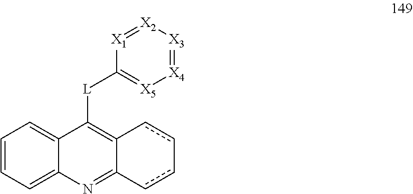

wherein:
each of X₁, X₂, X₃, X₄ and X₅ is independently selected from N or CR₆, wherein no more than two of X₁, X₂, X₃, X₄ or X₅ are N; each R₆ is independently selected from H, —OCH₃, —CH₃, or —CF₃; L is selected from —CH═CH—C(O)—, —CH₂—N(R₄)—C(O)—, —C(O)—CH₂—,
—C(O)—N(R₄)—, —C(O)—N(R₄)—CH₂—, —C(O)—N(R₄)—CH₂—CH₂—, —C(O)—N(R₄)—C(O)—, —C(O)—N(R₄)—N(R₅)—, —CH₂—N(R₄)—N(R₅)—, —N(R₄)—S(O)₂—, —S(O)₂—N(R₄)—, —N(R₄)—N(R₅)—C(O)—, —C(O)—N(R₄)—N(R₅)—C(O)—, —N(R₄)—N(R₅)—CH₂, —N(R₄)—N(R₅)—,

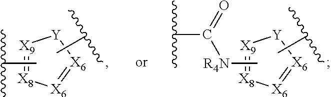

each of R₄ and R₅ is independently selected from H or CH₃;
Y is selected from O, S, or NR₄; each of X₆, X₇, X₈ and X9 is independently selected from CR₇, C, or N, wherein at least two of X₆, X₇, X₈ or X₉ are not N; each R₇ is independently selected from H or (C₁-C₃)-straight or branched alkyl; and the hashed bonds are either simultaneously present or simultaneously absent. In certain embodiments, when the hashed bonds are simultaneously present, L is —N(R₄)—N(R₅)—C(O)—, and each of X₂, X₃, and X₄ are —OCH₃, then R₄ is hydrogen.

In certain embodiments, when the hashed bonds are simultaneously absent and L is —N(R₄)—N(R₅)—C(O)—, both X₁ and X₅ are CR₆. In certain embodiments, L is selected from —C(O)—N(R₄)—N(R₅)—, —CH₂—N(R₄)—N(R₅)—, —N(R₄)—N(R₅)—C(O)—, —N(R₄)—N(R₅)—,

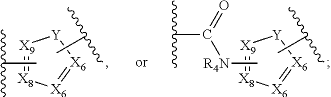

particularly —NH—NH—C(O)—, —NH—NH—, —N(CH₃)—NH—C(O)—, —CH₂—NH—NH—, —C(O)—NH—NH—,

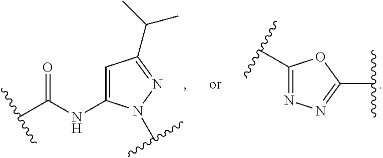

In certain embodiments, such as when L has one of the values described above, no more than one of X₁, X₂, X₃, X₄ and X₅ is N, for example, exactly one of X₁, X₂, X₃, X₄ and X₅ is N. In certain such embodiments, each of X₁, X₂, X₃, X₄ and X₅ is selected from N or CH. In other such embodiments, each of X₁, X₂, X₃, X₄ and X₅ is CR₆, such as where each R₆ is hydrogen. In a particular embodiment, X₁ and X₅ are CH and each of X₂, X₃, and X₄ is C—OCH₃.

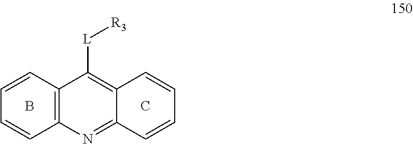

wherein:
Rings B and C are independently optionally substituted;
L is —NR₄R₅, —C(O)O—, —C(O)NR₄—, —NR₄C(O)—, —NR₄—NR₅—C(O)—, —C(O)—NR₄—NR₅— or —CHR₄═CHR₅—; and
R₃, R₄ and R₅ are independently —H, a substituted or unsubstituted alkyl group, a substituted or unsubstituted aryl group or a substituted or unsubstituted non-aromatic heterocyclic group.

In certain embodiments, L is —C(O)—NR₄—NR₅—. In certain embodiments, R₃ is a substituted or unsubstituted aryl group. In particular embodiments, L is —C(O)—NR₄—NR₅— and R₃ is a substituted or unsubstituted aryl group. Particular R₃ groups are substituted or unsubstituted phenyl or pyridyl groups, such as a pyridyl or an alkoxy-substituted phenyl group (e.g., a trialkoxy-substituted phenyl group such as 3,4,5-trimethoxyphenyl).

In certain embodiments, Ring B and/or Ring C is unsubstituted. Preferably, both Rings B and C are unsubstituted, such as when L is —C(O)—NR₄—NR₅— and/or R₃ is a substituted or unsubstituted aryl group. In certain embodiments, R₄ and/or R₅ are —H. Preferably, both R₄ and R₅ are —H. In particular embodiments, Rings B and C are unsubstituted when L is —C(O)—NR₄—NR₅— and/or R₃ is a substituted or unsubstituted aryl group. In certain embodiments, R₂ is selected from —H and —OH. In certain embodiments, R₂ is —H.

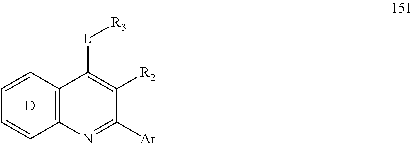

wherein:
Ring D is optionally substituted;
Ar is a substituted or unsubstituted aryl group;
R₂ is selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;
L is selected from —C(O)O—, —C(O)—, —C(O)N(R₄)—, —C(O)—N(R₄)—C(O)—, —C(O)—N(R₄)—N(R₅)—, —C(O)—N(R₄)—N(R₅)—C(O)—, —C(O)—N(R₄)—S(O)₂—, —N(R₄)C(O)—, —N(R₅)—S(O)₂—, —N(R₄)—S(O)₂—N(R₅), —N(R₄)(R₅)—, —N(R₄)—N(R₅)—C(O)—, —N(R₄)—C(O)—N(R₅)—, —N(R₄)—C(O)—N(R₅)—S(O)₂, —N(R₄)—C(S)—N(R₅)—, —N(R₄)—C(O)—CH₂—N(R₅)—, —N(R₄)—C(O)—CH═C(CH₃)—, —N(R₄)—C(═N—CN)—N(R₅)—, —N(R₄)—C(═NH)—N(R₅)—, —N(R₄)—, —N(R₄)—CH₂—C(O)—N(R₅)—, —CH₂—, —CH₂—N(R₄)—C(O)—, —CH₂—C(O)—N(R₄)—, —CH(R₄)═CH(R₅)—, —CH═CH—C(O)—, —N(R₄)—N(R₅)—, —CH₂—N(R₄)—N(R₅)—, —S(O)₂—N(R₄)—,

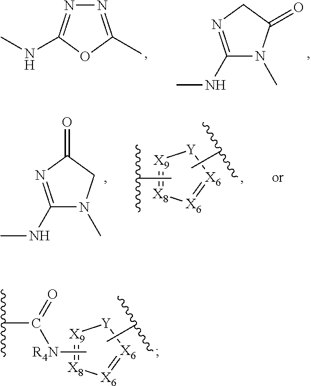

such as —NR₄R₅, —C(O)O—, —C(O)NR₄—, —NR₄C(O)—, —NR₄—NR₅—C(O)—, —C(O)—NR₄—NR₅— or —CHR₄═CHR₅—; each of R₃, R₄ and R₅ is independently selected from —H, a substituted or unsubstituted alkyl group, a substituted or unsubstituted aryl group or a substituted or unsubstituted non-aromatic heterocyclic group;
Y is selected from O, S, or NR₄;
each of X₆, X₇, X₈ and X₉ is independently selected from CR₇, C, or N, wherein at least two of X₆, X₇, X₈ or X₉ are not N;
each R₇ is independently selected from H or (C₁-C₃)-straight or branched alkyl; and
n is 1 or 2.

In certain embodiments, R₄ or R₅ when it appears in L is selected from H and (C₁-C₃)-straight or branched alkyl. In certain embodiments, R₄ and R₅ when they appear in L are H. In certain embodiments, R₂ is selected from H and OH. In certain embodiments, R₂ is H. In certain embodiments, R₃ is a substituted or unsubstituted non-aromatic heterocyclic group or a substituted or unsubstituted aryl group, such as a substituted or unsubstituted heteroaryl group.

In certain embodiments, R₃ is selected from —H, Cyc or (C₁-C₂)alkylene-Cyc, wherein when R₃ is —H, L is —C(O)O—; Cyc is selected from a substituted aryl group, an unsubstituted aryl group, a substituted non-aromatic heterocyclic group or an unsubstituted non-aromatic heterocyclic group; and each of R₄ and R₅ is independently selected from —H or —CH₃. In certain such embodiments, L and R₃ taken together form a moiety selected from C(O)—OH, C(O)—N(R₄)-Cyc, C(O)—N(R₄)—(CH₂)ₙ-CyC, N(R₄)—N(R₅)—C(O)—CyC, N(R₄)—N(R₅)-CyC, CH₂—N(R₄)—N(R₅)-Cyc, C(O)—N(R₄)—N(R₅)-Cyc, or

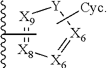

In particular embodiments, L and R₃ taken together form a moiety selected from —C(O)—OH, —C(O)—NH—(CH₂)ₙ-Cyc, —C(O)—NH-Cyc, —NH—NH—C(O)—CyC, —NH—NH-Cyc, —N(CH₃)—NH—C(O)-Cyc, —CH₂—NH—NH-CyC, —C(O)—NH—NH-CyC, or

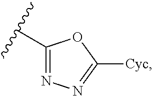

preferably —C(O)—OH, —C(O)—NH—(CH₂)ₙ-Cyc, —C(O)—NH-CyC, or —NH—NH—C(O)-Cyc, such as —C(O)—NH—(CH₂)ₙ-Cyc where Cyc is unsubstituted. Typically, Cyc is selected from pyridyl or morpholino. In other particular embodiments, L and R₃ taken together form —NH—NH—C(O)-Cyc; and Cyc is phenyl.

In particular embodiments, when L and R₃ are taken together to form C(O)—N(R₄)-Cyc, and Cyc is phenyl, the phenyl is monosubstituted with morpholino. In particular embodiments, when L and R₃ are taken together to form N(R₄)—N(R₅)—C(O)-Cyc and Cyc is a substituted phenyl, the substituted phenyl is not 3,4,5 trimethoxyphenyl or 4-N,Ndimethylaminophenyl. In particular embodiments, when L and R₃ are taken together to form C(O)—N(R₄)—(CH₂)₂-Cyc, Cyc is not piperidinyl or piperazinyl. In particular embodiments, when L and R₃ are taken together to form C(O)—N(R₄)—(CH₂)₂-Cyc and Cyc is morpholino, Ar is not furanyl.

In certain embodiments, Ar is unsubstituted. In certain such embodiments, Ar is selected from phenyl, pyridyl, thienyl, or furanyl. In certain embodiments, ring D is unsubstituted or monosubstituted, particularly when Ar is selected from phenyl, pyridyl, thienyl, or furanyl. When ring D is monosubstituted, the substituent is typically at the 6-position of the ring system. Typical substituents for ring D include a substituted or unsubstituted alkyl group, a substituted or unsubstituted aryl group, a substituted or unsubstituted non-aromatic heterocyclic group, halogen, —OR₄, —CN₅—CO₂R₄, —OCOR₄, —OCO₂R₄, —C(O)NR₄R₅, —OC(O)NR₄R₅, —C(O)R₄, —COR₄, —SR₄, —OSO₃H₅—S(O)ₙR₄, —S(O)ₙOR₄, —S(O)ₙNR₄R₅, —NR₄R₅, —NR₄C(O)OR₅, —NR₄C(O)R₅ and —NO₂. Preferred substituents include methyl and halo. In certain embodiments, L is —C(O)NR₄—. In certain such embodiments, R₃ is a substituted or unsubstituted heteroaryl group having at least one ring nitrogen atom or a substituted or unsubstituted non-aromatic heterocyclic group having at least one nitrogen atom and or a C₁₋₂ alkylene (e.g., unsubstituted alkylene) group substituted by substituted or unsubstituted heteroaryl group having at least one ring nitrogen atom or a substituted or unsubstituted non-aromatic heterocyclic group having at least one nitrogen atom.

In certain embodiments, L is —NR₄R₅—. In certain embodiments, R₃ is a substituted alkyl group or a cyclic group. When R₃ is a substituted alkyl group, it is preferably substituted with a cyclic group. When R₃ is a cyclic group, it is preferably an aryl group (e.g., phenyl) or a non-aromatic heterocyclic group (e.g., morpholino). In a particular embodiment, L is —C(O)NR₄— and R₃ is a substituted alkyl group or a cyclic group. When R₃ is a cyclic group or an alkyl group substituted with a cyclic group, the cyclic group is typically a phenyl or pyridyl group that is unsubstituted or substituted only at one or both of the positions adjacent to where R₃ attaches to L.

In other certain embodiments, R₃ is a cyclic group substituted at least one position that is not adjacent to the atom by which R₃ attaches to L. For example, if R₃ is a phenyl or pyridyl group, at least one substituent is meta ox para to the atom where R₃ attaches to L. In certain embodiments, R₁ is a substituted or unsubstituted heteroaryl group, such as a thienyl or furanyl group. In particular embodiments, R₁ is a substituted or unsubstituted heteroaryl group, such as a thienyl or furanyl group, when L is —C(O)NR₄— and/or R₃ is a substituted alkyl group or a cyclic group. For example, R₁, R₃ and L can have these values when R₃ is a cyclic group or an alkyl group substituted with a cyclic group, the cyclic group is typically a phenyl or pyridyl group that is unsubstituted or substituted only at one or both of the positions adjacent to where R₃ attaches to L.

In certain embodiments, Ring D is unsubstituted or is substituted with a halogen (e.g., Cl, Br, F, I) or an unsubstituted alkyl group (e.g., methyl, ethyl, propyl). In particular embodiments, Ring D is unsubstituted or is substituted with a halogen or an unsubstituted alkyl group when R₁ is a substituted or unsubstituted heteroaryl group, L is —C(O)NR₄— and/or R₃ is a substituted alkyl group or a cyclic group. In other particular embodiments, Ring D is substituted with a halogen or an unsubstituted alkyl group when R₁ is a substituted or unsubstituted heteroaryl group or a substituted or unsubstituted alkyl group, L is —C(O)NR₄— or —NR₄R₅— and/or R₃ is a substituted alkyl group or a cyclic group.

One group of compounds encompassed by formula (151) are represented by the formula:

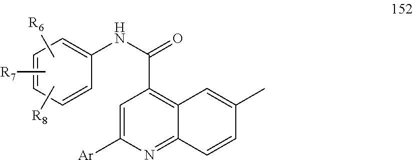

wherein:
Ar is selected from phenyl,

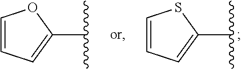

and each of R₆, R₇, and R₈ is independently selected from —H, —CF₃, —C₁-C₃ straight or branched alkyl, —O—(C₁-C₃ straight or branched alkyl), —O—CF₃, —N(C₁-C₃ straight or branched alkyl)₂, halo, morpholino, —(C₁-C₃ straight or branched alkyl)-morpholino, piperazinyl, —(C₁-C₃ straight or branched alkyl)-piperazinyl, -piperazinyl, —NH—S(O)₂—(C₁-C₃ straight or branched alkyl), or —NH—S(O)₂-phenyl, wherein the phenyl, piperazinyl or morpholino is optionally substituted with methyl. In —N(C₁-C₃ straight or branched alkyl)₂, the two alkyl groups may be the same or different.

In particular embodiments, R₆ is morpholino, —(C1-C3 straight or branched alkyl)-morpholino, piperazinyl, —(C1-C3 straight or branched alkyl)-piperazinyl, -piperazinyl, or —NH—S(O)₂-phenyl, wherein the phenyl, piperazinyl or morpholino is optionally substituted with methyl, and R₇ and R₈ are hydrogen. In other particular embodiments, each of R₆, R₇, and R₈ is independently selected from —H, —CF₃, -methyl, —O-methyl, —O—CF₃, —N(CH₃)₂, fluoro, morpholino, —CH₂—CH₂-morpholino, piperazinyl-CH₃, —NH—S(O)₂—CH₃, or —NH—S(O)₂-phenyl-CH₃.

In certain embodiments, L is selected from —C(O)O—, —C(O)—, —C(O)—N(R₄)—C(O)—, —C(O)—N(R₄)—N(R₅)—, —C(O)—N(R₄)—S(O)₂—, —N(R₄)C(O)—, —N(R₄)—S(O)₂—, —N(R₄)—S(O)₂—N(R₅), —N(R₄)(R₅)—, —N(R₄)—N(R₅)—C(O)—, —N(R₄)—C(O)—N(R₅)—, —N(R₄)—C(O)—N(R₅)—S(O)₂, —N(R₄)—C(S)—N(R₅)—, —N(R₄)—C(O)—CH₂—N(R₅)—, —N(R₄)—C(O)—CH═C(CH₃)—, —N(R₄)—C(═N—CN)—N(R₅)—, —N(R₄)—C(═NH)—N(R₅)—, —N(R₄)—, —N(R₄)—CH₂—C(O)—N(R₅)—, —CH₂—, —CH₂—N(R₄)—C(O)—, —CH₂—C(O)—N(R₄)—, —CH(R₄)═CH(R₅)—, —CH═CH—C(O)—, —N(R₄)—N(R₅)—, —CH₂—N(R₄)—N(R₅)—, —S(O)₂—N(R₄)—,

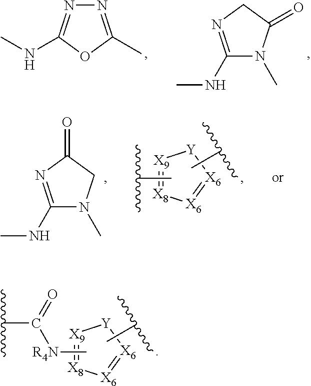

A particular group of compounds of the invention encompassed by formula (151) are represented by formula (153):

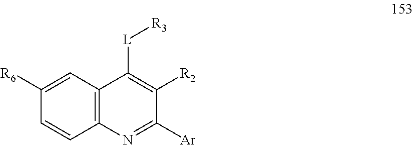

wherein:
R₆ is selected from —H, a substituted or unsubstituted alkyl group, a substituted or unsubstituted aryl group, a substituted or unsubstituted non-aromatic heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂. Preferred values of R₆ are a halogen or an unsubstituted alkyl group.

Suitable values of Ar, L, R₂ and R₃ are as described above.

For compounds of formulae 148-153, the following definitions apply:

An “alkyl group” is a straight chained, branched or cyclic non-aromatic hydrocarbon which is completely saturated. Typically, a straight chained or branched alkyl group has from 1 to about 20 carbon atoms, preferably from 1 to about 10, and a cyclic alkyl group has from 3 to about 10 carbon atoms, preferably from 3 to about 8. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An “alkenyl group” is a straight chained, branched or cyclic non-aromatic hydrocarbon which contains one or more double bonds. Typically, the double bonds are not located at the terminus of the alkenyl group, such that the double bond is not adjacent to another functional group.

An “alkynyl group” is a straight chained, branched or cyclic non-aromatic hydrocarbon which contains one or more triple bonds. Typically, the triple bonds are not located at the terminus of the alkynyl group, such that the triple bond is not adjacent to another functional group.

A “cyclic group” includes carbocyclic and heterocyclic rings. Such rings can be saturated or unsaturated, including aromatic. Heterocyclic rings typically contain 1 to 4 heteroatoms, although oxygen and sulfur atoms cannot be adjacent to each other. Aromatic (aryl) groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl. Non-aromatic heterocyclic rings are non-aromatic carbocyclic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl, along with the cyclic form of sugars.

A ring fused to a second ring shares at least one common bond. Suitable substituents on an alkyl, alkenyl, alkynyl, aryl, non-aromatic heterocyclic or aryl group (carbocyclic and heteroaryl) are those which do not substantially interfere with the ability of the disclosed compounds to have one or more of the properties disclosed herein. A substituent substantially interferes with the properties of a compound when the magnitude of the property is reduced by more than about 50% in a compound with the substituent compared with a compound without the substituent. Examples of suitable substituents include —OH, halogen (—Br, —Cl, —I and —F), —OR^{a}, —O—COR^{a}, —COR^{a}, —C(O)R^{a}, —CN, —NO², —COOH, —COOR^{a}, —OCO₂R^{a}, —C(O)NR^{a}R^{b}, —OC(O)NR^{a}R^{b}, —SO₃H, —NH₂, —NHR^{a}, —N(R^{a}R^{b}), —COOR^{a}, —CHO, —CONH₂, —CONHR³, —C0N(R^{a}R^{b}), —NHC0R^{a}, —NRC0R^{a}, —NHCONH₂, —NHCONRH, —NHC0N(R^{a}R^{b}), —NR^{c}CONH₂, —NR^{c}CONR³H, —NR^{c}C0N(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHRₐ, —SO₂NR^{a}R^{b}, —CH═CHR^{a}, —CH═CR^{a}R^{b}, —CR^{c}═CR^{a}R^{b}, CR^{c}═CHR^{a}, —CR^{c}═CR^{a}R^{b}, —CCR^{a}, —SH, —SOₖR^{a} (k is 0, 1 or 2), —S(O)ₖOR^{a} (k is 0, 1 or 2) and —NH—C(═NH)—NH₂. R^{a}-R^{d} are each independently an aliphatic, substituted aliphatic, benzyl, substituted benzyl, aromatic or substituted aromatic group, preferably an alkyl, benzylic or aryl group. In addition, —NR^{a}R^{b}, taken together, can also form a substituted or unsubstituted non-aromatic heterocyclic group. A non-aromatic hetcroc3′clic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group can have more than one substituent.

In certain embodiments, exemplary sirtuin-activating or sirtuin-inhibiting compounds are N-benzimidazolylalkyl-substituted compounds as disclosed in WO 2006/094209, hereby incorporated by reference in its entirety. Such exemplary compounds include compounds of formulae 154-159, described below:

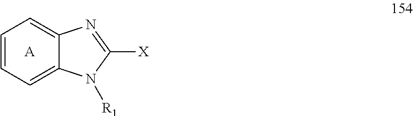

wherein:
Ring A is optionally substituted;
R₁ is —H, a substituted or unsubstituted alkyl group, a substituted or unsubstituted alkenyl group, a substituted or unsubstituted aryl group or a substituted or unsubstituted heterocyclic group;
X is —(CHR₂)ₘ—NHCO—R₃, —(CHR₂)ᵣ—NHCONR₄R₅, —(CH₂)ᵣNR₁ SO₂—R₃, —SR₃ or -arylene-R₂;
each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;
R₃ is a substituted or unsubstituted alkyl group, a substituted or unsubstituted aryl group or a substituted or unsubstituted heterocyclic group;
R₄ and R₅ are independently —H, a substituted or unsubstituted alkyl group, a substituted or unsubstituted aryl group or a substituted or unsubstituted non-aromatic heterocyclic group;
m is an integer from 2 to 12; n is 1 or 2; and r is an integer from 0 to 12.

Typically, Ring A is directly or indirectly substituted with a carboxy group. Ih an exemplary embodiment, such compounds are represented by formula (155):

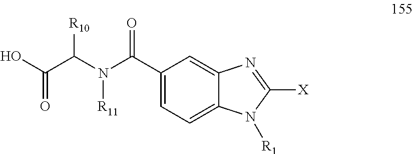

wherein:
R₁₀ is selected from the group consisting of —H, a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂; or
R₁₀ and R₁₁ taken together with the atoms to which they are attached form a non-aromatic heterocyclic ring; and R₁₁ is —H, a substituted or unsubstituted alkyl group, a substituted or unsubstituted aryl group or a substituted or unsubstituted heterocyclic group.

In certain embodiments, R₁₀ is a substituted or unsubstituted alkyl group (e.g., methyl). In certain embodiments, R₁₁ is —H. Typically, when R₁₁ is —H, R₁₀ is a substituted or unsubstituted alkyl group (e.g., methyl). In certain embodiments, R₁₀ and R₁₁ taken together with the atoms to which they are attached form a non-aromatic heterocyclic ring. Examples of such compounds are represented by formula (156):

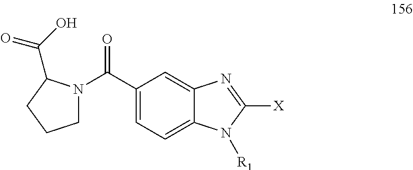

In certain embodiments, X in compounds represented by formulae (154), (155) and (156) is —(Cm₂X—NHCONR₄R₅. Typically, R₂ in such compounds is a substituted or unsubstituted alkyl group (e.g., methyl, ethyl, propyl, butyl, pentyl). Separately and in combination with these values of R₂, r is typically 1. Separately or in combination with these values of R₂ and r, R₄ is typically —H and R₅ is typically a substituted or unsubstituted alkyl (e.g., isopropyl) or alkenyl group.

In certain embodiments, X in compounds represented by formulae (154), (155) and (156) is —SR₃ and R₃ is a substituted or unsubstituted alkyl group. Typically, R₃ in such compounds is a carboxy-substituted alkyl group (e.g., carboxymethyl), particularly when Ring A is unsubstituted. In certain embodiments, X in compounds represented by formulae (154), (155) and (156) is —(CH₂)ᵣNR₁SO₂—R₃ and R₃ is a substituted or unsubstituted aryl group. In certain embodiments, X in compounds represented by formulae (154), (155) and (156) is -arylene-R₂, wherein the arylene is phenylene (e.g., unsubstituted phenylene). R₂ in such compounds is typically a substituted or unsubstituted C₁-C₆ alkyl group or a halogen.

In another embodiment, compounds of the invention are represented by formula (157):

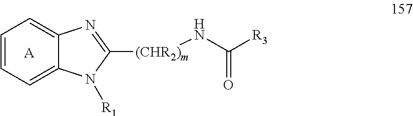

wherein:
Ring A is optionally substituted;
R₁ is —H, a substituted or unsubstituted alkyl group, a substituted or unsubstituted alkenyl group, a substituted or unsubstituted aryl group or a substituted or unsubstituted heterocyclic group;
each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)ₙOR₄, —S(O)ₙNR₄R₅, —NR₄R₅, —NR₄C(O)OR₅, —NR₄C(O)R₅ and —NO₂;
R₃ is a substituted or unsubstituted alkyl group, a substituted or unsubstituted aryl group or a substituted or unsubstituted heterocyclic group;
R₄ and R₅ are independently —H, a substituted or unsubstituted alkyl group, a substituted or unsubstituted aryl group or a substituted or unsubstituted heterocyclic group;
m is an integer from 2 to 12; and n is 1 or 2.

In certain embodiments, m is 2. In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group and m is 2.

In certain embodiments, R₁ is a substituted or unsubstituted alkyl group or a substituted or unsubstituted alkenyl group, such as ethyl, n-propyl, cyclopropylmethyl, 2-propenyl, benzyl and methoxyethyl. In particular embodiments, R₁ is a substituted or unsubstituted alkyl group or a substituted or unsubstituted alkenyl group when R₃ is a substituted or unsubstituted alkyl group and/or m is 2.

In certain embodiments, R₂ is —H or a substituted or unsubstituted aryl group, such as —H or a pyridyl group. In particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group and m is 2.

In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group and m is 2.

In certain embodiments, R₁ is a substituted or unsubstituted alkyl group or a substituted or unsubstituted alkenyl group, such as ethyl, n-propyl, cyclopropylmethyl, 2-propenyl, benzyl and methoxyethyl. In particular embodiments, R₁ is a substituted or unsubstituted alkyl group or a substituted or unsubstituted alkenyl group when R₃ is a substituted or unsubstituted alkyl group and/or m is 2. In certain embodiments, R₂ is —H or a substituted or unsubstituted aryl group, such as —H or a pyridyl group, hi particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group and m is 2.

In another embodiment, sirtuin-modulating compounds of the invention are represented by formula (158):

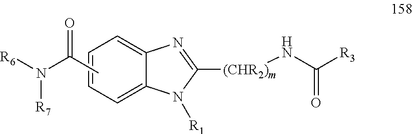

wherein:
R₁ is —H, a substituted or unsubstituted alkyl group, a substituted or unsubstituted aryl group or a substituted or unsubstituted heterocyclic group;
each R₂ is independently selected from the group consisting of a substituted or unsubstituted alkyl group, a substituted or unsubstituted aryl group, a substituted or unsubstituted heterocyclic group, halogen, —OR₄, —CN, —CO₂R₄, —OCOR₄, —OCO₂R₄, —C(O)NR₄R₅, —OC(O)NR₄R₅, —C(O)R₄, —COR₄, —SR₄, —OSO₃H, —S(O)ₙR₄, —S(O)₁₀R₄, —S(O)ₙNR₄R₅, —NR₄R₅, —NR₄C(O)OR₅, —NR₄C(O)R₅ and —NO₂;
R₃ is a substituted or unsubstituted alkyl group, a substituted or unsubstituted aryl group or a substituted or unsubstituted heterocyclic group; R₄ and R₅ are independently —H, a substituted or unsubstituted alkyl group, a substituted or unsubstituted aryl group or a substituted or unsubstituted heterocyclic group;
R₆ and R₇ are independently —H, a substituted or unsubstituted alkyl group, a substituted or unsubstituted aryl group or a substituted or unsubstituted heterocyclic group, or R₆ and R₇ taken together with the nitrogen atom to which they are attached form a heterocyclic ring; m is an integer from 2 to 12;
and n is 1 or 2.

In certain embodiments, R₆ is a substituted alkyl group, such as a carboxyalkyl group (e.g., 1-carboxyethyl). In certain embodiments, R₇ is —H. In particular embodiments, R₇ is —H and is a substituted alkyl group. In certain embodiments, R₆ and R₇ taken together with the nitrogen atom to which they are attached form a heterocyclic ring. Suitable heterocyclic rings include substituted or unsubstituted piperazine and pyrrolidine, such as piperazine or 2-carboxypyrrolidine.

In certain embodiments, m is 2. In particular embodiments, m is 2 and R₆ and R₇ have the values indicated above. In certain embodiments, R₃ is a substituted or unsubstituted alkyl group, such as a carboxyalkyl group (e.g., carboxyethyl, 2,2-dimethylpropyl). In particular embodiments, R₃ is a substituted or unsubstituted alkyl group when m is 2 and/or R₆ and R₇ have the values indicated above.

In certain embodiments, R₁ is a substituted or unsubstituted alkyl group or a substituted or unsubstituted alkenyl group, such as ethyl, n-propyl, cyclopropylmethyl, 2-propenyl, 2-propynyl, benzyl and methoxyethyl. In particular embodiments, R₁ is a substituted or unsubstituted alkyl group or a substituted or unsubstituted alkenyl group when R₃ is a substituted or unsubstituted alley! group, R₆ and R₇ have the values indicated above and/or m is 2.

In certain embodiments, R₂ is —H or a substituted or unsubstituted aryl group, such as —H or a pyridyl group. In particular embodiments, R₂ is —H or a substituted or unsubstituted aryl group when R₁ is a substituted or unsubstituted alkyl group or a substituted or unsubstituted alkenyl group, R₃ is a substituted or unsubstituted alkyl group, R₆ and R₇ have the values indicated above and/or m is 2. Preferred compounds are chosen such that R₂ is —H or a substituted or unsubstituted aryl group, R₁ is a substituted or unsubstituted alkyl group or a substituted or unsubstituted alkenyl group, R₃ is a substituted or unsubstituted alkyl group, R₆ and R₇ have the values indicated above and m is 2.

One group of compounds of the invention encompassed by formula (158) is represented by formula (159):

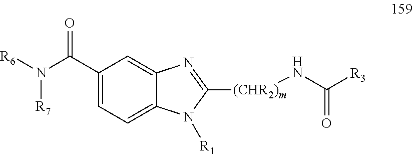

Definitions applicable to compounds of formulae 130-143 are also applicable to those compounds represented by formulae 154-159.

In certain embodiments, exemplary sirtuin-activating or sirtuin-inhibiting compounds are N-phenyl benzamide derivatives as disclosed in WO 2006/094236, hereby incorporated by reference in its entirety. Such exemplary compounds include compounds of formulae 160-162, described below:

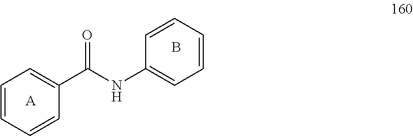

wherein:
Ring A is optionally substituted; and
Ring B is substituted with at least one carboxy or polycyclic aryl group.

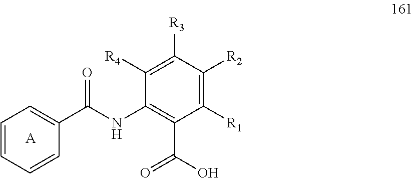

wherein:
Ring A is optionally substituted;
R₁, R₂, R₃ and R₄ are independently selected from the group consisting of —H, halogen, —OR₅, —CN₅—CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;
R₅ and R₆ are independently —H, a substituted or unsubstituted alkyl group, a substituted or unsubstituted aryl group or a substituted or unsubstituted heterocyclic group; and
n is 1 or 2.

In certain embodiments, R₁, R₂, R₃ and R₄ are independently selected from the group consisting of —H, —OR₅ and —SR₅, particularly —H and —OR₅ (e.g., —H, —OH, —OCH₃). Ring A is preferably substituted. Suitable substituents include halogens (e.g., bromine), acyloxy groups (e.g., acetoxy), aminocarbonyl groups (e.g., arylaminocarbonyl such as substituted, particularly carboxy-substituted, phenylaminocarbonyl groups) and alkoxy (e.g., methoxy, ethoxy) groups.

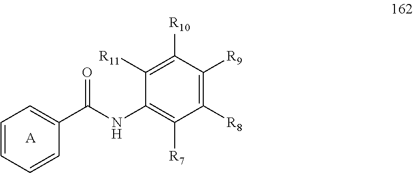

wherein:
Ring A is optionally substituted;
R₅ and R₆ are independently —H, a substituted or unsubstituted alkyl group, a substituted or unsubstituted aryl group or a substituted or unsubstituted heterocyclic group;
R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR_{S}, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;
R₈ is apolycyclic aryl group; and
n is 1 or 2.

In certain embodiments, one or more of R₇, R₉, R₁₀ and R₁₁ are —H. In particular embodiments, R₇, R₉, R₁₀ and R₁₁ are each —H. In certain embodiments, R₈ is a heteroaryl group, such as an oxazolo[4,5-b]pyridyl group. In particular embodiments, R₈ is a heteroaryl group and one or more of R₇, R₉, R₁₀ and R₁₁ are —H.

Ring A is preferably substituted. Suitable substituents include halogens (e.g., bromine), acyloxy groups (e.g., acetoxy), aminocarbonyl groups (e.g., arylaminocarbonyl, such as substituted, particularly carboxy-substituted, phenylaminocarbonyl groups) and alkoxy (e.g., methoxy, ethoxy) groups, particularly alkoxy groups. In certain embodiments, Ring A is substituted with at least one alkoxy or halo group, particularly methoxy.

In certain embodiments, Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain embodiments, Ring A is not substituted with a nitrile or pyrrolidyl group.

In certain embodiments, R₈ is a substituted or unsubstituted bicyclic heteroaryl group, such as a bicyclic heteroaryl group that includes a ring N atom and 1 to 2 additional ring heteroatoms independently selected from N, O or S. Preferably, R₈ is attached to the remainder of the compound by a carbon-carbon bond. In certain such embodiments, 2 additional ring heteroatoms are present, and typically at least one of the additional ring heteroatoms is O or S. In certain such embodiments, 2 total ring nitrogen atoms are present (with zero or one O or S present), and the nitrogen atoms are typically each in a different ring. In certain such embodiments, R₈ is not substituted with a carbonyl-containing moiety, particularly when R₈ is thienopyrimidyl or thienopyridinyl.

In certain such embodiments, R₈ is selected from oxazolopyridyl, benzothienyl, benzofuranyl, indolyl, quinoxalinyl, benzothiazolyl, benzooxazolyl, benzimidazolyl, quinolinyl, isoquinolinyl or isoindolyl. In certain such embodiments, R₈ is selected from thiazolopyridyl, imidazothiazolyl, benzooxazinonyl, or imidazopyridyl.

Particular examples of R₈, where [symbol] indicates attachment to the remainder of formula (162), include:

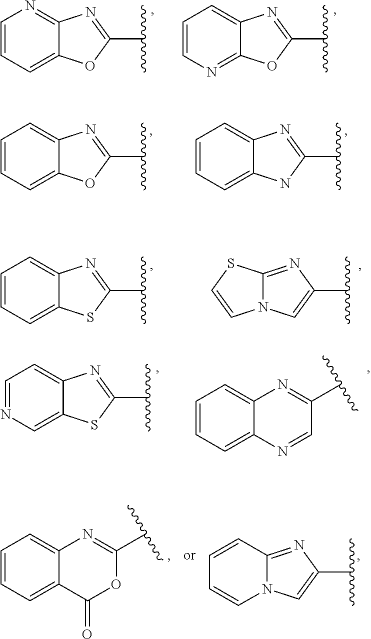

where up to 2 ring carbons not immediately adjacent to the indicated attachment point are independently substituted with O—C₁-C₃ straight or branched alkyl, C₁-C₃ straight or branched alkyl or halo, particularly C₁-C₃ straight or branched alkyl or halo. In certain embodiments, R₈ is

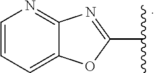

In certain embodiments, R₈ is

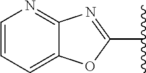

and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle. In certain such embodiments, Ring A is not simultaneously substituted at the 2- and 6-positions with O—(C₁-C₃ straight or branched alkyl). In certain such embodiments, Ring A is not simultaneously substituted at the 2-, 4- and 6-positions with O—(C₁-C₃ straight or branched alkyl). In certain such embodiments, Ring A is not simultaneously substituted at the 2-, 3-, and 4-positions with O—(C₁-C₃ straight or branched alkyl). In certain such embodiments, Ring A is not substituted at the 4-position with a 5 to 6-membered heterocycle. In certain such embodiments, Ring A is not singly substituted at the 3- or 4-position (typically 4-position) with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

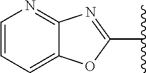

and Ring A is optionally substituted with up to 3 substituents independently selected from (C₁-C₃ straight or branched alkyl), (C₁-C₃ straight or branched haloalkyl, where a haloalkyl group is an alkyl group substituted with one or more halogen atoms), O—(C₁-C₃ straight or branched alkyl), N(C₁-C₃ straight or branched alkyl)₂, halo, or a 5 to 6-membered heterocycle. In certain such embodiments, Ring A is not singly substituted at the 3- or 4-position with O—(C₁-C₃ straight or branched alkyl). In certain such embodiments, Ring A is not substituted at the 4-position with O—(C₁-C₃ straight or branched alkyl) and at the 2- or 3-position with C₁-C₃ straight or branched alkyl.

In certain embodiments, R₈ is

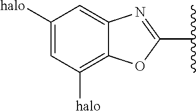

(e.g., where one or both halo is chlorine) and Ring A is optionally substituted with up to 3 substituents independently selected from (C₁-C₃ straight or branched alkyl), O—(C₁-C₃ straight or branched alkyl), N(C₁-C₃ straight or branched alkyl)₂, halo, or a 5 to 6-membered heterocycle, but not singly substituted at the 3-position with O—(C₁-C₃ straight or branched alkyl).

In certain embodiments, such as when R₈ has one of the values described above, Ring A is substituted with up to 3 substituents independently selected from chloro, methyl, O-methyl, N(CH₃)₂ or morpholino. In certain such embodiments, R₈ is selected from

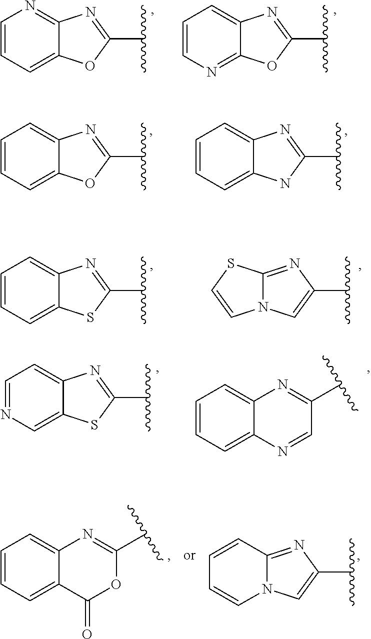

where up to 2 ring carbons not immediately adjacent to the indicated attachment point are independently substituted with C₁-C₃ straight or branched alkyl or halo; each of R₇, R₉, and R₁₁ is —H; and R₁₀ is selected from —H, —CH₂OH, —CO₂H, —CO₂CH₃, —CH₂-piperazinyl, CH₂N(CH₃)₂, —C(O)—NH—(CH₂)₂—N(CH₃)₂, or —C(O)-piperazinyl. In certain such embodiments, when R₈ is

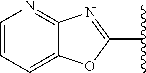

and Ring A is 3-dimethylaminophenyl, none of R₇, R₉, R₁₀ and R₁₁ is —CH₂—N(CH₃)₂ or —C(O)—NH—(CH₂)₂—N(CH₃)₂, and/or when R₈ is

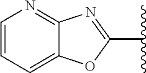

and Ring A is 3,4-dimethoxyphenyl, none of R₇, R₉, R₁₀ and R₁₁ is C(O)OCH₃ or C(O)OH.

In certain embodiments, such as when R₈ has one of the values described above and/or Ring A is optionally substituted as described above, at least one of R₇, R₉, R₁₀ and R₁₁ is —H. In certain such embodiments, each of R₇, R₉, R₁₀ and R₁₁ is —H.

In certain embodiments, R₇, R₉, R₁₀ and R₁₁ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl, —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2. In certain such embodiments, R₁₀ is selected from —C(O)OH, —N(CH₃)₂, —CH₂OH, —CH₂OCH₃, —CH₂-piperazinyl, —CH₂-methylpiperazinyl, —CH₂-pyrrolidyl, —CH₂-piperidyl, —CH₂-morpholino, —CH₂—N(CH₃)₂, —C(O)—NH—(CH₂)ₙ-piperazinyl, —C(O)—NH—(CH₂)ₙ-methylpiperazinyl —C(O)—NH—(CH₂)ₙ-pyrrolidyl, —C(O)—NH—(CH₂)ₙ-morpholino, —C(O)—NH—(CH₂)ₙ-piperidyl, or —C(O)—NH—(CH₂)ₙ—N(CH₃)₂, wherein n is 1 or 2, and each of R₇, R₉, and R₁₁ is H.

In certain embodiments, Ring A is substituted with a nitrile group or is substituted at the para position with a 5- or 6-membered heterocycle. Typical examples of the heterocycle include pyrrolidyl, piperidinyl and morpholinyl.

Definitions applicable to compounds of formulae 130-143 are also applicable to those compounds represented by formulae 160-162.

In certain embodiments, a sirtuin-inhibiting compound is nicotinamide, as described in WO 2006/086454, incorporated herein by reference in its entirety.

In certain embodiments, a sirtuin-inhibiting compound is valproic acid or a derivative thereof, as described in WO 2002/007722 and related WO 2003/024442, US 2004/0087652, US 2005/0038113, EP 1 293 205, EP 1 427 403 and EP 1 602 371, each of which is incorporated herein by reference in its entirety. Valproic acid (VPA) and exemplary valproic acid derivatives may be represented, for example, by the following formula (163):

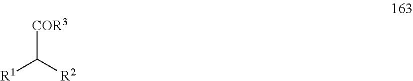

Derivatives of VPA are α-carbon branched carboxylic acids as described by formula 163 wherein R¹ and R² independently are a linear or branched, saturated or unsaturated aliphatic C₂-C₂₅, preferably C₃-C₂₅ hydrocarbon chain which optionally comprises one or several heteroatoms and which may be substituted, R³ is hydroxyl, halogen, alkoxy or an optionally alkylated amino group.

Different R¹ and R² residues give rise to chiral compounds. Usually one of the stereoisomers has a stronger teratogenic effect than the other one (Nau et al., 1991, Pharmacol. Toxicol. 69, 310-321) and the more teratogenic isomer more efficiently activates PPAR5 (Lampen et al., 1999, Toxicol. Appl. Pharmacol. 160, 238-249). Therefore, this isomer can be expected to inhibit histone deacetylases, such as sirtuins, more strongly. Racemic mixtures of these compounds, the less active isomers, and in particular, the more active isomers are contemplated as embodiments of the present invention.

The hydrocarbon chains R¹ and R² may comprise one or several heteroatoms (e.g., O, N, S) replacing carbon atoms in the hydrocarbon chain. This is due to the fact that structures very similar to that of carbon groups may be adopted by heteroatom groups when the heteroatoms have the same type of hybridization as a corresponding carbon group.

R¹ and R² may be substituted. Possible substituents include hydroxyl, amino, carboxylic and alkoxy groups as well as aryl and heterocyclic groups.

Preferably, R¹ and R² independently comprise 2 to 10, more preferably 3 to 10 or 5 to 10 carbon atoms. It is also preferred that R¹ and R² independently are saturated or comprise one double bond or one triple bond. In particular, one of the side chains (R¹) may preferably contain sp¹ hybridized carbon atoms in position 2 and 3 or heteroatoms which generate a similar structure. This side chain should comprise 3 carbon or heteroatoms but longer chains may also generate sirtuin inhibiting molecules. Also inclusion of aromatic rings or heteroatoms in R₂ is considered to generate compounds with sirtuin inhibitory activity because the catalytic site of the sirtuin protein apparently accommodates a wide variety of binding molecules. With the observation that teratogenic VPA derivatives are sirtuin inhibitors, also compounds which have previously been disregarded as suitable antiepileptic agents are also contemplated as sirtuin inhibitors. In particular, but not exclusively, compounds having a propinyl residue as R1 and residues of 7 or more carbons as R2, are considered (Lampen et al., 1999).

Preferably, the group “COR³” is a carboxylic group. Also derivatization of the carboxylic group has to be considered for generating compounds with potential sirtuin inhibitory activity. Such derivatives may be halides (e.g., chlorides), esters or amides. When R³ is alkoxy, the alkoxy group comprises 1 to 25, preferably 1-10 carbon atoms. When R³ is a mono- or di-alkylated amino group, the alkyl substituents comprise 1 to 25, preferably 1-10 carbon atoms. An unsubstituted amino group, however, is preferred.

According to the present invention also substances can be used which are metabolized to a compound as defined in formula 163 in the human organism or which lead to the release of a compound as defined in formula 163 for example by ester hydrolysis.

In a particular embodiment, the invention concerns the use of an α-carbon branched carboxylic acid as described by formula 163 or of a pharmaceutically acceptable salt thereof as an inhibitor of an enzyme having histone deacetylase (e.g., sirtuin) activity wherein R¹ is a linear or branched, saturated or unsaturated, aliphatic C5-25 hydrocarbon chain, R2 independently is a linear or branched, saturated or unsaturated, aliphatic C2-C5 hydrocarbon chain, R1 and R2 are optionally substituted with hydroxyl, amino, carboxylic, alkoxy, aryl and/or heterocyclic groups, and R3 is hydroxyl.

In yet another embodiment the invention concerns the use of an α-carbon branched carboxylic acid as described by formula 163 or of a pharmaceutically acceptable salt thereof as an inhibitor of an enzyme having histone deacetylase (e.g., sirtuin) activity wherein R1 is a linear or branched, saturated or unsaturated, aliphatic C3-25 hydrocarbon chain, and R2 independently is a linear or branched, saturated or unsaturated, aliphatic C2-C5 hydrocarbon chain, R1 or R2 comprise one or several heteroatoms (e.g., O, N, S) replacing carbon atoms in the hydrocarbon chain, R1 and R2 are optionally substituted with hydroxyl, amino, carboxylic, alkoxy, aryl and/or heterocyclic groups, and R³ is hydroxyl.

In yet another embodiment of the invention R1 and R2 do not comprise an ester group (—CO—O—). The atom of R1 which is next to the α-carbon of the carboxylic acid (derivative) of formula 163 and covalently linked to the a-carbon may be a carbon atom.

The atom of R2 which is next to the α-carbon of the carboxylic acid (derivative) of formula 163 and covalently linked to the α-carbon may be a carbon atom. R1 and R2 may be hydrocarbon chains comprising no heteroatoms O, N or S.

The compounds which are most preferably used according to the present invention are VPA, S-4-yn VPA, 2-EHXA (2-Ethyl-hexanoic acid).

In certain embodiments, a sirtuin-inhibiting compound is a compound as described in WO 2004/009536 and related US 2005/0176686, each of which is incorporated herein by reference in its entirety. Exemplary compounds as disclosed by these references may be represented by formula 164, below:

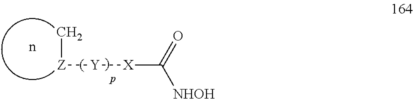

wherein:
n is a non-aromatic ring system containing two to six carbon atoms, wherein the ring system can contain one or two double bonds;
X is C, CH or CH2;
Y is selected from C, CH, CH2, S, NR, CH2-CH2, H2C—CH, HC—CH2, C—CH2, H2C—C or C—C; one or more of the hydrogen atoms can optionally be substituted by one or more substituents R′;
each of the dotted lines means a single, a double or triple bond with the exclusion of a combination of a triple with triple bond and a double with a triple bond;
R′ is independently H, —CN, alkyl, cycloalkyl, aminoalkyl, alkylamino, alkoxy, —OH, —SH, alkylthio, hydroxyalkyl, hydroxyalkylamino, halogen, haloalkyl, halo alkyloxy;
R is H, an alkyl or cycloalkyl group;
Z is CH, C, or P;
p is 0 or 1.

In certain embodiments in the compounds of formula 164, the ring n including Z can be cyclopentyl, cyclohexyl, cycloheptyl, cyclopent-1-enyl, cyclohex-1-enyl, cyclohept-1-enyl, cyclopent-2-enyl, cyclohex-2-enyl, cyclohept-2-enyl, cyclohex-3-enyl, or cyclohept-3-enyl. In another embodiment in the compounds of the formula 164, the ring n including Z is cyclopentyl or cyclohexyl, and Y is selected from CH, CH2, CH2—CH2, S, NR or p=0, and Z is CH or P. In another embodiment in the compounds of the formula 164, the ring n including Z is cyclopentyl or cyclohexyl, Y is selected from CH, CH2, CH2-CH2, or p=0 and Z is CH. In another embodiment, none of the carbon atoms of the alkyl groups is replaced by a group A.

Certain preferred compounds of formula 164 are: 3-Cyclopentyl-N-hydroxy-propionamide; 3-Cyclohexyl-N-hydroxy-propionamide; 4-Cyclohexyl-N-hydroxy-butyramide; and 2-Cycloheptyl-N-hydroxy-acetamide.

Regarding compounds of formula 164, the following definitions apply:

An alkyl group, if not stated otherwise, is preferably a linear or branched chain of 1 to 6 carbon atoms, preferably a methyl, ethyl, propyl, isopropyl, butyl, t-butyl, isobutyl, pentyl or hexyl group, a methyl, ethyl, isopropyl or t-butyl group being most preferred. The term “alkyl”, unless otherwise noted, is also meant to include those derivatives of alkyl defined in more detail below as “unsaturated alkyl”. An unsaturated alkyl group is one having one or more double bonds or triple bonds, preferably vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The alkyl group in the compounds of formula 164 can optionally be substituted by one or more substituents R′ being as defined above.

An cycloalkyl group denotes a non-aromatic ring system containing 3 to 8 carbon atoms, wherein one or more of the carbon atoms in the ring can be replaced by a group X, X being as defined above.

An alkoxy group denotes an O-alkyl group, the alkyl group being as defined above.

An alkylthio group denotes an S-alkyl group, the alkyl group being as defined above.

A hydroxyalkyl group denotes an HO-alkyl group, the alkyl group being as defined above.

An haloalkyl group denotes an alkyl group which is substituted by one to five preferably three halogen atoms, the alkyl group being as defined above; a CF3 group being preferred.

An haloalkyloxy group denotes an alkoxy group which is substituted by one to five preferably three halogen atoms, the alkoxy group being as defined above; an OCF3 group being preferred.

A hydroxyalkylamino group denotes an (HO-alkyl)₂-N-group or HO-alkyl-NH-group, the alkyl group being as defined above.

An alkylamino group denotes an HN-alkyl or N-dialkyl group, the alkyl group being as defined above.

An aminoalkyl group denotes an H2N-alkyl, monoalkylaminoalkyl, or dialkylaminoalkyl group, the alkyl group being as defined above.

A halogen group is chlorine, bromine, fluorine or iodine, fluorine being preferred.

In certain embodiments, a sirtuin-inhibiting compound is a compound as described in WO 2006/031894, incorporated herein by reference in its entirety. Exemplary compounds as disclosed by this reference may be represented by formulae 165-171, below:

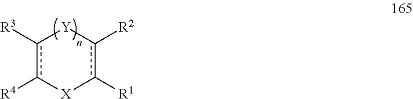

wherein:
R1 and R2, together with the carbons to which they are attached, form C5-C10 cycloalkyl, C5-C10 heterocyclyl, C5-C10 cycloalkenyl, C5-C10 heterocycloalkenyl, C6-C10 aryl, or C5-C10 heteroaryl, each of which may be optionally substituted with 1-5 R5; or R1 is H, S-alkyl, or S-aryl, and R2 is amidoalkyl wherein the nitrogen is substituted with alkyl, aryl, or arylalkyl, each of which is optionally further substituted with alkyl, halo, hydroxy, or alkoxy;
R3 and R4, together with the carbons to which they are attached, form C5-C10 cycloalkyl, C5-C10 heterocyclyl, C5-C10 cycloalkenyl, C5-C10 heterocycloalkenyl, C6-C10 aryl, or C5-C10 heteroaryl, each of which maybe optionally substituted with 1-5 R6;
each of R5 and R6 is, independently, halo, hydroxy, C1-C10 alkyl, C1-C6 haloalkyl, C1-C10 alkoxy, C1-C6 haloalkoxy, C6-C10 aryl, C5-C10 heteroaryl, C7-C12 aralkyl, C7-C12 heteroaralkyl, C3-C8 heterocyclyl, C2-C12 alkenyl, C2-C12 alkynyl, C5-C10 cycloalkenyl, C5-C10 heterocycloalkenyl, carboxy, carboxylate, cyano, nitro, amino, C1-C6 alkyl amino, C1-C6 dialkyl amino, mercapto, SO3H, sulfate, S(O)NH2, S(O)2NH2, phosphate, CrC4 alkylenedioxy, oxo, acyl, aminocarbonyl, C1-C6 alkyl aminocarbonyl, C1-C6 dialkyl aminocarbonyl, C1-C10 alkoxycarbonyl, C1-C10 thioalkoxycarbonyl, hydrazinocarbonyl, C1-C6 alkyl hydrazinocarbonyl, C1-C6 dialkyl hydrazinocarbonyl, hydroxyaminocarbonyl; alkoxyaminocarbonyl; or one of R5 or R6 and R7 form a cyclic moiety containing 4-6 carbons, 1-3 nitrogens, 0-2 oxygens and 0-2 sulfurs, which may be optionally substituted with oxo or C1-C6 alkyl;
X is NR7, O, or S; Y is NR7′, O or S;
- - - - represent optional double bonds;
each of R7 and R7′ is, independently, hydrogen, C1-C6 alkyl, C7-C12 arylalkyl, C7-C12 heteroarylalkyl; or R7 and one of R5 or R6 form a cyclic moiety containing 4-6 carbons, 1-3 nitrogens, 0-2 oxygens and 0-2 sulfurs, which may be optionally substituted with oxo or C1-C6 alkyl; and n is 0 or 1.

Non-limiting exemplary embodiments are as follows: n can be 1; X can be NR7 and Y can be NR7; R7 and R7′ can each be, e.g., hydrogen or CH3; one of R7 and R7′ can be hydrogen and the other can be CH3; R1 and R2 can form C5-C10 cycloalkenyl; R1 and R2 can form C6-C10 aryl; R1 and R2 can form C5-C10 cycloalkenyl, which may be substituted with R5, and R3 and R4 can form C6-C10 aryl, which may be substituted with R6; the cycloalkenyl double bond can be between the carbon attached to R1 and the carbon attached to R2; C5-C10 cycloalkenyl, e.g., C6 or C7 cycloalkenyl, can be substituted with R5 and C6-C10 aryl can be substituted with R6; R6 can be halo (e.g., chloro or bromo), C1-C6 alkyl (e.g., CH3), C1-C6 haloalkyl (e.g., CF3) or C1-C6 haloalkoxy (e.g., OCF3); R5 can be for example, C1-C6 alkyl substituted with a substituent such as an amino substituent, or aminocarbonyl (for example a substituted aminocarbonyl, substituted with substituents such an aryl, heteroaryl, cycloalkyl, heterocycloalkyl, aminocarbonyl, alkylaminocarbonyl, alkoxycarbonyl or other substituents. In each instances, the substituents can be further substituted with other substituents); n can be 0; R1 and R2 can form C5-C10 cycloalkenyl; R1 and R2 can form C6-C10 aryl; X can be NR7, and R7 can be, e.g., hydrogen or CH3; R1 and R2 can form C5-C10 cycloalkenyl, which may be substituted with R5, and R3 and R4 can form C6-C10 aryl, which may be substituted with R6; the cycloalkenyl double bond can be between the carbon attached to R1 and the carbon attached to R2; C5-C10 cycloalkenyl, e.g., C6 or C7 cycloalkenyl, can be substituted with R5 and C6-C10 aryl can be substituted with R6; R6 can be halo (e.g., chloro), C1-C6 alkyl (e.g., CH3), C1-C6 haloalkyl (e.g., CF3) or C1-C6 haloalkoxy (e.g., OCF3); R5 can be aminocarbonyl; R1 and R2 can form C5-C10 cycloalkenyl; R1 and R2 can form C6-C10 aryl; X can be NR7, and R7 can be, e.g., hydrogen or CH3; R1 and R2 can form C5-C10 cycloalkenyl, which may be substituted with R5, and R3 and R4 can form C6-C10 aryl, which may be substituted with R6; the cycloalkenyl double bond can be between the carbon attached to R1 and the carbon attached to R2; C5-C10 cycloalkenyl, e.g., C6 or C7 cycloalkenyl, can be substituted with R5 and C6-C10 aryl can be substituted with R6.

Compounds satisfying formula 165 may have formula 166 or formula 167:

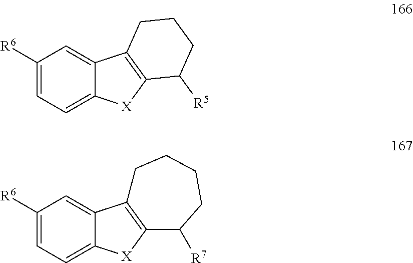

wherein:
R6 can be halo (e.g., chloro or bromo), C1-C6 alkyl (e.g., CH3), C1-C6 haloalkyl (e.g., CF3) or C1-C6 haloalkoxy (e.g., OCF3).
R5 can be aminocarbonyl.

Other compounds may satisfy any one of formulae 168, 169, 170 or 171:

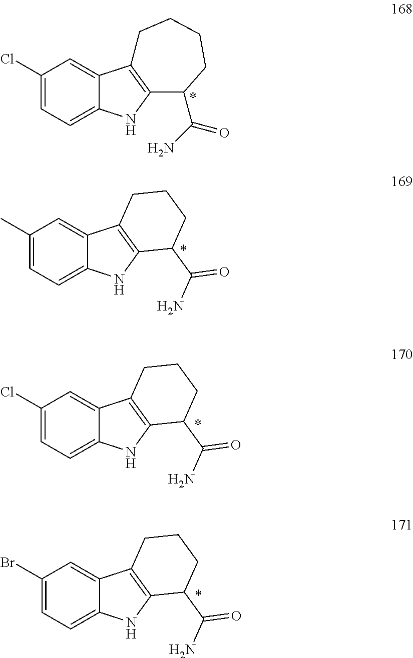

Definitions applicable to formulae 165-171 are as follows:

The term “alkyl” refers to a hydrocarbon chain that may be a straight chain or branched chain, containing the indicated number of carbon atoms. For example, C1-C12 alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl).

The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH2—, —CH2CH2—, and —CH2CH2CH2—. The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The terms “alkylamino” and “dialkylamino” refer to —NH(alkyl) and —NH(alkyl)2 radicals respectively. The term “aralkylamino” refers to a —NH(aralkyl) radical. The term “alkylaminoalkyl” refers to a (alkyl)NH-alkyl-radical; the term “dialkylaminoalkyl” refers to a (alkyl)₂N-alkyl-radical The term “alkoxy” refers to an —O-alkyl radical. The term “mercapto” refers to an —SH radical. The term “thioalkoxy” refers to an —S-alkyl radical. The term thioaryloxy refers to an —S-aryl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl. The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyls include, but are not limited to, tetrahydrofuranyl, tetrahydropyranyl, piperidinyl, morpholino, pyrrolinyl, pyrimidinyl, quinolinyl, and pyrrolidinyl. The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl. The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “oxo” refers to an oxygen atom, which forms a carbonyl when attached to carbon, an N-oxide when attached to nitrogen, and a sulfoxide or sulfone when attached to sulfur. The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). The terms “aminocarbonyl,” “alkoxycarbonyl,” hydrazinocarbonyl, and hydroxyaminocarbonyl refer to the radicals —C(O)NH2, —C(O)O(alkyl), —C(O)NH2NH2, and —C(O)NH2NH2, respectively. The term “amido” refers to a —NHC(O)— radical, wherein N is the point of attachment.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF3), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF3), halo, hydroxy, carboxy, carboxylate, cyano, nitro, amino, alkyl amino, SO3H, sulfate, phosphate, methylenedioxy (—O—CH2-O— wherein oxygens are attached to vicinal atoms), ethylenedioxy, oxo, thioxo (e.g., C═S), imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

In certain embodiments, a sirtuin-inhibiting compound is a compound as described in WO 2006/006171, incorporated herein by reference in its entirety. Exemplary compounds include compounds as listed in US 2004/0142859, incorporated herein by reference in its entirety; sirtinol and vitamin B3 [Luo, et al. (2001) Cell 107:137-148]; splitomicin [Bedalov, et al. (2001) Proc. Natl. Acad. Sci. 98, 15113-15118]; and M15 [Bitterman (2002) J. Biol. Chem. 277:45099-45107]. These chemical inhibitors are commercially available from various vendors, such as from Sigma (St. Louis, USA), Chembridge (San Diego, Calif., USA).

In certain embodiments, a sirtuin-inhibiting agents is an agent as described in WO 2005/0078091, incorporated herein by reference in its entirety. Exemplary agents include a siRNA, a dsRNA, a nucleic acid encoding such RNA, or a SIRT1 antisense RNA.

In certain embodiments, a sirtuin-inhibiting compound is a compound as described in WO 2005/062952 and related US 2005/0287597, each of which is incorporated herein by reference in its entirety. Exemplary compounds include suramin analogs such as NF279, NF023, and the like. NF023 is (8,8′-[carbonylbis(imino-3,1-phenylenecarbonylimino)]bis-1,3,5-naphthalene-trisulphonic acid); NF279 is (8,8′-[carbonylbis(imino-4,1-phenylenecarbonylimino-4,1-phenylenecarbonylimino)]bis-1,3,5-naphthalene-trisulphonic acid).

Other exemplary compounds include those represented by formulae 172-174, shown below:

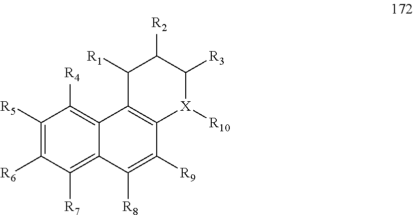

wherein:
X is C, O, N, or S;
each of R6, R7, and R8 is independently selected from a substituted or unsubstituted phenyl group; a substituted or unsubstituted, saturated linear or branched hydrocarbon group or chain (e.g., C1 to C8) including, e.g., methyl, ethyl, isopropyl, tert-butyl, heptyl, n-octyl, dodecyl, octadecyl, amyl, 2-ethylhexyl; an ether group, such as a methoxyl group; and an ethoxyl group;
each of R1, R3, and R4-R10 is independently selected from H; a halo (e.g., bromo, fluoro, chloro); a substituted or unsubstituted, saturated linear or branched hydrocarbon group or chain (e.g., C1 to C8) including, e.g., methyl, ethyl, isopropyl, tert-butyl, heptyl, n-octyl, dodecyl, octadecyl, amyl, 2-ethylhexyl; or an ether group, such as a methoxyl group or an ethoxyl group; a substituted or unsubstituted phenyl group; and a substituted or unsubstituted heteroaromatic group.

In some embodiments, a suitable sirtuin-inhibiting compound is a compound of formula 173:

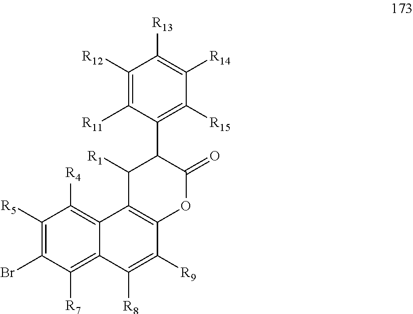

wherein:
R1, R4, R5, and R7-R15 is independently selected from H; a halo (e.g., bromo, fluoro, chloro); a substituted or unsubstituted, saturated linear or branched hydrocarbon group or chain (e.g., C1 to C8) including, e.g., methyl, ethyl, isopropyl, tert-butyl, heptyl, n-octyl, dodecyl, octadecyl, amyl, 2-ethylhexyl; or an ether group, such as a methoxyl group or an ethoxyl group; a substituted or unsubstituted phenyl group; and a substituted or unsubstituted heteroaromatic group.

In some embodiments, a suitable sirtuin-inhibiting compound is a compound of formula 174:

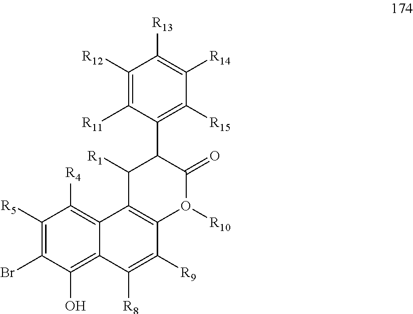

wherein:
R1, R4, R5, and R8-R15 is independently selected from H; a halo (e.g., bromo, fluoro, chloro); a substituted or unsubstituted, saturated linear or branched hydrocarbon group or chain (e.g., C1 to C8) including, e.g., methyl, ethyl, isopropyl, tert-butyl, heptyl, n-octyl, dodecyl, octadecyl, amyl, 2-ethylhexyl; or an ether group, such as a methoxyl group or an ethoxyl group; a substituted or unsubstituted phenyl group; and a substituted or unsubstituted heteroaromatic group.

Definitions applicable to compounds of formulae 172-174 include:

The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C1 to C8) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C1 to C7 alkoxy, C1 to C7 acyl, C1 to C7 acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group.

The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups. The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “trihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1] hept-2-ene, cyclohex-1-enyl, cyclopent-1-enyl, 1,4-cyclooctadienyl, and the like. The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C1 to C7 alkyl, C1 to C7 alkoxy, C1 to C7 acyl, C1 to C7 acyloxy, carboxy, oxycarboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C1 to C6 alkyl)carboxamide, protected N—(C1 to C6 alkyl)carboxamide, N,N-di(C1 to C6 alkyl)carboxamide, trifluoromethyl, N—((C1 to C6 alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di (halo) phenyl group such as 2-, 3-, or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2-, 3-, or 4-fluorophenyl and the like; a mono or di(hydroxy)phenyl group such as 2-, 3-, or 4-hydroxyphenyl, 2,4-dihydroxyphenyl, the protected-hydroxy derivatives thereof and the like; a nitrophenyl group such as 2-, 3-, or 4-nitrophenyl; a cyanophenyl group, for example, 2-, 3-, or 4-cyanophenyl; a mono- or di(alkyl)phenyl group such as 2-, 3-, or 4-methylphenyl, 2,4-dimethylphenyl, 2-, 3-, or 4-(isopropyl)phenyl, 2-, 3-, or 4-ethylphenyl, 2-, 3-, or 4-(n-propyl)phenyl and the like; a mono or di(alkoxy)phenyl group, for example, 2,6-dimethoxyphenyl, 2-, 3-, or 4-(isopropoxy)phenyl, 2-, 3-, or 4-(t-butoxy)phenyl, 3-ethoxy-4-methoxyphenyl and the like; 2-, 3-, or 4-trifluoromethylphenyl; a mono- or dicarboxyphenyl or (protected carboxy)phenyl group such as 2-, 3-, or 4-carboxyphenyl or 2,4-di(protected carboxy)phenyl; a mono- or di(hydroxymethyl)phenyl or (protected hydroxymethyl)phenyl such as 2-, 3-, or 4-(protected hydroxymethyl)phenyl or 3,4-di(hydroxymethyl)phenyl; a mono- or di(aminomethyl)phenyl or (protected aminomethyl)phenyl such as 2-, 3-, or 4-(aminomethyl)phenyl or 2,4-(protected aminomethyl)phenyl; or a mono- or di(N-(methylsulfonylamino))phenyl such as 2-, 3-, or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C1 to C4 alkoxy group, similarly, “lower alkylthio” means a C1 to C4 alkylthio group.

The term “(monosubstituted) amino” refers to an amino group with one substituent chosen from the group consisting of phenyl, substituted phenyl, alkyl, substituted alkyl, C1 to C4 acyl, C2 to C7 alkenyl, C2 to C7 substituted alkenyl, C2 to C7 alkynyl, C7 to C16 alkylaryl, C7 to C16 substituted alkylaryl and heteroaryl group. The (monosubstituted) amino can additionally have an amino-protecting group as encompassed by the term “protected (monosubstituted) amino.” The term “(disubstituted) amino” refers to amino groups with two substituents chosen from the group consisting of phenyl, substituted phenyl, alkyl, substituted alkyl, C1 to C7 acyl, C2 to C7 alkenyl, C2 to C7 alkynyl, C7 to C16 alkylaryl, C7 to C16 substituted alkylaryl and heteroaryl. The two substituents can be the same or different.

The term “heteroaryl (alkyl)” denotes an alkyl group as defined above, substituted at any position by a heteroaryl group, as above defined.

“Optional” or “optionally” means that the subsequently described event, circumstance, feature or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

In certain embodiments, a sirtuin-inhibiting compound is a compound as described in WO 2003/046207 and related US 2005/0079995, each of which is incorporated herein by reference in its entirety. Exemplary compounds include those of formulae 175-177.

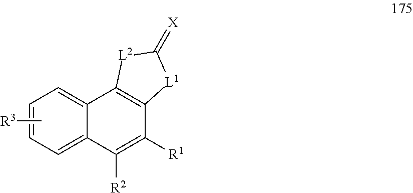

wherein:
X is a member selected from the group consisting of O and S;
L1 and L2 each represent members independently selected from the group consisting of O, S, ethylene and propylene, substituted with 0-2 R groups, wherein exactly one of the symbols L1 and L2 represents a member selected from the group consisting of O and S.

Each instance of the letter R of symbols L1 and L2 independently represents a member selected from the group consisting of C1-6 alkyl, C2-6 alkenyl and —CO2R4. The symbols R1 and R2 each represent members independently selected from the group consisting of hydrogen, C1-6 alkoxy, C0-6 alkoxy-aryl and hydroxy. Alternatively, the symbols R1 and R2 are taken together with the carbons to which they are attached to form a six-membered lactone ring.

The symbol R3 represents a member selected from the group consisting of hydrogen, C1-6 alkyl, aryl, —OR4, —NR4R4, —CO2R4, —C(O)R4, —C(O)NR4R4, —CN, —NO2 and halogen. Each instance of the symbol R4 independently represents a member selected from the group consisting of hydrogen and C1-6 alkyl.

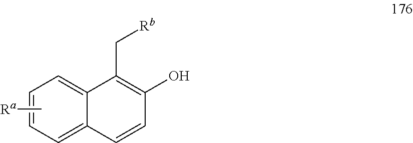

In formula 176, the symbol Ra is a member selected from the group consisting of hydrogen, C1-6 alkyl, aryl, —ORe, —NReRe, —CO2Re, —C(O)Re, —C(O)NR′Re, —CN, —NO2 and halogen, while the symbol Rb is a member selected from the group consisting of

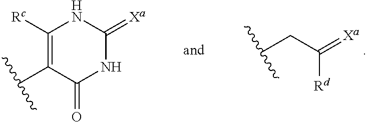

In the structures above, the symbol Xa represents a member selected from the group consisting of O, S and NRe, while the symbol Rc represents a member selected from the group consisting of hydrogen, C1-6 alkyl and aryl optionally substituted with a member selected from the group consisting of hydrogen, C1-6 alkyl, aryl, —ORe, —NReRe, —CN, —NO2 and halogen. The symbol Rd represents a member selected from the group consisting of hydrogen, C1-6 alkyl, aryl, —ORe, —NReRe and halogen. And, each instance of the symbol Re independently represents a member selected from the group consisting of hydrogen and C1-6 alkyl.

In certain embodiments, a structure satisfying formula 176 is that of formula 177:

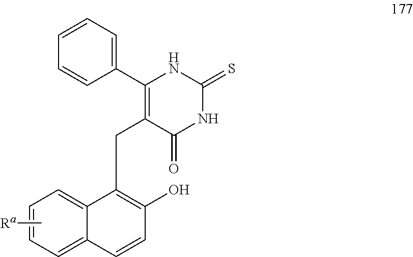

The following definitions are applicable to compounds of formulae 175-177:

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C1-C10 means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An “unsaturated alkyl group” is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Preferred alkyl groups are limited to hydrocarbon groups, and may be branched- or straight-chain. More preferred alkyl groups are unsubstituted.

The term “alkylene” by itself or as part of another substituent means a divalent radical derived from an alkane, as exemplified by —CH2CH2CH2CH2—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH2-CH2-O—CH3, —CH2-CH2—NH—CH3, —CH2-CH2-N(CH3)-CH3, —CH2—S—CH2-CH3, —CH2-CH2, —S(O)—CH3, —CH2-CH2-S(O)2-CH3, —CH═CH—O—CH3, —Si(CH3)3, —CH2-CH═N—OCH3, and —CH═CH—N(CH3)—CH3. Up to two heteroatoms may be consecutive, such as, for example, —CH2-NH—OCH3 And —CH2-O—Si(CH3)3. Similarly, the term “heteroalkylene” by itself or as part of another substituent means a divalent radical derived from heteroalkyl, as exemplified by —CH2-CH2-S—CH2CH2— and —CH2-S—CH2-CH2-NH—CH2—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo (C1-C4)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom (s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO2R′, —CONR′R″, —OC(O)NR′R″, —NR″C(O)R′, —NR′—C(O)NR″R″′, —NR″C(O)2R′, —NH—C(NH2)═NH, —NR′C(NH2)═NH, —NH—C(NH2)═NR′, —S(O)R′, —S(O)2R′, —S(O)2NR′R″, —CN and —NO2 in a number ranging from zero to (2 m′+1), where m′ is the total number of carbon atoms in such radical. R′, R″ and R′″ each independently refer to hydrogen, unsubstituted (C1-C8)alkyl and heteroalkyl, unsubstituted aryl, aryl substituted with 1-3 halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF3 and —CH2CF3) and acyl (e.g., —C(O)CH3, —C(O)CF3, —C(O)CH2CH3, and the like). Preferably, substituted alkyl groups are those having 3, 2 or 1 substituents selected from the group consisting of —OR′, —NR′R″, -halogen, —C(O)R′, —CO2R′, —CONR′R″, —CN and —NO2.

Similarly, substituents for the aryl and heteroaryl groups are varied and are selected from: -halogen, —OR′, —OC(O)R′, —NR′R″, —SR′, —R′, —CN, —NO2, —CO2R′, —CONR′R″, —C(O)R′, —OC(O)NR′R″, —NR″C(O)R′, —NR″C(O)₂R′, —NR′—C(O)NR″R′″, —NH—C(NH2)═NH, —NR′C(NH2)═NH, —NH—C(NH2)═NR′, —S(O)R′, —S(O)₂R′, —S(O)2NR′R″, —N3, —CH(Ph)2, perfluoro(C1-C4)alkoxy, and perfluoro(C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl. Preferably, substituted aryl groups are those having 1, 2 or 3 substituents selected from the group consisting of -halogen, —OR′, —NR′R″, —CN, —NO2, —CO2R′, —CONR′R″, —C(O)R′, —N3, perfluoro(C1-C4)alkoxy, and perfluoro(C1-C4)alkyl.

Two of the substituents on adjacent atoms of the aryl or heteroaryl ring may optionally be replaced with a substituent of the formula -T-C(O)—(CH2)q-U-, wherein T and U are independently —NH—, —O—, —CH2- or a single bond, and q is an integer of from 0 to 2. Alternatively, two of the substituents on adjacent atoms of the aryl or heteroaryl ring may optionally be replaced with a substituent of the formula -A-(CH2)r-B-, wherein A and B are independently —CH2—, —O—, —NH—, —S—, —S(O)—, —S(O)2—, —S(O)2NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH2), —X— (CH2)t-, where s and t are independently integers of from 0 to 3, and X is —O—, —NR′—, —S—, —S(O)—, —S(O)2—, or —S(O)2NR′—. The substituent R′ in —NR′— and —S(O)2NR′— is selected from hydrogen or unsubstituted (C1-C6)alkyl.

As used herein, the term “heteroatom” is meant to include oxygen (O), nitrogen (N) and sulfur (S).

As used herein, the term “lactone ring” refers to a five-, six- or seven-membered cyclic ester, such as

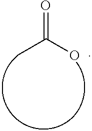

In certain embodiments, a sirtuin-inhibiting compound is a compound as described in WO 2006/096780 and related US 2006/0025337 and US 2006/0084135, each of which is incorporated herein by reference in its entirety. Exemplary compounds include those of formulae 178-185.

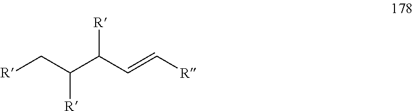

wherein:
R′ represents H, halogen, NO₂, SR, OR, NR₂, alkyl, aryl, aralkyl, or carboxy;
R represents H, alkyl, aryl, aralkyl, heteroaralkyl, —SO₃H, monosaccharide, oligosaccharide, glycofuranosyl, glycopyranosyl, glucuronosyl, or glucuronide; and
R″ represents alkyl, alkenyl, or alkynyl;

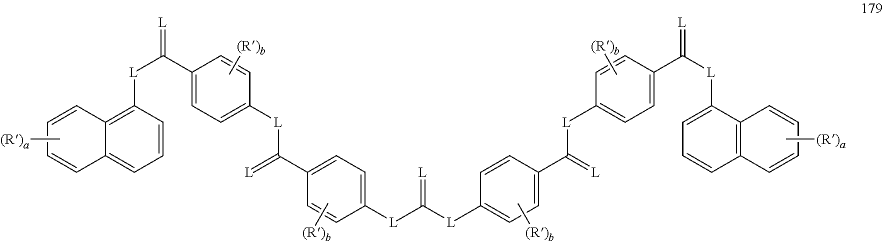

wherein:
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR₅ NR₂, alkyl, aryl, aralkyl, or carboxy;
a represents an integer from 1 to 7 inclusive; and
b represents an integer from 1 to 4 inclusive;

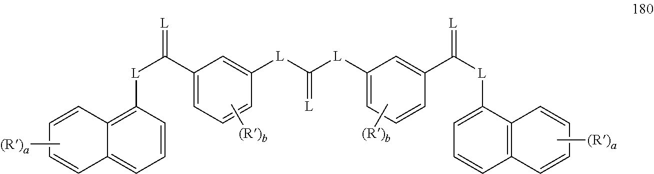

wherein:
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR, NR₂, alkyl, aryl, or carboxy;
a represents an integer from 1 to 7 inclusive; and
b represents an integer from 1 to 4 inclusive;

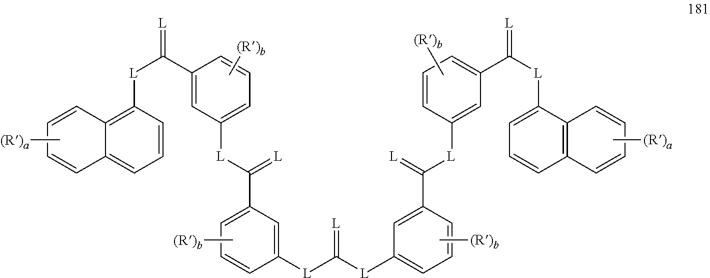

wherein:
L represents O, NR, or S;
R represents H, alkyl, aryl, aralkyl, heteroaralkyl, —SO₃H, monosaccharide, oligosaccharide, glycofuranosyl, glycopyranosyl, glucuronosyl, or glucuronide; and
R′ represents H, halogen, NO₂, SR, SO₃, OR, NR₂, alkyl, aryl, aralkyl, or carboxy;
a represents an integer from 1 to 7 inclusive; and
b represents an integer from 1 to 4 inclusive;

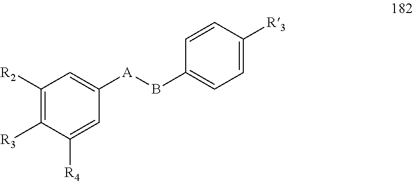

wherein:
R₂, R₃, and R₄ are H, OR, or O-alkyl; R represents H, —SO₃H, monosaccharide, oligosaccharide, glycofuranosyl, glycopyranosyl, glucuronosyl, or glucuronide; and
R′₃ is H or NO₂; and
A-B is an ethenylene or amido group.

In a further embodiment, the inhibiting compound is represented by formula 182 and the attendant definitions, wherein R₃ is OH, A-B is ethenylene, and R′₃ is H. In a further embodiment, the inhibiting compound is represented by formula 182 and the attendant definitions, wherein R₂ and R₄ are OH, A-B is an amido group, and R′₃ is H.

In a further embodiment, the inhibiting compound is represented by formula 182 and the attendant definitions, wherein R₂ and R₄ are OMe, A-B is ethenylene, and R′₃ is NO₂.

In a further embodiment, the inhibiting compound is represented by formula 182 and the attendant definitions, wherein R₃ is OMe, A-B is ethenylene, and R′₃ is H.

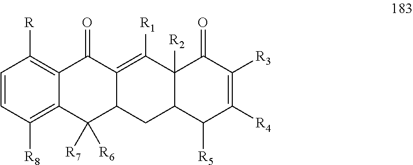

wherein:
R, R₁, R₂, R₃, R₄, R₅, R₆, R₇, and R₈ are H, hydroxy, amino, cyano, halide, OR₉, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₉ represents alkyl, —SO₃H, monosaccharide, oligosaccharide, glycofuranosyl, glycopyranosyl, glucuronosyl, or glucuronide.

In a further embodiment, the methods comprise a compound of formula 183 and the attendant definitions wherein R is OH. In a further embodiment, the methods comprise a compound of formula 183 and the attendant definitions wherein R₁ is OH. In a further embodiment, the methods comprise a compound of formula 183 and the attendant definitions wherein R₂ is OH.

In a further embodiment, the methods comprise a compound of formula 183 and the attendant definitions wherein R₃ is C(O)NH₂. In a further embodiment, the methods comprise a compound of formula 183 and the attendant definitions wherein R₄ is OH. In a further embodiment, the methods comprise a compound of formula 183 and the attendant definitions wherein R5 is NMe₂.

In a further embodiment, the methods comprise a compound of formula 183 and the attendant definitions wherein Re is methyl. In a further embodiment, the methods comprise a compound of formula 183 and the attendant definitions wherein R₇ is OH. In a further embodiment, the methods comprise a compound of formula 183 and the attendant definitions wherein R₈ is Cl.

In a further embodiment, the methods comprise a compound of formula 183 and the attendant definitions wherein R is OH and R₁ is OH. In a further embodiment, the methods comprise a compound of formula 183 and the attendant definitions wherein R is OH, R₁ is OH, and R₂ is OH.

In a further embodiment, the methods comprise a compound of formula 183 and the attendant definitions wherein R is OH, R₁ is OH, R₂ is OH, and R₃ is C(O)NH₂. In a further embodiment, the methods comprise a compound of formula 183 and the attendant definitions wherein R is OH, R₄ is OH, R₂ is OH, R₃ is C(O)NH₂, and R₄ is OH.

In a further embodiment, the methods comprise a compound of formula 183 and the attendant definitions wherein R is OH, R₁ is OH, R₂ is OH, R₃ is C(O)NH₂, R₄ is OH, and R₅ is NMe₂. In a further embodiment, the methods comprise a compound of formula 183 and the attendant definitions wherein R is OH, R₁ is OH, R₂ is OH, R₃ is C(O)NH₂, R₄ is OH, R₅ is NMe₂, and R₆ is methyl.

In a further embodiment, the methods comprise a compound of formula 183 and the attendant definitions wherein R is OH, R₁ is OH, R₂ is OH, R₃ is C(O)NH₂, R₄ is OH, R₅ is NMe₂, R₆ is methyl, and R₇ is OH. In a further embodiment, the methods comprise a compound of formula 183 and the attendant definitions wherein R is OH, R₁ is OH, R₂ is OH, R₃ is C(O)NH₂, R₄ is OH, R₅ is NMe₂, R₆ is methyl, R₇ is OH, and R₈ is

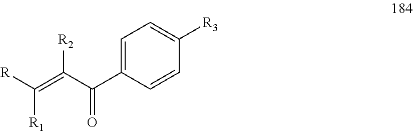

wherein:
R, R₁, R₂, and R₃ are H, hydroxy, amino, cyano, halide, OR₄, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and
R₄ represents alkyl, —SO₃H, monosaccharide, oligosaccharide, glycofuranosyl, glycopyranosyl, glucuronosyl, or glucuronide.

In a further embodiment, the methods comprise a compound of formula 184 and the attendant definitions wherein R is Cl. In a further embodiment, the methods comprise a compound of formula 184 and the attendant definitions wherein R₁ is H. In a further embodiment, the methods comprise a compound of formula 184 and the attendant definitions wherein R₂ is H.

In a further embodiment, the methods comprise a compound of formula 184 and the attendant definitions wherein R₃ is Br. In a further embodiment, the methods comprise a compound of formula 184 and the attendant definitions wherein R is C₁ and R₁ is H.

In a further embodiment, the methods comprise a compound of formula 184 and the attendant definitions wherein R is C₁, R₁ is H, and R₂ is H. In a further embodiment, the methods comprise a compound of formula 184 and the attendant definitions wherein R is C₁, R₁ is H, R₂ is H, and R₃ is Br.

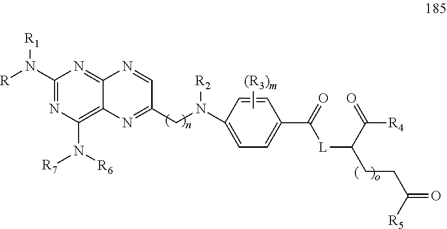

wherein:
R, R₁, R₂, R₆, and R₇ are H or a substituted or unsubstituted alkyl, aryl, aralkyl, heterocyclyl, heterocyclylalkyl, heteroaryl, or heteroaralkyl;
R₃, R₄, and R₅ are H, hydroxy, amino, cyano, halide, OR₆, ether, ester, amido, ketone, carboxylic acid, nitro, or a substituted or unsubstituted alkyl, aryl, aralkyl, heterocyclyl, heterocyclylalkyl, heteroaryl, or heteroaralkyl;
R₆ represents alkyl, —SO₃H, monosaccharide, oligosaccharide, glycofuranosyl, glycopyranosyl, glucuronosyl, or glucuronide;
L is O, NR, or S;
m is an integer from 0 to 4 inclusive; and n and o are integers from 0 to 6 inclusive.

In a further embodiment, the methods comprise a compound of formula 185 and the attendant definitions wherein R is H. In a further embodiment, the methods comprise a compound of formula 185 and the attendant definitions wherein R₁ is H. In a further embodiment, the methods comprise a compound of formula 185 and the attendant definitions wherein R₂ is methyl. In a further embodiment, the methods comprise a compound of formula 185 and the attendant definitions wherein m is 0. In a further embodiment, the methods comprise a compound of formula 185 and the attendant definitions wherein R₄ is OH.

In a further embodiment, the methods comprise a compound of formula 185 and the attendant definitions wherein R₅ is OH. In a further embodiment, the methods comprise a compound of formula 185 and the attendant definitions wherein Re is H. In a further embodiment, the methods comprise a compound of formula 185 and the attendant definitions wherein R₇ is H. In a further embodiment, the methods comprise a compound of formula 185 and the attendant definitions wherein L is NH. In a further embodiment, the methods comprise a compound of formula 185 and the attendant definitions wherein n is 1.

In a further embodiment, the methods comprise a compound of formula 185 and the attendant definitions wherein 0 is 1. In a further embodiment, the methods comprise a compound of formula 185 and the attendant definitions wherein R is H and R₁ is H. In a further embodiment, the methods comprise a compound of formula 185 and the attendant definitions wherein R is H, R₁ is H, and R₂ is methyl. In a further embodiment, the methods comprise a compound of formula 185 and the attendant definitions wherein R is H, R₁ is H, R₂ is methyl, and m is O.

In a further embodiment, the methods comprise a compound of formula 185 and the attendant definitions wherein R is H, R₁ is H, R₂ is methyl, m is 0, and R₄ is OH. In a further embodiment, the methods comprise a compound of formula 185 and the attendant definitions wherein R is H, R₁ is H, R₂ is methyl, m is 0, R₄ is OH, and R₅ is OH. In a further embodiment, the methods comprise a compound of formula 185 and the attendant definitions wherein R is H, R₁ is H, R₂ is methyl, m is 0, R₄ is OH, R₅ is OH, and R₆ is H.

In a further embodiment, the methods comprise a compound of formula 185 and the attendant definitions wherein R is H, R₁ is H, R₂ is methyl, m is 0, R₄ is OH, R₅ is OH, R₆ is H, and R₇ is H. In a further embodiment, the methods comprise a compound of formula 185 and the attendant definitions wherein R is H, R₁ is H, R₂ is methyl, m is 0, R₄ is OH, R₅ is OH, R₆ is H, R₇ is H, and L is NH. In a further embodiment, the methods comprise a compound of formula 185 and the attendant definitions wherein R is H, R₁ is H, R₂ is methyl, m is 0, R₄ is OH, R₅ is OH, R₆ is H, R₇ is H, L is NH, and n is 1.

Definitions applicable to compounds of formulae 178-185 are the same as those for compounds of formulae 41-66 herein.

In certain embodiments, a sirtuin-inhibiting compound is a compound as described in WO 2006/007411 and related WO 2005/002672, WO 2005/002555, WO 2005/065667, US 2006/0084085, US 2005/0136537, US 2005/0171027, US 2006/0111435 and US 2005/0096256, each of which is incorporated herein by reference in its entirety. Exemplary compounds include 178-185 described above and further include nicotinamide and analogs satisfying formulae 186-187:

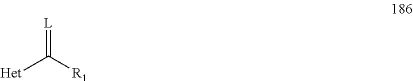

wherein:
L is O, NR, or S;
R is alkyl or phenyl;
R1 is —NH2, —O-alkyl, —N(R)2, or —NH(R);
and Het is heteroaryl or heterocycloalkyl.

Particular analogs that may be used include compounds of formula 186 and the attendant definitions, wherein L is O; compounds of formula 186 and the attendant definitions, wherein R1 is —NH2; compounds of formula 186 and the attendant definitions, wherein Het is selected from the group consisting of pyridine, furan, oxazole, imidazole, thiazole, isoxazole, pyrazole, isothiazole, pyridazine, pyrimidine, pyrazine, pyrrole, tetrahydrofuran, 1:4 dioxane, 1,3,5-trioxane, pyrrolidine, piperidine, and piperazine; compounds of formula 186 and the attendant definitions, wherein Het is pyridine; compounds of formula 186 and the attendant definitions, wherein L is O and R1 is —NH2; compounds of formula 186 and the attendant definitions, wherein L is O and Het is pyridine; compounds of formula 186 and the attendant definitions, wherein R1 is —NH2 and Het is pyridine; and compounds of formula I and the attendant definitions, wherein L is O, R1 is —NH2, and Het is pyridine.

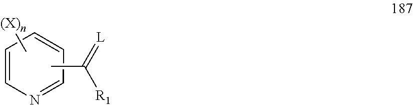

wherein:
L is O, NR, or S;
R is alkyl or phenyl;
R1 is —NH2, —O-alkyl, —N(R)2, or —NH(R);
X is H, alkyl, —O-alkyl, OH, halide, or NH2; and
n is an integer from 1 to 4 inclusive.

Particular analogs that may be used include compounds of formula 187 and the attendant definitions, wherein L is O; compounds of formula 187 and the attendant definitions, wherein R1 is —NH2; compounds of formula 187 and the attendant definitions, wherein X is H and n is 4; compounds of formula 187 and the attendant definitions, wherein L is O and R1 is —NH₂; compounds of formula 187 and the attendant definitions, wherein L is O, X is H, and n is 4; compounds of formula 187 and the attendant definitions, wherein R1 is —NH2, X is H, and n is 4; and compounds of formula 187 and the attendant definitions, wherein L is O, R1 is —NH2, X is H, and n is 4.

Definitions applicable to compounds of formulae 186-187 are the same as those for compounds of formulae 67-118, herein.

Other sirtuin-inhibiting compounds may include the following: nicotinamide (NAM), suranim; sphingosine; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:1861. Other inhibitors are 4-hydroxy-trans-stilbene; N-phenyl-(3,5-dihydroxy)benzamide; 3,5-Dihydroxy-4′-nitro-trans-stilbene; 4-Methyoxy-trans-stilbene; chlorotetracycline, 4-bromophenyl-3-chloro-propenone and methotrexane, which are described in WO 2005/002672, incorporated herein by reference in its entirety. Inhibitors are also described in WO 05/026112, incorporated herein by reference in its entirety. Other inhibitors, such as sirtinol and splitomicin, are described in Grozinger et al. (2001) J. Biol. Chem. 276:38837, Bedalov et al. (2001) PNAS 98:15113 and Hirao et al. (2003) J. Biol. Chem. 278:52773, each of which is incorporated herein by reference in its entirety. Analogs and derivatives of these compounds can also be used.

In certain embodiments, a sirtuin-inhibiting compound is a compound as described in WO 2005/002527 and related US 20050136429, each of which is incorporated herein by reference in its entirety. Exemplary compounds include those of formulae 175-177 and also those of formula 188:

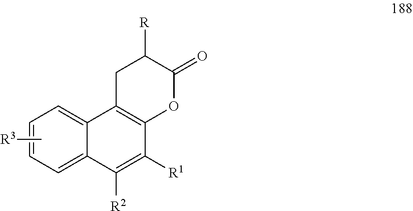

wherein:
R1 is a member selected from the group consisting of hydrogen, C1-6 alkoxy and C0-6 alkoxy-aryl;
R2 is a member selected from the group consisting of hydrogen and hydroxy;
R3 is a member selected from the group consisting of hydrogen and —OR4; and
R4 is C1-6 alkyl.
   In other embodiments, R1 is a member selected from the group consisting of C1-6 alkoxy, C0-6 alkoxy-aryl and hydroxy. For example, R1 may be a member selected from the group consisting of hydroxy, methoxy and benzyloxy. In another embodiment, the term aryl is a member selected from the group consisting of phenyl and naphthyl.

Definitions applicable to compounds of formulae 175-177 also apply to compounds of formula 188.

### II. COMBINATION THERAPIES

The compounds and methods of the present invention may be used in the context of a number of therapeutic and diagnostic applications. In order to increase the effectiveness of a treatment with the compositions of the present invention, such as other active compounds, it may be desirable to combine these compositions with other agents effective in the treatment of those diseases and conditions (secondary therapy). For example, the treatment of stroke (antistroke treatment) typically involves an antiplatelet (aspirin, clopidogrel, dipyridamole, ticlopidine), an anticoagulant (heparin, warfarin), or a thrombolytic (tissue plasminogen activator).

Various combinations may be employed; for example, an active compound, such as H2S, is “A” and the sirtuin-modulating compound is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the active compounds of the present invention to biological matter will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapies.

### III. SIRTUIN PROTEINS

As described above, sirtuin proteins are involved in diverse processes from regulation of gene silencing to DNA repair. Any active compound as described herein may modulate any one or more of such sirtuin-related activities. Potential applications of sirtuin modulators, including the active compounds described herein, include treatment regimes for HIV, neurodegenerative diseases (e.g., Alzheimer's disease), inflammatory diseases, cirrhosis, cancer, obesity, metabolic regulation, diabetes, expanding renewable stem cells, thasselemia, stress-related diseases and cardiovascular disease. Any active compound as described herein may be used for any sirtuin-related application known in the art.

The proteins encoded by members of the SIR gene family are highly conserved in a 250 amino acid core domain. Non-limiting examples of sirtuin proteins include SIRT1, SIRT2 and SIRT1, SIRT2 and SIRT3 mRNA sequences, as well as cDNA sequences encoding SIRT1, SIRT2 and SIRT3 polypeptides are known in the art.

The amino acid sequences of several SIRT1 polypeptides are publicly available. See, e.g., GenBank Accession Nos. Q96EB6, AAH12499, NP036370, and AAD40849 for human SIRT1 amino acid sequences; and GenBank Accession Nos. Q923E4 and NP₀₆₂₇₈₆ for mouse SIRT1 amino acid sequences.

The amino acid sequences of several SIRT2 polypeptides are publicly available. See, e.g., GenBank Accession Nos. NP_{—}085096, NP_{—}036369, AAH03547, and AAH03012 for human SIRT2 amino acid sequences; GenBank Accession Nos. AAH86545 and NP_{—}001008369 for rat SIRT2 amino acid sequences; and GenBank Accession No. NP071877 for a mouse SIRT2 amino acid sequence.

The amino acid sequences of several SIRT3 polypeptides are publicly available. See, e.g., GenBank Accession Nos. NP07878 and AAH25878 for mouse SIRT3 amino acid sequences; and NP_{—}036371, AAH01042, and AAD40851 for human SIRT3 amino acid sequences.

### IV. VARIOUS APPLICATIONS AND ADMINISTRATIONS

In certain embodiments, active compounds that increase the level and/or activity of a sirtuin protein may be used for a variety of therapeutic applications including, for example, increasing the lifespan of biological matter, and treating and/or preventing a wide variety of diseases and disorders (i.e., related to aging or stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, and/or flushing). Active compounds that increase the level and/or activity of a sirtuin protein may also be used for treating a disease or disorder in a biological matter that would benefit from increased mitochondrial activity, for enhancing muscle performance, for increasing muscle ATP levels, or for treating or preventing muscle tissue damage associated with hypoxia or ischemia.

In other embodiments, active compounds that decrease the level and/or activity of a sirtuin protein may be used for a variety of therapeutic applications including, for example, increasing cellular sensitivity to stress, increasing apoptosis, treatment of cancer, stimulation of appetite, and/or obesity treatment. As described further below, the methods comprise administering to a biological matter in need thereof an effective amount of an active compound (e.g., chalcogenide compounds or sirtuin-modulating compounds). In certain aspects, the active compound is a sirtuin-modulating compound and may be administered alone or in combination with other compounds, including chalcogenide compounds.

Pharmaceutical compositions of the present invention may include active compounds in any desired concentration. In particular embodiments, the concentration of active compound is optimized to be therapeutically effective for its intended purpose. In another embodiment, the concentration of sirtuin-modulating compound is optimized to be effective in reducing one or more undesired side-effects of chalcogenides. In another embodiment, the concentration of sirtuin-modulating compound is optimized to be effective in enhancing the effects of chalcogenides (i.e., enhancing lifespan and increasing survivability). In another embodiment, the concentration of a chalcogenide is optimized to be effective in reducing one or more undesired side-effects of sirtuin-modulating compounds. In another embodiment, the sirtuin-modulating compound and chalcogenide are co-administered and provide a synergistic action. In another embodiment, the sirtuin-modulating compound and chalcogenide are co-administered and inhibit undesired side-effects. In one embodiment, the sirtuin-modulating compound is administered before the chalcogenide. In one embodiment, the chalcogenide is administered before the sirtuin-modulating compound. The concentration may be readily optimized, e.g., depending upon the type of biological matter being treated and the route of administration, so as to deliver an effective amount in a convenient manner and over an appropriate time-frame.

The term “biological matter” refers to any living biological material (mammalian biological material in preferred embodiments) including cells, tissues, organs, and/or organisms, and any combination thereof. It is contemplated that longevity may be induced in a part of an organism (such as in cells, in tissue, and/or in one or more organs), whether that part remains within the organism or is removed from the organism, or the whole organism will be placed in a state of increased longevity. Moreover, it is contemplated in the context of cells and tissues that homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that longevity may be induced in isolated biological matter.

Alternatively, an organism or other biological matter may be in need of an active compound to enhance survivability. For instance, a patient may need treatment for an injury or disease or any other application discussed herein. They may be determined to be in need of enhanced survivability or treatment based on methods such as by taking a patient medical or family medical history.

The term “effective amount” means an amount that can achieve the stated result. In certain methods of the invention, an “effective amount” is, for example, an amount that induces longevity or increases the survival in the biological matter in need of longevity or in need of enhanced survival. In certain embodiments, an “effective amount” refers to an amount that modulates sirtuin activity. This can be determined (or assumed) based on comparison or previous comparison to untreated biological matter or biological matter treated with a different dosage or regimen that does not experience a difference in survivability.

Moreover, the effective amount can be expressed as a concentration with or without a qualification on length of time of exposure. In some embodiments, it is generally contemplated that to achieve other stated goals of the invention (e.g., modulate sirtuin activity to enhance survivability), the biological matter is exposed to an active compound for about, at least about, or at most about 5, 10, 15, 20, 25, 30, 35, 40, 45, 50, 55, 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. In certain embodiments, an active compound is provided periodically by providing or exposing biological matter 1, 2, 3, 4, 5, 6, 7, 8, 9, or 10 times every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, or any range derivable therein.

Furthermore, in some embodiments of the invention, biological matter is exposed to or provided with an active compound for a sustained period of time, where “sustained” means a period of time of at least about 2 hours. In other embodiments, biological matter may be exposed to or provided with an active compound on a sustained basis for more than a single day. In such circumstances, the biological matter is provided with an active compound on a continuously sustained basis. In certain embodiments, biological matter may be exposed to or provided with an active compound for about 2, 3, 4, 5, 6, 7, 8, 9, 10, 11, 12 or more hours (or any range derivable therein) for 2, 3, 4, 5, 6, 7 days, and/or 1, 2, 3, 4, 5 weeks, and/or 1, 2, 3, 4, 5, 6, 7, 8, 9, 10, 11, 12 months, and/or 1, 2, 3, 4, 5, 6, 7, 8, 9, 10, 11, 12, 13, 14, 15, 16, 17, 18, 19, 20 or more years (or any range derivable therein) continuously, intermittently (exposure on multiple occasions), or on a periodic basis (exposure on a recurring regular basis).

In some embodiments, biological matter may be exposed to or provided with an active compound at least before and during; before, during, and after; during and after; or solely after a particular injury, trauma, or treatment (for instance, surgery), adverse condition or other relevant event or situation. This exposure may or may not be sustained.

The dosages of an active compound on these different bases may the same or they may vary.

It is specifically contemplated that in some embodiments an active compound is provided to a subject by nebulizer. In further embodiments, the active compound is provided as a single dose to the subject. In some embodiments, a subject is given at least about 1,000, 2,000, 3,000, 4,000, 5,000, 6,000, 7,000, 8,000, 9,000, 10,000, 11,000, 12,000 or more ppm H2S gas. The exposure time may be any of the times discussed herein, including about or about at most 10, 9, 8, 7, 6, 5, 4, 3, 2, 1, 0.5, 0.1 minutes or less (or any range derivable therein).

A. Methods of Use

The present invention is based, on part, on the unexpected discovery that administration of a combination of sirtuin-modulating compounds and chalcogenides to a cell results in enhanced lifespan and a decrease in undesired side-effects as compared to administration of either sirtuin-modulating compounds or chalcogenides alone. Thus, the present invention provides methods of enhancing lifespan, increasing survivability and reducing cytotoxicity or undesired side-effects associated with administration of either sirtuin-modulating compounds or chalcogenides to biological material, e.g., cells, tissues, organs, organisms, and animals, which comprise administering either sirtuin-modulating compounds or chalcogenides in combination with the other.

According to the present invention, the combination of sirtuin-modulating compounds and chalcogenides counteracts, or neutralizes, undesirable pharmacological actions of sirtuin-modulating compounds or chalcogenides, including those that: i) exert harmful effects in mammals exposed thereto; or ii) impede, reverse, antagonize, or prevent the beneficial pharmacological effects of either sirtuin-modulating compounds, chalcogenides, or the combination thereof in mammals. These actions are known to those skilled in the art as “side effects” of drugs, meaning that the undesirable pharmacological actions of sirtuin-modulating compounds or chalcogenides are unwanted because they render less effective their known beneficial pharmacological or pharmaceutical actions. To the extent that sirtuin-modulating compounds and chalcogenides diminish the side effects of pharmaceutical use of sirtuin-modulating compounds or chalcogenides, while preserving their beneficial effects, the instant invention contemplates enhanced efficacy in mammals in need of sirtuin-modulating compounds or chalcogenides therapy that is derived from combining sirtuin-modulating compounds as a pharmaceutical intervention.

In addition, according to certain aspects of the present invention, it is contemplated that combinations of sirtuin-modulating compounds and chalcogenides have increased biological and therapeutic activity in the treatment and prevention of various diseases and conditions presently treated with either sirtuin-modulating compounds or chalcogenides. In certain embodiments, the combination of sirtuin-modulating compounds and chalcogenides may have either additive or synergistic effects, e.g., in protecting biological matter and tissue from injury and increasing lifespan or longevity. Accordingly, the present invention includes improved methods of treating diseases and disorders previously treated with sirtuin modulators, which comprise administering sirtuin-modulating compounds in combination with chalcogenides. Further, the present invention provides improved methods of enhancing cell survival, increasing longevity, or protecting cells or tissue from injury due to hypoxia or ischemia, which comprise administering chalcogenides in combination with sirtuin-modulating compounds. The invention further includes compositions comprising both sirtuin-modulating compounds and chalcogenides, as well as methods and devices for the preparation and administration of combinations of sirtuin-modulating compounds and chalcogenides to a subject.

Therefore, in some embodiments of the invention, longevity is induced. This can be accomplished by continuing to expose the biological matter to a chalcogenide or other active compound and/or exposing the biological matter to a nonphysiological temperature or another chalcogenide or other active compound and any combination or range derivable therein.

Furthermore, the term “provide” is used according to its ordinary and plain meaning to mean “to supply.” It is contemplated that an active compound may be provided to biological matter in one form and be converted by chemical reaction to its form as an active compound. The term “provide” encompasses the term “expose” in the context of the term “effective amount,” according to the present invention.

The amount of an active compound that is provided to biological matter can be about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 micromolar, millimolar, or molar, or any range derivable therein.

Survivability includes survivability when the matter is under adverse conditions—that is, conditions under which there can be adverse and nonreversible damage or injury to biological matter. Adverse conditions can include, but are not limited to, when oxygen concentrations are reduced in the environment (hypoxia or anoxia, such as at high altitudes or under water); when the biological matter is incapable of receiving that oxygen (such as during ischemia), which can be caused by i) reduced blood flow to organs (e.g., heart, brain, and/or kidneys) as a result of blood vessel occlusion (e.g., myocardial infarction, and/or stroke), ii) extracorporeal blood shunting as occurs during heart/lung bypass surgery (e.g., “pumphead syndrome” in which heart or brain tissue is damaged as a result of cardiopulmonary bypass), or iii) as a result of blood loss due to trauma (e.g., hemorrhagic shock or surgery); hypothermia, where the biological material is subjected to sub-physiological temperatures, due to exposure to cold environment or a state of low temperature of the biological material, such that it is unable to maintain adequate oxygenation of the biological materials; hyperthermia, whereby temperatures where the biological material is subjected to supra-physiological temperatures, due to exposure to hot environment or a state of high temperature of the biological material such as by a malignant fever; conditions of excess heavy metals, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease.

A chalcogenide compound or sirtuin-modulating compound either alone or in combination may be used for increase lifespan or any of these other embodiments may lead or provide their desired effect(s), in some embodiments, only when they are in the context of the biological matter (i.e., have no lasting effect) and/or they may provide for these effect(s) for more than 24 hours after the biological matter is no longer exposed to it. Moreover, this can also be the case when a combination of active compounds is used. It should be again noted that a chalcogenide may be a sirtuin modulator.

It is also contemplated that an active compound may be administered before, during, after, or any combination thereof, in relation to the onset or progression of an injurious insult or disease condition. In certain embodiments, pre-treatment of biological matter with an active compound is sufficient to enhance survivability and/or reduce damage from an injurious or disease insult. Pre-treatment is defined as exposure of the biological matter to the active compound before the onset or detection of the injurious or disease insult. Pre-treatment can be followed by termination of exposure at or near the onset of the insult or continued exposure after the onset of insult.

In certain embodiments, methods including pre-exposure to an active compound (i.e., pre-treatment) are used to treat conditions in which an injurious or disease insult is 1) scheduled or elected in advance, or 2) predicted in advance to likely occur. Examples meeting condition 1 include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples meeting condition 2 include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test.

Moreover, additional embodiments of the invention concern prevention of death or irreversible tissue damage from blood loss or other lack of oxygenation to cells or tissue, such as from lack of an adequate blood supply. This may be the result of, for example, actual blood loss, or it may be from conditions or diseases that prevent cells or tissue from being perfused (e.g., reperfusion injury), that cause blockage of blood to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, ITP), and atherosclerosis. Such conditions and diseases also include those that create essentially hypoxic or anoxic conditions for cells or tissue in an organism because of an injury, disease, or condition.

Biological matter may be provided with or exposed to an active compound through inhalation, injection, catheterization, immersion, lavage, perfusion, topical application, absorption, adsorption, or oral administration. Moreover, biological matter may be provided with or exposed to an active compound by administration to the biological matter intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

The present invention also concerns pharmaceutical compositions comprising a therapeutically effective amount of one or more active compounds. It is understood that such pharmaceutical compositions are formulated in pharmaceutically acceptable compositions. For example, the composition may include a pharmaceutically acceptable diluent.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefore.

“Therapeutically effective amount” refers to that amount of a compound of the invention that, when administered to a mammal, is sufficient to effect treatment, as defined below, of a disease or condition in the mammal. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest, e.g., tissue injury, in a mammal, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In certain embodiments, the pharmaceutical composition contains an effective dose of an active compound to provide when administered to a patient a Cₘₐₓ or a steady state plasma concentration of the active compound to produce a therapeutically effective benefit. In certain embodiments, the Cₘₐₓ or steady state plasma concentration to be achieved is about, at least about, or at most about 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 3000, 4000, 5000, 6000, 7000, 8000, 9000, 10000 micromolar or more, or any range derivable therein. In certain embodiments, such as with H2S, the desired Cₘₐₓ or steady state plasma concentration is about between 1 μM to about 10 mM, or between about 100 μM to about 1 mM, or between about 200 μM to about 800 μM. Appropriate measures may be taken to consider and evaluate levels of the compound already in the blood, such as sulfur.

In certain embodiments, the pharmaceutical composition provides an effective dose of H2S to provide when administered to a patient a Cₘₐₓ or a steady state plasma concentration of between 1 μM to 10 mM, between about 100 μM to about 1 mM, or between about 200 μM to about 800 μM. In relating dosing of hydrogen sulfide to dosing with sulfide salts, in typical embodiments, the dosing of the salt is based on administering approximately the same sulfur equivalents as the dosing of the H2S. Appropriate measures will be taken to consider and evaluate levels of sulfur already in the blood.

In certain embodiments, the composition comprises a gaseous form of one or more of the active compounds specified above. In another embodiment, the composition comprises a salt of one or more of these compounds. In one particular embodiment, a pharmaceutical composition comprises a gaseous form of Formula I or IV or a salt of Formula I or IV. A gaseous form or salt of H2S is specifically contemplated in some aspects of the invention. It is contemplated that the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, or more ppm, or any range derivable therein. Alternatively, the effective amount of gas(es) may be expressed as about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein, with respect to the concentration in the air to which the biological matter is exposed. Moreover, it is contemplated that with some embodiments, the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 parts per billion (ppb) or any range derivable therein. In particular embodiments, the amount of hydrogen selenide provided to biological matter is on this order of magnitude.

In some embodiments of the invention, the pharmaceutical composition is a liquid. As discussed elsewhere, the composition may be a liquid with the relevant compound(s) dissolved or bubbled into the composition. In some cases, the pharmaceutical composition is a medical gas. According to the United States Food and Drug Administration, “medical gases” are those gases that are drugs within the meaning of §201(g)(1) of the Federal Food, Drug and Cosmetic Act (“the Act”) (21 U.S.C. §321(g) and pursuant to §503(b)(1)(A) of the Act (21 U.S.C. §353(b)(1)(A) are required to be dispensed by prescription. As such, such medical gases require an appropriate FDA label. A medical gas includes at least one active compound.

The chalcogenide or other active compound may be or may be provided as a gas, semi-solid liquid (such as a gel or paste), liquid, or solid. It is contemplated that biological matter may be exposed to more than one such active compound and/or to that active compound in more than one state. Moreover, the active compound may be formulated for a particular mode of administration, as is discussed herein. In certain embodiments, the active compound is a pharmaceutically acceptable formulation for intravenous delivery. In certain embodiments, the active compound is in a pharmaceutically acceptable solid oral dosage form. In certain embodiments, more than one active compound are combined in a pharmaceutically acceptable solid oral dosage form.

In certain embodiments, the active compound is a gas. Moreover, it is specifically contemplated that the active compound is a chalcogenide compound as a gas. In some embodiments, the active compound is in a gas mixture comprising more than one gas. The other gas(es) is a non-toxic and/or a non-reactive gas in some embodiments. In some embodiments, the other gas is a noble gas (helium, neon, argon, krypton, xenon, radon, or ununoctium), nitrogen, nitrous oxide, hydrogen, or a mixture thereof. For instance, the non-reactive gas may simply be a mixture that constitutes “room air,” which is a mixture of nitrogen, oxygen, argon and carbon dioxide, as well as trace amounts of other molecules such as neon, helium, methane, krypton, and hydrogen. The precise amounts of each varies, though a typical sample might contain about 78% nitrogen, 21% oxygen, 0.9% argon, and 0.04% carbon dioxide. It is contemplated that in the context of the present invention, “room air” is a mixture containing about 75 to about 81% nitrogen, about 18 to about 24% oxygen, about 0.7 to about 1.1% argon, and about 0.02% to about 0.06% carbon dioxide. A gaseous active compound may be first diluted with a non-toxic and/or non-reactive gas prior to administration or exposure to biological matter. Additionally or alternatively, any gaseous active compound may be mixed with room air prior to administration or exposure to biological matter or the compound may be administered or exposed to the biological matter in room air.

In some instances, the gas mixture also contains oxygen. An active compound gas is mixed with oxygen to form an oxygen gas (O₂) mixture in other embodiments of the invention. Specifically contemplated is an oxygen gas mixture in which the amount of oxygen in the oxygen gas mixture is less than the total amount of all other gas or gases in the mixture.

In some embodiments, the invention concerns compositions and articles of manufacture that contain one or more active compounds. In certain embodiments, a composition has one or more of these active compounds as a gas that is bubbled in it so that the composition provides the compound to the biological matter when it is exposed to the composition. Such compounds may be gels, liquids, or other semi-solid material. In certain embodiments, a solution has a chalcogenide as a gas bubbled through it. It is contemplated that the amount bubbled in the gas will provide the appropriate amount of the compound to biological material exposed to the solution. In certain embodiments, the amount of gas bubbled into the solution is about, at least about, or at most about 0.5, 1.0, 1.5, 2.0. 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100 times or more, or any range derivable therein, than the amount to which the biological matter is effectively provided.

In certain embodiments, the ratio of either chalcogenide to sirtuin-modulating compound or sirtuin-modulating compound to chalcogenide is about, at least about, or at most about 1:1, 2:1, 2.5:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, 300:1, 310:1, 320:1, 330:1, 340:1, 350:1, 360:1, 370:1, 380:1, 390:1, 400:1, 410:1, 420:1, 430:1, 440:1, 450:1, 460:1, 470:1, 480:1, 490:1, 500:1 or more, or any range derivable therein.

Any embodiment involving “exposing” biological matter to an active compound may also be implemented so that biological matter is provided with the active compound or administered the active compound. The term “provide” is used according to its ordinary and plain meaning: “to supply or furnish for use” (Oxford English Dictionary), which, in the case of patients, may refer to the action performed by a doctor or other medical personnel who prescribes a particular active compound or administers it directly to the patient.

Any embodiment wherein a chalcogenide is discussed herein also alternatively contemplates the discussion of a combination of a chalcogenide and a sirtuin-modulating compound, and vice-versa. Therefore, embodiments comprising a chalcogenide also contemplate embodiments of a combination of a chalcogenide and a sirtuin-modulating compound.

B. Biological Matter

Biological matter contemplated for use with the present invention include material derived from invertebrates and vertebrates, including mammals; biological materials includes organisms. In addition to humans, the invention can be employed with respect to mammals of veterinary or agricultural importance including those from the following classes: canine, feline, equine, bovine, ovine, murine, porcine, caprine, rodent, lagomorph, lupine, and ursine. The invention also extends to fish and birds. Other examples are disclosed below.

Moreover, the type of biological matter varies. It can be cells, tissues and organs, as well as organisms for which different compositions, methods, and apparatuses have relevance. The nonprovisional U.S. patent application Ser. Nos. 10/971,576, 10/972,063, and 10/971,575 are hereby incorporated by reference in their entireties.

In some embodiments, the biological material is or comprises cells. It is contemplated that the cell may be any oxygen-utilizing cell. The cell may be eukaryotic or prokaryotic. In certain embodiments, the cell is eukaryotic. More particularly, in some embodiments, the cell is a mammalian cell. Mammalian cells contemplated for use with the invention include, but are not limited to those that are from a: human, monkey, mouse, rat, rabbit, hamster, goat, pig, dog, cat, ferret, cow, sheep, and horse.

Moreover, cells of the invention may be diploid, but in some cases, the cells are haploid (sex cells). Additionally, cells may be polyploid, aneuploid, or anucleate. The cell can be from a particular tissue or organ, such as one from the group consisting of: heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord. Moreover, the cell can also be characterized as one of the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

### V. THERAPEUTIC OR PREVENTATIVE APPLICATIONS

A. Hypoxia and Anoxia

Hypoxia is a common natural stress and several well conserved responses exist that facilitate cellular adaptation to hypoxic environments. To compensate for the decrease in the capacity for aerobic energy production in hypoxia, the cell must either increase anaerobic energy production or decrease energy demand (Hochachka et al., 1996). Examples of both of these responses are common in metazoans and the particular response used depends, in general, on the amount of oxygen available to the cell.

In mild hypoxia, oxidative phosphorylation is still partially active, so some aerobic energy production is possible. The cellular response to this situation, which is mediated in part by the hypoxia-inducible transcription factor, HIF-1, is to supplement the reduced aerobic energy production by upregulating genes involved in anaerobic energy production, such as glycolytic enzymes and glucose transporters (Semenza, 2001; Guillemin et al., 1997). This response also promotes the upregulation of antioxidants such as catylase and superoxide dismutase, which guard against free radical-induced damage. As a result, the cell is able to maintain near normoxic levels of activity in mild hypoxia.

In an extreme form of hypoxia, referred to as “anoxia”—defined here as <0.001 kPa O₂—oxidative phosphorylation ceases and thus the capacity to generate energy is drastically reduced. In order to survive in this environment, the cell must decrease energy demand by reducing cellular activity (Hochachka et al., 2001). For example, in turtle hepatocytes deprived of oxygen, a directed effort by the cell to limit activities such as protein synthesis, ion channel activity, and anabolic pathways results in a 94% reduction in demand for ATP (Hochachka et al., 1996). In zebrafish (Danio rerio) embryos, exposure to anoxia leads to a complete arrest of the heartbeat, movement, cell cycle progression, and developmental progression (Padilla et al., 2001). Similarly, C. elegans respond to anoxia by entering into suspended animation, in which all observable movement, including cell division and developmental progression, ceases (Padilla et al., 2002; Van Voorhies et al., 2000). C. elegans can remain suspended for 24 hours or more and, upon return to normoxia, will recover with high viability. This response allows C. elegans to survive the hypoxic stress by reducing the rate of energetically expensive processes and preventing the occurrence of damaging, irrevocable events such as aneuploidy (Padilla et al., 2002; Nystul et al., 2003).

B. Trauma

In certain embodiments, the present invention may find use in the treatment of patients who are undergoing, or who are susceptible to trauma. Trauma may be caused by external insults, such as burns, wounds, amputations, gunshot wounds, or surgical trauma, internal insults, such as stroke or heart attack that result in the acute reduction in circulation, or reductions in circulation due to non-invasive stress, such as exposure to cold or radiation. On a cellular level, trauma often results in exposure of cells, tissues and/or organs to hypoxia, thereby resulting in induction of programmed cell death, or “apoptosis.” Systemically, trauma leads to the induction of a series of biochemical processes, such as clotting, inflammation, hypotension, and may give rise to shock, which if it persists may lead to organ dysfunction, irreversible cell damage and death. Biological processes are designed to defend the body against traumatic insult; however they may lead to a sequence of events that proves harmful and, in some instances, fatal.

The present invention also contemplates methods for inducing tissue regeneration and wound healing by prevention/delay of biological processes that may result in delayed wound healing and tissue regeneration. In this context, in scenarios in which there is a substantial wound to the limb or organism, the methods of the invention of can aid in the wound healing and tissue regeneration process by managing the biological processes that inhibit healing and regeneration.

In addition to wound healing and hemorrhagic shock discussed below, methods of the invention can be implemented to prevent or treat trauma such as cardiac arrest or stroke. The invention has particular importance with respect to the risk of trauma from emergency surgical procedures, such as thoractomy, laparotomy, and splenic transection.

1. Wound Healing

In many instances, wounds and tissue damage are intractable or take excessive periods of time to heal. Examples are chronic open wounds (diabetic foot ulcers and stage 3 & 4 pressure ulcers), acute and traumatic wounds, flaps and grafts, and subacute wounds (i.e., dehisced incisions). This may also apply to other tissue damage, for example burns and lung damage from smoke/hot air inhalation.

Previous experiments show hibernation to be protective against injury (e.g., pin's in brains), therefore it may have healing effects. Consequently, this technology may be useful in the control of wound healing processes, by bringing the tissue into a more metabolically controlled environment. More particularly, the length of time that cells or tissue are treated can vary depending on the injury. In some embodiments of the invention, biological matter is exposed to an active compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months or more.

2. Hematologic Shock (Hemorrhagic Shock)

Shock is a life-threatening condition that progresses rapidly when interventions are delayed. Shock is a state in which adequate perfusion to sustain the physiologic needs of organ tissues is not present. This is a condition of profound haemodynamic and metabolic disturbance characterized by failure of the circulatory system to maintain adequate perfusion of vital organs. It may result from inadequate blood volume (hypovolaemic shock), inadequate cardiac function (cardiogenic shock) or inadequate vasomotor tone, also referred to as distributive shock (neurogenic shock, septic shock, anaphylactic shock). This often results in rapid mortality of the patient. Many conditions, including sepsis, blood loss, impaired autoregulation, and loss of autonomic tone, may produce shock or shocklike states. The present invention is anticipated to prevent detrimental effects of all the above states of shock, and sustain the life of the biological matter undergoing such shock.

In hemorrhagic shock, blood loss exceeds the body's ability to compensate and provide adequate tissue perfusion and oxygenation. This is frequently due to trauma, but may also be caused by spontaneous hemorrhage (e.g., gastrointestinal bleeding, childbirth), surgery, and other causes. Most frequently, clinical hemorrhagic shock is caused by an acute bleeding episode with a discrete precipitating event. Less commonly, hemorrhagic shock may be seen in chronic conditions with subacute blood loss.

The biology of lethal hemorrhage and the physiological events that lead to shock and ultimately death are not fully understood. However, there are mechanisms through which H2S could reduce the lethal effects of ischemic hypoxia. Hydrogen sulfide inhibits cytochrome C oxidase and could reduce oxygen demand by inhibiting this enzyme³. Decreased oxygen demand may reduce the deleterious effects of low oxygen levels including a reduction of metabolic acidosis. Furthermore, tissue sulfhydryl levels decrease during shock (Beck et al., 1954). Exogenous H2S may prevent this hyposulfidic state and maintain sulfur homeostasis.

Hydrogen sulfide is naturally produced in animals and exhibits potent biological activities (Kamoun, 2004). Most proteins contain disulfide linked cysteine residues, and the reversible conversion from free thiol to disulfide can regulate specific enzyme activities (Ziegler, 1985). Furthermore, sulfide is electronegative and exhibits high affinity for transition metals. Proteins containing transition metal atoms, such as cytochrome oxidase, can be profoundly affected by H2S. And finally, metabolism of H2S into other molecules containing reduced sulfur increases the number of thiols that may exhibit specific biological activity. In addition to (or perhaps because of) these potential modes of action, H2S may exert effects on cardiopulmonary, neuroendocrine, immune, and/or hemostatic systems that ultimately prove beneficial in injury and disease.

U.S. Provisional Application Ser. No. 60/793,520, filed on Apr. 20, 2006, describes the treatment of shock and is hereby incorporated by reference.

C. Cardioplegia and Coronary Heart Disease

In certain embodiments, the present invention may find use as solutions for the treatment of coronary heart disease (CHD) including a use for cardioplegia for cardiac bypass surgery (CABG).

CHD results from athlerosclerosis, a narrowing and hardening of the arteries that supply oxygen rich blood to the heart muscle. The arteries harden and become narrow due to the buildup of plaque on the inner walls or linings of the arteries. Blood flow to the heart is reduced as plaque narrows the coronary arteries. This decreases the oxygen supply to the heart muscle. This may manifest in 1) angina, which is chest pain or discomfort that happens when the heart is not getting enough blood; 2) heart attack, which can occur when a blood clot suddenly cuts off most or all blood supply to part of the heart and cells in the heart muscle that do not receive enough oxygen-carrying blood begin to die, potentially causing permanent damage to the heart muscle; 3) heart failure, which is when the heart is unable to pump blood effectively to the rest of the body; arrhythmias, which are changes in the normal rhythm of the heartbeats.

About 10% of CHD patients will undergo coronary artery bypass graft (CABG) surgery. Patients with severe narrowing or blockage of the left main coronary artery or those with disease involving two or three coronary arteries are generally considered candidates for bypass surgery. In CABG, the surgeon uses a portion of a healthy vessel (either an artery or a vein) from another part of the body to create a detour (or bypass) around the blocked portion of the coronary artery. Patients typically receive from 1 to 5 bypasses in a given operation. During the procedure, generally the heart is placed in a state of paralysis, known as cardioplegia (CP), during which a heart-lung machine artificially maintains circulation. Patients are under general anesthesia during the operation, which usually lasts between 3 to 6 hours.

Approximately 13% of all patients will be re-admitted to the hospital within 30 days due to reasons related to the CABG. Hannan et al., 2003; Mehlhorn et al., 2001. One of the main reasons for re-admission is heart failure, presumably due to ischemic damage during the surgery.

Recent advances in cardiac surgery have centered upon optimization of cardioplegic parameters in the hope of preventing postoperative ventricular dysfunction and improving overall outcome. Cohen et al., 1999.

Cardioplegic solutions are perfused through the vessels and chambers of the heart and cause its intrinsic beating to cease, while maintaining the viability of the organ. Cardioplegia (paralysis of the heart) is desirable during open-heart surgery and during the procurement, transportation, and storage of donor hearts for use in heart transplantation procedures.

Despite the protective effects provided by the current methods for inducing cardioplegia, there is still some degree of ischemic-reperfusion injury to the myocardium. Ischemic-reperfusion injury during cardiac bypass surgery results in poor outcomes (both morbidity and mortality), especially due to an already weakened state of the heart. Myocardial ischemia results in anaerobic myocardial metabolism. The end products of anaerobic metabolism rapidly lead to acidosis, mitochondrial dysfunction, and myocyte necrosis. High-energy phosphate depletion occurs almost immediately, with a 50 percent loss of ATP stores within 10 minutes. Reduced contractility occurs within 1 to 2 minutes, with development of ischemic contracture and irreversible injury after 30 to 40 minutes of normothermic (37° C.) ischemia.

Reperfusion injury is a well-known phenomenon following restoration of coronary circulation. Reperfusion injury is characterized by abnormal myocardial oxidative metabolism. In addition to structural changes created during ischemia, reperfusion may produce cytotoxic oxygen free radicals. These oxygen free radicals play a significant role in the pathogenesis of reperfusion injury by oxidizing sarcolemmal phospholipids and thus disrupting membrane integrity. Oxidized free fatty acids are released into the coronary venous blood and are a marker of myocardial membrane phospholipid peroxidation. Protamine induces complement activation, which activates neutrophils. Activated neutrophils and other leukocytes are an additional source of oxygen free radicals and other cytotoxic substances.

The present invention provides methods and compositions for inducing cardioplegia that will provide greater protection to the heart during bypass surgery. In certain embodiments, the present invention provides a cardioplegic solution comprising H2S (or another active compound) dissolved in solution or bubbled as a gas in the solution. In some embodiments, the invention further comprises at least a first device, such as a catheter or cannula, for introducing an appropriate dose of the cardioplegic solution to the heart. In certain aspects, the invention further comprises at least a second device, such as a catheter or cannula, for removing the cardioplegic solution from the heart.

Bypass surgery typically last for 3-6 hours, however, complications and multiple vessel CABG can extend the duration to 12 hours or longer. It is contemplated that the heart would be treated during the surgery. Thus, in some embodiments of the invention, the heart is exposed to an active compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18 hours or more, and any range or combination therein.

D. Reducing Damage from Cancer Therapy

Cancer is a leading cause of mortality in industrialized countries around the world. The most conventional approach to the treatment of cancer is by administering a cytotoxic agent to the cancer patient (or treatment ex vivo of a tissue) such that the agent has a more lethal effect on the cancer cells than normal cells. The higher the dose or the more lethal the agent, the more effective it will be; however, by the same token, such agents are all that more toxic (and sometimes lethal) to normal cells. Hence, chemo- and radiotherapy are often characterized by severe side effects, some of which are life threatening, e.g., sores in the mouth, difficulty swallowing, dry mouth, nausea, diarrhea, vomiting, fatigue, bleeding, hair loss and infection, skin irritation and loss of energy (Curran, 1998; Brizel, 1998).

Treatment of virtually any hyperproliferative disorder, including benign and malignant neoplasias, non-neoplastic hyperproliferative conditions, pre-neoplastic conditions, and precancerous lesions, is contemplated. Such disorders include restenosis, cancer, multi-drug resistant cancer, primary psoriasis and metastatic tumors, angiogenesis, rheumatoid arthritis, inflammatory bowel disease, psoriasis, eczema, and secondary cataracts, as well as oral hairy leukoplasia, bronchial dysplasia, carcinomas in situ, and intraepithelial hyperplasia. In particular, the present invention is directed at the treatment of human cancers including cancers of the prostate, lung, brain, skin, liver, breast, lymphoid system, stomach, testicles, ovaries, pancreas, bone, bone marrow, gastrointestine, head and neck, cervix, esophagus, eye, gall bladder, kidney, adrenal glands, heart, colon and blood. Cancers involving epithelial and endothelial cells are also contemplated for treatment.

Generally, chemo- and radiotherapy are designed to reduce tumor size, reduce tumor cell growth, induce apoptosis in tumor cells, reduce tumor vasculature, reduce or prevent metastasis, reduce tumor growth rate, accelerate tumor cell death, and kill tumor cells. The goals of the present invention are no different. Thus, it is contemplated that one will combine compositions of the present invention with secondary anti-cancer agents (secondary agents) effective in the treatment of hyperproliferative disease. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Secondary anti-cancer agents include biological agents (biotherapy), chemotherapy agents, and radiotherapy agents. More generally, these other compositions are provided in a combined amount effective to kill or inhibit proliferation of the cancer or tumor cells, while at the same time reducing or minimizing the impact of the secondary agents on normal cells. This process may involve contacting or exposing the cells with an active compound and the secondary agent(s) at the same time. This may be achieved by contacting the cell with a single composition or pharmacological formulation that includes both agents, or by contacting or exposing the cell with two distinct compositions or formulations, at the same time, wherein one composition includes an active compound and the other includes the second agent(s).

Alternatively, the active compound therapy may precede or follow the secondary agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In certain embodiments, it is envisioned that biological matter will be treated for between about 2 and about 4 hours while the cancer treatment is being administered. In some embodiments of the invention, biological matter is exposed to an active compound for about, at least about, or at most about 30 seconds, 1, 2, 3, 4, 5, 10, 15, 20, 25, 30, 35, 40, 45, 50, 55 minutes, 1, 2, 3, 4, 5, 6 hours or more, and any range or combination therein.

Various combinations may be employed; the active compound is “A” and the secondary anti-cancer agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the active compounds of the present invention to a patient will follow general protocols for the administration of chemotherapeutics, taking into account the toxicity, if any, of the compound. It is expected that the treatment cycles would be repeated as necessary. It also is contemplated that various standard therapies, as well as surgical intervention, may be applied in combination with the above-described anti-cancer therapy. It is further contemplated that any combination treatment contemplated for use with an active compound and a non-active compound (such as chemotherapy), may be applied with respect to multiple active compounds.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a composition of the invention (for example, a hypoxic antitumor compound) or a chemotherapeutic or radiotherapeutic agent is delivered to a target cell or are placed in direct juxtaposition with the target cell. In combination therapy, to achieve cell killing, both agents may be delivered to a cell in a combined amount effective to kill the cell or prevent it from dividing.

E. Neurodegeneration

The present invention may be used to treat neurodegenerative diseases. Neurodegenerative diseases are characterized by degeneration of neuronal tissue, and are often accompanied by loss of memory, loss of motor function, and dementia. With dementing diseases, intellectual and higher integrative cognitive faculties become more and more impaired over time. It is estimated that approximately 15% of people 65 years or older are mildly to moderately demented. Neurodegenerative diseases include Parkinson's disease; primary neurodegenerative disease; Huntington's Chorea; stroke and other hypoxic or ischemic processes; neurotrauma; metabolically induced neurological damage; sequelae from cerebral seizures; hemorrhagic shock; secondary neurodegenerative disease (metabolic or toxic); Alzheimer's disease, other memory disorders; or vascular dementia, multi-infarct dementia, Lewy body dementia, or neurodegenerative dementia.

Evidence shows that the health of an organism, and especially the nervous system, is dependent upon cycling between oxidative and reductive states, which are intimately linked to circadian rhythms. That is, oxidative stress placed upon the body during consciousness is cycled to a reductive environment during sleep. This is thought to be a large part of why sleep is so important to health. Certain neurodegenerative disease states, such as Huntington's disease and Alzheimer's disease, as well as the normal processes of aging have been linked to a discord in this cycling pattern. There is also some evidence that brain H2S levels are reduced in these conditions (Eto et al., 2002).

The present invention can be used to regulate and control the cycling between the oxidative and reduced states, for example, to prevent or reverse the effects of neurodegenerative diseases and processes. Controlling circadian rhythms can have other applications, for example, to adjust these cycling patterns after traveling from one time zone to another, so as to adjust to the new time zone. Furthermore, reduced metabolic activity overall has been shown to correlate with health in aged animals and humans. Therefore, the present invention would also be useful to suppress overall metabolic function to increase longevity and health in old age. It is contemplated that this type of treatment would likely be administered at night, during sleep for period of approximately 6 to 10 hours each day. This could require daily treatment for extended periods of time from months to years.

F. Aging

Furthermore, aging itself may be thoroughly or completely inhibited for the period of time when the biological matter is in that state. Thus the present invention may inhibit aging of biological material, with respect to extending the amount of time the biological material would normally survive and/or with respect to progression from one developmental stage of life to another.

G. Blood Disease

A number of blood diseases and conditions may be addressed using compositions and methods of the invention. These diseases include, but are not limited to, thalassemia and sickle cell anemia.

1. Thalassemia

Normal hemoglobin contains two alpha and two beta globin polypeptide (protein) chains, each bound to an iron containing heme ring. Thalassemia is a group of conditions in which there is an imbalance of alpha and beta chains leading to the unpaired chains precipitating on the normally fragile red blood cell membrane, leading to cell destruction. This leads to severe anemia that the marrow tries to compensate for by trying to make more red cells. Unfortunately due to toxicity from unpaired chains this process is very inefficient leading to massive expansion of the marrow space and spread of blood making to other parts of the body. This and the anemia lead to major toxicities. Several models exist as to why unpaired globin chains are so damaging but many entail that increased free radicals generated by the iron attached to the unpaired globin chains are central to the early destruction of the red cells. Thus any intervention that might decrease the oxidative damage from these free radicals could increase red cell lifespan, improve the anemia, lead to decreased need for making red cells, and less damage from marrow expansion and spread.

It is estimated that over 30,000 children are born with severe thalassemia each year, of which it is estimated that most living in developed countries live into their twenties, while in third world countries (where the majority of patients live) most die as young children. Based on the current results in other model systems presented here, it expected that exposing animals with thalassemia to sulfides will increase their red cells' ability to withstand oxidative damage, leading to prolonged red cell survival.

2. Sickle Cell Disease

Normal hemoglobin (HbA) contains two alpha and two beta globin polypeptide (protein) chains, each bound to an iron containing heme ring. In sickle cell disease (SCD; also called sickle cell anemia) is a group of conditions in which a mutant beta chain leads to an altered hemoglobin (HbS). Upon deoxygention HbS can polymerize (crystallize) and precipitate damaging the normally fragile red blood cell membrane, leading to cell destruction and anemia low red blood cells (RBC). In addition cells with polymerized HbS change shape (sickle) and become sticky and activate mechanisms leading to coagulation and blockage of blood flow. This can lead to hypoxic damage of the surrounding tissue resulting in pain, organ dysfunction and eventually premature death. Decreased stores of sulfur containing antioxidants are noted in patients. In addition oxidative damage and increased reactive oxygen species (ROS) have been implicated in crystallization, RBC membrane damage and tissue damage related to inadequate blood flow. Sulfides have been implicated in “re-charging” antioxidant stores, and potentially minimizing oxidative damage. There are reasons to think sulfides could prevent problems at several stages of sickle cell pathology. Furthermore, given the ability of active compounds to protect from hypoxia in other systems, suggests that it should also protect animals and humans subjected to the adverse conditions posed by this disease state.

It is contemplated that any agent or solution used with a biological sample that is living and that will be used as a living material will be pharmaceutically acceptable or pharmacologically acceptable. The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared either as liquid solutions or suspensions; solid forms suitable for solution in, or suspension in, liquid prior to use can also be prepared.

### VI. MODES OF ADMINISTRATION AND PHARMACEUTICAL COMPOSITIONS

A. Administration

The routes of administration of a active compound will vary, naturally, with the location and nature of the condition to be treated, and include, e.g., inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation. As detailed below, active compounds may be administered as medical gases by inhalation or intubation, as injectable liquids by intravascular, intravenous, intra-arterial, intracerobroventicular, intraperitoneal, subcutaneous administration, as topical liquids or gels, or in solid oral dosage forms.

Moreover, the amounts may vary depending on the type of biological matter (cell type, tissue type, organism genus and species, etc.) and/or its size (weight, surface area, etc.). It will generally be the case that the larger the organism, the larger the dose. Therefore, an effective amount for a mouse will generally be lower than an effective amount for a rat, which will generally be lower than an effective amount for a dog, which will generally be lower than an effective amount for a human. The effective amountof active compound to achieve the desired result in biological matter depends on the dosage form and route of administration. For inhalation, in some embodiments effective concentrations are in the range of 50 ppm to 500 ppm, delivered continuously. For intravenous administration, in some embodiments effective concentrations are in the range of 0.05 to 50 milligrams per kilogram of body weight delivered continuously.

Similarly, the length of time of administration may vary depending on the type of biological matter (cell type, tissue type, organism genus and species, etc.) and/or its size (weight, surface area, etc.) and will depend in part upon dosage form and route of administration. In particular embodiments, an active compound is provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. An active compound may be administered in a single dose or multiple doses, with varying amounts of time between administered doses.

In the case of transplant, the present invention may be used pre- and or post-operatively to render host or graft materials quiescent. In a specific embodiment, a surgical site may be injected or perfused with a formulation comprising a chalcogenide. The perfusion may be continued post-surgery, for example, by leaving a catheter implanted at the site of the surgery.

B. Injectable Compositions and Formulations

The preferred methods for the delivery of active compounds of the present invention are inhalation, intravenous injection, perfusion of a particular area, and oral administration. However, the pharmaceutical compositions disclosed herein may alternatively be administered parenterally, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Solutions of the active compounds may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

C. Intravenous Formulations

In one embodiment, active compounds of the invention may be formulated for parenteral administration (e.g., intravenous, intra-arterial). In the cases where the active compound is a gas at room temperature, a solution containing a known and desired concentration of the gas molecule dissolved in a liquid or a solution for parenteral administration is contemplated. Preparation of the active compound solution may be achieved by, for example, contacting (e.g., bubbling or infusing) the gas with the solution to cause the gas molecules to dissolve in the solution. Those skilled in the art will recognize that the amount of gas that dissolves in the solution will depend on a number of variables including, but not limited to, the solubility of the gas in the liquid or solution, the chemical composition of the liquid or solution, its temperature, its pH, its ionic strength, as well as the concentration of the gas and the extent of contacting (e.g., rate of and duration of bubbling or infusing). The concentration of the active compound in the liquid or solution for parenteral administration can be determined using methods known to those skilled in the art. The stability of the active compound in the liquid or solution can be determined by measuring the concentration of the dissolved active compound after varying intervals of time following preparation or manufacture of the solution, where a decrease in the concentration of the compound compared to the starting concentration is indicative of loss or chemical conversion of the active compound.

In some embodiments, there is a solution containing a chalcogenide compound is produced by dissolving a salt form of the chalcogenide into sterile water or saline (0.9% sodium chloride) to yield a pharmaceutically acceptable intravenous dosage form. The intravenous liquid dosage form may be buffered to a certain pH to enhance the solubility of the chalcogenide compound or to influence the ionization state of the chalcogenide compound. In the cases of hydrogen sulfide or hydrogen selenide, any of a number of salt forms known to those skilled in the art may suffice, including, but not limited to, sodium, calcium, barium, lithium, or potassium. In another preferred embodiment, sodium sulfide or sodium selenide is dissolved in sterile phosphate buffered saline and the pH is adjusted to 7.0 with hydrochloric acid to yield a solution of known concentration which can be administered to a subject intravenously or intrarterially.

It is contemplated that in some embodiments, a pharmaceutical composition of the invention is a saturated solution with respect to the active compound. The solution can be any pharmaceutically acceptable formulation, many of which are well known, such as Ringer's solution. In certain embodiments, the concentration of the active compound is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 M or more, or any range derivable therein (at standard temperature and pressure (STP)). With H2S, for example, in some embodiments, the concentration can be from about 0.01 to about 0.5 M (at STP). It is specifically contemplated the above concentrations may be applied with respect to carbon monoxide and carbon dioxide in a solution separately or together.

Furthermore, when administration is intravenous, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the solution is specified by volume, depending on the concentration of the solution. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

In some embodiments, the solution of the active compound for parenteral administration is prepared in a liquid or solution in which the oxygen has been removed prior to contacting the liquid or solution with the active compound. Certain chalcogenide compounds (e.g., hydrogen sulfide, hydrogen selenide), are not stable in the presence of oxygen due to their ability to react chemically with oxygen, leading to their oxidation and chemical transformation. Oxygen can be removed from liquids or solutions using methods known in the art, including, but not limited to, application of negative pressure (vacuum degasing) to the liquid or solution, or contacting the solution or liquid with a reagent which causes oxygen to be bound or “chelated”, effectively removing it from solution.

In another embodiment, the solution of the active compound for parenteral administration may be stored in a gas-tight container. This is particularly desirable when the oxygen has previously been removed from the solution to limit or prevent oxidation of the active compound. Additionally, storage in a gas-tight container will inhibit the volatilization of the gas or other active compound from the liquid or solution, allowing a constant concentration of the dissolved active compound to be maintained. Gas-tight containers are known to those skilled in the art and include, but are not limited to, “i.v. bags” comprising a gas impermeable construction material, or a sealed glass vial. To prevent exposure to air in the gas-tight storage container, an inert gas, such as nitrogen or argon, may be introduced into the container prior to closure.

D. Topical Formulations and Uses Thereof

Methods and compositions of the present invention are useful for inducing survivability in superficial layers of the skin and oral mucosa, including, but not limited to, hair follicle cells, capillary endothelial cells, and epithelial cells of the mouth and tongue. Radiation therapy and chemotherapy for the treatment of cancer damage normal cells in the hair follicles and oral mucosa, leading to the unintended, but debilitating side effects of cancer therapy, hair loss and oral mucositis, respectively. Treatment with compounds of the present invention in hair follicle cells and/or the vascular cells that supply blood to the hair follicles may slow, limit or prevent damage to hair follicle cells and the resultant hair loss that accompanies radiation therapy and chemotherapy, or other alopecia, male-pattern baldness, female-pattern baldness, or other absence of the hair from skin areas where it normally is present. Treatment with compounds of the present invention in oral epithelial and mesenchymal cells may slow, limit or prevent damage to cells lining the mouth, esophagus and tongue and the resultant painful condition of oral mucositis.

In certain embodiments the active compound is administered topically. This is achieved by formulating the active compound in a cream, gel, paste, or mouthwash and applying such formulation directly to the areas that require exposure to the active compound (e.g., scalp, mouth, tongue, throat).

The topical compositions of this invention can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

Possible pharmaceutical preparations that can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

E. Solid Dosage Forms

The formulations may conveniently be presented in unit dosage form and may be prepared by any methods practiced in the art of pharmacy. In general, formulations are prepared by bringing the active ingredients into association with finely divided solid carriers, liquid carriers, or both, and then, if necessary or desired, shaping the product. Formulations useful in the practice of the present invention which are suitable for oral administration may be presented as discrete units such as capsules, cachets, or tablets containing a predetermined amount of the active ingredient; as a powder or granules; or as a solution or suspension in an aqueous or non-aqueous liquid. Preferred unit-dosage forms are liquid formulations for injection or oral administration, and tablets, lozenges, capsules or cachets, also suitable for oral administration.

Compressed tablets may be prepared by compressing with suitable means the active ingredients in a free-flowing form such as a powder or granules, optionally mixed with a binder, lubricant, diluent, preservative, surface-active or dispersing agent. Molded tablets may be prepared with suitable molding means such as punching or compressing the active ingredient and any binders or fillers in a tabletting machine. A mixture of the powdered compound moistened with an inert liquid diluent may also be used. Tablets may be optionally coated or scored and may be formulated so as to provide slow or controlled release of the active ingredient contained in the tablet. Tablets may optionally contain other ingredients, such as additional therapeutic agents. Soft shell gelatin capsules used as pharmaceutical coatings are suitable for orally administered formulations of this invention, also.

Pharmaceutical compositions include solid dosage forms in which the active compound is trapped, or sequestered, in a porous carrier framework that is capable of achieving a crystalline, solid state. Such solid dosage forms with the capacity for gas storage are known in the art and can be produced in pharmaceutically acceptable forms (e.g., Yaghi et al. 2003). A particular advantage of such a pharmaceutical composition pertains to chalcogenide compounds (e.g., hydrogen sulfide, carbon monoxide, hydrogen selenide), which can be toxic to certain mammals at certain concentrations in their free form. In certain embodiments, the compound may be formulated for oral administration (see: Remington's Pharmaceutical Sciences (2005); 21st Edition, Troy, David B. Ed. Lippincott, Williams and Wilkins).

F. Catheters

In certain embodiments, a catheter is used to provide an active compound to an organism. Of particular interest is the administration of such an agent to the heart or vasculature system. Frequently, a catheter is used for this purpose. Yaffe et al., 2004 discusses catheters particularly in the context of suspended animation, though the use of catheters were generally known prior to this publication.

G. Further Delivery Devices or Apparatuses

In some embodiments it is contemplated that methods or compositions will involve a specific delivery device or apparatus. Any method discussed herein can be implemented with any device for delivery or administration including, but not limited, to those discussed herein.

For topical administration of active compounds of the invention may be formulated as solutions, gels, ointments, creams, suspensions, etc. as are well-known in the art. Systemic formulations may include those designed for administration by injection or infusion, e.g., subcutaneous, intravenous, intramuscular, intrathecal or intraperitoneal injection, as well as those designed for transdermal, transmucosal, oral or pulmonary administration.

For oral administration, the active compounds of the invention to be formulated as tablets, pills, dragees, capsules, liquids, gels, syrups, slurries, suspensions and the like, for oral ingestion by a patient to be treated or oral liquid preparations such as, for example, suspensions, elixirs and solutions.

For buccal administration, the compositions may take the form of tablets, lozenges, etc. formulated in conventional manner. Other intramucosal delivery might be by suppository or intranasally.

For nasal administration, a suitable formulation may include a carrier comprising a solid, coarse powder having particulate size averaging 20 to 500 microns in diameter. Such a formulation would be administered by rapid inhalation through the nasal passage, for example, from a container of the powder held close to the nose. Suitable formulations including a liquid carrier might include aqueous or oily solutions of the active ingredient. A preferred system of delivery for nasal administration is a nasal spray.

For administration directly to the lung by inhalation the compound of invention may be conveniently delivered to the lung by a number of different devices. For example,

Metered-Dose Inhalers (MDIs): a Metered Dose Inhaler (“MDP”) which utilizes canisters that contain a suitable low boiling propellant, e.g., dichlorodifluoromethane, trichlorofluoromethane, dichlorotetrafluoroethane, carbon dioxide or other suitable gas may be used to deliver the compound of invention directly to the lung. MDI devices are available from a number of suppliers such as 3M Corporation (e.g., on the world wide web at 3m.com/us/healthcare/manufacturers/dds/pdf/dd_valve_canister_brochure.pdf-), Nasacort from Aventis (e.g., world wide web at products.sanofi-aventis.us/Nasacort_HFA/nasacort_HFA.html-63k-), Boehringer Ingelheim, (e.g., world wide web at boehringer-ingelheim.com/corporate/home/download/r_and_d2003.pdf) Aerobid from Forest Laboratories, (e.g., world wide web at frx.com/products/aerobid.aspx) Glaxo-Wellcome, (for example, on the world wide web at .gsk.com/research/newmedicines/newmedicines_pharma.html) and Schering Plough, (world wide web at .schering-plough.com/schering_plough/pc/allergy_respiratory.jsp).

Dry Powder Inhalers (DPIs): DPI devices typically use a mechanism such as a burst of gas to create a cloud of dry powder inside a container, which may then be inhaled by the patient. DPI devices are also well known in the art and may be purchased from a number of vendors which include, for example, Foradil aerolizer from Schering Corporation, (e.g., world wide web .spfiles.com/piforadil.pdf) Advair Diskus from Glaxo-Wellcome. (e.g., world wide web at us.gsk.com/products/assets/us_advair.pdf-) A popular variation is the multiple dose DPI (“MDDPI”) system, which allows for the delivery of more than one therapeutic dose. MDDPI devices are available from companies such as Plumicort Turbuhaler from AstraZeneca, (e.g., world wide web at twistclickinhale.com/ GlaxoWellcome, world wide web at us.gsk.com/products/assets/us_advair.pdf-) and Schering Plough, (e.g., world wide web at .schering-plough.com/schering_plough/pc/allergy_respiratory.jsp). It is further contemplated that such devices, or any other devices discussed herein, may be altered for single use.

Electrohydrodynamic (EHD) aerosol delivery: EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807. EHD aerosol devices may more efficiently deliver drugs to the lung than existing pulmonary delivery technologies.

Nebulizers: Nebulizers create aerosols from liquid drug formulations by using, for example, ultrasonic energy to form fine particles that may be readily inhaled Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974), Intal nebulizer solution by Aventis, (e.g., world wide web at .fda.gov/medwatch/SAFETY/2004/feb_PI/Intal_Nebulizer_PI.pdf).

For administration of a gas directly to the lungs by inhalation various delivery methods currently available in the market for delivering oxygen may be used. For example, a resuscitator such as an ambu-bag may be employed (see U.S. Pat. Nos. 5,988,162 and 4,790,327). An ambu-bag consists of a flexible squeeze bag attached to a face mask, which is used by the physician to introduce air/gas into the casualty's lungs.

A portable, handheld medicine delivery device capable producing atomized agents that are adapted to be inhaled through a nebulizer by a patient suffering from a respiratory condition. In addition, such delivery device provides a means wherein the dose of the inhaled agent can be remotely monitored and, if required altered, by a physician or doctor. See U.S. Pat. No. 7,013,894. Delivery of the compound of invention may be accomplished by a method for the delivery of supplemental gas to a person combined with the monitoring of the ventilation of the person with both being accomplished without the use of a sealed face mask such as described in U.S. Pat. No. 6,938,619. A pneumatic oxygen conserving device for efficiently dispensing oxygen or other gas used during respiratory therapy such that only the first part of the patient's breath contains the oxygen or other therapeutic gas. (See U.S. Pat. No. 6,484,721). A gas delivery device is used which is triggered when the patient begins to inhale. A tail of gas flow is delivered to the patient after the initial inhalation timed period to prevent pulsing of gas delivery to the patient. In this manner gas is only delivered to the patient during the first portion of inhalation preventing gas from being delivered which will only fill the air passageways to the patient's lungs. By efficiently using the oxygen, cylinder bottles of oxygen used when a patient is mobile will last longer and be smaller and easier to transport. By pneumatically delivering the gas to the patient no batteries or electronics are used.

All the devices described here may have an exhaust system to bind or neutralize the compound of invention.

Transdermal administration of the compound of the invention can be achieved by medicated device or patch which is affixed to the skin of a patient. The patch allows a medicinal compound contained within the patch to be absorbed through the skin layers and into the patient's blood stream. Such patches are commercially available as Nicoderm CQ patch from Glaxo Smithkline, (world wide web at nicodermcq.com/NicodermCQ.aspx\) and as Ortho Evra from Ortho-McNeil Pharmaceuticals, (world wide web at ortho-mcneilpharmaceutical.com/healthinfo/womenshealth/products/orthoevra.html).

Transdermal drug delivery reduces the pain associated with drug injections and intravenous drug administration, as well as the risk of infection associated with these techniques. Transdermal drug delivery also avoids gastrointestinal metabolism of administered drugs, reduces the elimination of drugs by the liver, and provides a sustained release of the administered drug. Transdermal drug delivery also enhances patient compliance with a drug regimen because of the relative ease of administration and the sustained release of the drug.

Other modifications of the patch include the Ultrasonic patch which is designed with materials to enable the transmission of ultrasound through the patch, effecting the delivery of medications stored within the patch, and to be used in conjunction with ultrasonic drug delivery processes (see U.S. Pat. No. 6,908,448). Patch in a bottle (U.S. Pat. No. 6,958,154) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See U.S. Pat. No. 6,464,687.

PUMPS and Infusion Devices: An infusion pump or perfusor infuses fluids, medication or nutrients into a patient's circulatory system. Infusion pumps can administer fluids in very reliable and inexpensive ways. For example, they can administer as little as 0.1 mL per hour injections (too small for a drip), injections every minute, injections with repeated boluses requested by the patient, up to maximum number per hour (e.g. in patient-controlled analgesia), or fluids whose volumes vary by the time of day. Various types of infusion devices have been described in the following patent applications before the United States Patent and Trademark Office. These include but are not limited to U.S. Pat. Nos. 7,029,455, 6,805,693, 6,800,096, 6,764,472, 6,742,992, 6,589,229, 6,626,329, 6,355,019, 6,328,712, 6,213,738, 6,213,723, 6,195,887, 6,123,524 and 7,022,107. In addition, infusion pumps are also available from Baxter International Inc. (world wide web at baxter.com/products/medication_management/infusion_pumps/), Alaris Medical Systems (world wide web at alarismed.com/products/infusion.shtml) and from B Braun Medical Inc. (world wide web at bbraunusa.com/index.cfm?uuid=001AA837D0B759A1E34666434FF604ED).

Oxygen/Gas bolus delivery device: Such a device for delivering gas to Chronic Obstructive Pulmonary Disease (COPD) patients is a available from Tyco Healthcare (world wide web at. tycohealth-ece.com/files/d0004/ty_zt7ph2.pdf). It can also be used to deliver the compound of invention. The above device is cost-effective, lightweight, inconspicuous and portable.

“Patch in a bottle” (U.S. Pat. No. 6,958,154) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Implantable Drug Delivery System: Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See U.S. Pat. No. 6,464,687.

The contents of each of the cited patents and web addresses discussed in this section are hereby incorporated by reference.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Adaptation to H2S Increases Thermotolerance and Lifespan in C. elegans

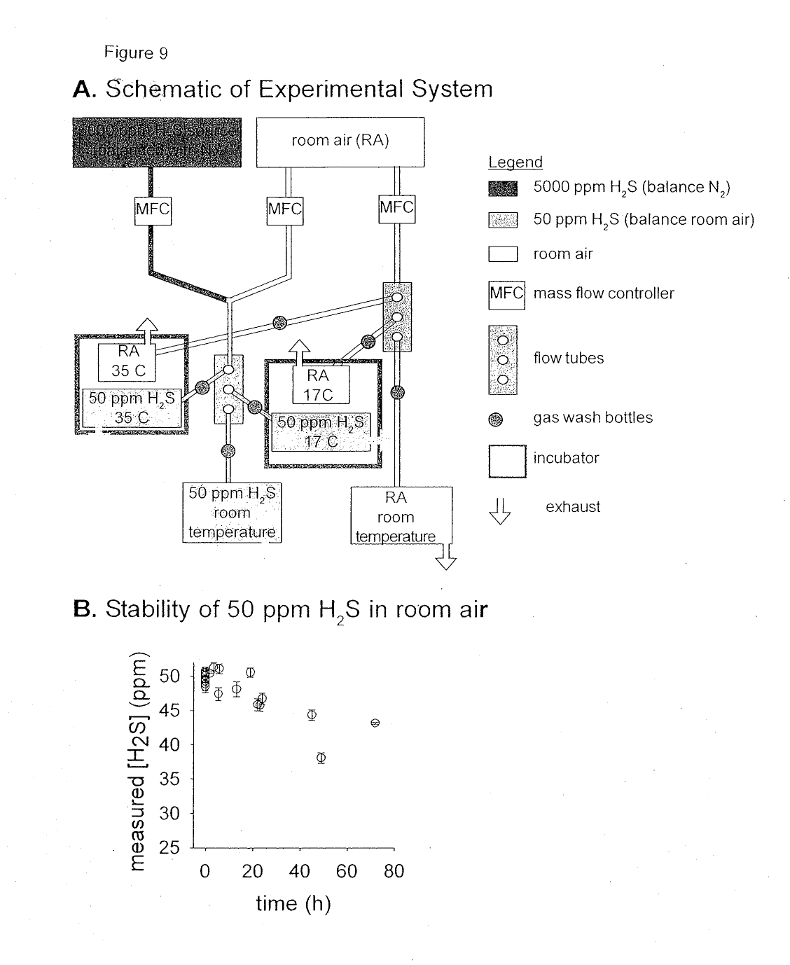

C. elegans were not adversely affected when grown in atmospheres containing 50 ppm H2S (0.005%) in room air (hereafter referred to simply as H2S; FIG. 9). 50 ppm H2S was chosen because this concentration of H2S has been shown to affect mammalian physiology (Blackstone et al., 2005), but it was not apparently toxic to the worms.

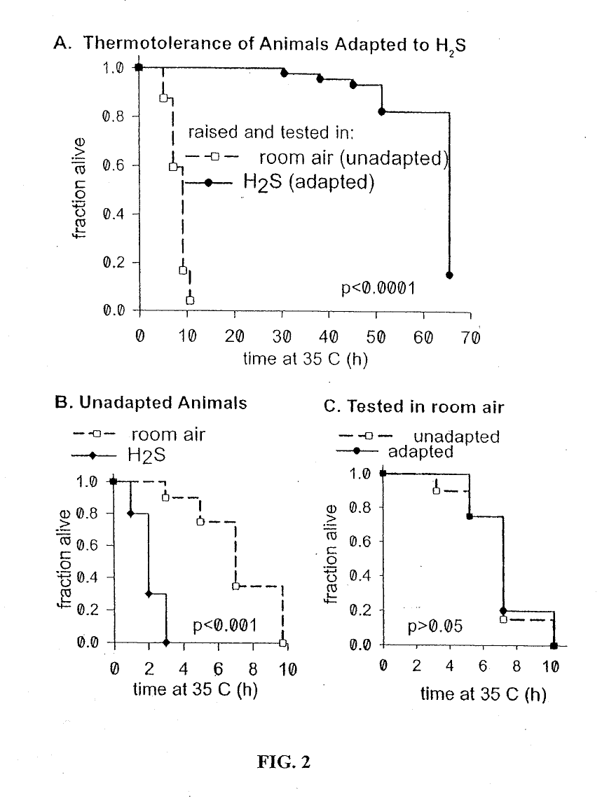

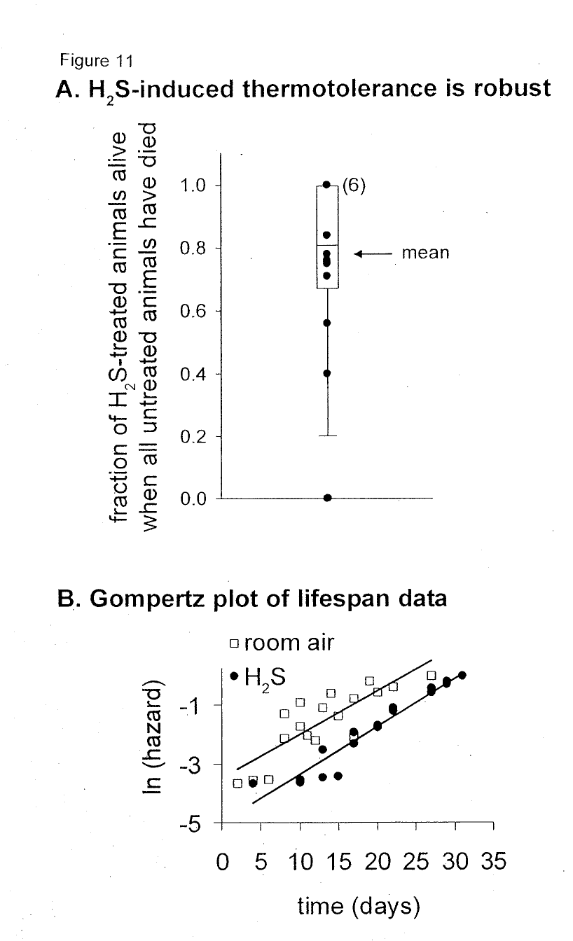

The inventors hypothesized that the physiological alterations of adaptation to H2S may have lead to increased lifespan and thermotolerance. Adaptation to H2S results in increased thermotolerance in C. elegans (FIG. 2). At high temperature, adapted animals typically have a mean survival time up to 8 times longer than unadapted controls. Although the maximum extension of survival time observed varied between experiments, the effect was quite robust with an average of 77-80% of H2S-treated animals alive when all untreated animals had died (15 independent experiments; FIG. 11). Int this experiment, animals were raised in H2S and challenged with high temperature in the presence of H2S. Unadapted animals were generally more sensitive to thermal stress in the presence of H2S, demonstrating that H2S does not act directly to prevent damage associated with thermal stress (FIG. 2B). Moreover, unlike thermotolerance induced by prior stress such as heat shock (Lithgow et al., 1995) or azide (Massie et al., 2003), continuous exposure to H2S is required for thermotolerance of adapted animals (FIG. 2C). These data suggest that H2S exposure initiates physiological alterations, one manifestation of which is increased survival at high temperatures. In other words, these data show that adaptation to H2S enables thermotolerance, and suggest that this phenotype can be distinguished from tolerance to high concentrations of H2S.

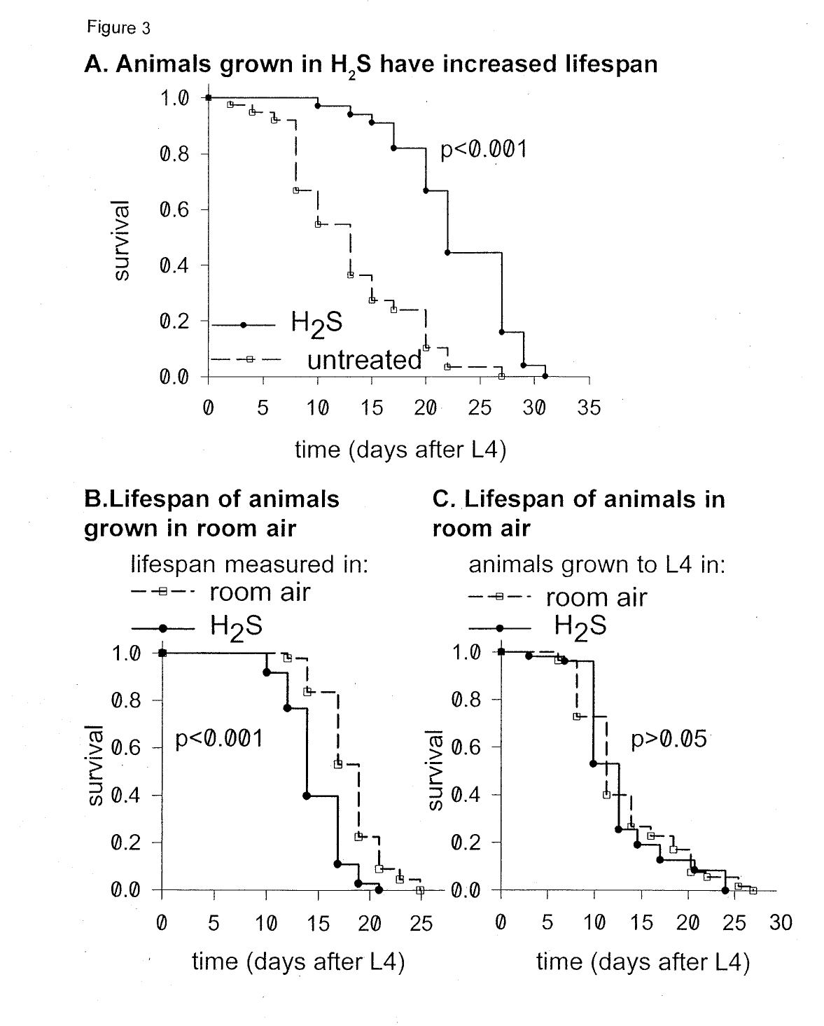

In C. elegans, resistance to high temperature is often correlated with increased lifespan (Lithgow et al., 1995). Indeed, the inventors observed that animals adapted to H2S were long-lived compared to controls (FIG. 3). The mean lifespan of adapted animals grown in H2S was 9.6 days greater than the untreated population, an increase of 70%. Maximum lifespan was also lengthened, as H2S-treated animals reached 75% mortality 10 days after the control population. The inventors have also observed that the life expectancy increase appears to be an overall increase, as well as an increase in “adult” life expectancy (post-sexual maturity). Increased lifespan is not observed when animals are moved to the H2S-containing atmosphere at the beginning of the lifespan experiment (as L4 larvae), indicating that this effect requires prior adaptation to H2S (FIG. 3B). In fact, the lifespan of these animals is slightly shorter than untreated controls. These data suggest that H2S cannot act post-developmentally to slow the rate of aging (see also FIG. 11). Lifespan extension requires continuous presence of H2S in the atmosphere, as animals grown in H2S but moved to room air have normal lifespan (FIG. 3C), indicating that H2S exposure solely during development is insufficient for increased lifespan. The inventors conclude that the increase in lifespan is a feature of the physiological alterations resulting from the adaptation to H2S.

Increased thermotolerance and lifespan of adapted animals does not appear to be associated with reduced metabolic activity. Animals raised in low H2S are visually indistinguishable from untreated controls, and produce similar numbers of progeny (227±18 when moved to H2S compared to 208±16 in room air; p>0.05, n=5 in each group). Neither embryonic nor post-embryonic development is delayed in adapted animals (Table 1). In addition, the rate of egg-laying is not noticeably changed by H2S (Table 2).

TABLE 1
Developmental Rates

| | embryogenesis^{a} | post-embryonic^{b} |
|---|---|---|
| | hours ± SEM (n) | hours ± SEM (n) |
| unadapted^{c} | 12.6 ± 0.4 (43) | 49.6 ± 0.2 (15) |
| adapted^{d} | 13.3 ± 0.4 (34) | 49.3 ± 0.2 (15) |
| clk-1(qm30)^{e} | 16.9 ± 2.0 (17)* | 69.0 ± 0.3 (15)* |

^{a}Median time for 2-cell embryos to hatch at room temperature from one representative experiment.
^{b}Median time for starved first-stage larvae to become gravid adults after return to food in one representative experiment.
^{c}Wild-type animals in room air.
^{d}Wild-type animals in 50 ppm H₂S.
^{e}This mutant has been reported to develop slowly (wong ref).
*significantly different from wild-type, unadopted controls p < 0.05.

TABLE 2
Rate of Egg-Laying in H₂S

| | room air^{a} | H₂S | 2% O₂ | 2% O₂ + H₂S |
|---|---|---|---|---|
| unadapted^{b} | 7.8 ± 1.5 | 8.3 ± 3.0 | 4.2 ± 1.3 | 4.5 ± 1.6 |
| adapted^{c} | nd | 7.8 ± 2.0 | 4.9 ± 1.4 | 4.7 ± 1.2 |
| clk-1(qm30) | 4.2 ± 0.6* | 3.2 ± 1.1* | 2.4 ± 0.8* | 2.5 ± 1.0* |

^{a}Average rate of egg-laying in one representative experiment in embryos per hour ± SD. n = 10 individuals, nd; not done
^{b}Wild-type animals grown in room air.
^{c}Wild-type animals in 50 ppm H₂S.
*signficanly different from wild-type unadapted controls in the same column (p < 0.05 by two-tailed t-test).

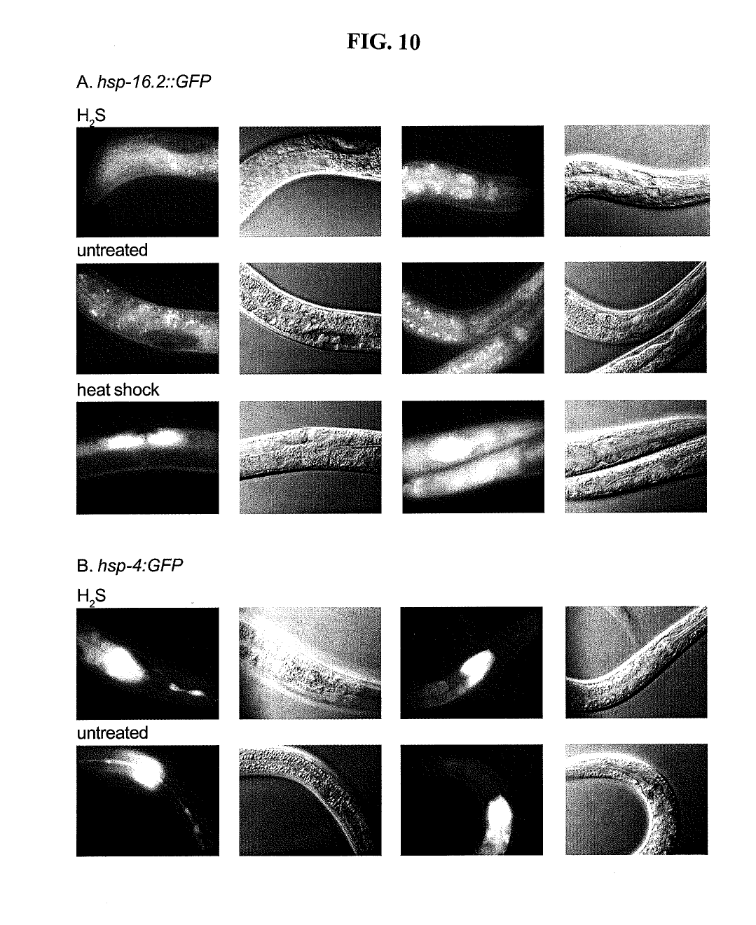

The rate of egg-laying is tightly correlated with oocyte production (McCarter et al., 1999, an energetically expensive activity that is a sensitive readout of metabolic capacity. Consistent with this, the inventors observe a two-fold decrease in the rate of egg-laying when ambient O₂ tension is reduced to 2% (from 21% O₂ in air), a perturbation that decreases the metabolic rate of worms by ˜=30% (Van Voorhies and Ward, 2000). H2S does not further alter the rate of egg-laying in environments with reduced ambient O₂ (Table 2). These data contrast with hypometabolic phenotypes commonly observed in nematodes with defective mitochondrial function (Dillin et al., 2002; Feng et al., 2001; Wong et al., 1995; Hansen et al., 2005). These data demonstrate that apparent metabolic output is not appreciably changed in animals raised in H2S; however, the inventors cannot definitively conclude that mitochondrial energy production has not been affected in these conditions. In addition, H2S exposure does not induce expression of several transgenes driven by heat-shock promoters, including hsp-16.2::GFP and hsp-4::GFP (FIG. 10; Rea et al., 2005; Hong et al., 2004, Calfon et al., 2002; Shen et al., 2001). Together, these data indicate that animals grown in H2S are as healthy as untreated controls, and that in these conditions at this concentration of H2S does not affect apparent metabolic rate.

The inventors also have observed that animals exposed early to H2S reach sexual maturity at the same time with no delays as wild-types, and are otherwise normal. Although the inventors cannot definitively conclude that mitochondrial energy production has not been affected in these conditions, these data demonstrate that overall metabolic output is not appreciably decreased.

Most genes that influence lifespan can be categorized into one of three genetically-independent pathways in C. elegans (reviewed in Kenyon 2005; Wolff and Dillin, 2006; Hekimi et al., 2001). The inventors considered the possibility that exposure to H2S, and associated physiological alterations, may modulate one or more of these pathways. To evaluate this possibility, the inventors tested whether exposure to H2S caused increased thermotolerance in mutant animals with defects in these pathways.

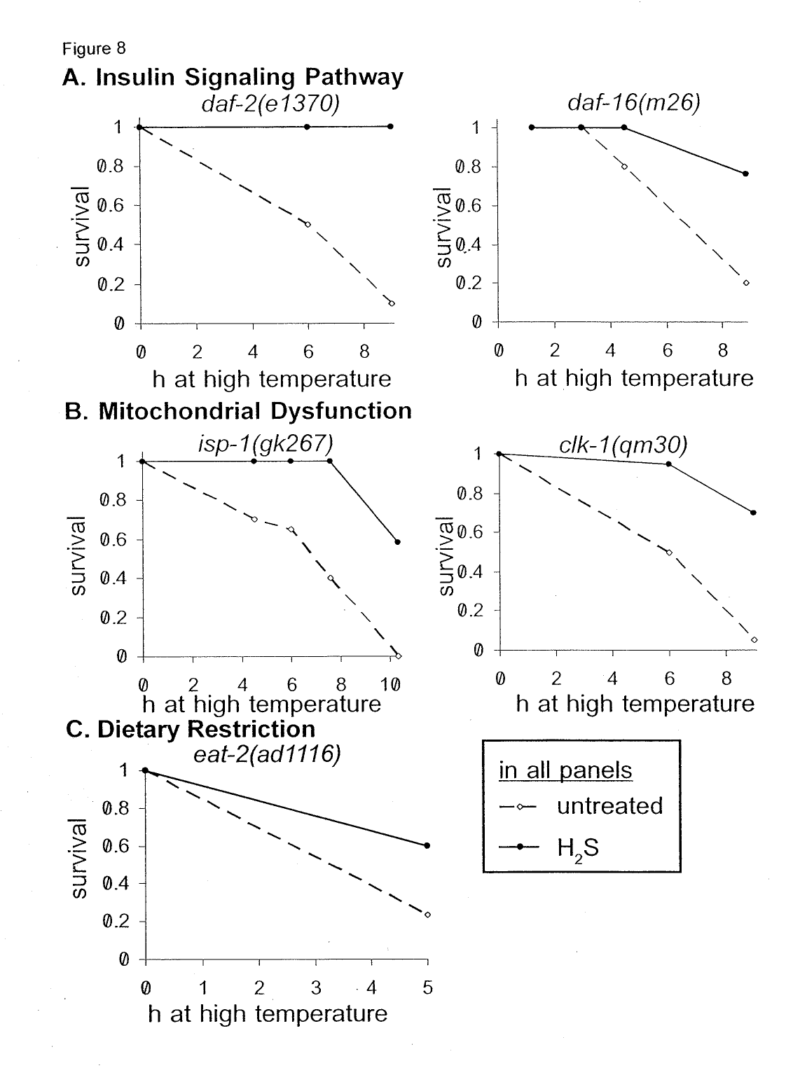

In C. elegans the insulin/IGF signaling (IIS) pathway regulates the decision to enter into an alternative larval stage, the dauer, upon exposure to unfavorable conditions such as high population density, low food or high temperature (Golden and Riddle, 1984). Mutations in the insulin-like receptor DAF-2 that reduce IIS increase the probability of entry into the dauer state, and in adults increase thermotolerance and lengthen lifespan even without entry into dauer (Gems et al., 1998; Kenyon et al., 1993; Dillin et al., 2002). All known phenotypes of daf-2 mutants can be suppressed by mutations in the DAF-16 FOXO transcription factor (Kenyon 2005; Gems et al., 1998; Kenyon et al., 1993). DAF-16 is required for all phenotypes of daf-2. The inventor's data suggest that exposure to H2S does not result in decreased IIS. First, H2S exposure starting in adulthood does not increase lifespan (FIG. 3B), whereas RNAi of daf-2 starting in adulthood is sufficient to increase lifespan (Dillin et al., 2002). Second, daf-2 mutant animals are more resistant to high temperature when grown in H2S (FIG. 8A). Third, H2S does not induce entry into the dauer state in wild-type worms, as post-embryonic development time is not extended, nor does it prevent entry into or exit from dauer in daf-2(e1370) mutants (data not shown). Finally, daf-16 mutant animals become thermotolerant upon exposure to H2S (FIG. 8A). These data suggest that the mechanism by which H2S increases lifespan and thermotolerance are independent of the IIS pathway.

Reduction of mitochondrial function is a well-established mechanism for increasing lifespan of C. elegans (Woff and Dillin, 2006). In vitro, H2S is an inhibitor of cytochrome c oxidase, the terminal enzyme in the electron transport chain (Beauchamp et al., 1984). However, hypometabolic phenotypes were not observed in animals grown in H2S (Table 1), suggesting that mitochondrial function is not grossly affected in these conditions. In addition, depletion of mitochondrial components by RNAi only during development increases lifespan (Dillin et al., 2002), whereas animals grown in H2S but moved to room air as adults are not long-lived (FIG. 3C). These data suggest that H2S exposure has characteristics distinct from mitochondrial dysfunction. In support of this premise, isp-1 and clk-1 mutant animals, which have defects in mitochondrial function and are long-lived (Feng et al., 2001; Wong et al., 1995), become more resistant to high temperature when grown in H2S (FIG. 8B). The inventors conclude from these data that the effect of H2S on lifespan is mediated by a genetic mechanism distinct from mitochondrial dysfunction.

Reduced caloric intake, or dietary restriction (DR), extends lifespan in a wide range of organisms (reviewed in Walker et al., 2005). C. elegans that have reduced rates of pharyngeal pumping are long-lived, likely as a result of DR. These animals appear thin and pale, develop slowly and have reduced fecundity, which are phenotypes not observed in animals adapted to H2S (Table 1; Hansen et al., 2005; Shibata et al., 2000). Furthermore, DR can increase lifespan when initiated in adults (Klass, 1977; Houthoofd et al., 2003; Kaeberlein et al., 2006), wheras H2S exposure cannot (FIG. 3B). Therefore, the inventors considered it unlikely that H2S acts through the DR pathway. Consistent with this interpretation, eat-2(ad1116) mutant animals that are long-lived due to DR (Lakowski and Hekimi, 1998) become thermotolerant upon adaptation to H2S (FIG. 8C). The inventors conclude that the adaptation to H2S alters the physiology of worms in a manner distinct from DR, suggesting that it acts through a separate mechanism.

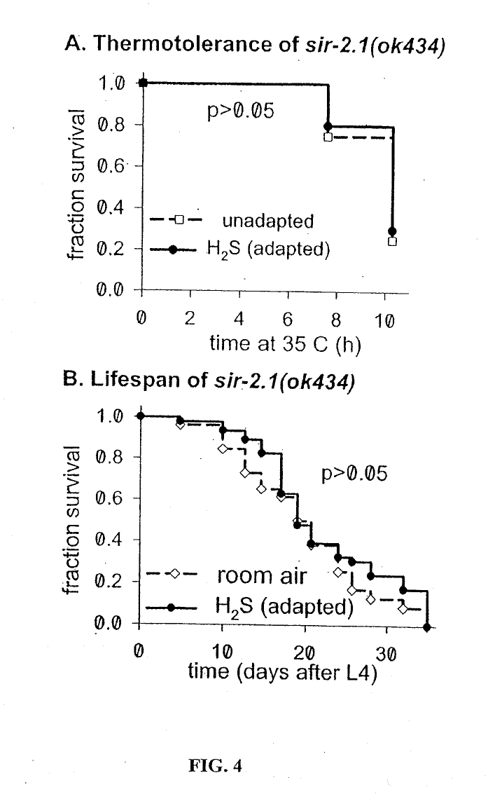

In addition to, but perhaps overlapping with, these genetically-defined pathways, Sir2 homologues influence lifespan in many organisms including C. elegans (Bordone and Guarente, 2005; Longo and Kennedy, 2006; Guarente, 2005; Guarente and Picard, 2005). Overexpression of the C. elegans Sir2 homologue, sir-2.1, increases lifespan of C. elegans by 18-50% (Tissenbaum and Guarente, 2001). The data indicate that sir-2.1 is required for increased thermotolerance and lifespan upon exposure to H2S. In contrast to wild-type (FIGS. 2A and 3A), the thermotolerance and lifespan of nematodes harboring a deletion in the sir-2.1 gene is unchanged when the animals are grown in H2S (FIG. 4). However, the inventors consider it unlikely that H2S results in increased lifespan as a result of increased SIR-2.1 expression. H2S effects on lifespan are independent of daf-16 (FIG. 8A), whereas lifespan extension by overexpression of sir-2.1 requires DAF-16 activity (Tissenbaum and Guarente, 2001). Indeed, sir-2.1 transcript levels in animals grown in H2S are indistinguishable from untreated controls as measured by qRT-PCR, and animals overexpressing sir-2.1 become more thermotolerant when grown in H2S (FIG. 11). The inventors conclude that H2S modulates SIR-2.1 activity to impart increased thermotolerance and lifespan in a manner distinct from sir-2.1 overexpression. The fact that these phenotypes require sir-2.1 further argue that the H2S effect is distinct from DR, as increased lifespan resulting from DR does not require sir-2.1 (Hansen et al., 2007). Moreover, the finding that SIR-2.1 activity is required for increased thermotolerance and lifespan in H2S further suggests that these phenotypes do not result from non-specific metabolic suppression.

Sir2 homologues are NAD+-dependent deacetylase enzymes that may have a variety of substrates (Haigis and Guarente, 2006). This raises the possibility that H2S shifts redox homeostasis, thereby increasing the available NAD+ (or the NAD+/NADH ratio) and resulting in increased SIR-2.1 activity (Longo and Kennedy, 2006; Blander and Guarente, 2004). Alternatively, H2S may directly modify SIR-2.1 to alter its activity (Ziegler, 1985). It is also possible that SIR-2.1 is indirectly activated by some other aspect of H2S-induced physiological alterations. Whatever the mechanism by which H2S-induced physiological alterations are translated into the phenotype of increased lifespan, these studies raise the possibility that endogenous H2S naturally regulates SIR-2.1 activity. It may be that Sir2 homologues are involved in mediating the physiolgical alterations observed in mammals exposed to exogenous H2S.

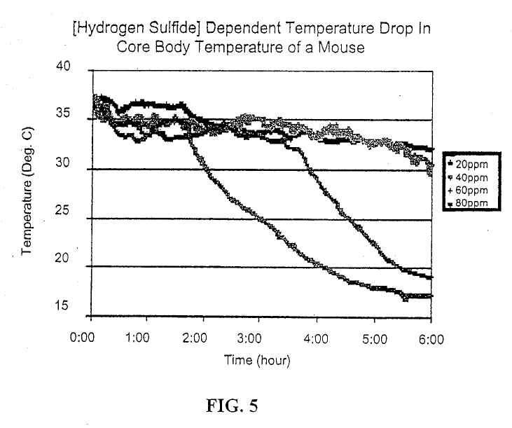

In summary, this data demonstrates that C. elegans adapt to H2S and this adaptation results in physiological alterations that are manifested as increased resistance to thermal stress, increased lifespan and tolerance to otherwise lethal H2S concentrations. Thus, H2S expands the range of conditions in which C. elegans can survive. Perhaps endogenous H2S naturally regulates SIR-2.1 activity to coordinate response to environmental changes. Mice exposed to H2S also show dramatic changes in physiology that protects them against otherwise lethal hypoxia (see FIGS. 5-7). These results suggest that H2S might be a useful therapeutic agent for many pathological states. Defining how organisms adapt to H2S may yield insights into similar mechanisms in higher organisms, including humans, with potentially wide-ranging implications in both basic research and clinical practice.

### Example 2

### Compositions Enhance Survival Under Hypoxic and Ischemic Conditions

In one set of experiments, compositions were tested in male C57BL/6 jugular vein catheterized (JVC) mice, 5-6 weeks old (Taconic), by infusing the animals with the liquid sulfide liquid compositions using 1 mL or 5 mL Luer-Lok syringes (Becton Dickison). An IPTT-300 transponder from Bio Medic Data Systems (BMDS) was used to monitor body temperature. The transponder was injected subcutaneous (S.C.) into the back of the animals at least 24 hours prior to the experiment. A DAS-6008 data acquisition module from BMDS recorded body temperature of the mouse via the transponder, and data was input into a computer spreadsheet and plotted against time.

Each mouse was dosed with liquid compositions through the in-dwelling catheter using an infusion pump (Harvard Apparatus). The mouse was infused until the temperature chip implanted in the skin registered a body temperature of 33° C. If the mouse showed signs of distress before the temperature dropped to 33° C., then the infusion was stopped for 10 minutes and restarted at a rate lower than the previous rate. Once the animal's temperature dropped to 33° C. or below, the infusion was stopped and the mouse was transferred into a hypoxic atmosphere (4.0% O₂) together with a control mouse.

The closed glass chamber was perfused with air and nitrogen at a continuous flow to achieve the desired hypoxic atmosphere of 4% O₂. If the mouse treated with test article survived 60 minutes in the hypoxic atmosphere, it was transferred back to room air, and its recovery was monitored for 24 hours by recording the subcutaneous temperature and by behavioral observation.

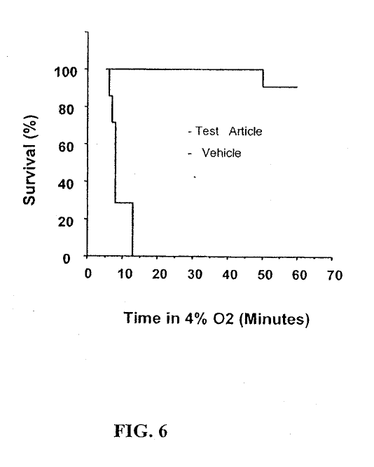

The control mouse typically died within 6-15 minutes. Mice infused with either sodium sulfide (effective dose 0.79 mmol/kg), sodium thiomethoxide (effective dose 4.61 mmol/kg), or sodium thiocyanate (effective dose 4.67 mmol/kg) survived exposure to lethal hypoxia for 60 minutes. See FIG. 6. A mouse infused with cysteamine (effective dose 7.58 mmol/kg) survived in lethal hypoxia for 45 minutes; a mouse infused with cysteamine-5-phosphate sodium salt survived in lethal hypoxia for 31 minutes; and a mouse infused with tetrahydrothiopyran-4-ol survived in lethal hypoxia for 15 minutes. These survival rates are compared to the survival rate of a control mouse, which typically died within 6-15 minutes in the hypoxic environment.

In comparison, certain other test compounds identified in the primary screen as having the ability to lower body temperature did not protect from lethal hypoxia. Thioacetic acid, selenourea, and phosphorothioic acid S-(2-((3-aminopropyl)amino)ethyl)ester all reduced body temperature, but did not enhance survival in hypoxia. 2-Mercapto-ethanol, thioglycolic acid, and 2-mercaptoethyl ether all reduced body temperature but were toxic at the effective temperature reducing dose. Thiourea, dimethyl sulfide, sodium selenide, sodium methane sulfinate, N-acetyl-L-cysteine did not reduce subcutaneous temperature at the highest doses given in this study. Dimethylsulfoxide was excluded because the effective dose (10% DMSO) was too high to be considered for pharmaceutical purposes.

These studies establish that the screening procedures developed may be successfully used to identify compounds capable of protecting animals subjected to lethal hypoxia. In addition, the results of these studies indicate that the identified compounds, as well as other compounds to be identified using this procedure, may be used to protect patients from injury resulting from hypoxic and ischemic injury.

### Example 3

### Mouse Body Core Temperature Dependency on H2S Concentration

In order to determine the concentration of hydrogen sulfide sufficient for the loss of thermoregulation, the inventors exposed mice to a range of hydrogen sulfide concentrations (20 ppm, 40 ppm, 60 ppm, and 80 ppm) (FIG. 5). While 20 ppm and 40 ppm of hydrogen sulfide were sufficient to cause a drop in the core body temperature of a mouse, this was minor compared to the drop seen with 60 ppm and 80 ppm of hydrogen sulfide. From this experiment, the inventor concluded that the loss of thermogenesis is directly dependent upon the concentration of hydrogen sulfide given to the mice.

### Example 4

### An Animal Pretreatment Study

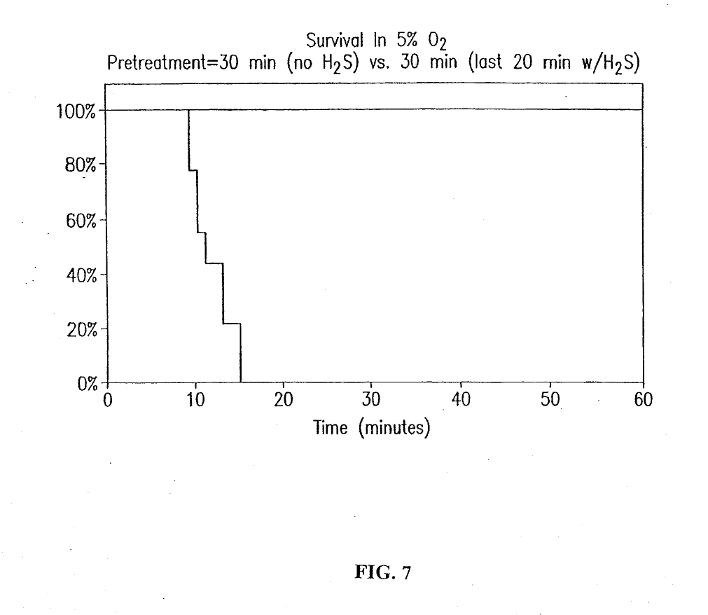

To determine the effect of H2S pre-treatment alone on survivability under hypoxic conditions (without continuous H2S exposure during hypoxia), mice were exposed to either 30 minutes of room air (No PT) or 10 minutes of room air followed by 20 minutes of 150 ppm H2S in room air (PT) before exposure 5% O₂ (5%), and their survival time determined. Experiments were stopped at 60 minutes, and animals still alive were returned to their cage. As shown in FIG. 7, all of the mice in a cohort of animals pre-exposed to 150 ppm H2S in room air for 20 minutes survived subsequent exposure to 5% O₂, while all of the control animals exposed to room air alone had died within 15 minutes of exposure to 5% O₂. Thus, pre-exposure of mice to H2S establishes a physiological state in the mice that allows prolonged survival to otherwise lethal hypoxia. The protection observed in H2S pre-treated mice far exceeds the known protective effect of whole body hypoxia preconditioning that has been reported in the literature, in which survivability in 5% O₂ was extended only twofold (Zhang et al. 2004). Although not shown in FIG. 7, some H2S pre-treated mice were able to survive for more than four hours in 5% O₂ and were able to recover with no noticeable motor or behavioral deficits.

These data demonstrate that exposure to H2S has a pharmacological effect in which survival in otherwise lethal hypoxia is greatly enhanced. In this context, the pharmacological effects of H2S depend on dose levels and duration of exposure to H2S, parameters that one skilled in the art can vary to achieve optimum survivability to lethal hypoxia. One skilled in the art will appreciate that the route of administration (e.g., inhaled versus parenteral administration) can also be varied to achieve the desired effect of lethal hypoxia tolerance in a mammal. In addition, the pharmacological effect can be observed either when H2S exposure is limited to pre-treatment or is extended into the period of hypoxia. Likewise, the timing of exposure to H2S relative to the onset of lethal hypoxia can be varied to maximize the enhanced survivability. These data are consistent with the hypothesis that reduction in oxygen demand resulting from pretreatment with an active compound, such as an oxygen antagonist, allows survival in reduced oxygen supply that is otherwise lethal to the animal.

These data demonstrate that H2S pretreatment alone prevents additional reductions in metabolic activity typically associated with a transition to lethal hypoxia, thereby enhancing survival under hypoxic conditions. In addition, these data support a model wherein pre-exposure of biological matter to active compounds is sufficient to enhance survivability and/or reduce damage from injuries or disease insults.

### Example 5

### Preparation of Colloidal Sulfur

A method for preparing colloidal sulfur is provided. The method is loosely based on Monaghan and Garai, 1924. Colloidal sulfur may be provided to biological matter in any manner as described herein. The preparation involves the removal of thiol sulfur from thiosulfate using acid in the presences of serum proteins to form elemental sulfur molecules (S₆-S₂₀).

To one volume 2M sodium thiosulfate (Na₂S₂O₄), 2 volumes water and 1/10 volume serum are added. One volume 2N metaphosphoric acid is then added, and the mixture is allowed to react for up to 10 minutes. The pH of the mixture is then neutralized using sodium hydroxide (NaOH), followed by overnight dialysis against noinial saline (0.9%).

### Example 6

### Materials and Methods for FIGS. 2, 3, 4 and 8

Growing Nematodes in H2S-Containing Atmospheres. Bristol strain N2 (wild-type) and mutant nematode strains were grown at room temperature on nematode growth medium (NGM) plates seeded with live E. coli OP50 food (48). Mutant strains obtained from the C. elegans genetic stock center were as follows: CB130, daf-2(e1370); DR26, daf-16(m26); VC520, isp-1(gk267); MQ130, clk-1(qm30); DA1116, eat-2(ad1116); VC199, sir-2.1(ok434). Plates were maintained in atmospheric chambers sealed with Dow Corning Vacuum Grease (Sigma). Care was taken to ensure that cultures did not starve. Chambers were continuously perfused with room air or 50 ppm H2S that was freshly mixed into room air (diagrammed in FIG. 9). Gasses were hydrated using gas wash bottles (Fisher), and moved through ⅛ inch outer diameter FEP tubing (Cole Parmer) with connections by snap connectors (Cole Parmer), stainless steel quick connect fittings or compression fittings (Seattle Fluid Systems). The H2S-containing atmospheres were constructed by mixing H2S from a 5000 ppm H2S (balanced with N2) source tank with room air using mass flow controllers (model number 810 and Smart-Trak Series 100; Sierra Instruments). All compressed gas mixtures used in this study were obtained from Byrne Gas and were certified standard to within 2% of indicated concentration. Flow tubes (Aalborg) were used to distribute the gas mixture to different chambers. Gas flow rate was 100 cc/m to small boxes (100-300 mL) and 800 cc/m to the large boxes (1-3 L) used to culture nematode strains at room temperature. At these flow rates, the gaseous environment of the atmospheric chambers is exchanged every 20-30 minutes. The concentration of H2S was monitored using a custom-built H2S detector (Jose Rivera, Facilities Engineering, Fred Hutchinson Cancer Research Center) containing a 3-electrode electrochemical Surecell H2S detector (Sixth Sense). The detector was zeroed with room air and spanned with 100 ppm H2S before each use. Data were collected using Chart software with a Powerlab data acquisition unit (ADInstruments) and analyzed with EXCEL. The concentration of H2S measured was consistently within 10 ppm of the reported value and was stable from day to day. The H2S-containing atmospheres did not alter the pH of the NGM plates.

To monitor the stability of 50 ppm H2S in room air, exhaust from atmospheric chambers was collected in a Tedlar gas sampling bag, which was then left at room temperature. The concentration of H2S was measured at various times, taking one measurement per second for at least 100 seconds. The error bars in FIG. 9 represent one standard deviation of the average measurement. These experiments show that oxidation of H2S in room air occurs slowly (FIG. 9B), with no change in H2S concentration over 24 h.

Brood size measurement. To determine the number of viable progeny produced by nematodes, individual fourth-stage larvae (L4) were transferred to NGM plates with OP50 food at room temperature. Animals were moved daily until they quit laying fertilized eggs. Progeny were counted as L4/young adult.

Measuring Developmental Rates. The time required for embryonic development was determined by measuring the time required for 2-cell embryos to hatch. 2-cell embryos were isolated from log-phase adults as described (Nystul and Roth, 2004). Briefly, adults were chopped with a razor blade and approximately 20 2-cell embryos were moved to NGM plates without food by mouth pipette. The number of embryos that had hatched was monitored every 45-60 minutes beginning 6-8 h after embryos were picked. Embryos that did not hatch after 36 hours were considered dead and were not included in the analysis. Median time of development was determined by log-rank analysis in SigmaStat (Systat). Data from one representative experiment for both embryonic and post-embryonic development are shown in Table 1, though each experiment was repeated several times with similar results.

Post-embryonic development was measured as the time required for starved first-stage larvae (L1) to become gravid, egg-laying adults. Starved L1 were isolated by picking 30-50 adults from each population into 10 μL hypochlorite solution (2.5 N KOH, 5% NaOCl) on a small (unseeded) NGM plate. After 5 minutes, 1 mL M9 buffer (Brenner, 1974) was added to the plate and the embryos were returned to the atmospheric chambers. After 24-36 h, starved L1 were moved onto NGM plates with OP50, returned to the chamber and allowed to develop at room temperature. After 30-48 hours, individual larvae were moved to NGM plates with a 10 μL spot of OP50. Each worm was monitored every 6-12 hours until it began laying eggs (intervals became closer as time progressed and other animals became gravid). If more than one embryo had been laid, the time that the first egg was laid was determined assuming that one egg was laid every 15 minutes for wildtype, and 30 minutes for the clk-1 (qm30) mutants. This value was determined empirically by counting the number of embryos laid by each worm for the 6 hour period after it began egg-laying. Data were analyzed using log-rank analysis in SigmaStat (Systat).

The rate of egg-laying was determined for first-day gravid adults from populations cultured in each condition (room air±50 ppm H2S). Animals were picked as L4 from mixed-stage populations and allowed to develop for 20-30 hours in the same conditions at room temperature. Individual worms were then placed onto NGM plates with a 10 μL spot of OP50 food. The number of embryos laid in 3-5 hours was counted to determine the rate of egg-laying. To create an atmosphere with 2% O₂, N2 was mixed with 5% O₂ balanced with N2. Smart-Trak series 100 mass flow controllers (Sierra Instruments) were used to mix the gas and to split it into 2 atmospheric chambers. H2S was then added to the 2% O2 that flowed into one of the chambers using a model 810 mass flow controller (Sierra Instruments). Student's t-test was used to determine if the rate of egg-laying varied significantly between conditions, assuming two-tailed distributions with unequal variance (EXCEL). In each experiment, 10-15 individuals were included in each group. The data shown in Table 2 are from one experiment that is representative of at least three independent assays.

Thermotolerance Assay. Cultures of nematodes were established in 50 ppm H2S or room air control conditions and maintained for at least a week prior to thermotolerance measurement. Care was taken to prevent the population from starving. Nematodes were picked from these mixed-stage populations as L4 larvae and allowed to develop for 24-48 h at room temperature; however, treated and control animals were always the same age. For temperature sensitive daf-2(e1370) mutants (30), cultures were maintained in H2S containing environments at 17 C and moved to room temperature as L4. Groups of 20-30 animals were transferred to NGM plates without food and then placed into an atmospheric chamber perfused with the indicated gas at high temperature in an Echotherm 1N35 incubator (Torrey Pines Scientific, NIST traceable) or a VWR incubator model 2005 (VWR International). A ring of palmitic acid around the edge of the plate helped prevent the worms escaping the surface of the agar. Temperature was maintained at 34.5±1 C, although the temperature was raised slightly to facilitate experiments with thermotolerant strains. In these experiments, the high temperature was chosen so that controls died in less than 10 h (for example, in FIG. 8A, daf-2(e1370) animals were tested at 36.5 C). HOBO U10 data loggers that were calibrated to a NISTtraceable thermometer (Onset Corporation) were used to monitor the temperature in each chamber. In every experiment, the temperature of the room air and H2S-containing chambers was the same. Plates were removed to count the number of animals that had died every few hours. Nematodes were considered dead and removed from the plate when they no longer responded to prodding with a platinum wire. Kaplan-Meyer log-rank tests with the program SigmaStat (Systat) were used to evaluate statistical significance. Animals that could not be accounted for were censored from the analysis. Each assay was repeated at least twice with similar results.

Quantitative RT-PCR. To monitor sir-2.1 transcript levels, quantitative RT-PCR was performed essentially as described (Van Guist et al., 2005). In short, RNA was purified from synchronous L4 populations (5 replicates were grown in control room air conditions and 4 were grown in H2S) with Trizol, cleaned by phenol:chloroform extraction and isopropanol precipitation and then cDNA synthesis was performed using the ProtoScript kit (New England Biolabs) with random hexanucleotide primers. Each reaction was performed in duplicate. Two different primer sets were used to amplify the sir-2.1 cDNA. A control primer set specific for genomic sir-2.1 did not amplify a product from the cDNA template. Agarose gel electrophoresis was used to ensure that the product of the PCR reaction was of the correct size.

### Example 7

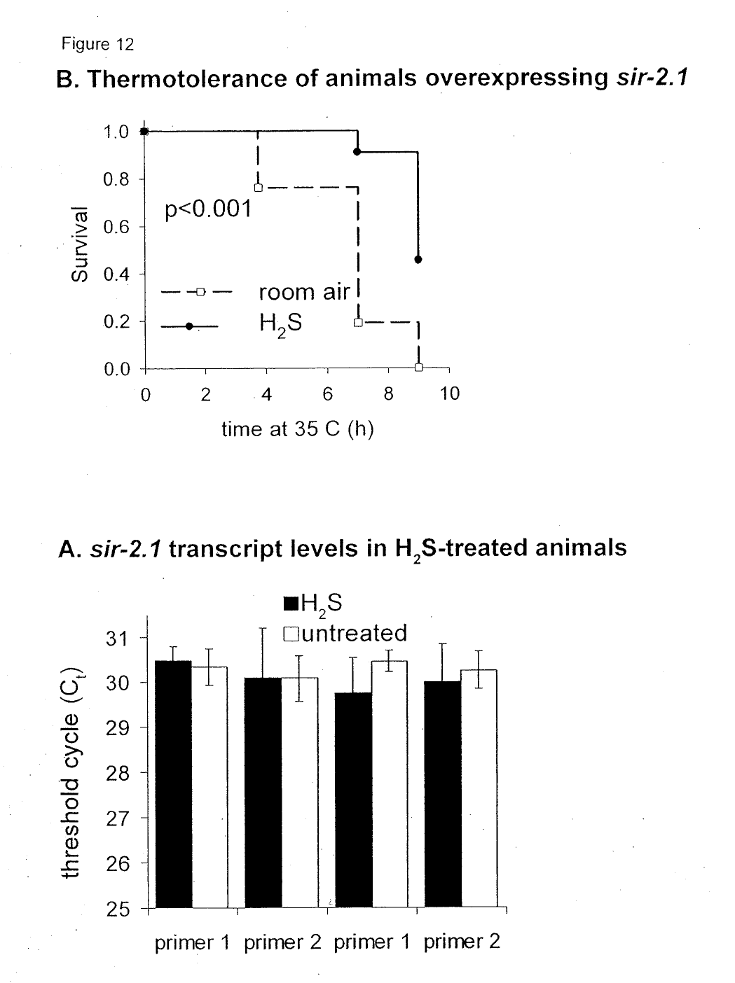

### Materials and Methods for FIGS. 9-12

To monitor the stability of 50 ppm H2S in room air, exhaust from atmospheric chambers was collected in a Tedlar gas sampling bag, which was then left at room temperature. The concentration of H2S was measured at various times, taking one measurement per second for at least 100 seconds. The error bars in FIG. 9 represent one standard deviation of the average measurement. These experiments show that oxidation of H2S in room air occurs slowly (FIG. 9B), with no change in H2S concentration over 24 h.

To monitor sir-2.1 transcript levels, quantitative RT-PCR was performed essentially as described (Van Gilst, 2005). In short, RNA was purified from synchronous L4 populations (5 replicates were grown in control room air conditions and 4 were grown in H2S) with Trizol, cleaned by phenol:chloroform extraction and isopropanol precipitation and then cDNA synthesis was performed using the ProtoScript kit (New England Biolabs) with random hexanucleotide primers. Each reaction was performed in duplicate. Two different primer sets were used to amplify the sir-2.1 cDNA. A control primer set specific for genomic sir-2.1 did not amplify a product from the cDNA template. Agarose gel electrophoresis was used to ensure that the product of the PCR reaction was of the correct size.

To evaluate if H2S induced expression of stress-inducible transgenes, populations of each strain were maintained in H2S for at least two generations. Strains used were as follows: TJ375, gpIs1[hsp-16.2::GFP]; SJ4005, zcIs4[hsp-4::GFP]; BC10066, sEX900[hsp-3::GFP]; BC10060, sEX10060[hsp-70::GFP]; BC10064, sEX10064[hsp-6::GFP]; CF1553, muIs84[pAD76(sod-3::GFP)]; BC10068, sEX10068[stc-1::GFP]. Fourth-stage larvae were anesthetized with levamisole and mounted on a pad of 2% agarose in M9. GFP fluorescence was visualized with a Zeiss microscope and images were collected with an Axiocam camera. Exposure time was fixed manually so that it was the same for H2S-treated and control nematodes.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,989,816
U.S. Pat. No. 3,995,444
U.S. Pat. No. 4,034,753
U.S. Pat. No. 4,186,565
U.S. Pat. No. 4,266,573
U.S. Pat. No. 4,292,817
U.S. Pat. No. 4,442,856
U.S. Pat. No. 4,444,762
U.S. Pat. No. 4,447,415
U.S. Pat. No. 4,473,637
U.S. Pat. No. 4,502,295
U.S. Pat. No. 4,559,258
U.S. Pat. No. 4,723,974
U.S. Pat. No. 4,745,759
U.S. Pat. No. 4,798,824
U.S. Pat. No. 4,828,976
U.S. Pat. No. 4,938,961
U.S. Pat. No. 4,951,482
U.S. Pat. No. 5,066,578
U.S. Pat. No. 5,157,930
U.S. Pat. No. 5,217,860
U.S. Pat. No. 5,231,025
U.S. Pat. No. 5,285,657
U.S. Pat. No. 5,326,706
U.S. Pat. No. 5,370,989
U.S. Pat. No. 5,395,314
U.S. Pat. No. 5,399,363
U.S. Pat. No. 5,405,742
U.S. Pat. No. 5,434,045
U.S. Pat. No. 5,466,468
U.S. Pat. No. 5,470,738
U.S. Pat. No. 5,476,763
U.S. Pat. No. 5,543,158
U.S. Pat. No. 5,552,267
U.S. Pat. No. 5,568,910
U.S. Pat. No. 5,569,579
U.S. Pat. No. 5,580,781
U.S. Pat. No. 5,599,659
U.S. Pat. No. 5,636,643
U.S. Pat. No. 5,641,515
U.S. Pat. No. 5,645,081
U.S. Pat. No. 5,693,462
U.S. Pat. No. 5,699,793
U.S. Pat. No. 5,719,174
U.S. Pat. No. 5,736,397
U.S. Pat. No. 5,739,169
U.S. Pat. No. 5,752,929
U.S. Pat. No. 5,801,005
U.S. Pat. No. 5,830,880
U.S. Pat. No. 5,846,945
U.S. Pat. No. 5,912,019
U.S. Pat. No. 5,952,168
U.S. Pat. No. 6,013,256
U.S. Pat. No. 6,046,046
U.S. Pat. No. 6,054,261
U.S. Pat. No. 6,057,148
U.S. Pat. No. 6,100,082
U.S. Pat. No. 6,187,529
U.S. Pat. No. 6,365,338
U.S. Pat. No. 6,490,880
U.S. Pat. No. 6,492,103
U.S. Pat. No. 6,524,785
U.S. Pat. No. 6,552,083
U.S. Pat. No. 6,602,277
U.S. Pat. No. 6,790,603
U.S. patent application Ser. No. 10/971,575,
U.S. patent application Ser. No. 10/971,576
U.S. patent application Ser. No. 10/972,063
U.S. Prov. Appln. 60/513,458
U.S. Prov. Appln. 60/548,150
U.S. Prov. Appln. 60/557,942
Alam, Antioxid Redox Signal. 4(4):559-62, 2002.
Amersi et al., Hepatology, 35(4):815-823, 2002.
Austin-Ward and Villaseca, Revista Medica de Chile, 126(7):838-845, 1998.
Barton & Ollis, Oxford, UK, Jones (Ed.), Pergamon Press, 3:373-487, 1979.
Baskin and Wang, Tetrahedron Lett., 43:8479-8483, 2002.
Baskin et al., Org. Lett., 4:4423, 2002.
Beauchamp et al., Crit. Rev. Toxicol. 13:25, 1984.
Beck et al., Proc. Soc. Exp. Biol. Med. 86:823, 1954.
Behnke, et al., (2006) Appl. Environ. Microbiol. 72:3626-3636.
Behringer et al., Crit. Care Med., 31(5):1523-1531, 2003.
Bellamy et al., Crit. Care Med., 24(2 Suppl):S24-47, 1996.
Bernard et al., J. Thorac. Cardiovasc. Surg. 90:235-242, 1985.
Bernard et al., N. Engl. J. Med., 346(8):557-563, 2002.
Bian, et al., (2006) J Pharmacol Exp Ther 316:670-8.
Blackstone et al., Science, 308:518, 2005.
Blackstone, E. & Roth, M. B. (2007) Shock 27:370-2.
Blander and Guarente, (2004) Annual Review of Biochemistry 73:417-435.
Bordone and Guarente, (2005) Nature Reviews Molecular Cell Biology 6:298-305.
Boyce and Ham, J. Invest. Dermatol., 81:335-405, 1983.
Boyce and Ham, J. Tissue Culture Methods, 9:83-93, 1985.
Brenner (1974) Genetics 77:71-94.
Briese, Neurosci. Biobehav. Rev., 22(3):427-436, 1998.
Brizel, Seminars Radiation Oncol., 8(4Suppl):17-20, 1998.
Brouard et al., J. Biol. Chem., 277(20):17950-17961, 2002.
Bukowski et al., Clinical Cancer Res., 4(10):2337-2347, 1998.
Burns & Murphey, Arch. Biochem. Biophys., 339:33-39, 1997.
Burns et al., Arch. Biochem. Bipophys, 10:60-68, 1995.
Cairns et al., J. Am. Chem. Soc., 74:3982, 1952.
Calfon et al., (2002) Nature 415:92-96.
Chasteen and Bentley, Chem. Rev., 103(1):1-25, 2003.
Christodoulides et al., Microbiology, 144(Pt 11):3027-3037, 1998.
CIIT (Chemical Industry Institute of Toxicology), In: 90 day vapor inhalation toxicity study of hydrogen sulfide, Toxigenics, 420-0710, 1983.
Clive et al., J. Org. Chem., 47:1641, 1982.
Cloarec & Charette, Org. Lett., 6:4731, 2004.
Cohen et al., Appl. Environ. Micro., 51:398-407, 1986.
Cohen et al., Ann. Thorac. Surg., 67(5):1489-1491, 1999.
Curran, Seminars Radiation Oncol., 8(4Suppl):2-4, 1998.
Davidson et al., J. Immunother., 21(5):389-398, 1998. Davis (1994)
Demuynck and Vialle, Bulletin de la Societe Chimique de France, 4:1213-1218, 1967
Demuynck et al., Bulletin de la Societe Chimique de France, 3366-3367, 1966.
Demuynck et al., Bulletin de la Societe Chimique de France, 8:2748-2754, 1967.
Dhanasekaran et al., J. Biol. Chem., 279:37575-37587, 2004.
Dillin et al., (2002) Science 298:2398-2401.
Dillin et al., (2002) Science 298:830-834.
Dillman, Cancer Biother. Radiopharm., 14(1):5-10, 1999.
Dittmer and Hoey, In: The Chemistry of Sulphinic Acids, Esters, and Their Derivatives, Wiley: Chichester, U.K., 239-273, 1990.
Donaldson and Anderson, J. Clin. One. 23:2586-2587, 2005.
Dorman et al., Neurotoxicol. Teratol., 22(1):71-84, 2000.
Dulak et al., Antioxid. Redox Signal, 4(2):229-240, 2002.
Duus, In Comprehensive Organic Chemistry: The Synthesis and Reactions of Organic Compounds, 1^{st} Ed., 1994.
Eto et al., Biochem. Biphys. Res. Commun., 293:1483-1488, 2002.
Feng et al., (2001) Dev Cell 1:633-44.
Fenchel and Finlay (1995) Ecology and Evolution in Anoxic Worlds (Oxford University Press', Oxford).
Ganther, Carcinogenesis 20(9):1657-66 (1999).
Gems et al., (1998) Genetics 150:129-155.
Gilbert et al., LANCET, 355:375-376, 2000.
Gladysz et al., J. Org. Chem., 43:1204, 1987.
Glass, Phosph. Sulfur Silicon Rel. Elem., 136, 137, 138:159-174, 1998.
Golden and Riddle (1984) Developmental Biology 102:368-378.
Gompertz, B. (1825) Phil Trans Royal Soc, 513-585.
Gorman et al., Toxicology, 187(1):25-38, 2003.
Goubern et al., (2007) Faseb J.
Guarente, L. (2005) Mechanisms of Ageing and Development 126:923-928.
Guarente, L. & Picard, F. (2005) Cell 120:473-482.
Guillemin et al., Cell, 89(1):9-12, 1997.
Haigis and Guarente (2006) Genes Dev. 20:2913-2921.
Hanibuchi et al., Intl. J. Cancer, 78(4):480-45, 1998.
Hannan et al., JAMA, 290(6):773-780, 2003.
Hansen, M., Hsu, A. L., Dillin, A. & Kenyon, C. (2005) PLoS Genet. 1:119-28.
Hansen et al., (2007) Aging Cell 6, 95-110.
Harris, J. Org. Chem., 25:225, 1960.
Harris, J. Org. Chem., 30:2190, 1965.
Hays, In: Studies of the Effects of Atmospheric Hydrogen Sulfide in Animals, thesis dissertation, University of Missouri-Columbia, 1972.
Hekimi et al., (2001) Trends Genet. 17:712-8.
Hellstrand et al., Acta Oncologica, 37(4):347-353, 1998.
Higuchi and Fukamachi, Folia Pharmacologica Japonica, 73(3):307-319, 1977.
Hobert et al., Organometallics, 20:1370, 2001.
Hochachka et al., Comp. Biochem. Physiol. B Biochem. Mol. Biol., 130(4):435-459, 2001.
Hochachka et al., Proc. Natl. Acad. Sci. USA, 93(18):9493-9498, 1996.
Hong et al., (2004) J Mol Biol 344:369-381.
Houthoofd et al., (2003) Exp Gerontol 38:947-54.
Hui and Hashimoto, Infection Immun., 66(11):5329-5336, 1998.
Hwang and Greenberg, Biochemistry, 38:14248, 1999.
Hyspler et al., J. Chromatography, 770:255-259, 2002.
Innicenti et al., Bioorg. Med. Chem. Lett. 14:5769 (2004).
Jiang et al., Am. J. Physiol. Cell Physiol., 280:1140-1150, 2001.
Johnson, T. E. (1990) Science 249:908-912.
Ju et al., J. Neuropathol. Exp. Neurol., 59(3):241-50, 2000.
Kaeberlein et al., (2006) Aging Cell.
Kamoun, Amino Acids 26:243, 2004.
Kapulkin et al., (2005) FEBS Lett 579, 3063-3068.
Kelso et al., J. Biol. Chem., 276:4588-4596, 2001.
Kenyon (2005) Cell 120:449-460.
Kenyon et al., (1993) Nature 366:461-4.
Khan et al., Toxicol. Applied Pharmacol., 103:482-490, 1990.
Khazaeli et al. (1998) Mech Ageing Dev, 105:301-317.
Kilburn and Warshaw, Toxicology Indust. Health, 11(2):185-197, 1995.
Kilburn, Environ. Health, 54(3):150, 1999
Kilburn, Environ. Res., 81(2):92-99, 1999.
Klass (1977) Mech Ageing Dev 6, 413-29.
Klayman, D. L.; Gunther, W. H. H. Eds, Wiley Interscience, New York, 1973, Chapter IV; Chapter VI; Chapter VII; Chapter VIII; Chapter IX.
Knapp and Darout, Org. Lett., 7:203, 2005.
Kontou et al., J. Agricultural and Food Chem., 52:1212, 2004.
Krauth-Siegel et al. (1989) Biochem Soc Trans 17:315-7.
Kuroda et al., Transplantation, 46(3):457-460, 1988.
Kuroda et al., Transplantation, 46(3):457-460, 1988.
Lai et al., Biochemistry, 40:4904-4910, 2001.
Lakowski and Hekimi, (1998) PNAS 95:13091-13096.
Langer et al., Biochemistry 33:14034, 1994.
Langer et al., Biochemistry, 33:10867, 1997.
Ledingham et al., Circulation, 82(2):IV351-358, 1990.
Ledingham et al., J. Thorac. Cardiobasc. Surg., 93:240-246, 1987.
Lee et al., J. Nuc. Med. 26:72, 1985.
Lithgow et al. (1995) PNAS 92:7540-7544.
Liu et al., J. Org. Chem., 67:9267, 2002.
Longo and Kennedy (2006) Cell 126:257-268.
Lowicka and Beltowski (2007) Pharmacol Rep 59:4-24.
Lundgren-Eriksson et al., Anticancer Res. 2001 Sep-Oct; 21(5):3269-74.
Massie et al. (2003) Cell Stress Chaperones 8:1-7.
McCarter et al., (1999) Developmental Biology 205:111-128.
Mehlhorn et al., Cardiovasc Surg., 9(5):482-486, 2001.
Menasche et al., Eur. J. Cardio. Thorax. Surg., 8:207-213, 1994.
Michaels et al., Circulation, 106(23):e187-190, 2002.
Monaghan and Garai, Practical Ther. 20:24-26 (1924).
Mugesh et al., Chem. Rev., 101:2125, 2001.
Murai and Kato, In: Organoselenium Chemistry: Modern Developments in Organic Synthesis, Wirth (Ed.), Springer, N.Y., Vo. 28, 2000.
Murai, et al., J. Org. Chem., 66:8101, 2001.
Netherton and Fu, Org. Lett., 3:4295, 2001.
Noguchi et al., Biochemistry, 42:11642, 2003.
Nogueira et al., Chem. Rev., 104:6255, 2004.
Nystul et al., Science, 302(5647):1038-1041, 2003.
Nystul and Roth (2004) PNAS 101:9133-9136.
O'Brien and Vetter (1990) J Exp Biol 149:133-48.
O'Sullivan et al., J. Am. Chem. Soc., 126:2194, 2004.
Olojo et al., J. Phys. Chem. A, 108:1018, 2004.
Otterbein et al., Am. J. Physiol. Lung Cell Mol. Physiol., 279(6):L1029-L1037, 2000.
Otterbein et al., Trends Immunol., 24(8):449-455, 2003.
Padilla et al., Molec. Biology of the Cell, 13:1473-1483, 2002.
Padilla et al., Proc. Natl. Acad. Sci. USA, 98(13):7331-7335, 2001.
Partlo et al., Neurotoxicology, 22(2):177-189, 2001.
PCT Appln. WO 94/17178
Petersen, Biochemica et Biophysica Acta, 460:299-307, 1977.
Pietras et al., Oncogene, 17(17):2235-2249, 1998.
Porcu and Chiarugi, Trends Pharm. Sci (TRIPS) 26:94-193, 2005.
Powell and Somero (1986) Science 233:563-566.
Punch et al., 2001
Qin et al., Proc. Natl. Acad. Sci. USA, 95(24):14411-14416, 1998.
Quirante et al., J. Org. Chem., 67:2323, 2002.
Rager et al., NC Med. J., 65(1):18-25, 2004.
Reigan et al., J. Med. Chem., 48:392, 2005.
Remington's Pharmaceutical Sciences, 15^{th} ed., pages 1035-1038 and 1570-1580, Mack Publishing Company, Easton, Pa., 1980.
Remington's Pharmaceutical Sciences (2005); 21^{st} Edition, Troy, David B. Ed. Lippincott, Williams and Wilkins.
Rea et al., (2005) Nat Genet. 37:894-898.
Rogers et al., Mamm. Genome, 8:711-713, 1997.
Ryter and Otterbein, BioEssays, 26:270-280, 2004.
Seburg and Squires, Intl. J. Mass Spectrometry Ion Proc., 167/168:541, 1997.
Semenza, Cell, 98(3):281-284, 1999.
Semenza, Trends Mol. Med., 7(8):345-350, 2001. Shaw (1996)
Shawali et al., J. Org. Chem., 61:4055, 2001.
Shen et al., (2001) Cell 107:893-903.
Shen et al., J. Agric. Food Chem., 50:2644, 2002.
Shibata et al., (2000) Genetics 154:635-646.
Smith et al., Eur. J. Biochem., 263:709-716, 1999.
Soledad et al., Org. Lett., 3:1213, 2001.
Steudel, Chem. Rev., 102:3905, 2002.
Stipanuk (2004) Annual Review of Nutrition 24:539-577.
Struve et al., Neurotoxicology, 22(3):375-385, 2001.
Sundarrajan et al., Macromolecules, 35:3331, 2002.
Supuran et al., Med. Res. Rev., 23(2):146-189, 2003.
Sweeney, In: A Survey of Compounds from the Antiradiation Drug Development Program of the U.S. Army Medical Research and Development Command. Walter Reed Army Institute of Research, Washington D.C., 1979.
Teodoro and OFarrell, EMBO J., 22(3):580-587, 2003.
The Hypothermia After Cardiac Arrest Study Group et al., 2002.
Tisherman, Crit. Care Med., 32(2):S46-S50, 2004.
Tissenbaum and Guarente (2001) Nature 410:227-230.
Urano et al., (2002) J Cell Biol 158:639-646.
Van Gilst et al., (2005) PNAS, 102:13496-13501.
Van Voorhies et al., J. Exp. Biol., 203(Pt 16):2467-2478, 2000.
Walker et al., (2005) Mech Ageing Dev 126:929-37.
Wang, FASEB J., 16(13):1792-1798, 2002. Wolff and Dillin (2006) Exp Gerontol.
Wong et al., (1995) Genetics 139:1247-1259.
Yaffe et al., Crit. Care Med., 32(2):S51-55, 2004.
Yaghi et al., Nature, 423(6941):705-714, 2003.
Yang et al., J. Agric. Food Chem., 52:7051, 2004.
Yong and Searcy (2001) Comp Biochem Physiol B Biochem Mol Biol 129:129-37.
Yoshikawa et al., J. Biochem. (Tokyo), 71:859-872, 1972.
Zhang et al., J. Appl. Physiol. 96(1):392-397, 2004.
Zhang et al., J. Org. Chem. 63:5314, 1998.
Ziegler, Ann. Rev. Biochem. 54:305, 1985.

1.-2. (canceled)

3. A method of enhancing lifespan in biological matter comprising administering to the biological matter a sirtuin-modulating compound in combination with a chalcogenide.

4. The method of claim 3, wherein the biological matter comprises cells.

5.-7. (canceled)

8. The method of claim 3, wherein the chalcogenide is sulfide.

9. The method of claim 3, wherein the sirtuin-modulating compound is selected from the group comprising formula 1-188.

10. The method of claim 3, wherein the sirtuin-modulating compound is selected from the group consisting of nicotinic acid, resveratrol, butein, fisetin, piceatannol, isoliquiritigenin and quercetin.

11. (canceled)

12. A method for enhancing lifespan in biological matter comprising providing to the biological matter a chalcogenide of formula (I) or (IV) or salt or prodrug thereof, wherein formula (I) and (IV) comprise: 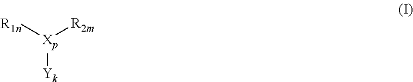
wherein X is N, O, Po, S, Se, or Te;
wherein Y is N or O;
wherein R₁ is H, C, lower alkyl, a lower alcohol, or CN;
wherein R₂ is H, C, lower alkyl, or a lower alcohol, or CN;
wherein n is 0 or 1;
wherein m is 0 or 1;
wherein k is 0, 1, 2, 3, or 4; and,
wherein p is 1 or 2; 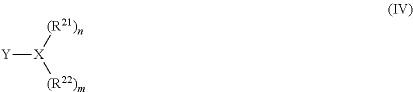
wherein:
X is N, O, P, Po, S, Se, Te, O—O, Po—Po, S—S, Se—Se, or Te—Te;
n and m are independently 0 or 1;
R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹ herein, or Y is 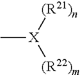
wherein X, R²¹ and R²², are as defined herein.

13. The method of claim 12, wherein X is sulfur.

14. The method of claim 12, wherein the chalcogenide is a sulfide salt.

15. The method of claim 14, wherein the salt is a sulfide salt selected from the group consisting of sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS) and barium sulfide (BaS).

16. The method of claim 12, wherein the chalcogenide is selected from the group consisting of H2S, H2Se, H₂Te and H₂Po.

17.-38. (canceled)

39. A method for enhancing survivability of biological matter comprising administering to the matter an effective amount of a composition having one or more compounds with formula (I) and/or formula (IV), or a salt or prodrug thereof, in combination with a sirtuin-modulating compound.

40.-42. (canceled)

43. A method for extending longevity to biological matter under adverse conditions comprising administering to the biological matter an effective amount of a chalcogenide in combination with a sirtuin-modulating compound, wherein damage is prevented or reduced.

44. The method of claim 43, wherein the biological matter is treated with the combination of an active compound and the sirtuin-modulating compound.

45. The method of claim 44, wherein the active compound is sulfide.

46. (canceled)

47. A method of preventing an organism from bleeding to death comprising providing to the bleeding organism an effective amount of a chalcogenide in combination with a sirtuin-modulating compound to prevent death.

48. The method of claim 47, wherein the organism goes into hemorrhagic shock.

49. The method of claim 47, wherein the chalcogenide is a sulfur-containing compound.

50. The method of claim 47, wherein the chalcogenide is formula (I) or a salt or prodrug thereof, wherein formula (I) comprises 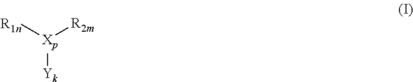
wherein X is S;
wherein k is 0
wherein m is 0;
wherein n is 0 or 1; and
wherein R₁ is H.

51.-58. (canceled)

59. A method of modulating sirtuin activity in biological matter comprising providing the biological matter with a chalcogenide of formula (I) or (IV) or salt or prodrug thereof, wherein formula (I) and (IV) comprise: 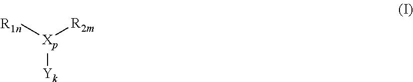
wherein X is N, O, Po, S, Se, or Te;
wherein Y is N or O;
wherein R₁ is H, C, lower alkyl, a lower alcohol, or CN;
wherein R₂ is H, C, lower alkyl, or a lower alcohol, or CN;
wherein n is 0 or 1;
wherein m is 0 or 1;
wherein k is 0, 1, 2, 3, or 4; and,
wherein p is 1 or 2; 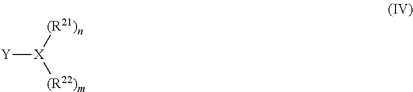
wherein:
X is N, O, P, Po, S, Se, Te, O—O, Po—Po, S—S, Se—Se, or Te—Te;
n and m are independently 0 or 1;
R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidine, hydrazino, hydrazide, hydroxyl, alkoxy, aryloxy, hetroaryloxy, cyloalkyloxy, carbonyloxy, alkylcarbonyloxy, haloakylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹ herein, or Y is 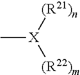
wherein X, R²¹ and R²², are as defined herein.

60. The method of claim 59, further comprising providing the biological matter with a sirtuin-modulating compound.

61. The method of claim 59, wherein X is sulfur.

62. The method of claim 59, wherein the chalcogenide is a sulfide salt.

63. The method of claim 62, wherein the salt is a sulfide salt selected from the group consisting of sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS) and barium sulfide (BaS).

64. The method of claim 59, wherein the chalcogenide is selected from the group consisting of H2S, H2Se, H₂Te and H₂Po.
